(19) United States
(12) Patent Application Publication
(10) Pub. No.: US 2025/0129388 A1
(43) Pub. Date: Apr. 24, 2025

(54) TEMPERATURE REGULATED CRISPR-CAS SYSTEMS AND METHODS OF USE THEREOF

(71) Applicant: The Regents of the University of California, Oakland, CA (US)

(72) Inventors: Jennifer A. Doudna, Berkeley, CA (US); Steven E. Jacobsen, Agoura Hills, CA (US); Zheng Li, Los Angeles, CA (US); Basem Al-Shayeb, Berkeley, CA (US)

(21) Appl. No.: 18/689,041

(22) PCT Filed: Aug. 31, 2022
(86) PCT No.: PCT/US22/75781

§ 371 Date: Mar. 4, 2024

Related U.S. Application Data
(60) Provisional application No. 63/242,946, filed on Sep. 10, 2021.

(51) Int. Cl. C12N 15/90; C12N 9/22; C12N 15/11; C12N 15/82

(52) U.S. Cl. CPC C12N 15/907; C12N 9/22; C12N 15/11; C12N 15/8213; C12N 2310/20

(57) ABSTRACT

The present disclosure relates to CRISPR-Cas systems that utilize Cas12L for binding to and modifying nucleic acids in eukaryotic cells in a temperature regulated manner. Methods and compositions for using these CRISPR-Cas systems for editing nucleic acids for use in disease treatment and prevention in eukaryotic cells are provided herein.

### CROSS-REFERENCE

This application claims the benefit of U.S. Provisional Patent Application No. 63/242,946, filed Sep. 10, 2021, which application is incorporated herein by reference in its entirety.

### STATEMENT REGARDING FEDERALLY SPONSORED RESEARCH

This invention was made with government support under Grant Number 1752814 awarded by the National Science Foundation. The government has certain rights in the invention.

### INCORPORATION-BY-REFERENCE OF MATERIAL ELECTRONICALLY SUBMITTED

A Sequence Listing is provided herewith as a Sequence Listing XML, “BERK-453WO_SEQUENCE_LISTING” created on Aug. 29, 2022, and having a size of 214 KB. The contents of the Sequence Listing XML are incorporated by reference herein in their entirety.

### INTRODUCTION

Clustered Regularly Interspaced Short Palindromic Repeats (CRISPR)-Cas systems comprise a CRISPR-associated (Cas) effector polypeptide and a guide nucleic acid. Such CRISPR-Cas systems can bind to and modify a targeted nucleic acid. The programmable nature of these CRISPR-Cas effector systems has facilitated their use as a versatile technology for use in, e.g., gene editing. There is a need in the art for CRISPR-Cas systems that can provide temperature-regulated gene editing.

### SUMMARY

The present disclosure relates to CRISPR-Cas systems that utilize Cas12L for editing nucleic acids in eukaryotic cells in a temperature regulated manner. Methods and compositions for using these systems for editing nucleic acids in eukaryotic cells are provided herein.

### BRIEF DESCRIPTION OF THE DRAWINGS

FIG. 1 illustrates the position of guide RNAs (gRNAs) of Cas12L designed to target the AtPDS3 gene. Five guide RNAs were designed for Cas12L to target the AtPDS3 gene, with gRNA positions shown.

FIGS. 2A-2B show counts of next generation sequencing reads with indels of the AtPDS3 gene of protoplasts transfected either with Cas12L ribonucleoproteins (RNPs) or a negative control plasmid. The reads with indels of each pattern which has reads counts larger or equal to 100 in amplicon sequencing of each protoplast transfection were pool analyzed for the 5 guide RNAs tested. FIG. 2A: The reads count with indels from protoplasts transfected with Cas12L RNPs with the 5 guide RNAs were pooled and plotted. For each guide RNA, 3 replicate samples for 23° C., 28° C. and 32° C. incubation respectively were used. FIG. 2B: The reads count with indels from control protoplasts (transfected with HBT-GFP plasmid) amplified for the 5 guide RNA targeting region were pooled and plotted. For each guide RNA, 1 sample for 23° C., 28° C. and 32° C. incubation, respectively, were used.

FIGS. 3A-3E show percentages of edited next generation sequencing reads from Arabidopsis mesophyll protoplasts that were transfected with Cas12L RNPs and then incubated at different temperatures for 48 hours. FIG. 3A shows the percentage of edited reads from protoplasts transfected with Cas12L RNPs with guide RNA 54 and then incubated at 23° C., 28° C., or 32° C. for 48 hours. FIG. 3B shows the percentage of edited reads from protoplasts transfected with Cas12L RNPs with guide RNA 56 and then incubated at 23° C., 28° C., or 32° C. for 48 hours. FIG. 3C shows the percentage of edited reads from protoplasts transfected with Cas12L RNPs with guide RNA 58 and then incubated at 23° C., 28° C., or 32° C. for 48 hours. FIG. 3D shows the percentage of edited reads from protoplasts transfected with Cas12L RNPs with guide RNA 60 and then incubated at 23° C., 28° C., or 32° C. for 48 hours. FIG. 3E shows the percentage of edited reads from protoplasts transfected with Cas12L RNPs with guide RNA 62 and then incubated at 23° C., 28° C., or 32° C. for 48 hours.

FIG. 4A-4Z, 4AA-4ZZ, and 4AAA-4FFF provide amino acid sequences of Cas12L polypeptides.

FIG. 5A-5B provide the locations of active site residues of the RuvC-I, RuvC-II, and RuvC-III domains of Cas12L polypeptides.

FIG. 6A-6P provide an alignment of amino acid sequences of Cas12L polypeptides depicted in FIG. 4A-4Z, 4AA-4ZZ, and 4AAA-4FFF (SEQ ID Nos: 1-11, 11, 12-25, 1, 26-36, 56 and 37-55, respectively).

FIG. 7A-7C provide an alignment of amino acid sequences of selected Cas12L polypeptides depicted in FIG. 4A-4Z, 4AA-4ZZ, and 4AAA-4FFF (SEQ ID Nos: 2, 27, 36, 39, 42, 49 and 53, respectively).

FIG. 8 provides an alignment of nucleotide sequences of the repeat region of Cas-Lambda (Cas12L) guide RNAs (SEQ ID Nos: 92-104, respectively). A consensus sequence is provided (SEQ ID NO:71).

FIG. 9 provides the nucleotide sequence of a UBQ10 promoter (SEQ ID NO:72).

FIG. 10 depicts trans-cleavage activity of Cask was tested against ssRNA poly-U reporter molecules using single-stranded DNA (ssDNA) activators.

FIG. 11 depicts trans-cleavage activity of Cask against DNase Alert Fluorophore-Quencher reporters using ssDNA activators.

FIG. 12 depicts trans-cleavage activity of Cask against DNase Alert Fluorophore-Quencher reporters using ssDNA activators and using gRNAs of different Cask orthologs.

FIG. 13 depicts trans-cleavage activity of Cask against DNase Alert Fluorophore-Quencher reporters using ssDNA activators and using gRNAs for Cask containing sequential mismatches.

FIG. 14 depicts double-stranded DNA (dsDNA) cleavage position and kinetics, and cleavage positions compared to CasΦ polypeptides and Cas12a polypeptides.

FIG. 15 depicts data showing that Casλ processes its own crRNA to form mature gRNA, uses the same RuvC active site that is used for DNA cleavage to process its crRNA, and processes the RNA within the spacer region.

FIG. 16 presents data similar to that in FIG. 15, but with a different RNA (Repeat-Spacer with a few additional base pairs at the 5′ end, as opposed to Repeat-Spacer-Repeat) to verify the results of FIG. 15, and also using different percentages of gels (10% and 20%) to show a higher sequence resolution by nucleotide.

FIG. 17 depicts Casλ mature gRNA, compared to CasΦ and Cas12a gRNA.

FIG. 18 depicts domain organization for Casλ1 unbound to the gRNA or DNA, and structure of the protein with charged residues that would interact with the gRNA.

FIG. 19A-19D depict genome editing using Casλ RNPs in human, Arabidopsis, and wheat cells.

### DEFINITIONS

“Heterologous,” as used herein, means a nucleotide sequence or an amino acid sequence that is not found in the native nucleic acid or protein, respectively. For example, relative to a subject CRISPR-Cas effector polypeptide, a heterologous polypeptide comprises an amino acid sequence from a protein other than the CRISPR-Cas effector polypeptide. As another example, a CRISPR-Cas effector polypeptide can be fused to an active domain from a non-CRISPR-Cas effector polypeptide; the sequence of the active domain can be considered a heterologous polypeptide (it is heterologous to the CRISPR-Cas effector polypeptide). As another example, in a guide nucleic acid, a heterologous guide nucleotide sequence (present in a targeting segment) that can hybridize with a target nucleotide sequence (target region) of a target nucleic acid is a nucleotide sequence that is not found in nature in a guide nucleic acid together with a binding segment that can bind to a CRISPR-Cas effector polypeptide of the present disclosure. For example, in some cases, a heterologous target nucleotide sequence (present in a heterologous targeting segment) is from a different source than a binding nucleotide sequence (present in a binding segment) that can bind to a CRISPR-Cas effector polypeptide of the present disclosure. For example, a guide nucleic acid may comprise a guide nucleotide sequence (present in a targeting segment) that can hybridize with a target nucleotide sequence present in a eukaryotic target nucleic acid. A guide nucleic acid of the present disclosure can be generated by human intervention and can comprise a nucleotide sequence not found in a naturally-occurring guide nucleic acid.

The term “naturally-occurring” as used herein as applied to a nucleic acid, a protein, a cell, or an organism, refers to a nucleic acid, cell, protein, or organism that is found in nature.

The terms “polynucleotide” and “nucleic acid,” used interchangeably herein, refer to a polymeric form of nucleotides of any length, either ribonucleotides or deoxynucleotides or combinations thereof. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The terms “polynucleotide” and “nucleic acid” should be understood to include, as applicable to the embodiment being described, single-stranded (such as sense or antisense) and double-stranded polynucleotides.

The terms “polypeptide,” “peptide,” and “protein”, are used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence.

Polypeptides as described herein also include polypeptides having various amino acid additions, deletions, or substitutions relative to the native amino acid sequence of a polypeptide of the present disclosure. In some embodiments, polypeptides that are homologs of a polypeptide of the present disclosure contain non-conservative changes of certain amino acids relative to the native sequence of a polypeptide of the present disclosure. In some embodiments, polypeptides that are homologs of a polypeptide of the present disclosure contain conservative changes of certain amino acids relative to the native sequence of a polypeptide of the present disclosure, and thus may be referred to as conservatively modified variants. A conservatively modified variant may include individual substitutions, deletions or additions to a polypeptide sequence which result in the substitution of an amino acid with a chemically similar amino acid. Conservative substitution tables providing functionally similar amino acids are well-known in the art. Such conservatively modified variants are in addition to and do not exclude polymorphic variants, interspecies homologs, and alleles of the disclosure. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). A modification of an amino acid to produce a chemically similar amino acid may be referred to as an analogous amino acid.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence similarity can be determined in a number of different manners. To determine sequence identity, sequences can be aligned using the methods and computer programs, including BLAST, available over the world wide web at ncbi.nlm.nih.gov/BLAST. See, e.g., Altschul et al. (1990), J. Mol. Biol. 215:403-10. Another alignment algorithm is FASTA, available in the Genetics Computing Group (GCG) package, from Madison, Wisconsin, USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, California, USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970).

“Recombinant,” as used herein, means that a particular nucleic acid (DNA or RNA) is the product of various combinations of cloning, restriction, and/or ligation steps resulting in a construct having a structural coding or non-coding sequence distinguishable from endogenous nucleic acids found in natural systems. Generally, DNA sequences encoding the structural coding sequence can be assembled from cDNA fragments and short oligonucleotide linkers, or from a series of synthetic oligonucleotides, to provide a synthetic nucleic acid which is capable of being expressed from a recombinant transcriptional unit contained in a cell or in a cell-free transcription and translation system. Such sequences can be provided in the form of an open reading frame uninterrupted by internal non-translated sequences, or introns, which are typically present in eukaryotic genes. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “DNA regulatory sequences”, below). Similarly, the term “recombinant” polypeptide refers to a polypeptide which is not naturally occurring, e.g., is made by the artificial combination of two otherwise separated segments of amino sequence through human intervention. Thus, e.g., a polypeptide that comprises a heterologous amino acid sequence is recombinant.

Thus, e.g., the term “recombinant” polynucleotide or “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate expression of a coding sequence and/or production of an encoded polypeptide in a host cell.

The term “transformation” is used interchangeably herein with “genetic modification” and refers to a permanent or transient genetic change induced in a cell following introduction of new nucleic acid (e.g., DNA exogenous to the cell) into the cell. Genetic change (“modification”) can be accomplished either by incorporation of the new nucleic acid into the genome of the host cell, or by transient or stable maintenance of the new nucleic acid as an episomal element. Where the cell is a eukaryotic cell, a permanent genetic change is generally achieved by introduction of new DNA into the genome of the cell. In prokaryotic cells, permanent changes can be introduced into the chromosome or via extrachromosomal elements such as plasmids and expression vectors, which may contain one or more selectable markers to aid in their maintenance in the recombinant host cell. Suitable methods of genetic modification include viral infection, transfection, conjugation, protoplast fusion, electroporation, particle gun technology, calcium phosphate precipitation, direct microinjection, and the like. The choice of method is generally dependent on the type of cell being transformed and the circumstances under which the transformation is taking place (i.e. in vitro, ex vivo, or in vivo). A general discussion of these methods can be found in Ausubel, et al, Short Protocols in Molecular Biology, 3rd ed., Wiley & Sons, 1995.

“Operably linked” refers to a juxtaposition wherein the components so described are in a relationship permitting them to function in their intended manner. For instance, a promoter is operably linked to a coding sequence if the promoter affects its transcription or expression. As used herein, the terms “heterologous promoter” and “heterologous control regions” refer to promoters and other control regions that are not normally associated with a particular nucleic acid in nature. For example, a “transcriptional control region heterologous to a coding region” is a transcriptional control region that is not normally associated with the coding region in nature.

The use of the terms “a,” “an,” and “the,” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the embodiments of the disclosure and does not pose a limitation on the scope of the disclosure unless otherwise claimed. No language in the specification should be construed as indicating any non-claimed element as essential to the practice of the embodiments of the disclosure.

Reference to “about” a value or parameter herein refers to the usual error range for the respective value readily known to the skilled person in this technical field. Reference to “about” a value or parameter herein includes (and describes) aspects that are directed to that value or parameter per se. For example, description referring to “about X” includes description of “X.”

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

The terms “isolated” and “purified” as used herein refers to a material that is removed from at least one component with which it is naturally associated (e.g., removed from its original environment). The term “isolated,” when used in reference to an isolated protein, refers to a protein that has been removed from the culture medium of the host cell that expressed the protein. As such an isolated protein is free of extraneous or unwanted compounds (e.g., nucleic acids, native bacterial or other proteins, etc.).

It is understood that aspects and embodiments of the present disclosure described herein include “comprising,” “consisting,” and “consisting essentially of” aspects and embodiments.

It is to be understood that one, some, or all of the properties of the various embodiments described herein may be combined to form other embodiments of the present disclosure. These and other aspects of the present disclosure will become apparent to one of skill in the art. These and other embodiments of the present disclosure are further described by the detailed description that follows.

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N. Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Gene Transfer Vectors for Mammalian Cells (J. M. Miller and M. P. Calos, eds., 1987); PCR: The Polymerase Chain

### DETAILED DESCRIPTION

The present disclosure relates to CRISPR-Cas systems that utilize Cas12L for editing nucleic acids in eukaryotic cells in a temperature regulated manner. Methods and compositions for using these systems for editing nucleic acids in eukaryotic cells are provided herein. A “Cas12L” polypeptide is also referred to herein as a “Casλ” polypeptide or a “Cas-lambda” polypeptide.

In some cases, the eukaryotic cells are plant cells. Thus, the present disclosure relates to CRISPR-Cas systems that utilize a Cas12L polypeptide and a guide nucleic acid for editing nucleic acids in plants. Methods and compositions for using these systems for editing nucleic acids in plants are provided herein.

In general, a Cas12L polypeptide is capable of forming a ribonucleoprotein (RNP) complex by binding to or otherwise interacting with a guide nucleic acid (e.g., a guide RNA (gRNA)). The Cas12L-gRNA ribonucleoprotein complex is capable of being targeted to a target nucleic acid via base pairing between the guide RNA and a target nucleotide sequence in the target nucleic acid that is complementary to the sequence of the guide RNA. The guide RNA thus provides the specificity for targeting a particular target nucleic. Once the Cas12L-gRNA ribonucleoprotein complex has come into association with a target nucleic acid by virtue of the targeting of the RNP complex to that target nucleic acid by the guide RNA, the Cas12L protein is able to have activity at that target nucleic acid and accordingly edit the target nucleic acid.

Accordingly, the present disclosure provides RNA-guided CRISPR-Cas effector polypeptides for use in CRISPR-based targeting systems in eukaryotic cells, where the CRISPR-Cas systems provide for temperature dependent (temperature regulated) gene editing in eukaryotic cells. As an example, the present disclosure provides Cas12L polypeptides for use in CRISPR-based targeting systems in plants. Provided herein are Cas12L polypeptides, nucleic acids encoding the same, compositions containing the same, and methods of using the same to e.g. edit a target nucleic acid. The present disclosure provides ribonucleoprotein complexes containing a Cas12L polypeptide and a guide RNA which may be used to e.g. edit a target nucleic acid. The present disclosure provides methods of modifying a target nucleic acid in plants using a Cas12L polypeptide and a guide RNA. The present disclosure also provides guide RNAs that bind to and provide target sequence specificity to Cas12L polypeptides. Provided herein are guide RNAs that can bind or otherwise interact with Cas12L polypeptides, nucleic acids encoding the same, compositions containing the same, and methods of using the same to e.g. edit a target nucleic acid.

### Methods of Modifying a Target Nucleic Acid in a Eukaryotic Cell

The present disclosure provides methods of modifying a target nucleic acid in a eukaryotic cell. The methods comprise contacting the target nucleic acid in the eukaryotic cell with: a) a Cas12L polypeptide; and b) a Cas12L guide nucleic acid, where such contacting results in modification of the target nucleic acid, and where the contacting is carried out at a temperature of from about 25° C. to about 40° C. (e.g., from about 25° C. to about 28° C., from about 28° C. to about 30° C., from about 28° C. to about 32° C., from about 30° C. to about 32° C., from about 30° C. to about 37° C., from about 32° C. to about 34° C., from about 30° C. to about 34° C., from about 34° C. to about 37° C., or from about 37° C. to about 40° C.).

In some cases, modification of a target nucleic acid does not substantially occur at a temperature of less than 28° C. For example, in some cases, modification of a target nucleic acid does not substantially occur at a temperature of from about 17° C. to about 25° C., or from about 25° C. to about 28° C. In some cases, modification of a target nucleic acid occurs, if at all, at less than 75%, less than 50%, less than 25%, less than 10%, or less than 5%, of the extent to which the modification of the target nucleic acid occurs when the modification is conducted at 32° C. For example, in a population of eukaryotic cells, each containing the target nucleic acid, at least 50%, at least 60%, at least 70%, at least 75%, at least 80%, at least 85%, at least 90%, at least 95%, at least 98%, or at least 99%, of the cells would, following contact at 32° C. with a Cas12L polypeptide and a Cas12L guide nucleic acid, contain a modification of the target nucleic acid, which modification was effected by the Cas12L polypeptide (together with the Cas12L guide nucleic acid); while, if the contacting was carried out at a temperature of less than 28° C. (e.g., from 17° C. to 28° C., from 25° C. to 28° C., or from 17° C. to 25° C.), less than 50%, less than 45%, less than 40%, less than 35%, less than 30%, less than 25%, less than 20%, less than 15%, less than 10%, or less than 5%, of the eukaryotic cells would contain a modification of the target nucleic acid.

A target nucleic acid can be present in any of a variety of eukaryotic cells; i.e., a method of the present disclosure can be carried out in a variety of eukaryotic cells. Examples of eukaryotic cells in which a method of the present disclosure can be carried out include, e.g., a plant cell, an insect cell, an arthropod cell, a mammalian cell, a fish cell, a fungal cell, a yeast cell, an amphibian cell, and an avian cell. Suitable cells include cells of members of the kingdom Protista, including, but not limited to, algae (e.g., green algae, red algae, glaucophytes, cyanobacteria); fungus-like members of Protista, e.g., slime molds, water molds, etc.; animal-like members of Protista, e.g., flagellates (e.g., Euglena), amoeboids (e.g., amoeba), sporozoans (e.g, Apicomplexa, Myxozoa, Microsporidia), and ciliates (e.g., Paramecium). Suitable cells include cells of members of the kingdom Fungi, including, but not limited to, members of any of the phyla: Basidiomycota (club fungi; e.g., members of Agaricus, Amanita, Boletus, Cantherellus, etc.); Ascomycota (sac fungi, including, e.g., Saccharomyces); Mycophycophyta (lichens); Zygomycota (conjugation fungi); and Deuteromycota Suitable cells include cells members of the kingdom Plantae, including, but not limited to, members of any of the following divisions: Bryophyta (e.g., mosses), Anthocerotophyta (e.g., hornworts), Hepaticophyta (e.g., liverworts), Lycophyta (e.g., club mosses), Sphenophyta (e.g., horsetails), Psilophyta (e.g., whisk ferns), Ophioglossophyta, Pterophyta (e.g., ferns), Cycadophyta, Gingkophyta, Pinophyta, Gnetophyta, and Magnoliophyta (e.g., flowering plants). Suitable cells include cells of members of the kingdom Animalia, including, but not limited to, members of any of the following phyla: Porifera (sponges); Placozoa; Orthonectida (parasites of marine invertebrates); Rhombozoa; Cnidaria (corals, anemones, jellyfish, sea pens, sea pansies, sea wasps); Ctenophora (comb jellies); Platyhelminthes (flatworms); Nemertina (ribbon worms); Ngathostomulida (jawed worms)p Gastrotricha; Rotifera; Priapulida; Kinorhyncha; Loricifera; Acanthocephala; Entoprocta; Nemotoda; Nematomorpha; Cycliophora; Mollusca (mollusks); Sipuncula (peanut worms); Annelida (segmented worms); Tardigrada (water bears); Onychophora (velvet worms); Arthropoda (including the subphyla: Chelicerata, Myriapoda, Hexapoda, and Crustacea, where the Chelicerata include, e.g., arachnids, Merostomata, and Pycnogonida, where the Myriapoda include, e.g., Chilopoda (centipedes), Diplopoda (millipedes), Paropoda, and Symphyla, where the Hexapoda include insects, and where the Crustacea include shrimp, krill, barnacles, etc.; Phoronida; Ectoprocta (moss animals); Brachiopoda; Echinodermata (e.g. starfish, sea daisies, feather stars, sea urchins, sea cucumbers, brittle stars, brittle baskets, etc.); Chaetognatha (arrow worms); Hemichordata (acorn worms); and Chordata. Suitable members of Chordata include any member of the following subphyla: Urochordata (sea squirts; including Ascidiacea, Thaliacea, and Larvacea); Cephalochordata (lancelets); Myxini (hagfish); and Vertebrata, where members of Vertebrata include, e.g., members of Petromyzontida (lampreys), Chondrichthyces (cartilaginous fish), Actinopterygii (ray-finned fish), Actinista (coelocanths), Dipnoi (lungfish), Reptilia (reptiles, e.g., snakes, alligators, crocodiles, lizards, etc.), Aves (birds); and Mammalian (mammals). Suitable plants include any monocotyledon and any dicotyledon.

In some cases, the cell is a unicellular organism in vitro. In some cases, the cell is a unicellular organism in vitro. In some cases, the cell is obtained from a multicellular organism and is cultured as a unicellular entity in vitro. In some cases, the cell is present in a multicellular organism in vivo.

In some cases, a eukaryotic cell (e.g., a multicellular organism comprising the eukaryotic cell) is modified to include a Cas12L polypeptide and a Cas12L guide nucleic acid, where temperature is used to control activity of the Cas12L polypeptide in the context of gene drive. For example, at a first temperature (e.g. from about 17° C. to about 25° C. or from about 17° C. to about 28° C.), the gene drive does not occur. However, at a second temperature (e.g., from about 25° C. to about 40° C. (e.g., from about 25° C. to about 28° C., from about 28° C. to about 30° C., from about 28° C. to about 32° C., from about 30° C. to about 32° C., from about 30° C. to about 37° C., from about 32° C. to about 34° C., from about 30° C. to about 34° C., from about 34° C. to about 37° C., or from about 37° C. to about 40° C.), gene drive occurs. Such temperature-dependent activity can be used to control populations such as mosquitoes, fruit flies, and the like.

### Gene Editing in Plant Cells

The following description applies to plant cells. However, similar temperature control of Cas12L-mediated gene editing can be carried out in any of a variety of eukaryotic cells.

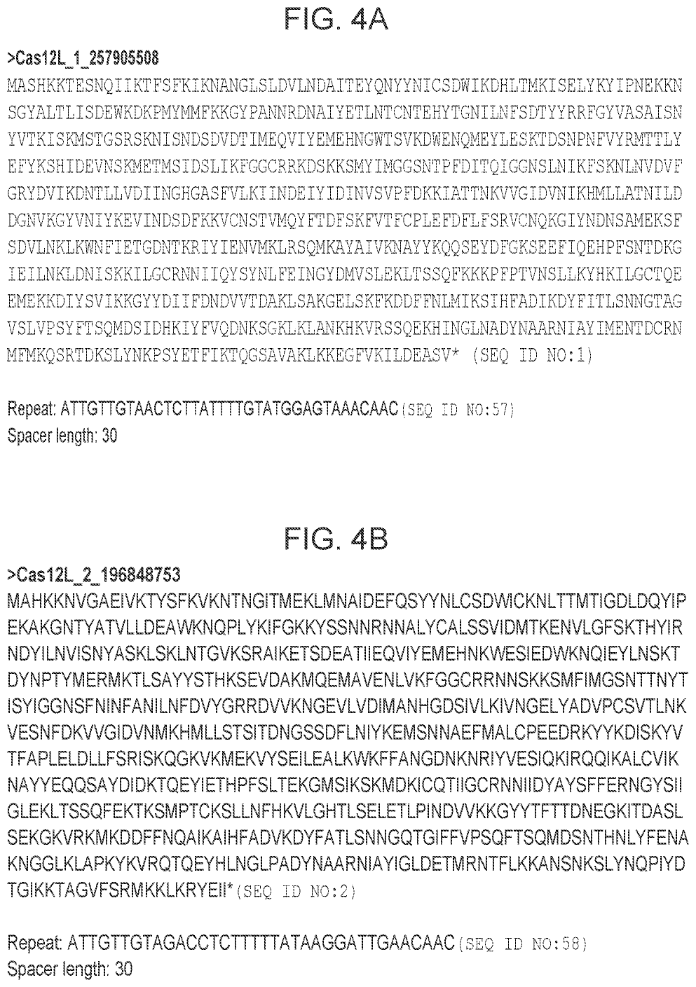

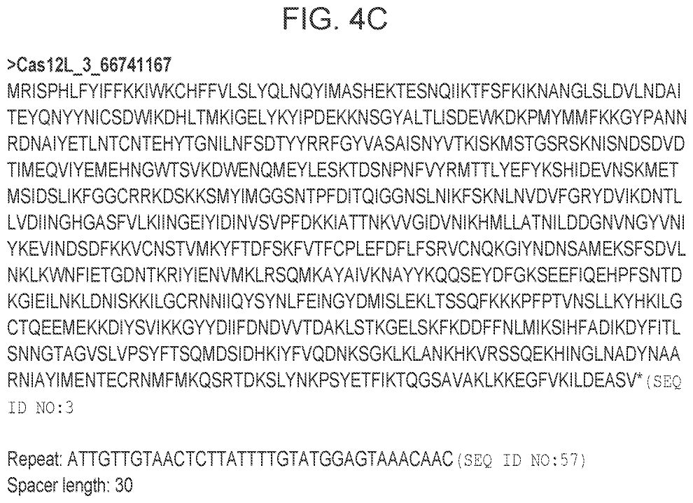

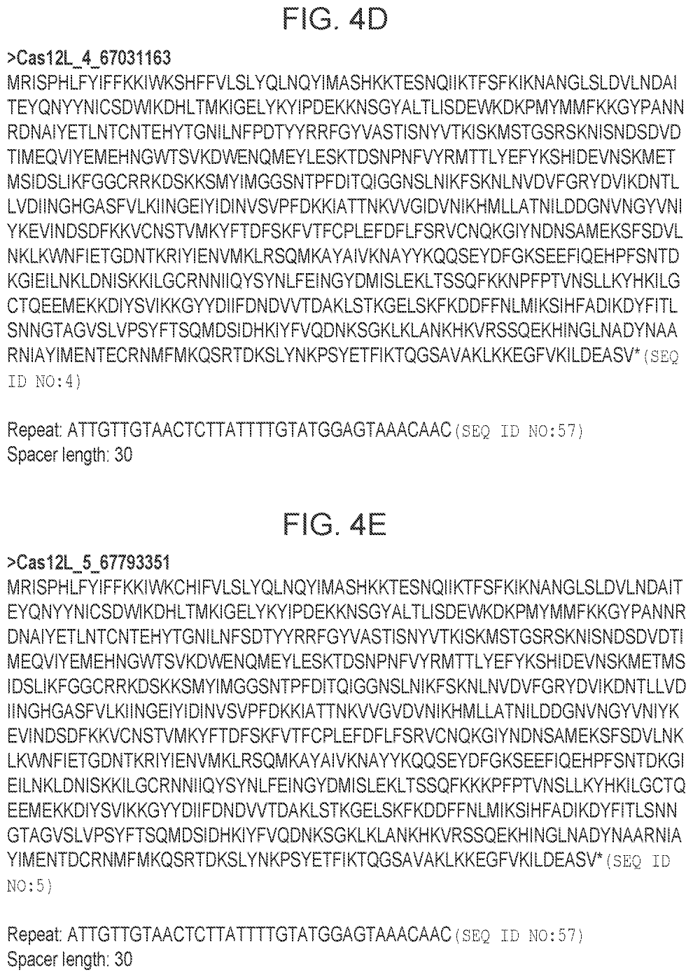

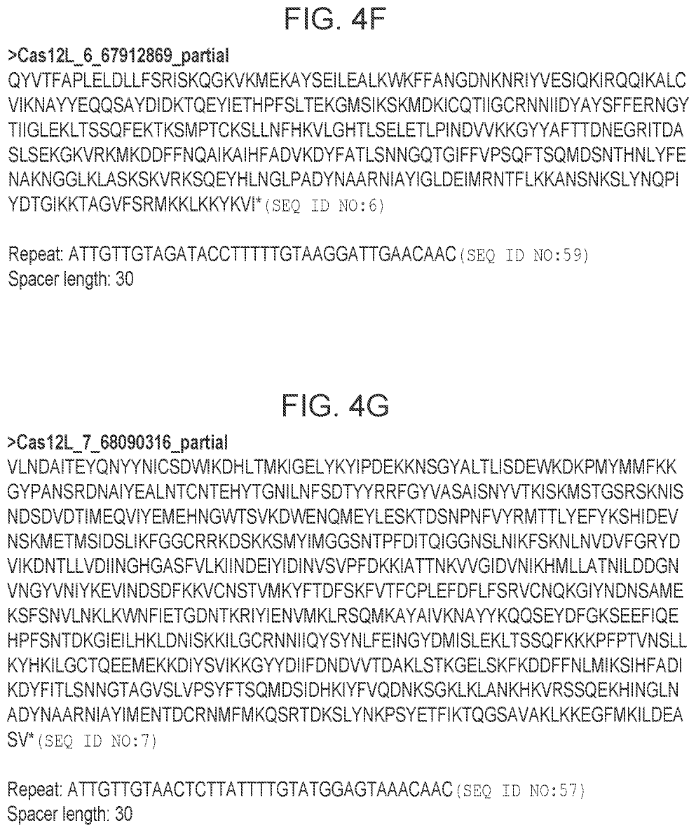

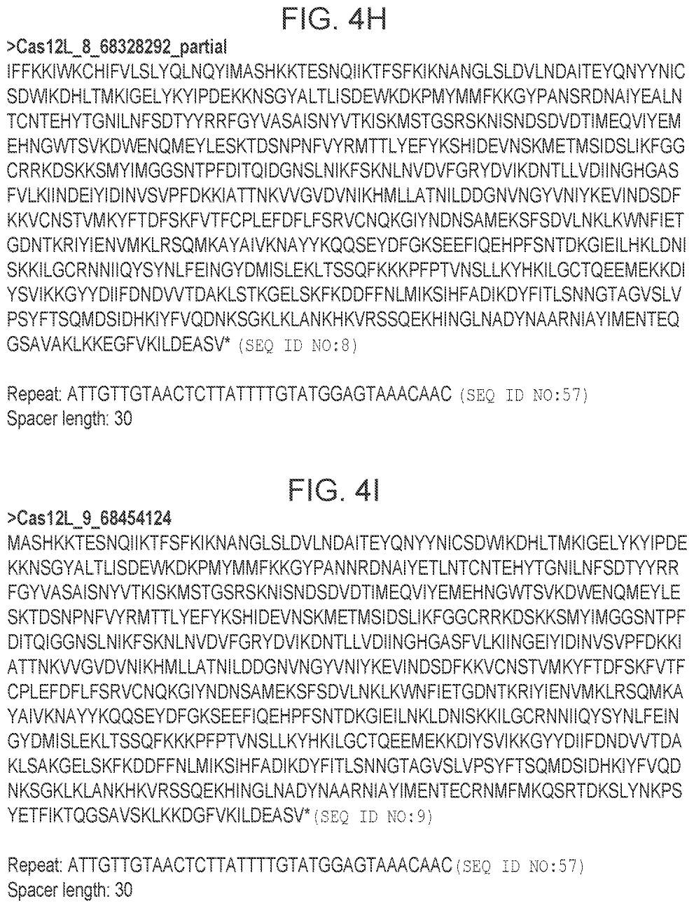

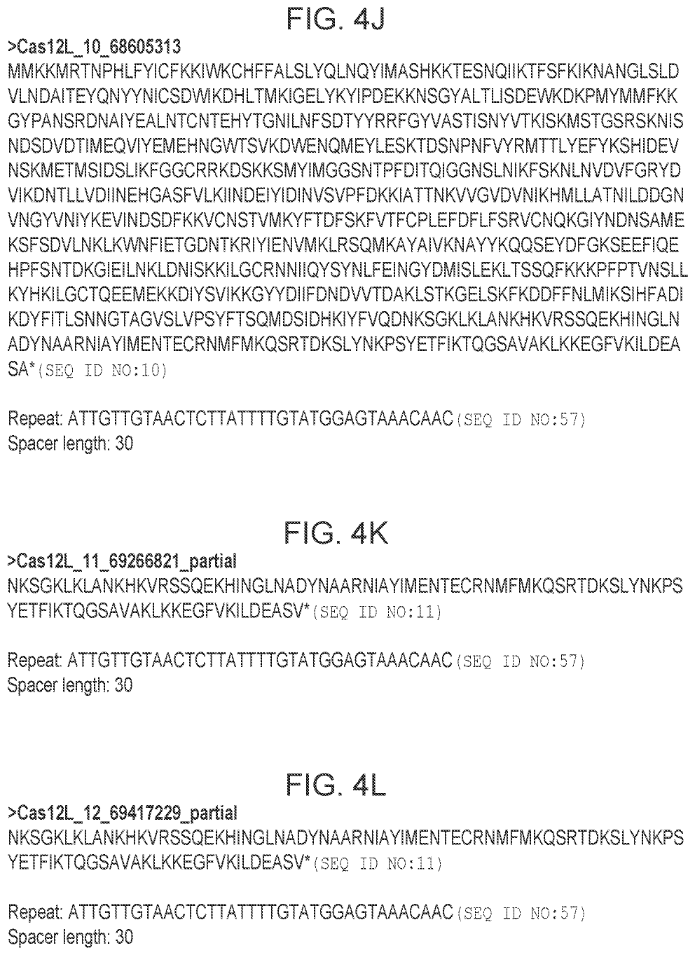

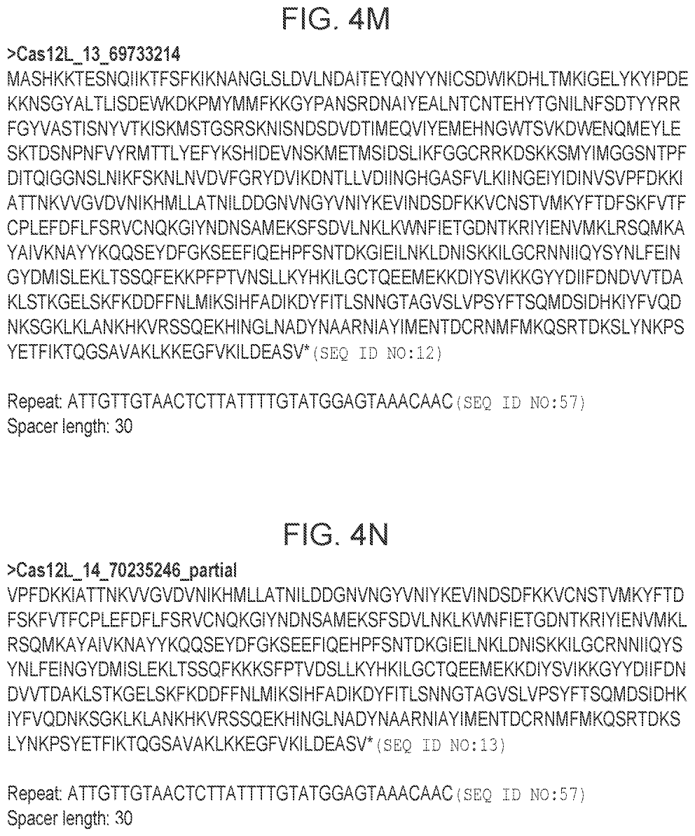

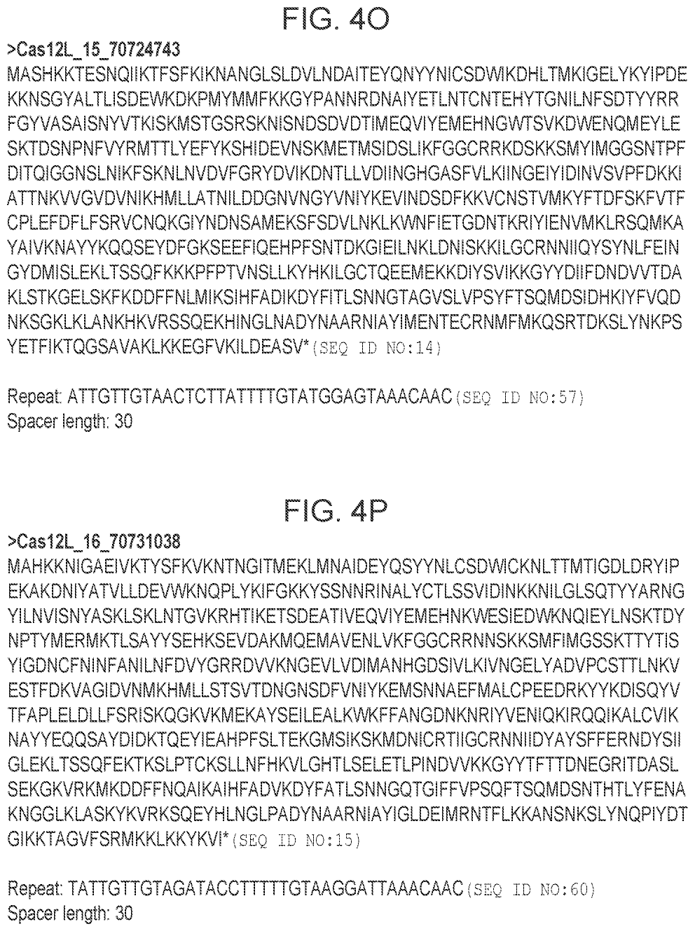

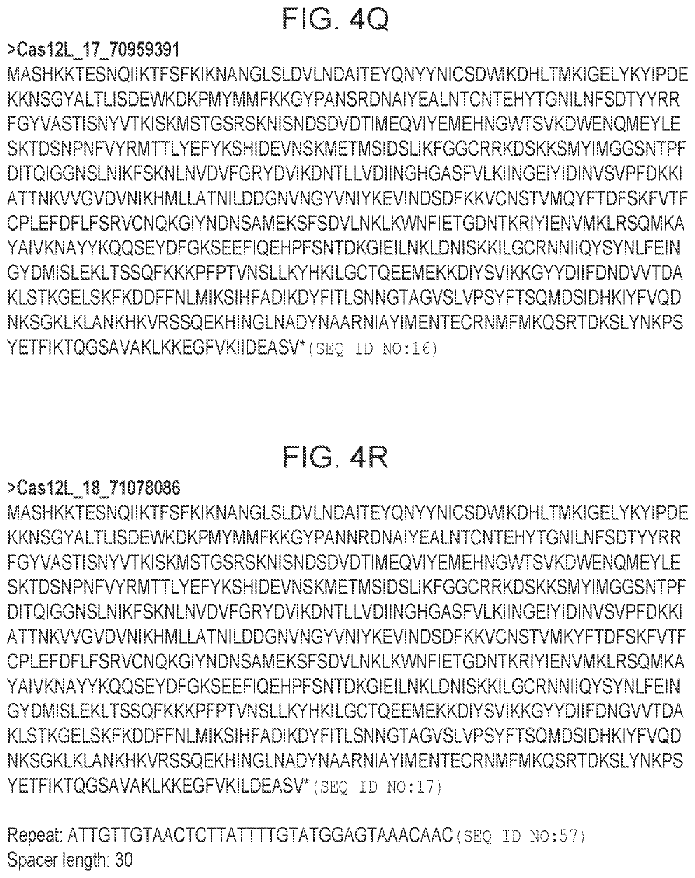

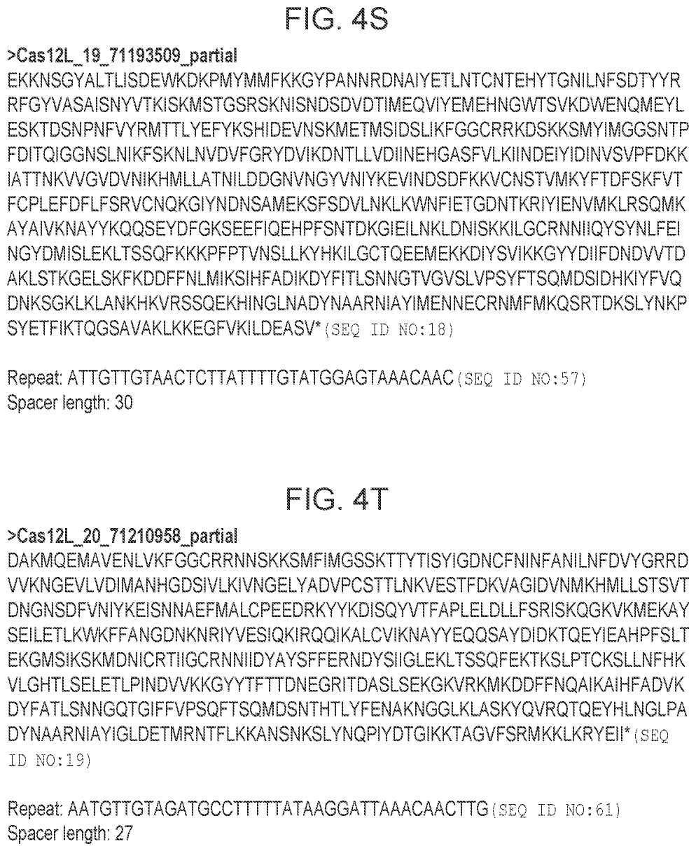

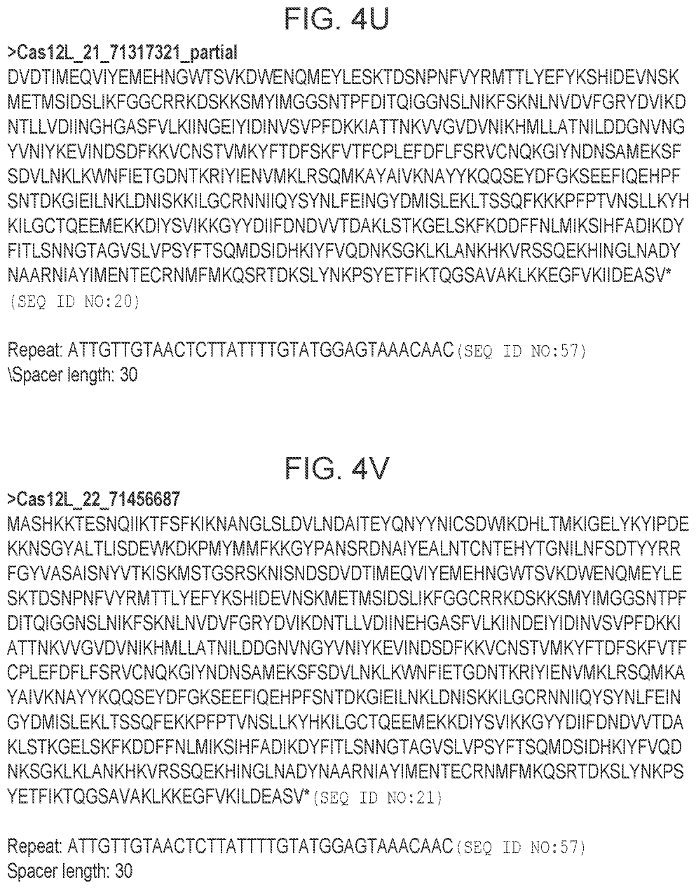

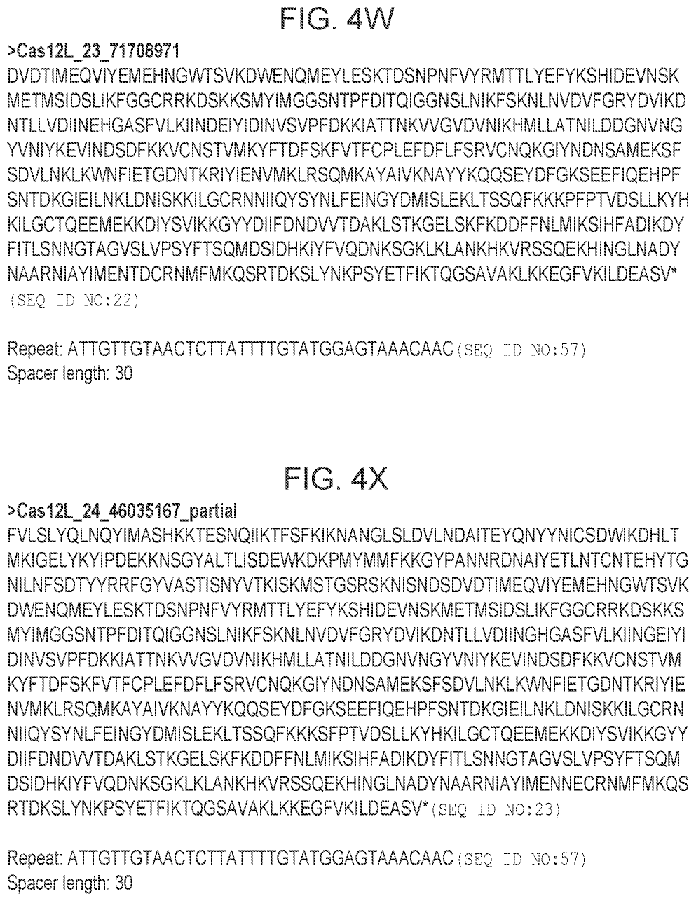

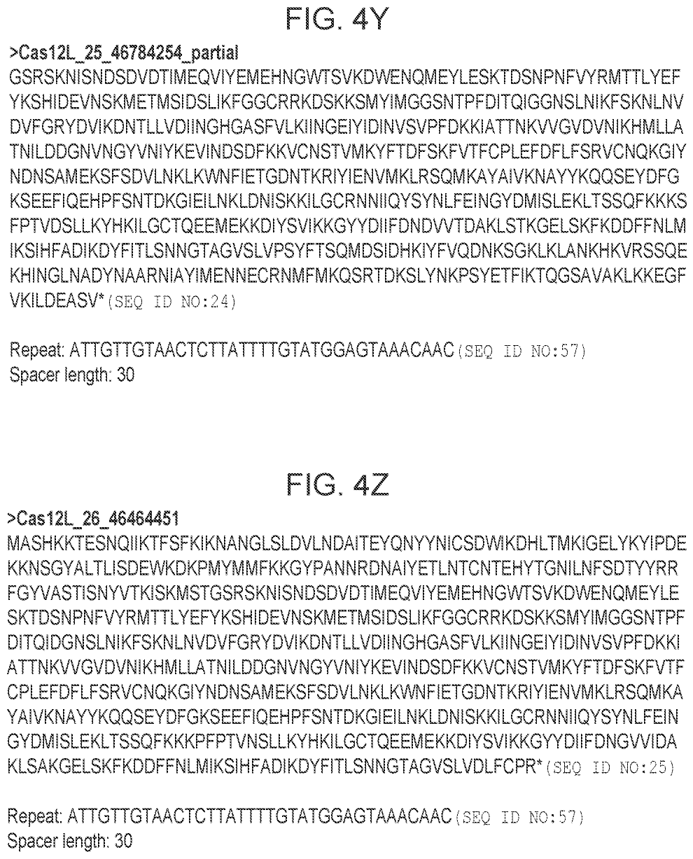

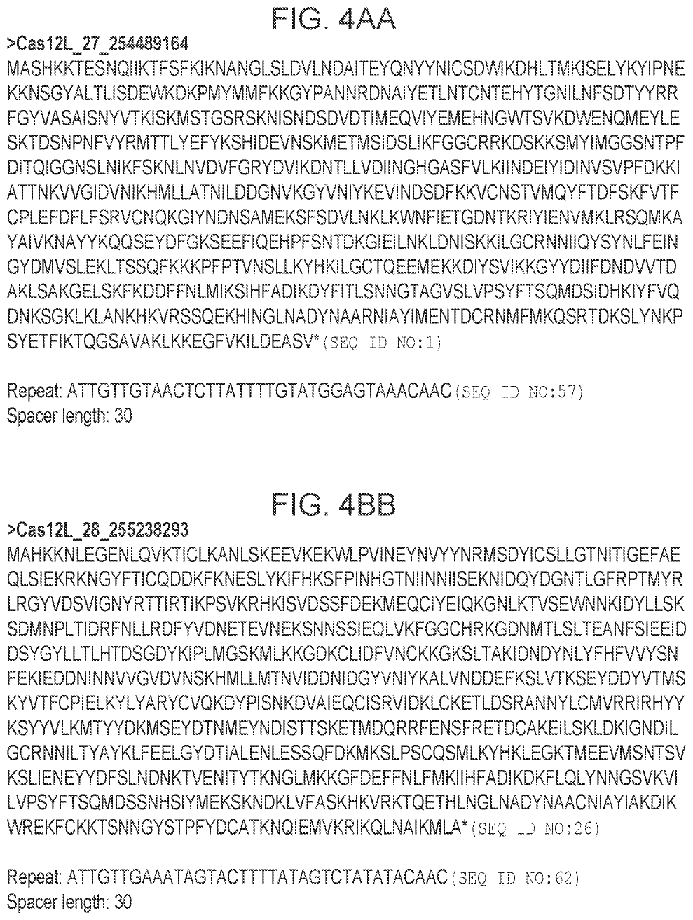

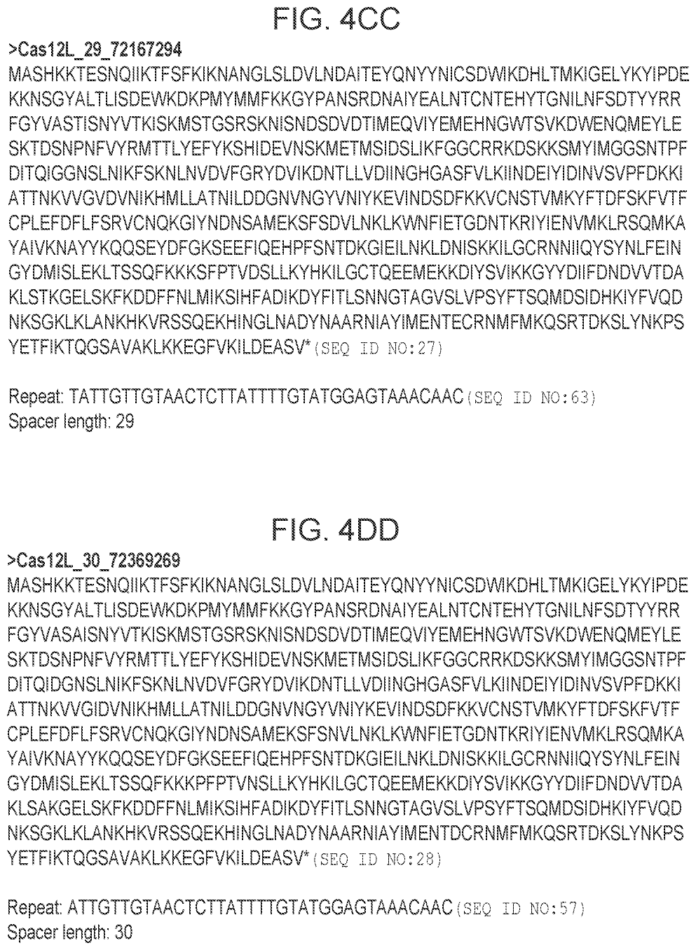

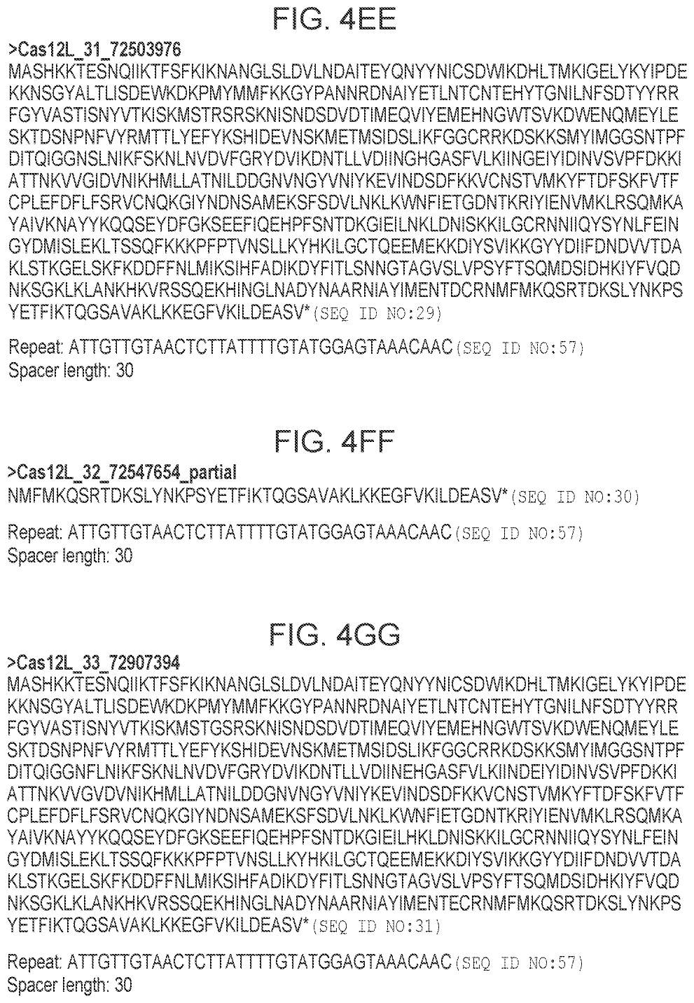

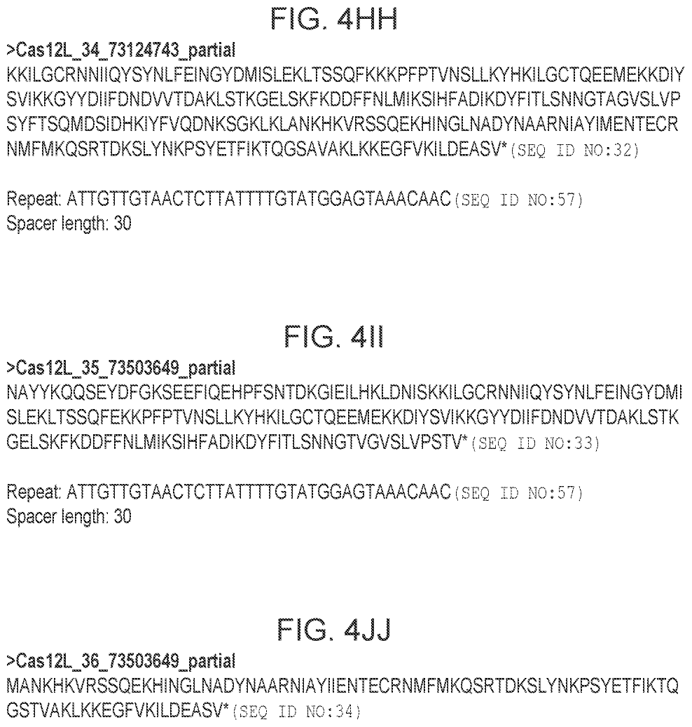

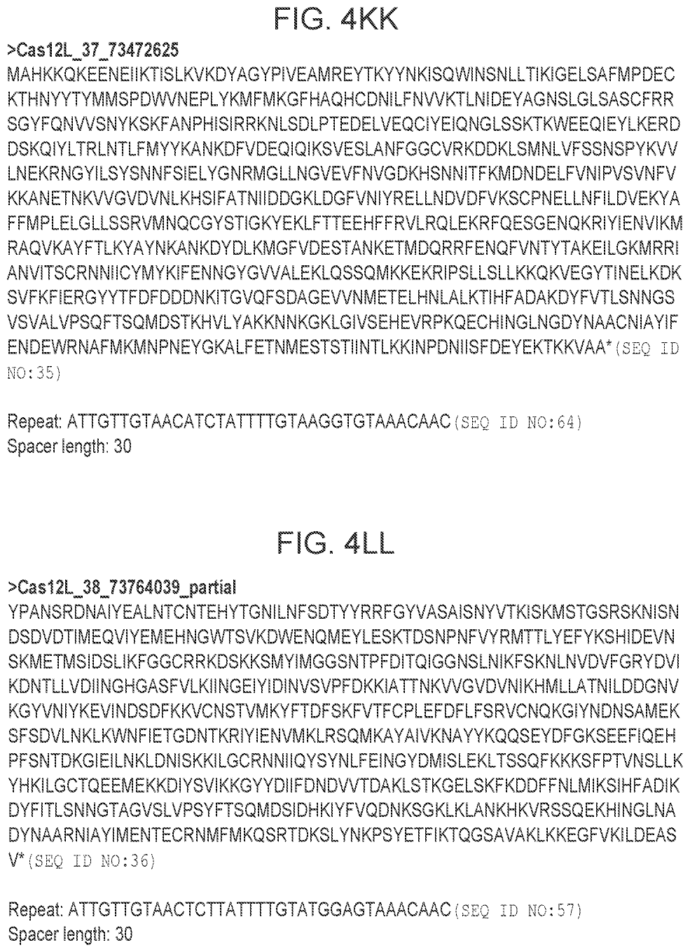

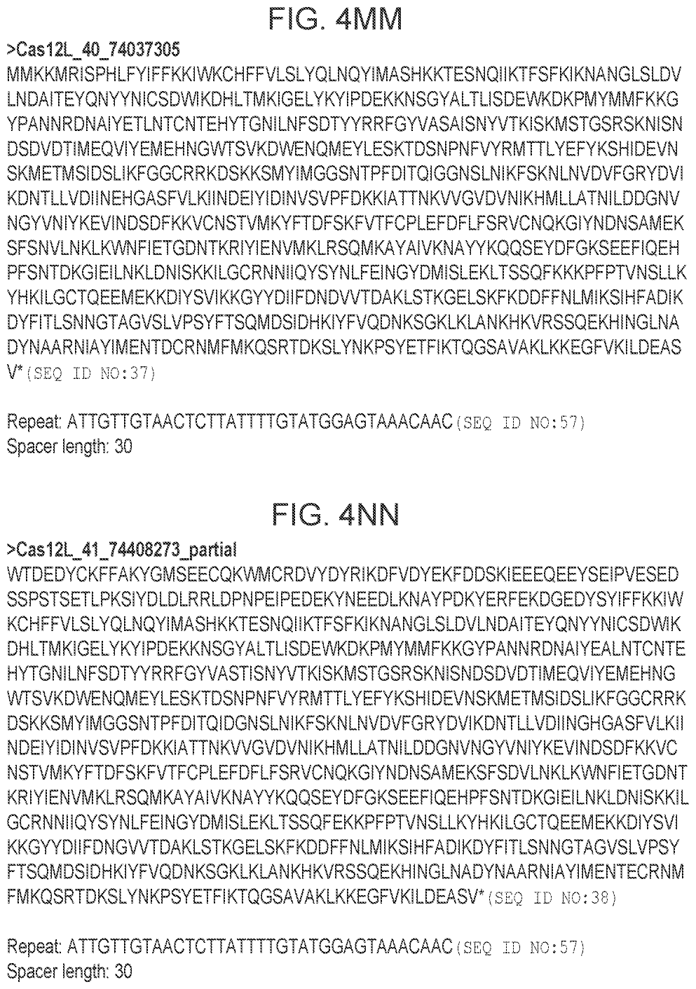

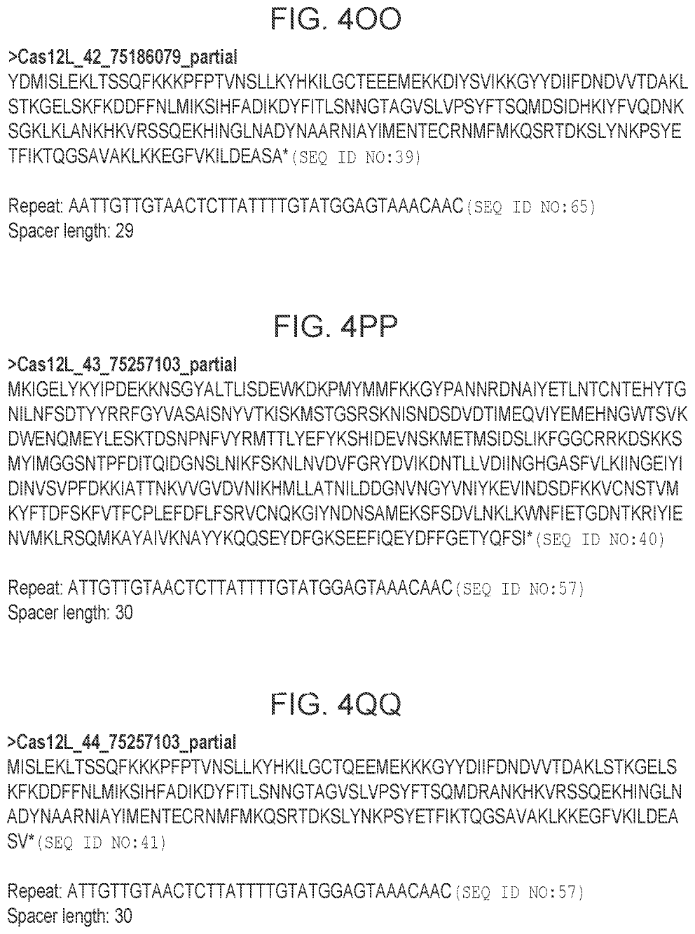

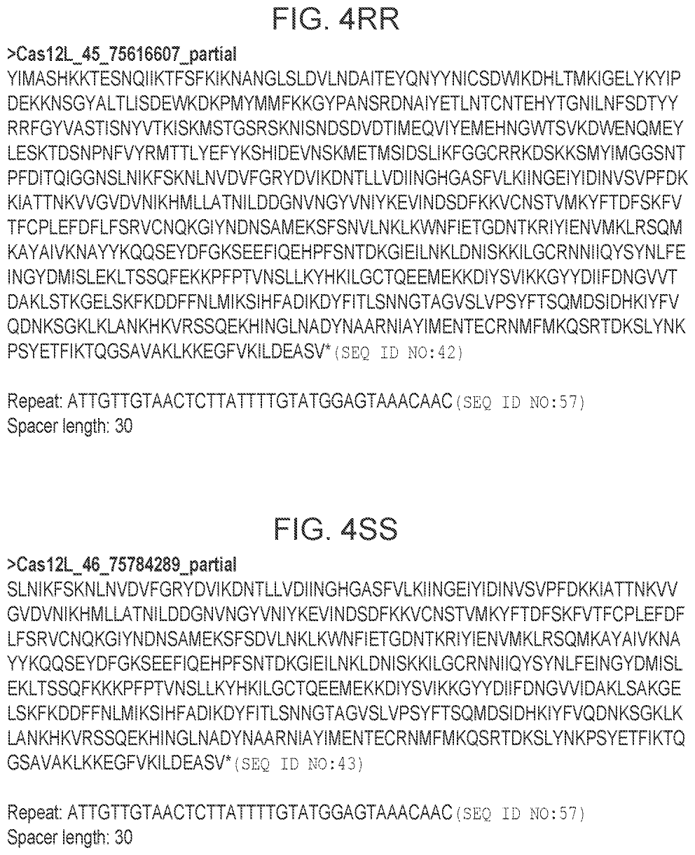

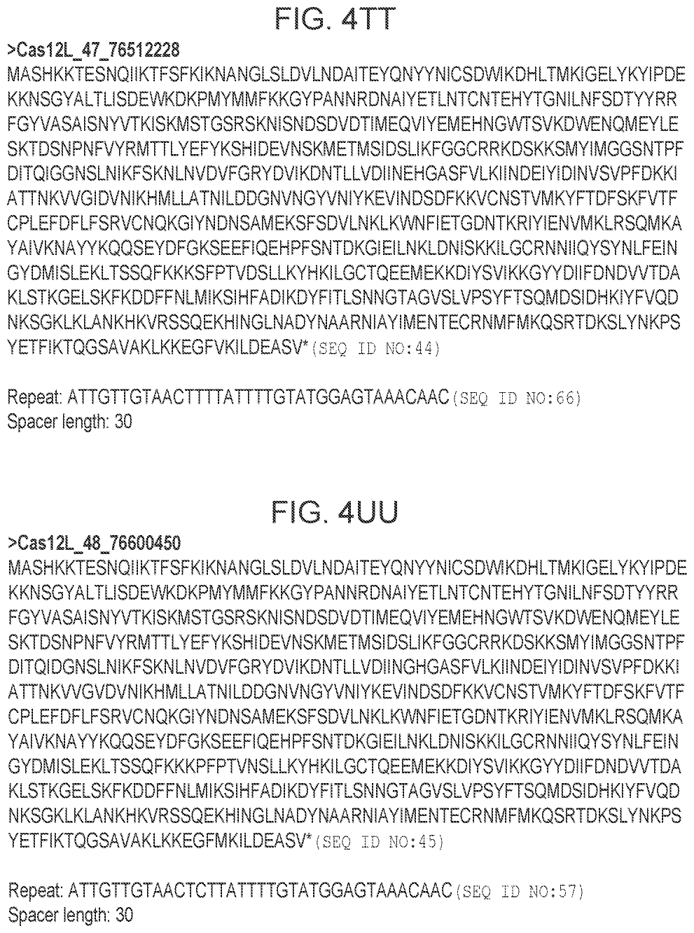

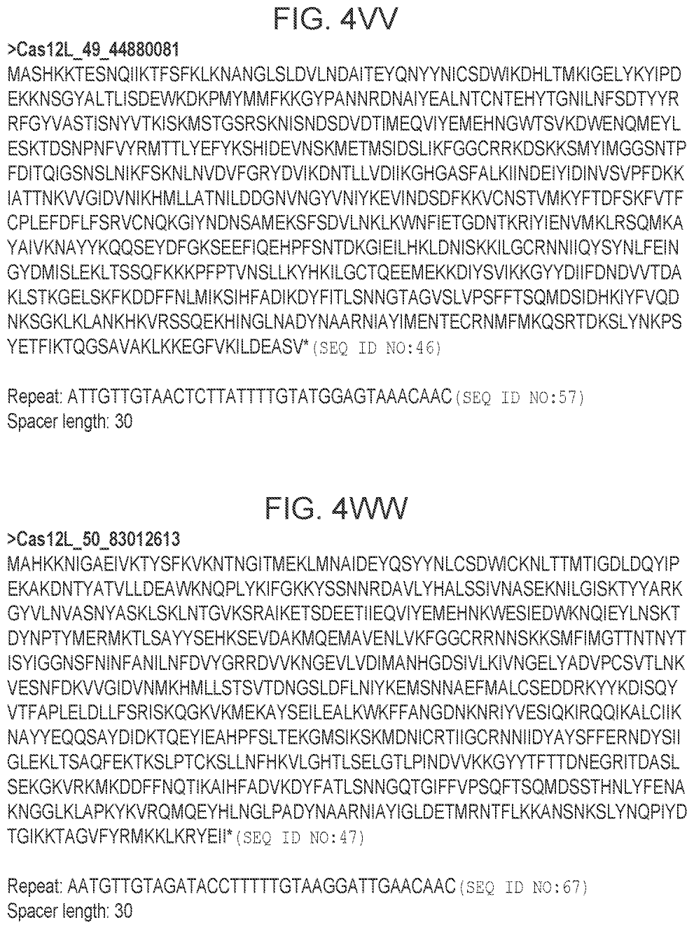

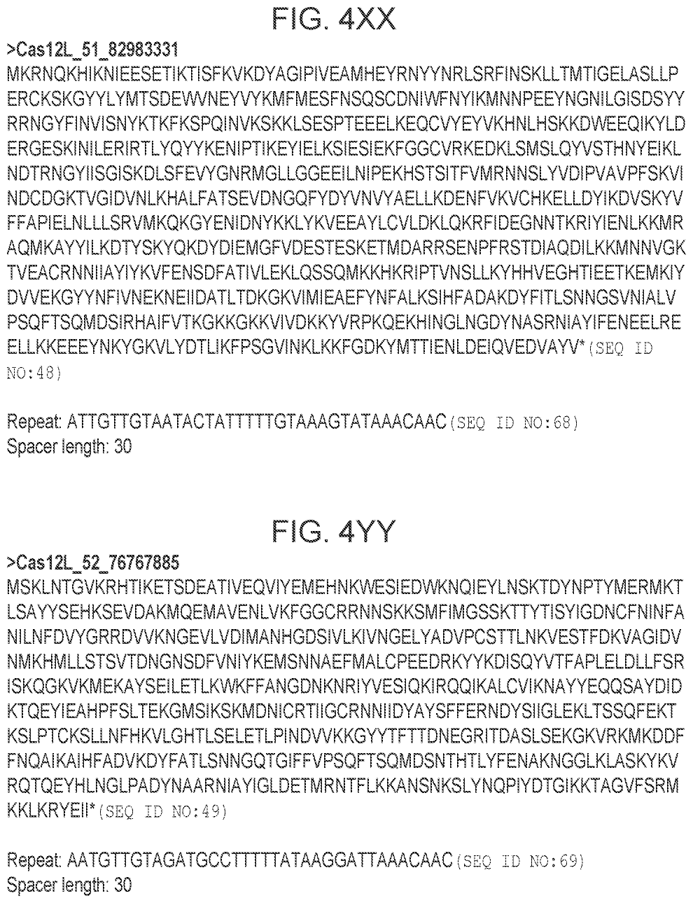

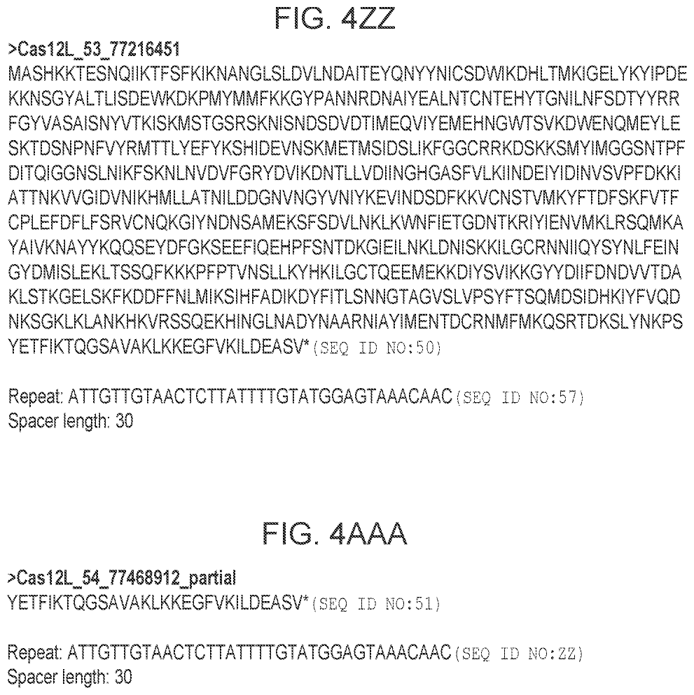

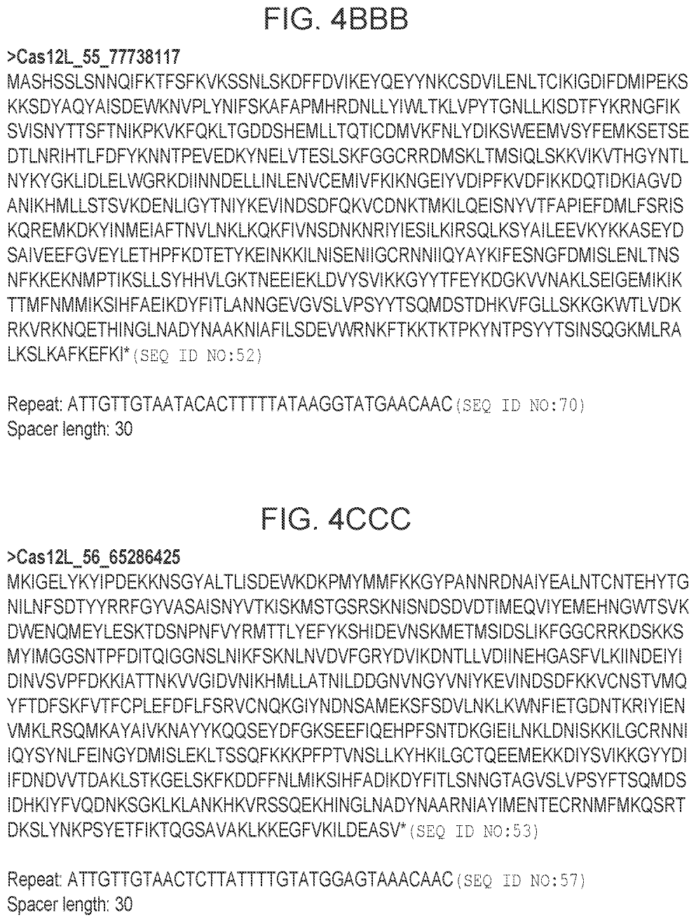

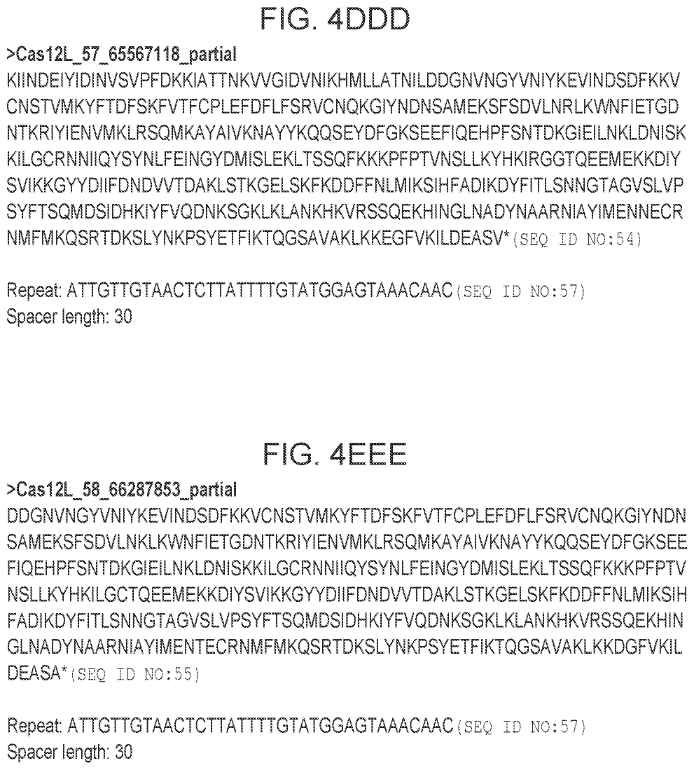

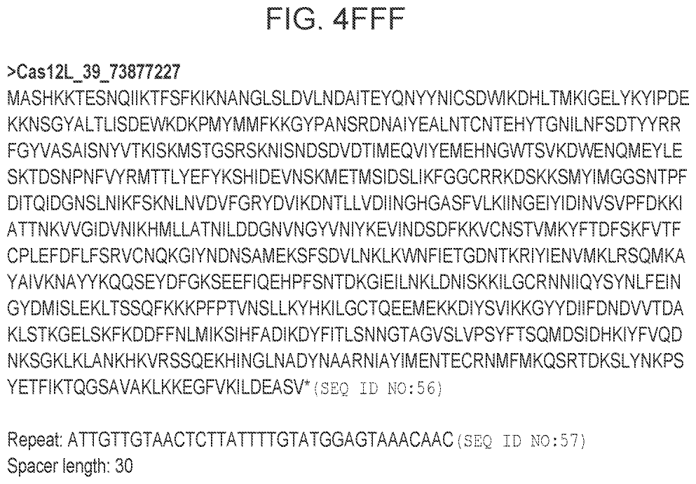

In one aspect, the present disclosure provides a method for modifying a target nucleic acid in a plant cell, the method including: a) introducing into a plant cell a Cas12L polypeptide and a guide RNA, and b) cultivating the plant cell under conditions whereby the Cas12L polypeptide and guide RNA are present as a complex that targets the target nucleic acid to generate a modification in the target nucleic acid. In some cases, the Cas12L polypeptide comprises an amino acid sequence having at least 50%, at least 60%, at least 70%, at least 80%, at least 85%, at least 90%, at least 95%, at least 98%, at least 99%, or 100%, amino acid identity to the amino acid sequence depicted in any one of FIG. 4A-4Z, 4AA-4ZZ, and 4AAA-4FFF. In some cases, the Cas12L polypeptide includes one or more nuclear localization signals (NLS). In some cases, at least one of the one or more nuclear localization signals is an SV40-type NLS. In some cases, the Cas12L polypeptide and the guide RNA are encoded in one or more recombinant nucleic acids in the plant cell; i.e., a recombinant nucleic acid comprising a nucleotide sequence encoding the Cas12L polypeptide and/or the guide RNA. In some cases, one of more of the recombinant nucleic acids include at least one intron. In some cases, the nucleotide sequence encoding the Cas12L polypeptide and/or the nucleotide sequence encoding the guide RNA is operably linked to a promoter that is functional in plants. In some cases, the promoter is a UBQ10 promoter. In some cases, the UBQ10 promoter includes a nucleic acid sequence that is at least 80% identical to SEQ ID NO:85. In some embodiments that may be combined with any of the preceding embodiments, expression of the guide RNA is driven by an RNA Polymerase II promoter (i.e., the nucleotide sequence encoding the guide RNA is operably linked to an RNA Polymerase II (“Pol II”) promoter). In some cases, the Pol II promoter is a CmYLCV promoter or a 2×35S promoter. In some cases, the promoter comprises a nucleic acid sequence that is at least 80% identical to SEQ ID NO:87 or SEQ ID NO:88. In some cases that may be combined with any of the preceding embodiments, the plant cell is cultivated at a temperature in the range of about 23° C. to about 37° C. In some embodiments that may be combined with any of the preceding embodiments, the plant cell is cultivated at a temperature in the range of about 20° C. to about 25° C. In some embodiments that may be combined with any of the preceding embodiments, the modification includes a deletion of one or more nucleotides in the target nucleic acid. In some embodiments that may be combined with any of the preceding embodiments, the deletion includes deletion of 3-15 nucleotides in the target nucleic acid. In some cases, the deletion includes deletion of 9 nucleotides in the target nucleic acid. In some embodiments that may be combined with any of the preceding embodiments, the target nucleic acid sequence is located in a region of repressive chromatin. In some embodiments that may be combined with any of the preceding embodiments, the target nucleic acid sequence is located in a region of open chromatin. In some cases, the modification includes an insertion of one or more nucleotides in the target nucleic acid. In some cases, the modification includes a combination of insertions of one or more nucleotides into, and deletions of one or more nucleotides from, the target nucleic acid. In some embodiments that may be combined with any of the preceding embodiments, the modification may include a combination of insertions and deletions of 3-15 nucleotides in the target nucleic acid. In some embodiments that may be combined with any of the preceding embodiments, the guide RNA is recombinantly fused to a ribozyme. In some embodiments that may be combined with any of the preceding embodiments, the plant cell comprises a genetic background that exhibits reduced susceptibility to transgene silencing.

In another aspect, the present disclosure provides a recombinant vector including a nucleic acid sequence that includes a promoter that is functional in plants and that encodes a Cas12L polypeptide and a guide RNA. In some embodiments, the Cas12L polypeptide comprises an amino acid sequence having at least 50%, at least 60%, at least 70%, at least 80%, at least 85%, at least 90%, at least 95%, at least 98%, at least 99%, or 100%, amino acid identity to the amino acid sequence depicted in any one of FIG. 4A-4Z, 4AA-4ZZ, and 4AAA-4FFF. In some embodiments that may be combined with any of the preceding embodiments, the Cas12L polypeptide includes a nuclear localization signal (NLS). In some embodiments, the nuclear localization signal is an SV40-type NLS. In some embodiments that may be combined with any of the preceding embodiments, the nucleic acid sequence includes at least one intron. In some embodiments, the promoter is a UBQ10 promoter. In some embodiments, the UBQ10 promoter includes a nucleic acid sequence that is at least 80% identical to SEQ ID NO:85. In some embodiments that may be combined with any of the preceding embodiments, expression of the guide RNA is driven by an RNA Polymerase II promoter. In some embodiments, the RNA Polymerase II promoter is a CmYLCV promoter or a 2×35S promoter. In some embodiments, the promoter comprises a nucleic acid sequence that is at least 80% identical to SEQ ID NO:87 or SEQ ID NO:88. In some embodiments that may be combined with any of the preceding embodiments, the guide RNA is recombinantly fused to a ribozyme.

In another aspect, the present disclosure provides a plant cell including a Cas12L polypeptide and a guide RNA, wherein the Cas12L polypeptide and guide RNA are capable of existing in a complex that targets a target nucleic acid to generate a modification in the target nucleic acid. In some embodiments, the Cas12L polypeptide includes an amino acid sequence having at least 50%, at least 60%, at least 70%, at least 80%, at least 85%, at least 90%, at least 95%, at least 98%, at least 99%, or 100%, amino acid identity to the amino acid sequence depicted in any one of FIG. 4A-4Z, 4AA-4ZZ, and 4AAA-4FFF. In some embodiments that may be combined with any of the preceding embodiments, the Cas12L polypeptide includes a nuclear localization signal (NLS). In some embodiments, the nuclear localization signal is an SV40-type NLS. In some embodiments that may be combined with any of the preceding embodiments, the Cas12L polypeptide and guide RNA are encoded from one or more recombinant nucleic acids in the plant cell. In some embodiments, one of more of the recombinant nucleic acids include at least one intron. In some embodiments, one of more of the recombinant nucleic acids include a promoter that is functional in plants. In some cases, the promoter is a UBQ10 promoter. In some embodiments, the UBQ10 promoter includes a nucleic acid sequence that is at least 80% identical to SEQ ID NO:85. In some embodiments that may be combined with any of the preceding embodiments, expression of the guide RNA is driven by an RNA Polymerase II promoter. In some embodiments, the RNA Polymerase II promoter is a CmYLCV promoter or a 2×35S promoter. In some embodiments, the promoter comprises a nucleic acid sequence that is at least 80% identical to SEQ ID NO:87 or SEQ ID NO:88. In some embodiments that may be combined with any of the preceding embodiments, the plant cell is cultivated at a temperature in the range of about 23° C. to about 37° C. In some embodiments that may be combined with any of the preceding embodiments, the plant cell is cultivated at a temperature in the range of about 20° C. to about 25° C. In some embodiments that may be combined with any of the preceding embodiments, the modification includes a deletion of one or more nucleotides in the target nucleic acid. In some embodiments that may be combined with any of the preceding embodiments, the deletion includes deletion of 3-15 nucleotides in the target nucleic acid. In some embodiments, the deletion includes deletion of 9 nucleotides in the target nucleic acid. In some cases, the modification includes an insertion of one or more nucleotides into the target nucleic acid (e.g., an insertion of from 3 to 15 nucleotides). In some cases, the modification includes a combination of an insertion of one or more nucleotides into, and a deletion of one or more nucleotides from, the target nucleic acid. In some embodiments that may be combined with any of the preceding embodiments, the target nucleic acid sequence is located in a region of repressive chromatin. In some embodiments that may be combined with any of the preceding embodiments, the target nucleic acid sequence is located in a region of open chromatin. In some embodiments that may be combined with any of the preceding embodiments, the guide RNA is recombinantly fused to a ribozyme. In some embodiments that may be combined with any of the preceding embodiments, the plant cell comprises a genetic background that exhibits reduced susceptibility to transgene silencing.

In another aspect, the present disclosure provides a plant including a plant cell of any one of the preceding embodiments, wherein the plant includes a modified nucleic acid. In some embodiments, the modification includes a deletion of one or more nucleotides in the nucleic acid. In some embodiments that may be combined with any of the preceding embodiments, the deletion includes deletion of 3-15 nucleotides. In some embodiments, the deletion includes deletion of 9 nucleotides. In some cases, the modification includes an insertion of one or more nucleotides into the target nucleic acid (e.g., an insertion of from 3 to 15 nucleotides). In some cases, the modification includes a combination of an insertion of one or more nucleotides into, and a deletion of one or more nucleotides from, the target nucleic acid.

In another aspect, the present disclosure provides a progeny plant of the plant of any one of the preceding embodiments, wherein the progeny plant includes a modified nucleic acid. In some embodiments, the modification includes a deletion of one or more nucleotides in the nucleic acid. In some embodiments that may be combined with any of the preceding embodiments, the deletion includes deletion of 3-15 nucleotides. In some embodiments, the deletion includes deletion of 9 nucleotides. In some cases, the modification includes an insertion of one or more nucleotides into the target nucleic acid (e.g., an insertion of from 3 to 15 nucleotides). In some cases, the modification includes a combination of an insertion of one or more nucleotides into, and a deletion of one or more nucleotides from, the target nucleic acid.

The temperature difference in editing activity of a Cas12L polypeptide can be used to control the various functions and features of a plant cell, such as reproduction, flowering, flower color, ripening, disease resistance, pathogen resistance, and the like. For example, in some cases, a method of the present disclosure comprises: a) contacting a target nucleic acid in a plant cell with: i) a Cas12L polypeptide; and ii) a Cas12L guide nucleic acid; b) maintaining a plant cell for a first period of time at a first temperature of from about 17° C. to about 25° C., wherein the target nucleic acid is substantially not modified by the Cas12L polypeptide; and c) maintaining the plant cell for a second period of time at a second temperature of from about 25° C. to about 37° C., wherein the target nucleic acid is modified by the Cas12L polypeptide. As another example, in some cases, a method of the present disclosure comprises: a) contacting a target nucleic acid in a plant cell with: i) a Cas12L polypeptide; and ii) a Cas12L guide nucleic acid; b) maintaining the plant cell for a first period of time at a first temperature of from about 25° C. to about 37° C. (or from about 25° C. to about 40° C.), wherein the target nucleic acid is modified by the Cas12L polypeptide; and c) maintaining a plant cell for a second period of time at a second temperature of from about 17° C. to about 25° C., wherein the target nucleic acid is substantially not modified by the Cas12L polypeptide.

In some cases, the modification results in repression of expression of a target nucleic acid (e.g., silencing of a target nucleic acid). In some cases, the modification is deletion of all or a portion of a target nucleic acid. In some cases, the modification includes an insertion of one or more nucleotides into the target nucleic acid. In some cases, the modification includes a combination of an insertion of one or more nucleotides into, and a deletion of one or more nucleotides from, the target nucleic acid. In some cases, the modification results in expression of a target nucleic acid. In some cases, the modification results in expression of a target nucleic acid, where the target nucleic acid is an endogenous plant nucleic acid. In some cases, the modification results in expression of a target nucleic acid, where the target nucleic acid is heterologous to the plant cell (e.g., the target nucleic acid is a transgene or an exogenous nucleic acid).

For example, where the modification results in repression of expression of a target nucleic acid (e.g., silencing of a target nucleic acid), in some cases, the modification results in repression of expression of a gene product in a pigment production pathway that provides for a change in color of a flower, a bract, a leaf, or another plant part. Pigment production pathway gene products include those involved in an anthocyanin synthesis pathway (e.g., anthocyanin-5-acyltransferase; chalcone synthase; chalcone isomerase; flavanone 3-hydroxylase; flavonoid 3′-hydroxylase; flavonoid 3′,5′-hydroxylase; flavonoid 3-O-glucosyltransferase; anthocyanidin synthase; any of a variety of enzymes that modify anthocyanidin, such as glucosyltransferases, acyltransferases, and methyltransferases; and the like; see, e.g., Liu et al. (2018) Front. Chem. 6:52); a betalain synthesis pathway (e.g., dihydroxyphenylalanine (DOPA) 4,5-dioxygenase; cyclic-DOPA 5-O-glucosyltransferase; and the like); a carotenoid synthesis pathway; and the like. See, e.g., Tanaka et al. (2008) Plant J. 54:733.

As one non-limiting example, at a first temperature (e.g., a temperature of from about 17° C. to about 25° C.), the bract of a poinsettia is green, and at a second temperature (e.g., a temperature of from about 28° C. to about 37° C., or from about 28° C. to about 40° C.), the bract of the poinsettia is red.

In some cases, the target nucleic acid comprises a nucleotide sequence encoding a pigment production pathway enzyme. At a first temperature of from about 17° C. to about 25° C., the target nucleic acid is not modified by the Cas12L polypeptide; thus. the plant or the plant part will contain the pigment produced as a result of activity of the pigment production pathway. At a second temperature of from about 25° C. to about 37° C. or from about 25° C. to about 40° C., the target nucleic acid is modified by the Cas12L polypeptide; thus, the plant or the plant part lacks the pigment that would normally be produced by action of the pigment production pathway.

In other cases, the target nucleic acid is an endogenous nucleic acid or a transgene encoding a negative regulator of a pigment production pathway. At a first temperature of from about 17° C. to about 25° C., the target nucleic acid is not modified by the Cas12L polypeptide; thus, the pigment production pathway is blocked by the negative regulator and the pigment is not produced. At a second temperature of from about 28° C. to about 37° C. or from about 28° C. to about 40° C., the target nucleic acid is modified by the Cas12L polypeptide, thus allowing the pigment production pathway to function and change of the color of the plant or the plant part.

In some cases, where the modification results in repression of expression of a target nucleic acid (e.g., silencing of a target nucleic acid), in some cases, the modification results in repression of expression of a gene product in fruit ripening. Target nucleic acids include, e.g., Colorless non-ripening (CNR), nonripening (NOR), ripening inhibitor (RIN), DNA demthylase-2 (DML2), and ethylene insensitive-3 (EIN3). See, e.g., Wang et al. (2002) Plant Cell 14 Suppl: S131. As one non-limiting example, at a first temperature (e.g., a temperature of from about 17° C. to about 25° C.), the fruit of a plant is unripe, and at a second temperature (e.g., a temperature of from about 28° C. to about 37° C.), the fruit of the plant ripens.

In some cases, the target nucleic acid is a nucleic acid in a fruit, where the nucleic acid comprise a nucleotide sequence encoding an ethylene production pathway enzyme or signaling pathway polypeptide. At a first temperature of from about 17° C. to about 25° C., the target nucleic acid is not modified by the Cas12L polypeptide; thus, the fruit continues the ripening process. At a second temperature of from about 28° C. to about 37° C. or from about 28° C. to about 40° C., the target nucleic acid is modified by the Cas12L polypeptide; thus, the ripening process in the fruit is slowed down.

In other cases, the target nucleic acid is an endogenous nucleic acid or a transgene encoding a negative regulator of ethylene production or signaling pathway. At a first temperature of from about 17° C. to about 25° C., the target nucleic acid is not modified by the Cas12L polypeptide; thus, the production or signaling of ethylene is blocked, resulting in slower ripening of the fruit. At a second temperature of from about 28° C. to about 37° C. or from about 28° C. to about 40° C., the target nucleic acid is modified by the Cas12L polypeptide, thus allowing the fruit to ripen.

As another example, the modification results in expression of a transgene that confers resistance to insects or disease (e.g., a fungal disease, a bacterial disease), where the expression of such transgene occurs at a second temperature (e.g., a temperature of from about 28° C. to about 37° C.) and does not substantially occur at a first temperature (e.g., a temperature of from about 17° C. to about 25° C.). In some cases, the transgene is a plant disease resistance gene. Plant defenses are often activated by specific interaction between the product of a disease resistance gene in the plant and the product of a corresponding avirulence (Avr) gene in the pathogen. A plant can be genetically modified with a transgene that confers resistance to specific pathogen strains. For example: i) the tomato Cf-9 gene confers resistance to Cladosporium fulvum; ii) the tomato Pto gene confers resistance to Pseudomonas syringae; iii) the Arabidopsis RSP2 gene confers resistance to Pseudomonas syringae; and the like. A plant that is genetically modified with a transgene, and that is “resistant” to a disease-causing pathogen, is one that is more resistant (e.g., at least 20%, at least 30%, at least 40%, at least 50%, at least 60%, at least 70%, or at least 80% more resistant) to the disease-causing pathogen as compared to the wild type plant (a plant of the same species that does not comprise the transgene). In some cases, the transgene is a nucleic acid comprising a nucleotide sequence encoding a Bacillus thuringiensis (Bt) polypeptide, a derivative thereof, or a synthetic polypeptide modeled after a Bt polypeptide. Examples of suitable Bt polypeptides include a Bt delta-endotoxin polypeptide. In some cases, the transgene comprises a nucleotide sequence encoding a peticidal polypeptide, where non-limiting examples of such pesticidal polypeptides include, e.g., insecticidal proteins from Pseudomonas sp. such as PSEEN3174 (Monalysin (2011) PLoS Pathogens 7:1-13); insecticidal proteins from Photorhabdus sp. and Xenorhabdus sp.; a PIP-1 polypeptide; an AfIP-1A and/or AfIP-1B polypeptide; a PHI-4 polypeptide; a PIP-47 polypeptide; a PIP-72 polypeptide; a PtIP-50 polypeptide; a PtIP-65 polypeptide; a PtIP-83 polypeptide; a PtIP-96 polypeptide; a delta-endotoxin such as a Cry1, Cry2, Cry3, Cry4, Cry5, Cry6, Cry7, Cry8, Cry9, Cry10, Cry11, Cry12, Cry13, Cry14, Cry15, Cry16, Cry17, Cry18, Cry19, Cry20, Cry21, Cry22, Cry23, Cry24, Cry25, Cry26, Cry27, Cry 28, Cry 29, Cry 30, Cry31, Cry32, Cry33, Cry34, Cry35, Cry36, Cry37, Cry38, Cry39, Cry40, Cry41, Cry42, Cry43, Cry44, Cry45, Cry 46, Cry47, Cry49, Cry 51, or Cry55 class of delta-endotoxin genes of B. thuringiensis; a Cry1A polypeptide (see, e.g., U.S. Pat. Nos. 5,880,275 and 7,858,849); a DIG-3 polypeptide (see, e.g., U.S. Pat. Nos. 8,304,604 and 8,304,605); a DIG-1I polypeptide (see, e.g., U.S. Pat. Nos. 8,304,604 and 8,304,605); a Cry1B polypeptide; a Cry1C polypeptice; a Cry1F polypeptide; a Cry2 polypeptide (see, e.g., U.S. Pat. No. 7,064,249); a Cry3A polypeptide; a Cry4 polypeptide; a Cry5 polypeptide; a Cry6 polypeptide; a Cry8 polypeptide; a Cry9 polypeptide; a Cry46 protein, a Cry 51 protein, a Cry binary toxin; a TIC901 or related toxin; an AXMI-027, AXMI-036, or AXMI-038 polypeptide (see, e.g., U.S. Pat. No. 8,236,757); a vegetative insecticidal protein (Vip; see, e.g., Gupta et al. (2021) Front. Microbiol. 12:659736); and the like. In some cases, the transgene is a nucleic acid comprising a nucleotide sequence encoding an insect-specific polypeptide that, upon expression, disrupts the physiology of the affected pest; where such polypeptides include, e.g., an insect diuretic hormone receptor, an allatostatin, and the like. In some cases, the transgene is a nucleic acid comprising a nucleotide sequence encoding an enzyme involved in the modification, including the post-translational modification, of a biologically active molecule; for example, a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase, or a glucanase.

As an example, the modification can result in expression of a transgene, where the transgene is a nucleic acid comprising a nucleotide sequence encoding a lectin, where the nucleotide sequence is operably linked to a plant-specific promoter, e.g., a phloem-specific promoter, or the like. As another example, the modification can result in expression of a transgene, where the transgene is a nucleic acid comprising a nucleotide sequence encoding a ω-ACTX-Hv1a toxin (Hvt) (a component of the venom of the Australian funnel web spider Hadronyche versuta (Khan et al. (2006) Transgenic Res. 15:349). As another example, the modification can result in expression of a transgene, where the transgene is a nucleic acid comprising a nucleotide sequence encoding a lectin and a nucleotide sequence encoding Hvt. Such a transgene can confer broad-spectrum resistance against lepidopteran (e.g., Helicoverpa armigera and Spodoptera litura) and hemipteran (e.g., Myzus persicae, Phenacoccus solenopsis, and Bemisia tabaci) insect pests. See, e.g., Rauf et al. (2019) Nature Scientific Reports 9:6745

In some cases, the modification results in increased expression of an endogenous plant gene product that has insecticidal activity. Such endogenous plant proteins include, e.g., lectins, ribosome-inactivating proteins, enzymes inhibitors, arcelins, chitinases, ureases, and modified storage proteins. See, e.g., Carlini and Grossi-de-Si (2002) Toxicon. 40:1515. For example, in some cases, the modification results in increased expression of an endogenous jasmonic acid pathway protein.

As another example, a transgene can be a nucleic acid comprising a nucleotide sequence encoding an enzyme that cleaves a protein of a plant pathogen. For example, a transgene can be a nucleic acid comprising a nucleotide sequence encoding a plant apoplastic subtilisin-like protease, such as tomato P69B, which is able to cleave a secreted protein PC2 from the potato late blight pathogen Phytophthora infestans, thus triggering downstream immune responses. See, e.g., Wang et al. (2021) New Phytol. 229:3424.

As another example, a transgene can be a nucleic acid comprising a nucleotide sequence encoding an inhibitory RNA, such as a microRNA or a long double-stranded RNA, that inhibits an RNA of a plant pathogen. For example, a transgene can be a nucleic acid comprising a nucleotide sequence encoding TAS1c-siR483 and TAS2-siR453, which targets the RNA produced by BC1G_10728, BC1G_10508 and BC1G_08464 genes of the fungal pathogen Botrytis cinerea. See, e.g., Cai et al. (2018) Science 360:1126.

In some cases, the target nucleic acid comprises a nucleotide sequence encoding a polypeptide that provides for resistance to a disease (by plant pathogen such as fungus or a bacterium) or for resistance to an insect (e.g., an insect that causes plant pathology). At a first temperature of from about 17° C. to about 25° C., the target nucleic acid is not modified by the Cas12L polypeptide; thus, the plant is resistant to the fungus, bacterium, or insect. At a second temperature of from about 28° C. to about 37° C. or from about 28° C. to about 40° C., the target nucleic acid is modified by the Cas12L polypeptide; thus, the plant is susceptible to the fungus, bacterium, or insect.

In other cases, the target nucleic acid is an endogenous nucleic acid or a transgene comprising a nucleotide sequence encoding a negative regulator of a disease resistance or insect resistance gene or pathway. At a first temperature of from about 17° C. to about 25° C., the target nucleic acid is not modified by the Cas12L polypeptide; thus, the plant is susceptible to the fungus, bacterium, or insect. At a second temperature of from about 28° C. to about 37° C. or from about 28° C. to about 40° C., the target nucleic acid is modified by the Cas12L polypeptide; thus, the polypeptide that provides for resistance is produced and the plant is resistant to the fungus, bacterium, or insect.

As another example, the modification results in expression of a transgene that confers resistance to an herbicide. For example, in some cases, the transgene is a nucleic acid comprising a nucleotide sequence encoding a polypeptide that confers resistance to an herbicide, such as an imidazolinone or a sulfonylurea, that inhibits the growing point or meristem; such polypeptides include, e.g., a mutant ALS or a mutant AHAS enzyme. As another example, in some cases, the transgene is a nucleic acid comprising a nucleotide sequence encoding a polypeptide that confers resistance to glyphosphate, e.g., where resistance can be conferred by a mutant 5-enolpyruvl-3-phosphikimate synthase gene (EPSP).

As another example, the modification controls male sterility/fertility. Examples include, e.g., a transgene that is a nucleic acid comprising a nucleotide sequence encoding barstar (an inhibitor of barnase), e.g., where the nucleotide sequence is operably linked to an anther-specific promoter or a pollen-specific promoter (see, e.g., Roque et al. (2019) Front. Plant Sci. 10:819); a a transgene that is a nucleic acid comprising a nucleotide sequence encoding barnase (Paul et al., (1992) Plant Mol. Biol. 19:611-622); and the like. Another example includes a transgene encoding a deacetylase gene under the control of a tapetum-specific promoter. Other male sterility genes include, e.g., MAC1, EMS1, and GNE2 (Sorensen et al. (2002) Plant J. 29:581-594). Further examples of male sterility genes include CMS-D2-2, CMS-hir, CMS-D8, CMS-D4, and CMS-C1.

In some cases, the target nucleic acid comprises a nucleotide sequence that encodes a male reproductive pathway polypeptide. At a first temperature of from about 17° C. to about 25° C., the target nucleic acid is not modified by the Cas12L polypeptide; thus, the plant is fertile. At a second temperature of from about 28° C. to about 37° C. or from about 28° C. to about 40° C., the target nucleic acid is modified by the Cas12L polypeptide; thus, the plant is male sterile.

In other cases, the target nucleic acid is an endogenous nucleic acid or a transgene comprising a nucleotide sequence encoding a negative regulator of the male reproductive pathway. At a first temperature of from about 17° C. to about 25° C., the target nucleic acid is not modified by the Cas12L polypeptide; thus, the male reproductive pathway is blocked, resulting in a male sterile phenotype. At a second temperature of from about 28° C. to about 37° C. or from about 28° C. to about 40° C., the target nucleic acid is modified by the Cas12L polypeptide; thus, the male reproductive pathway is allowed to function and the plant is fertile.

### Target Nucleic Acids

A Cas12L polypeptide can be targeted to a specific target nucleic acid to modify the target nucleic acid. As described above, Cas12L is targeted to a target nucleic acid based on its association/complex with a guide RNA that is able to hybridize with the particular target nucleotide sequence in the target nucleic acid. In this sense, the guide RNA provides the targeting functionality to target a particular target nucleotide sequence in a target nucleic acid. Various types of nucleic acids may be targeted to e.g. modulate their expression, as will be readily apparent to one of skill in the art.

Certain aspects of the present disclosure relate to targeting a target nucleic acid with a Cas12L polypeptide such that the Cas12L polypeptide is able to enact enzymatic activity at the target nucleic acid. In some cases, a Cas12L polypeptide/gRNA complex is targeted to a target nucleic acid and introduces an edit/modification into the target nucleic acid. In some cases, the edit/modification is to introduce a single-stranded break or a double stranded break into the nucleic acid backbone of the target nucleic acid.

Certain aspects of the present disclosure relate to target sites on target nucleic acids. A target site generally refers to a location of a target nucleic acid that is capable of being bound by a Cas12L/gRNA complex and subjected to the activity of a Cas12L polypeptide or variant thereof. In some cases, the target site may include both the nucleotide sequence hybridized with a guide RNA as well as at least 5, at least 10, at least 15, at least 20, at least 25, at least 30, at least 35, at least 40, at least 45, or at least 50 nucleotides or more on the 3′ side, the 5′ side, or both the 3′ and 5′ side of the nucleotide sequence in the target nucleic acid that is hybridized with a guide RNA. In some embodiments, the target site may contain at least 30, at least 40, at least 50, at least 60, at least 70, at least 80, at least 90, at least 100, at least 125, at least 150, at least 175, or at least 200 or more nucleotides.

In some cases, a Cas12L polypeptide is targeted to a particular locus. A locus generally refers to a specific position on a chromosome or other nucleic acid molecule. A locus may contain, for example, a polynucleotide that encodes a protein or an RNA. A locus may also contain, for example, a non-coding RNA, a gene, a promoter, a 5′ untranslated region (UTR), an exon, an intron, a 3′ UTR, or combinations thereof. In some cases, a locus may contain a coding region for a gene.

In some cases, a Cas12L polypeptide is targeted to a gene. A gene generally refers to a polynucleotide that encodes a gene product (for example, a polypeptide or a noncoding RNA). A gene may contain a promoter, an enhancer sequence, a leader sequence, a transcriptional start site, a transcriptional stop site, a polyadenylation site, one or more exons, one or more introns, a 5′ UTR, a 3′ UTR, or combinations thereof. A gene sequence may contain a polynucleotide sequence encoding a promoter, an enhancer sequence, a leader sequence, a transcriptional start site, a transcriptional stop site, a polyadenylation site, one or more exons, one or more introns, a 5′ UTR, a 3′ UTR, or combinations thereof.

The target nucleic acid sequence may be located within the coding region of a target gene or upstream or downstream thereof. Moreover, the target nucleic acid sequence may reside endogenously in a target gene or may be inserted into the gene, e.g., heterologous, for example, using techniques such as homologous recombination. For example, a target gene of the present disclosure can be operably linked to a control region, such as a promoter, that contains a sequence that can be recognized by a guide RNA of the present disclosure such that a Cas12L polypeptide may be targeted to that sequence. In some embodiments, the target sequence may be a promoter or other regulatory region.

The target nucleic acid sequence may be located in a region of chromatin. In some embodiments, the target nucleic acid sequence to be edited by a Cas12L polypeptide may be in a region of open chromatin or similar region of DNA that is generally accessible to transcriptional machinery. Regions of open chromatin may be characterized by nucleosome depletion, nucleosome disruption, accessibility to transcriptional machinery, and/or a transcriptionally active state. Regions of open chromatin will be readily understood and identifiable by one of skill in the art. Editing a target nucleic acid sequence that is in a region of open chromatin may result in improved editing efficiency by the Cas12L polypeptide as compared to a corresponding control nucleic acid sequence (e.g. one that is present in a region of more closed, repressive, and/or transcriptionally inactive chromatin).

Target genes or nucleic acid regions to be edited by a Cas12L polypeptide of the present disclosure will be readily apparent to those of skill in the art depending on the particular application and/or purpose. For example, genes with particular agricultural importance may be edited/modified according to the methods of the present disclosure. Exemplary genes to be edited/modified may include, for example, those involved in light perception (e.g. PHYB, etc.); those involved in the circadian clock (e.g. CCA1, LHY, etc.); those involved in flowering time (e.g. CO, FT, etc.); those involved in meristem size (e.g. WUS, CLV3, etc.); those involved in plant architecture (S, SP, TFL1, SFT, etc.); those involved in ripening (e.g., genes in the ethylene production pathway); those involved in flower color; those involved in bract color; and those involved in embryogenesis, chromatin structure, stress response, growth and development, etc.

In some cases, the target nucleic acid is one that provides for resistance to an antimicrobial agent. Examples of such antimicrobial agents include penicillin, a cephalosporin, a monobactam, a carbapenem, a macrolide, an aminoglycoside, a quinolone, a sulfonamide, a tetracycline, a glycopeptide, a lipoglycopeptide, an oxazolidinone, a rifamycin, a tuberactinomycin, chloramphenicol, metronidazole, tinidazole, nitrofurantoin, teicoplanin, telavancin, linezolid, cycloserine 2, bacitracin, polymyxin B, viomycin, and capreomycin. In some cases, the target nucleic acid is one that provides for resistance to an antifungal agent, where examples of antifungal agents include an allylamine, an imidazole, a triazole, a thiazole, a polyene, and an echinocandin. In some cases, the target nucleic acid is one that provides for resistance to an insecticidal agent, where examples of insecticidal agents include a chloronicotinyl, a neonicotinoid, a carbamate, an organophosphate, a pyrethroid, an oxadiazine, a spinosyn, a cyclodiene, an organochlorine, a fiprole, a mectin, a diacylhydrazine, a benzoylurea, an organotin, a pyrrole, a dinitroterpenol, a METI, a tetronic acid, a tetramic acid, and a pthalamide.

In some cases, the target nucleic acid provides for resistance to a plant pathogen. In some cases, the plant pathogen is a bacterium, a fungus, a parasitic insect, a parasitic nematode, or a parasitic protozoan.

In some cases, the target nucleic acid is endogenous to the plant where the expression of one or more genes is modulated according to the methods described herein. In some cases, the target nucleic acid is a transgene of interest that has been inserted into a plant. Suitable target nucleic acids will be readily apparent to one of skill in the art depending on the particular need or outcome. The target nucleic acid sequence may be in e.g. a region of euchromatin (e.g. highly expressed gene), or the target nucleic acid sequence may be in a region of heterochromatin (e.g. centromere DNA).

In some cases, the target nucleic acid may be in a region of repressive chromatin. Repressive chromatin generally refers to regions of chromatin where transcription is repressed or otherwise generally transcriptionally inactive. Exemplary regions of repressive chromatin include, for example, regions with repressive DNA methylation, compact chromatin, and/or no transcription).

In some cases, a Cas12L polypeptide can be used to create mutations in plants that result in reduced or silenced expression of a target gene. In some cases, a Cas12L polypeptide can be used to create functional “overexpression” mutations in a plant by releasing repression of the target gene expression as a consequence of a modification that results in transcriptional activation of the target nucleic acid. Release of gene expression repression, which may lead to activation of gene expression, may be of a structural gene, e.g., one encoding a protein having for example enzymatic activity, or of a regulatory gene, e.g., one encoding a protein that in turn regulates expression of a structural gene.

In some cases, a Cas12L polypeptide can be used to control an endogenous biosynthetic pathway in a plant cell. In some cases, a Cas12L polypeptide can be used to control a heterologous biosynthetic pathway in a plant cell. Examples of biosynthetic pathways that can be controlled using a Cas12L polypeptide (together with a Cas12L guide nucleic acid) include, e.g., biosynthetic pathways involved in psychoactive alkaloid production (e.g., for reducing opium production by Papaver soniferum); biosynthetic pathways for production of cannabidiol; biosynthetic pathways for production of tetrahydrocannabinol; a phytic acid production pathway; and the like.

In some cases, a Cas12L polypeptide is used to control an endogenous glucosinolate production pathway. In some cases, the Cas12L polypeptide inhibits an endogenous glucosinolate production pathway, but only at a higher temperature (e.g., from about 25C to about 32C), where such higher temperature, and only just prior to (e.g., one week, two weeks, or three weeks) harvest of a vegetable intended for human consumption, where the vegetable is produced by the plant.

### Cas12L Effector Polypeptides

Certain aspects of the present disclosure relate to Cas12L polypeptides and their use in facilitating the editing/modification of a target nucleic acid. Cas12L polypeptides generally function as RNA-guided DNA-binding proteins. Cas12L polypeptides may have endonuclease activity which can facilitate modification/editing of a target nucleic acid.

A Cas12L polypeptide (this term is used interchangeably with the term “Cas12L protein”) can bind and/or modify (e.g., cleave, nick, methylate, demethylate, etc.) a target nucleic acid and/or a polypeptide associated with target nucleic acid (e.g., methylation or acetylation of a histone tail) (e.g., in some cases, the Cas12L protein includes a fusion partner with an activity, and in some cases, the Cas12L protein provides nuclease activity). In some cases, the Cas12L protein is a naturally-occurring protein (e.g., naturally occurs in bacteriophage). In other cases, the Cas12L protein is not a naturally-occurring polypeptide (e.g., the Cas12L protein is a variant Cas12L protein, a fusion Cas12L protein, and the like).

Assays to determine whether given protein interacts with a Cas12L guide RNA can be any convenient binding assay that tests for binding between a protein and a nucleic acid. Suitable binding assays (e.g., gel shift assays) will be known to one of ordinary skill in the art (e.g., assays that include adding a Cas12L guide RNA and a protein to a target nucleic acid). Assays to determine whether a protein has an activity (e.g., to determine if the protein has nuclease activity that cleaves a target nucleic acid and/or some heterologous activity) can be any convenient assay (e.g., any convenient nucleic acid cleavage assay that tests for nucleic acid cleavage). Suitable assays (e.g., cleavage assays) will be known to one of ordinary skill in the art.

A naturally occurring Cas12L protein functions as an endonuclease that catalyzes a double strand break at a specific sequence in a targeted double stranded DNA (dsDNA). The sequence specificity is provided by the associated guide RNA, which hybridizes to a target sequence within the target DNA. The naturally occurring Cas12L guide RNA is a crRNA, where the crRNA includes (i) a guide sequence that hybridizes to a target sequence in the target DNA and (ii) a protein binding segment which includes a stem-loop (hairpin—dsRNA duplex) that binds to the Cas12L protein.

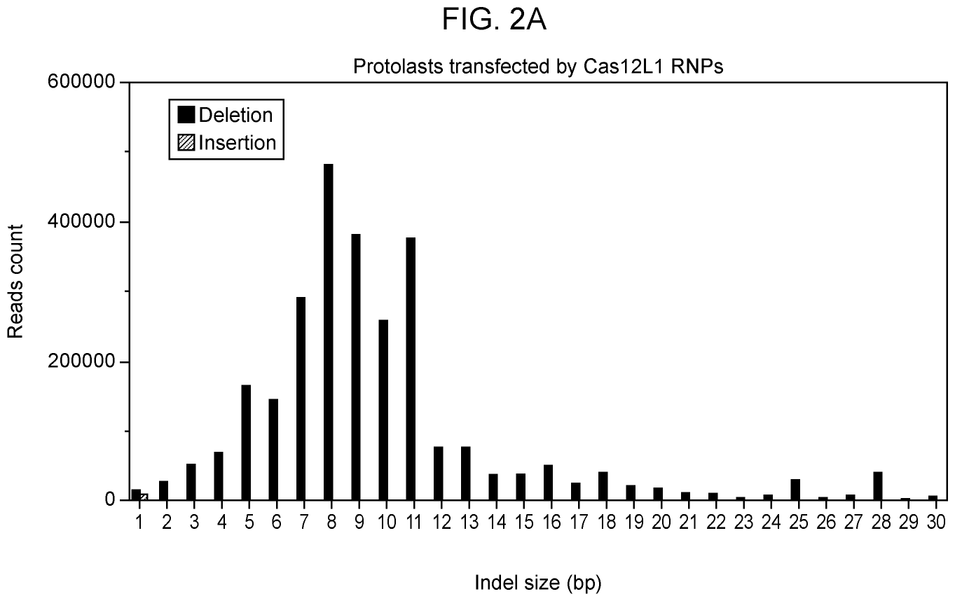

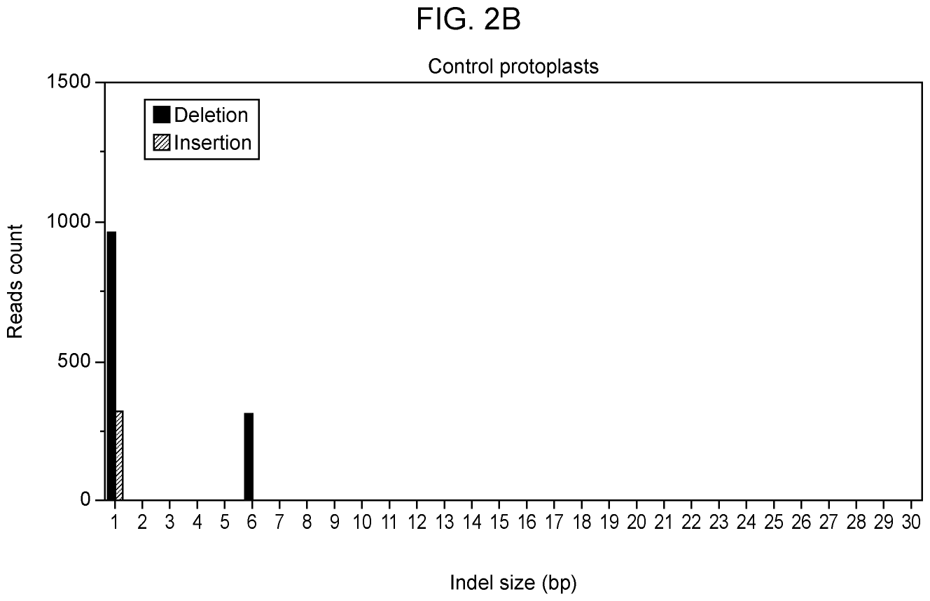

In some cases, a Cas12L polypeptide suitable for use in a subject method and/or composition is (or is derived from) a naturally occurring (wild type) protein. Examples of naturally occurring Cas12L proteins are depicted in FIG. 4A-4Z, FIG. 4AA-4ZZ, and FIG. 4AAA-4FFF. In some cases, a Cas12L protein (of the subject compositions and/or methods) includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with any one of the Cas12L amino acid sequences depicted in FIG. 4 (e.g., any one of FIG. 4A-4Z, FIG. 4AA-4ZZ, and FIG. 4AAA-4FFF). In some cases, a FIG. 4A-4Z, FIG. 4AA-4ZZ, and FIG. 4AAA-4FFF protein (of the subject compositions and/or methods) includes an amino acid sequence depicted in FIG. 2 (e.g., any one of FIG. 4A-4Z, FIG. 4AA-4ZZ, and FIG. 4AAA-4FFF).

In some cases, a Cas12L protein (of the subject compositions and/or methods) has more sequence identity to an amino acid sequence depicted in FIG. 4 (e.g., any one of FIG. 4A-4Z, FIG. 4AA-4ZZ, and FIG. 4AAA-4FFF) than to any of the following: Cas12a proteins, Cas12b proteins, Cas12c proteins, Cas12d proteins, Cas12e proteins, Cas12 g proteins, Cas12h proteins, and Cas12i proteins. In some cases, a Cas12L protein (of the subject compositions and/or methods) includes an amino acid sequence having a RuvC domain (which includes the RuvC-I, RuvC-II, and RuvC-III domains) that has more sequence identity to the RuvC domain of an amino acid sequence depicted in FIG. 2 (e.g., the RuvC domain of any of the Cas12L amino acid sequences depicted in FIG. 2) than to the RuvC domain of any of the following: Cas12a proteins, Cas12b proteins, Cas12c proteins, Cas12d proteins, Cas12e proteins, Cas12 g proteins, Cas12h proteins, and Cas12i proteins.

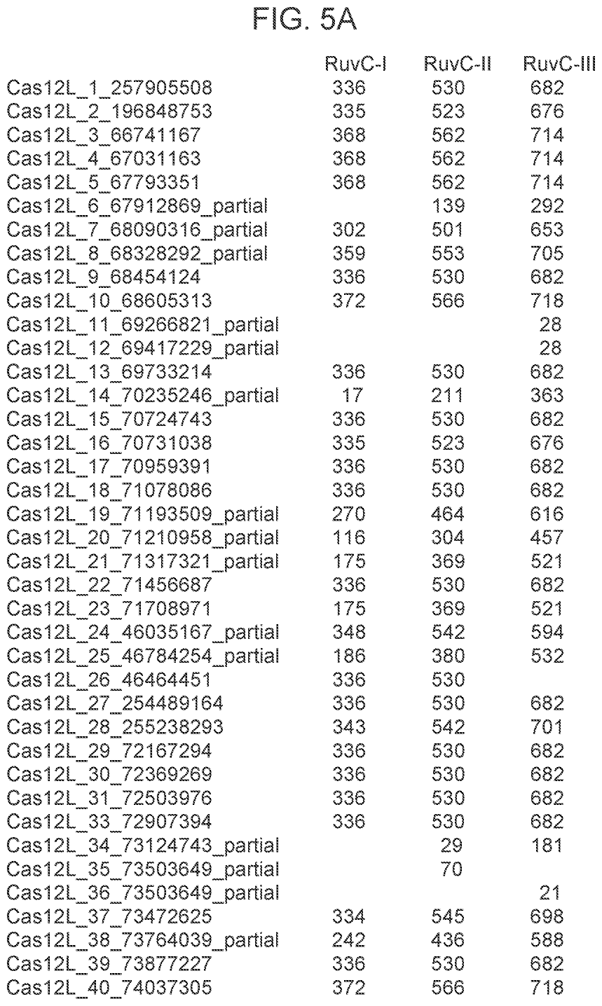

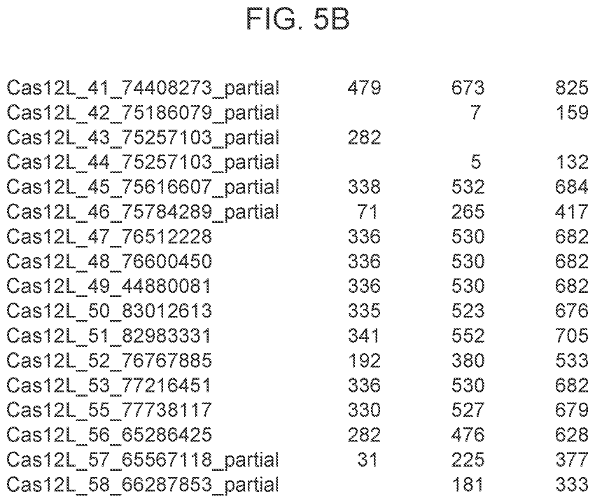

FIG. 5A-5B provides the locations of active site residues present in RuvC domains of various Cas12L polypeptides depicted in FIG. 4A-4Z, FIG. 4AA-4ZZ, and FIG. 4AAA-4FFF. For example, active site residues of Cas12L_1_257905508 (FIG. 4A) are amino acid residues 336, 530, and 682.

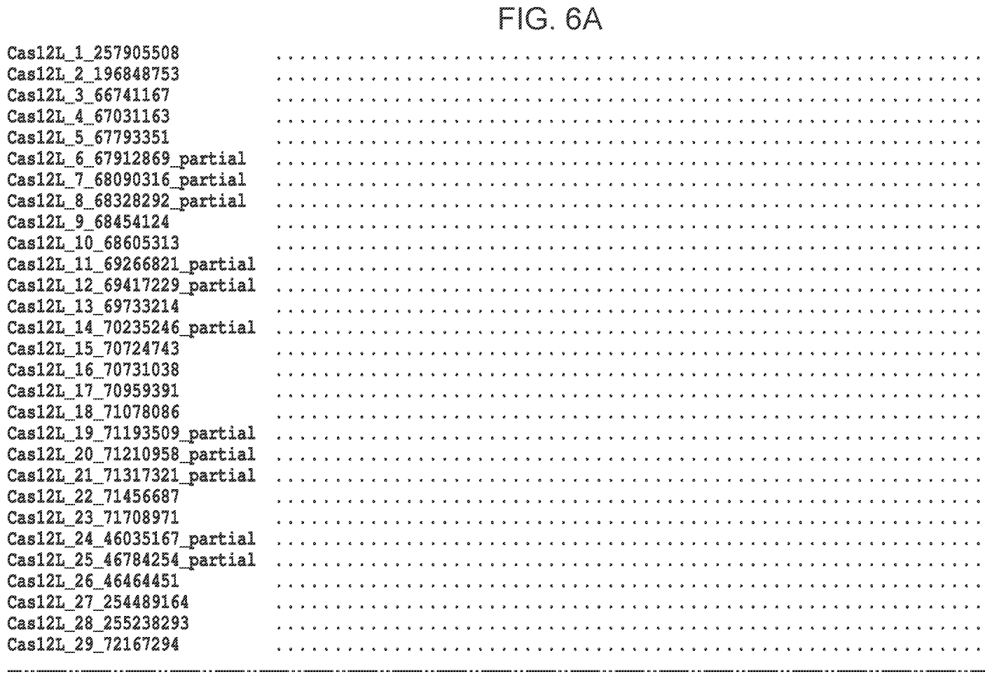

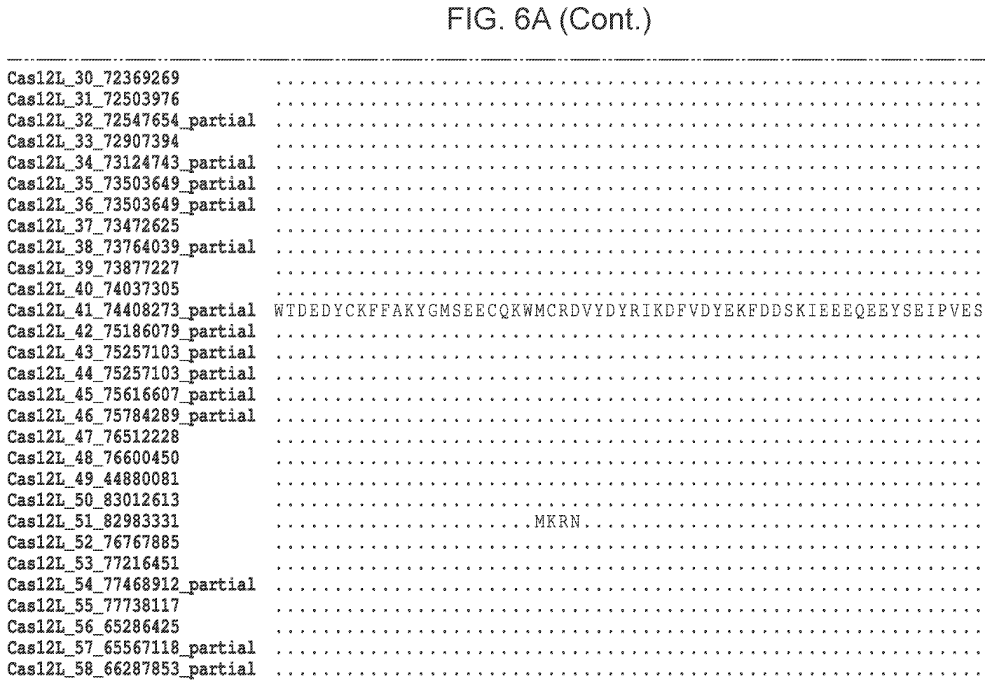

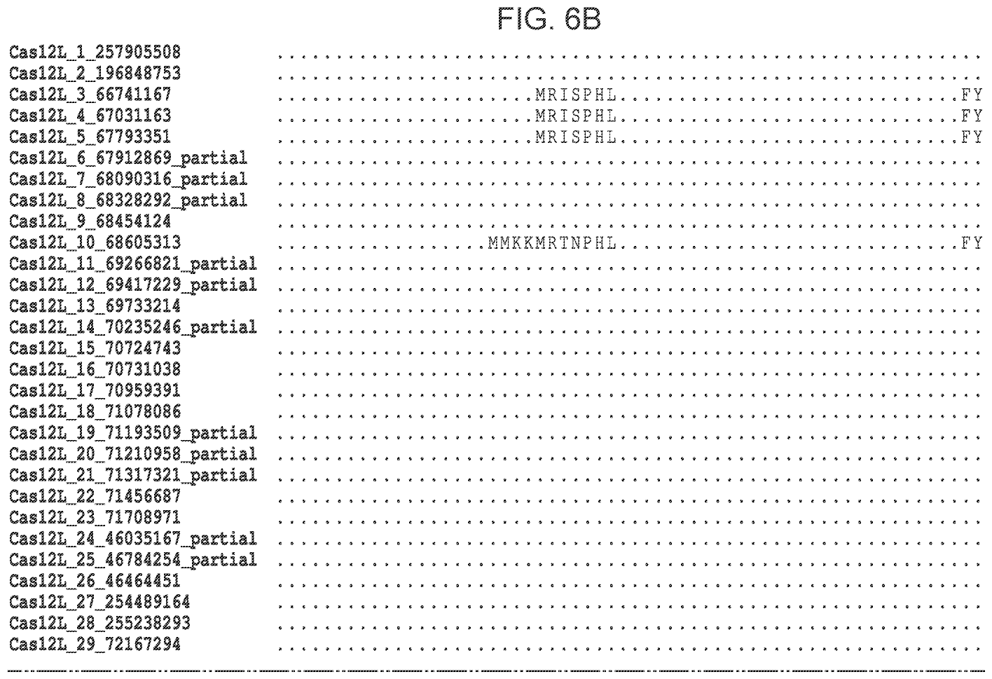

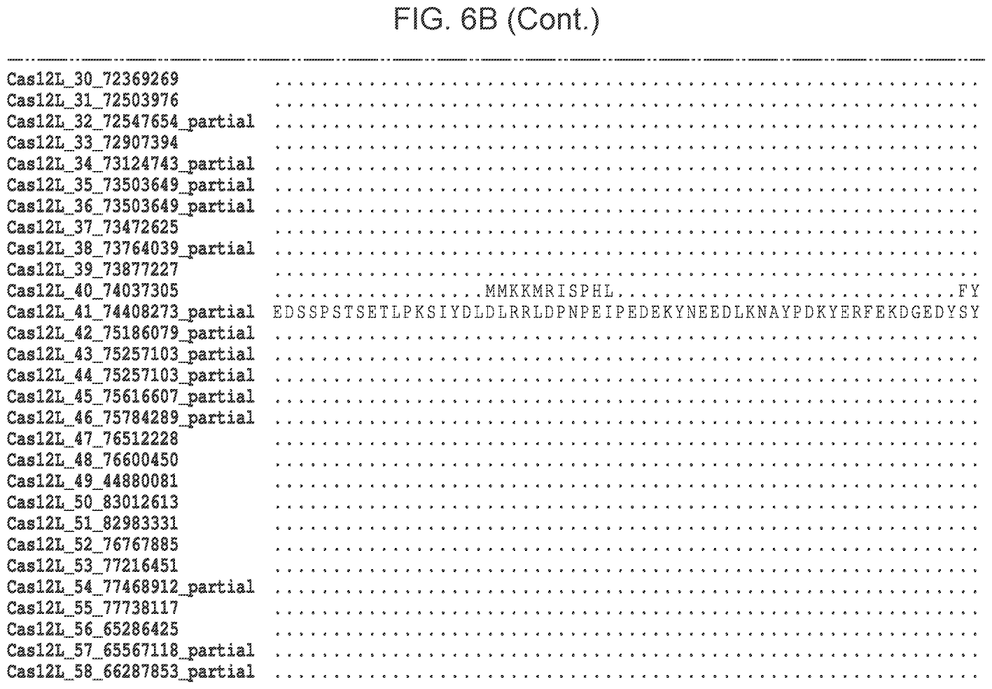

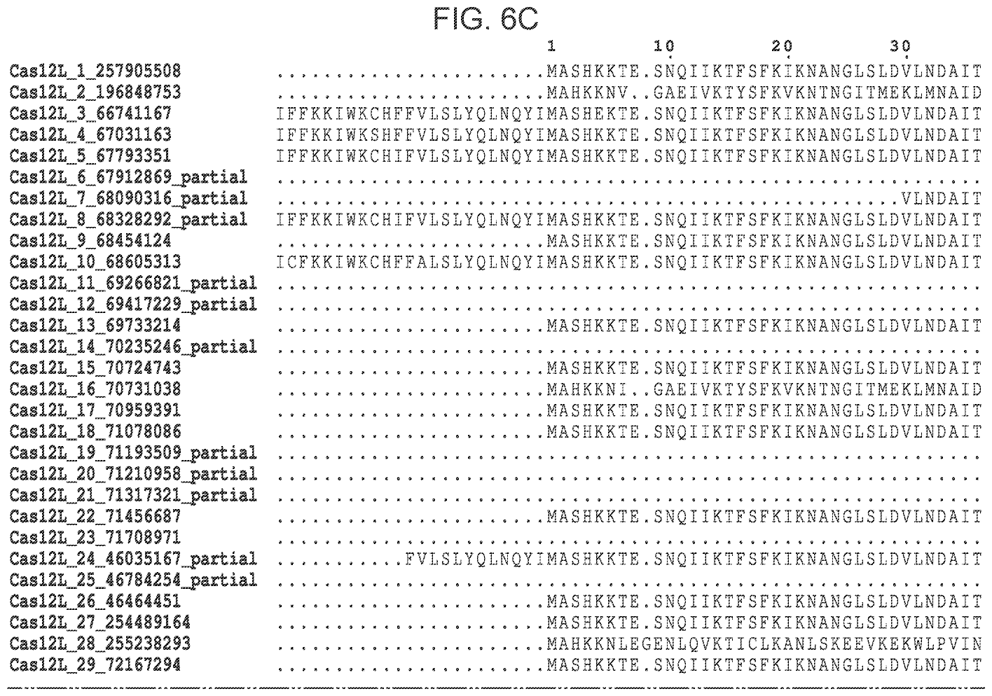

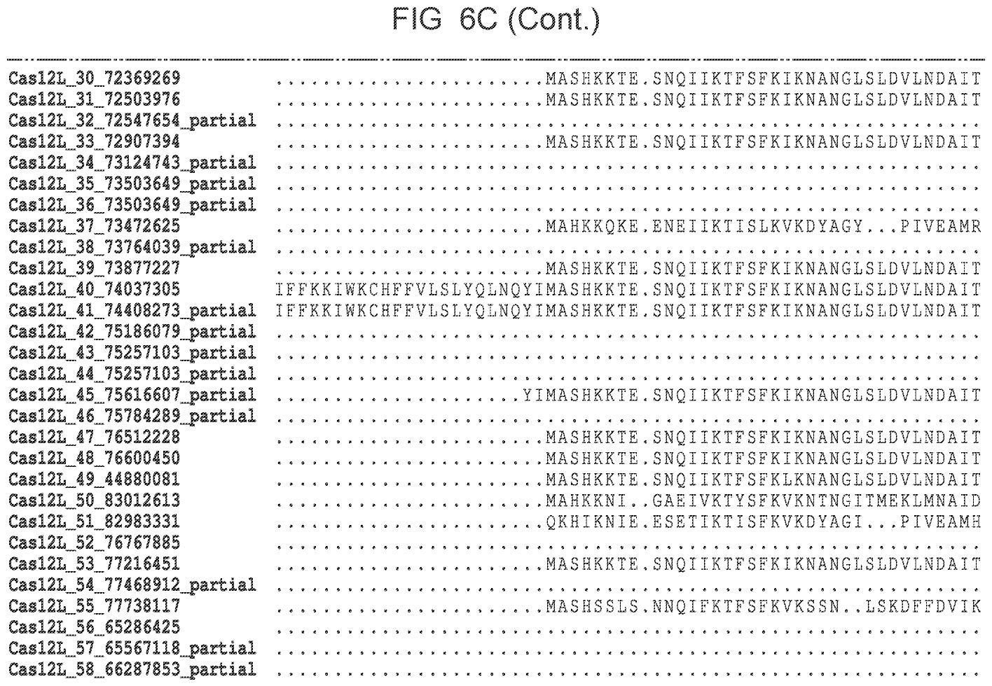

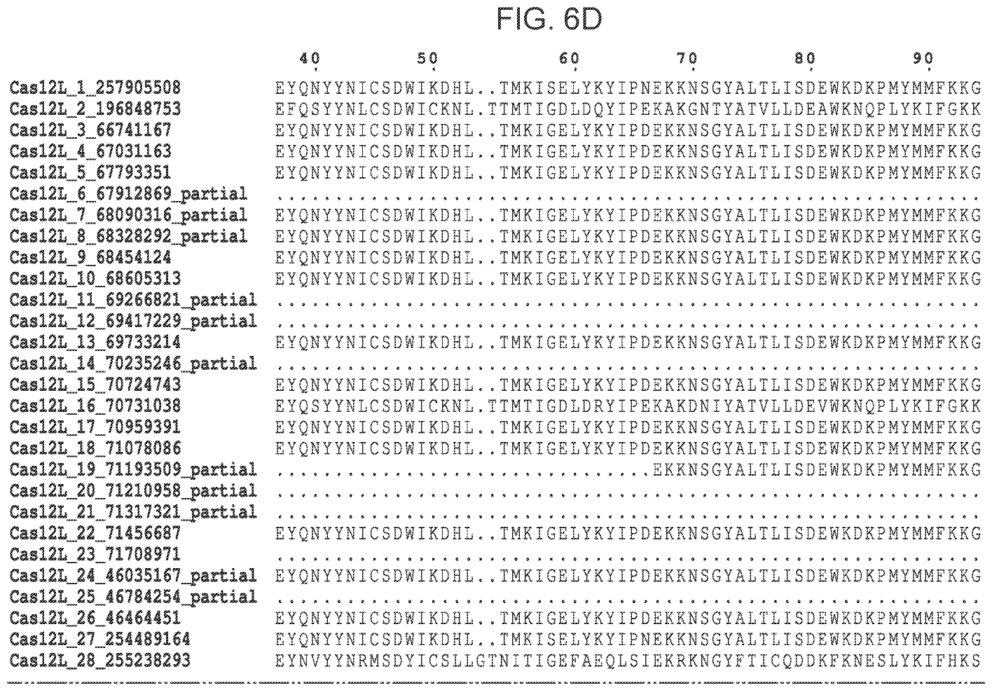

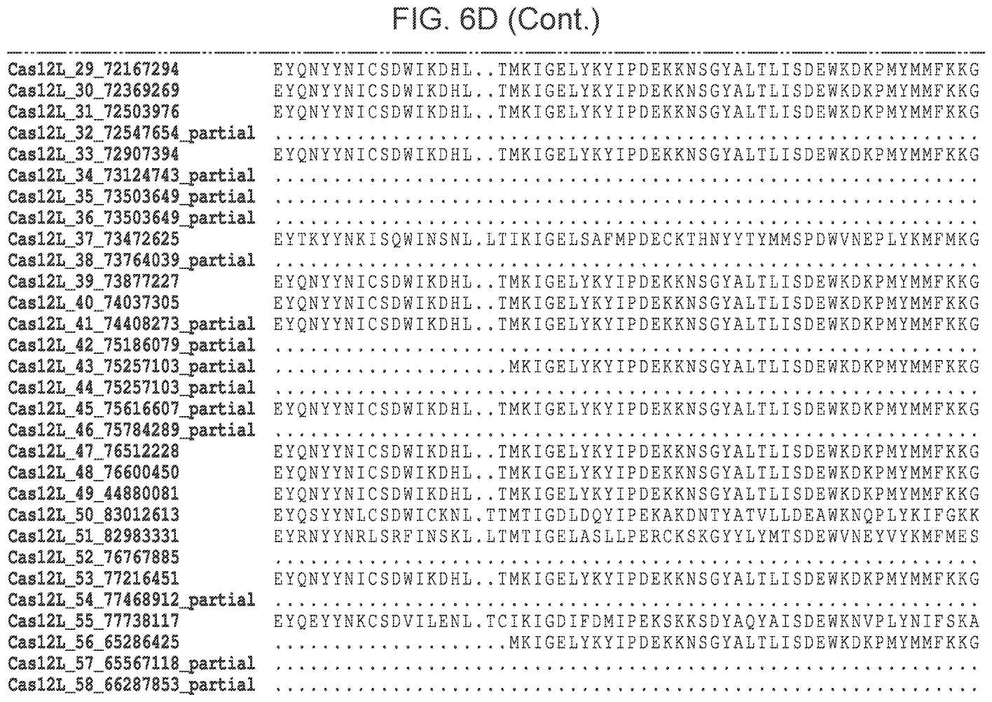

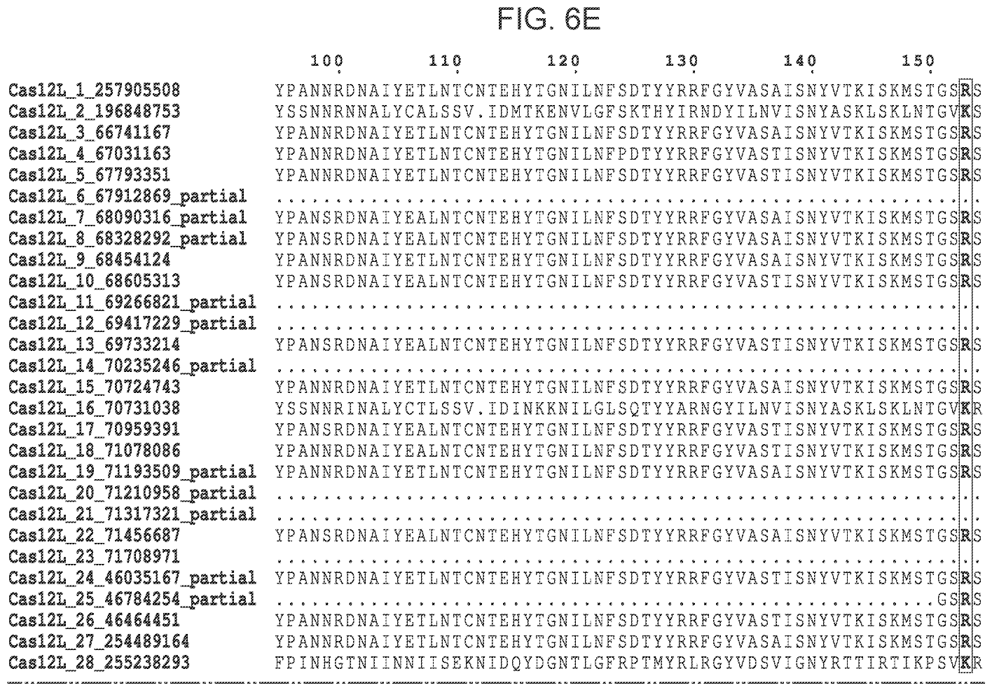

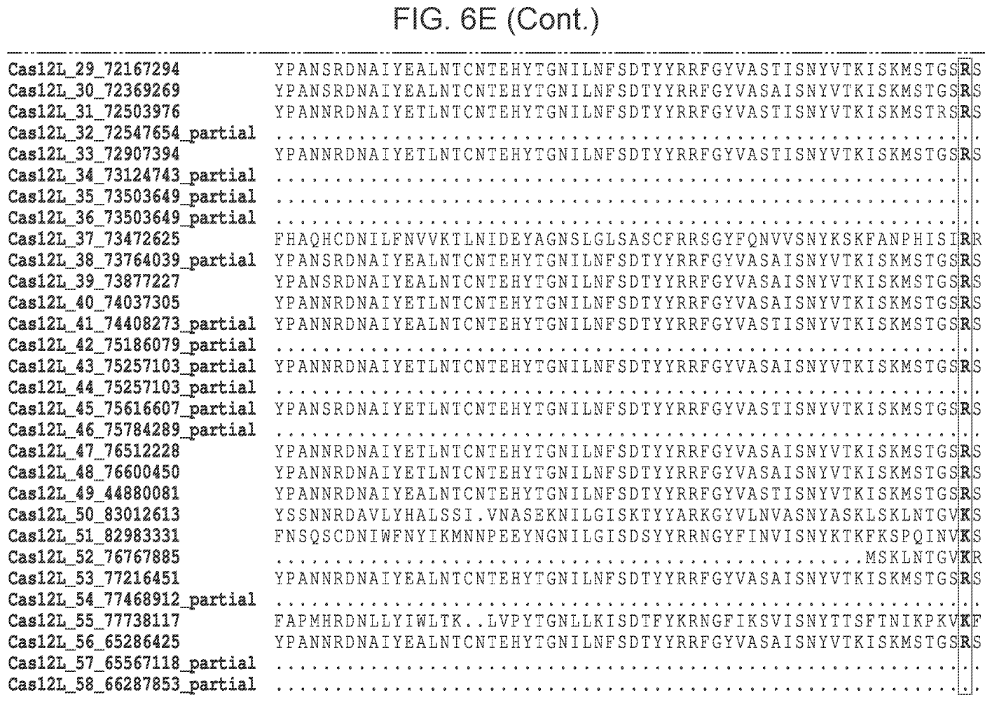

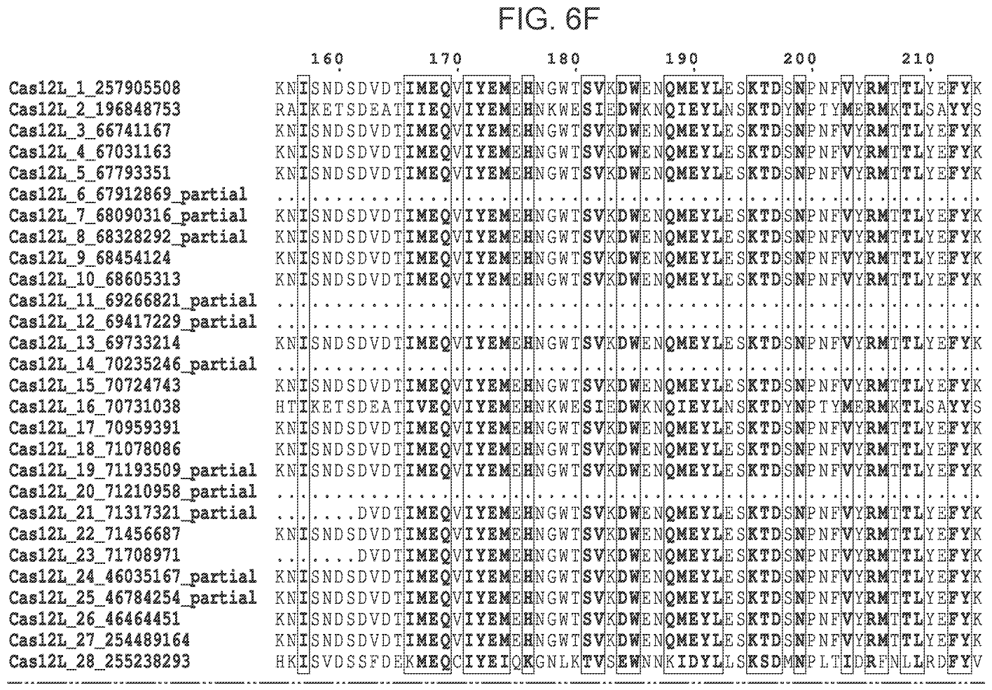

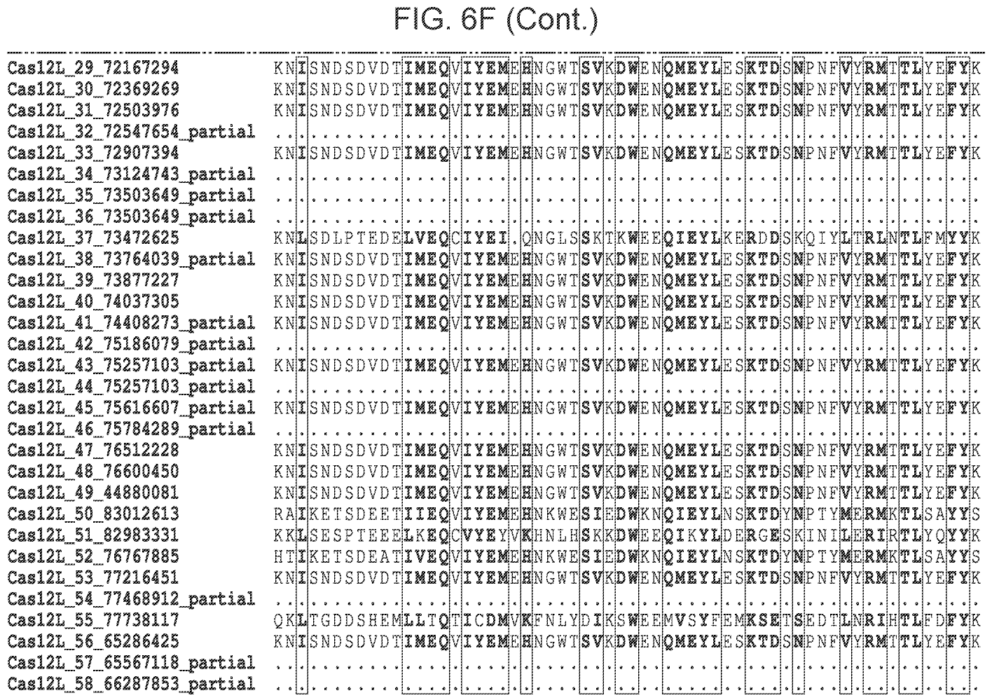

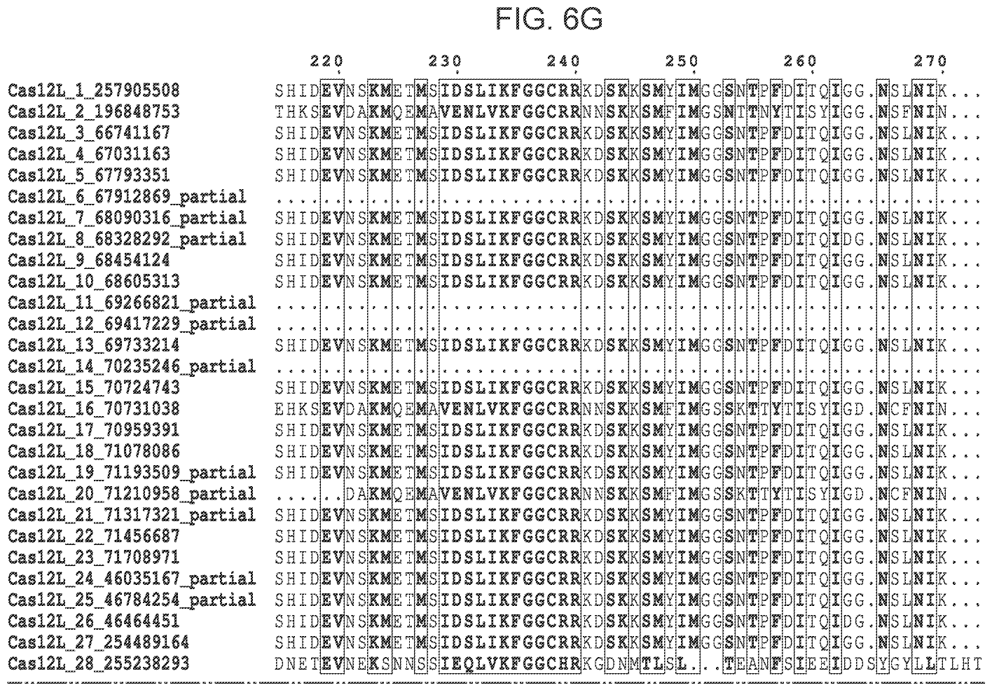

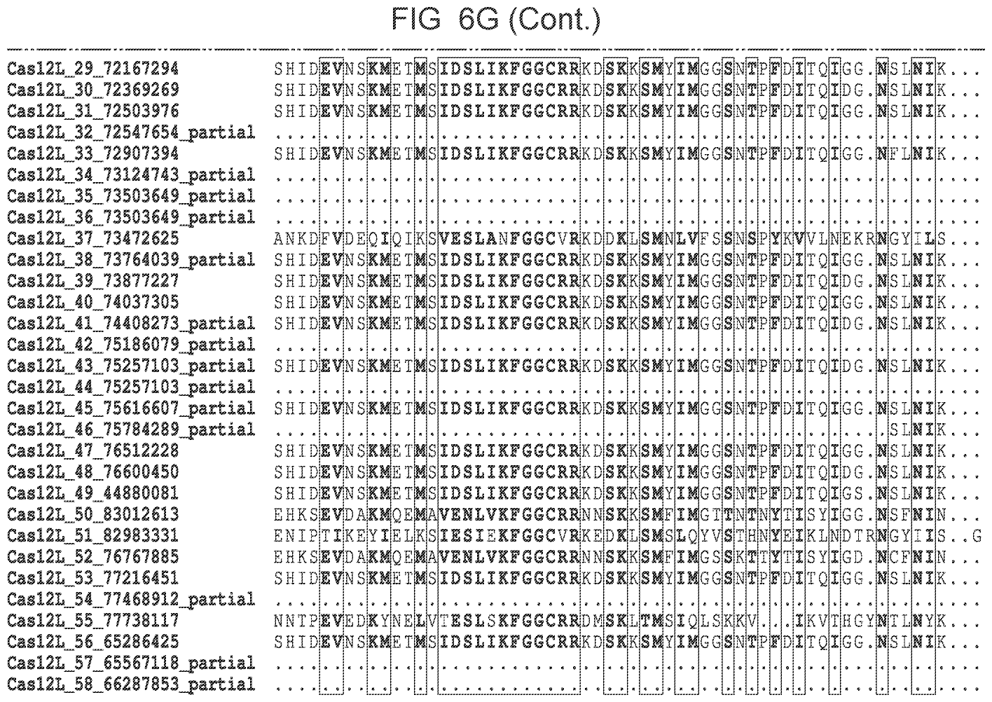

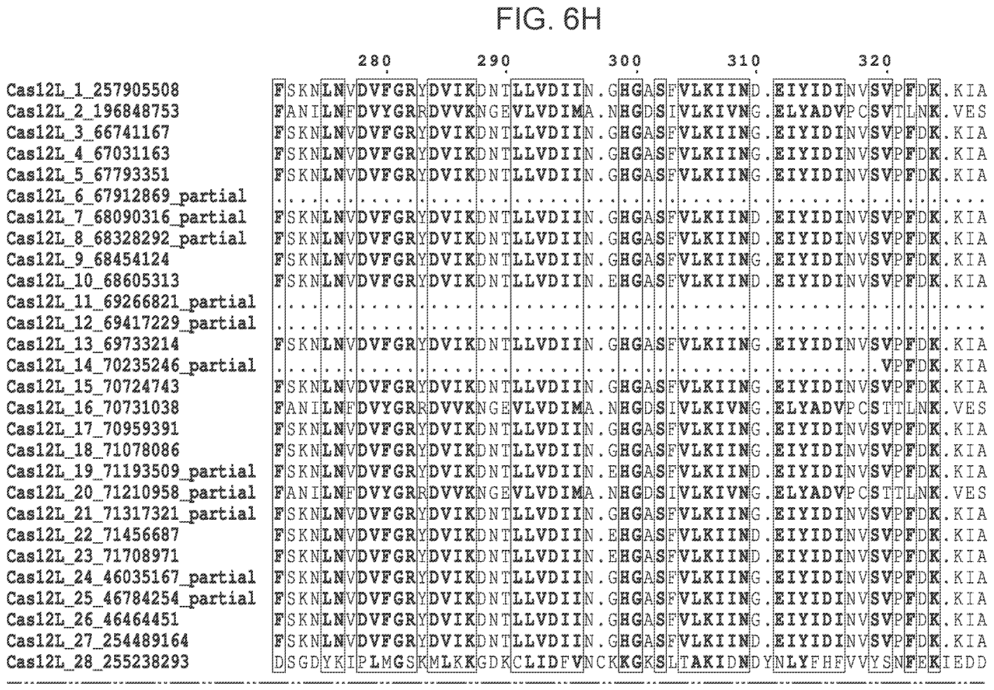

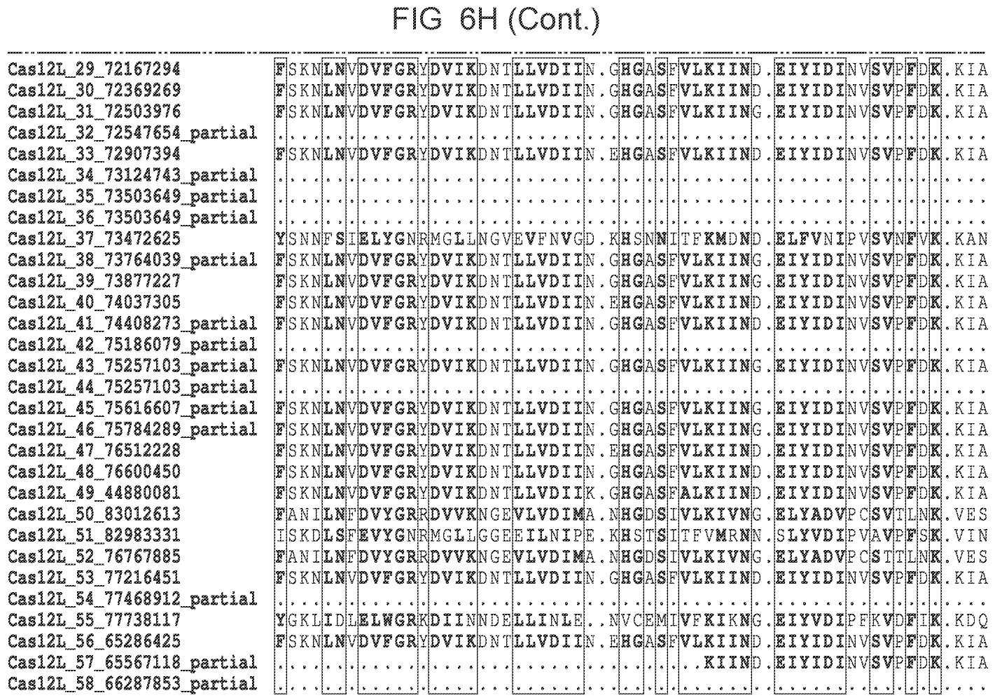

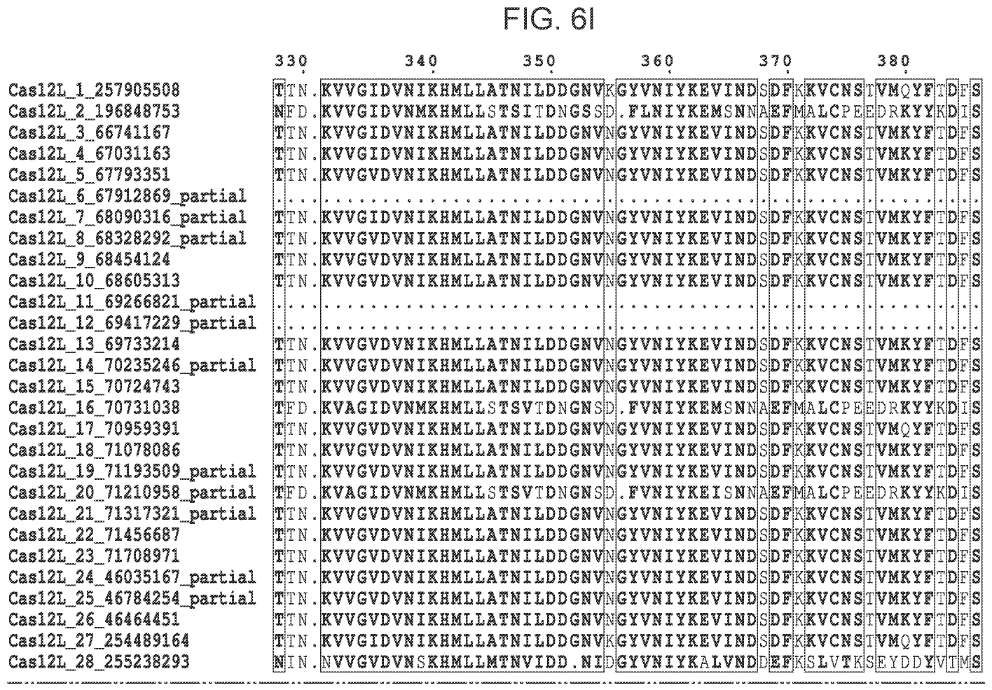

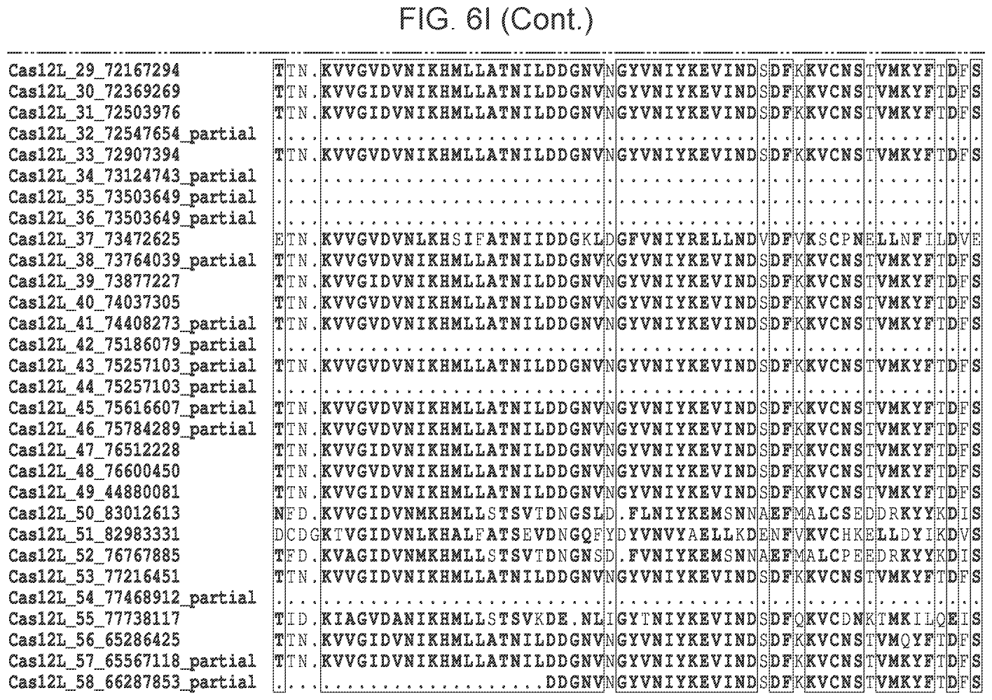

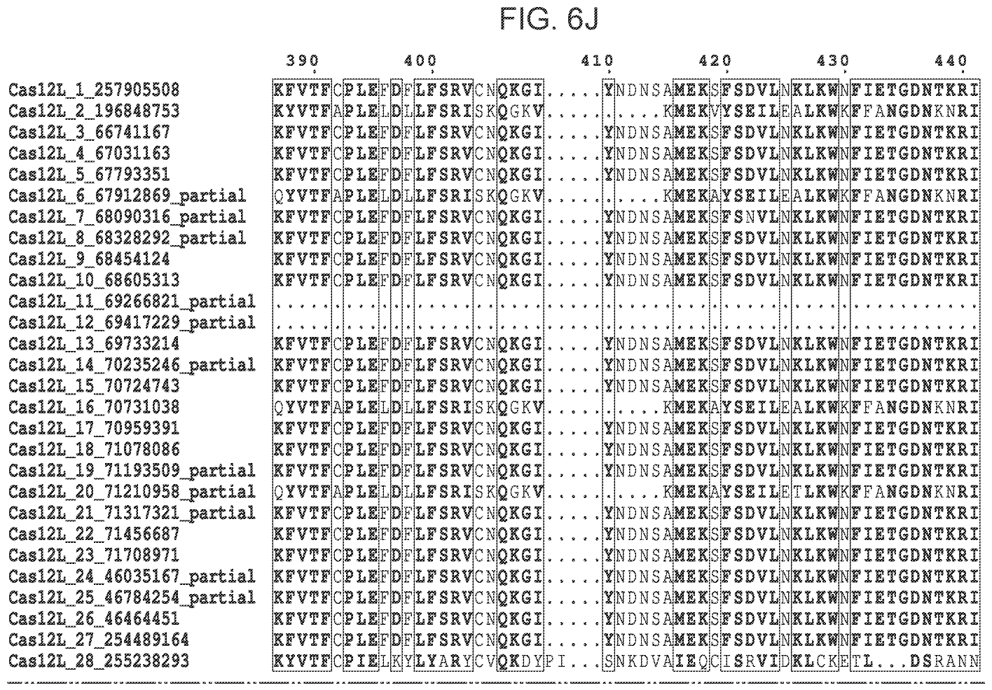

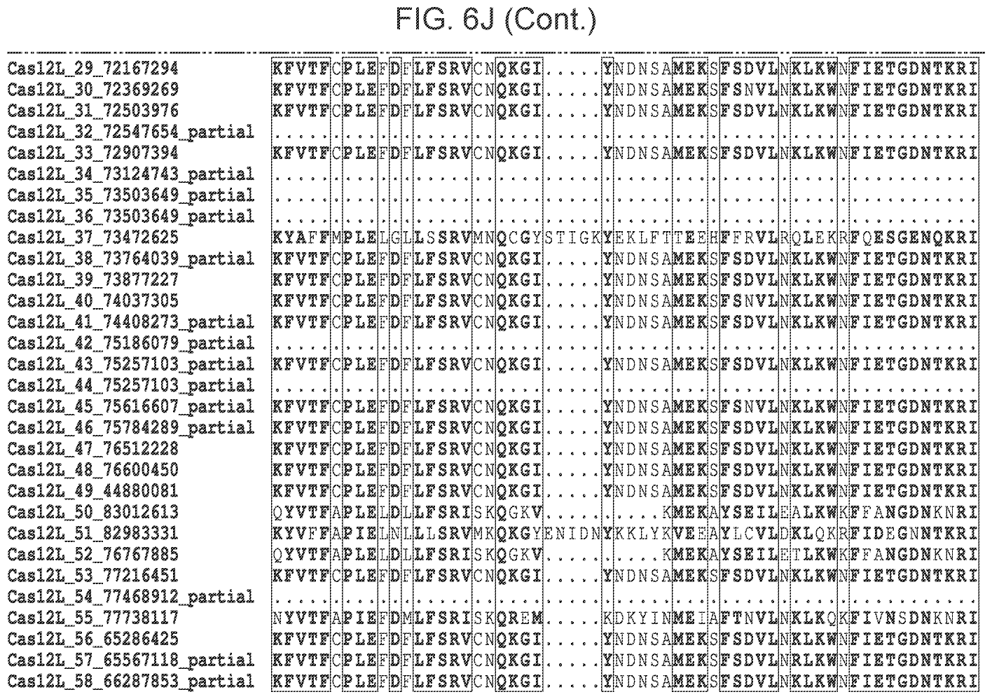

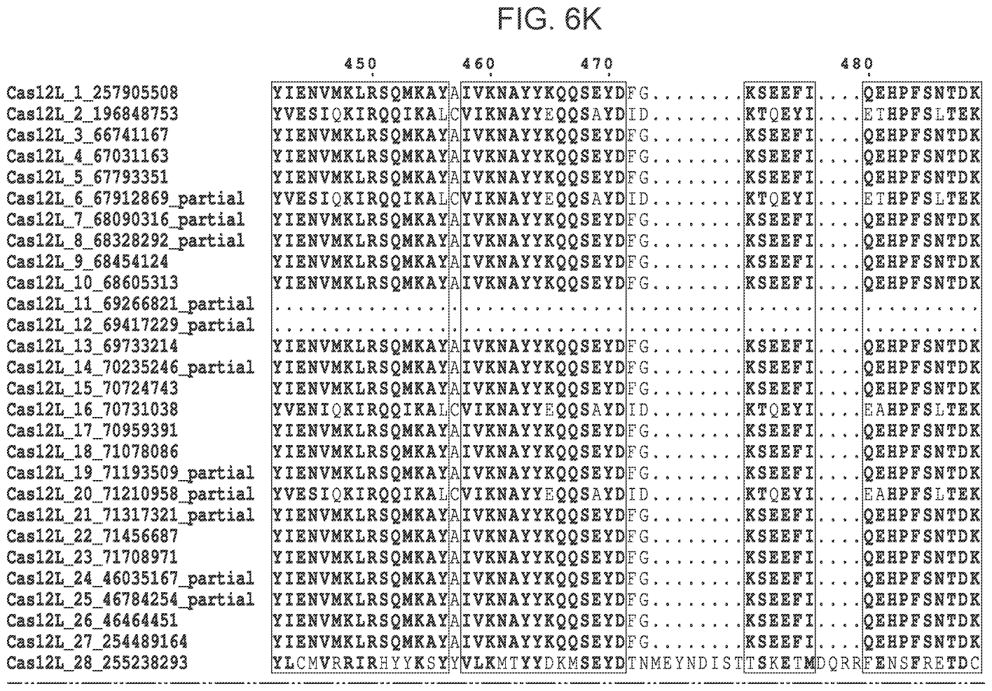

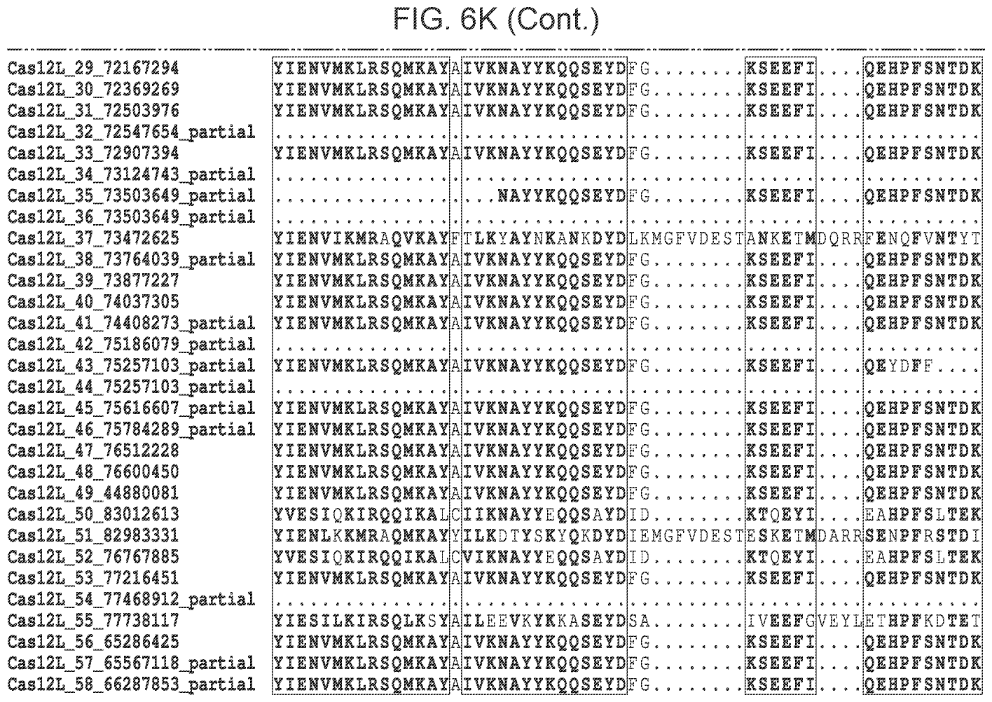

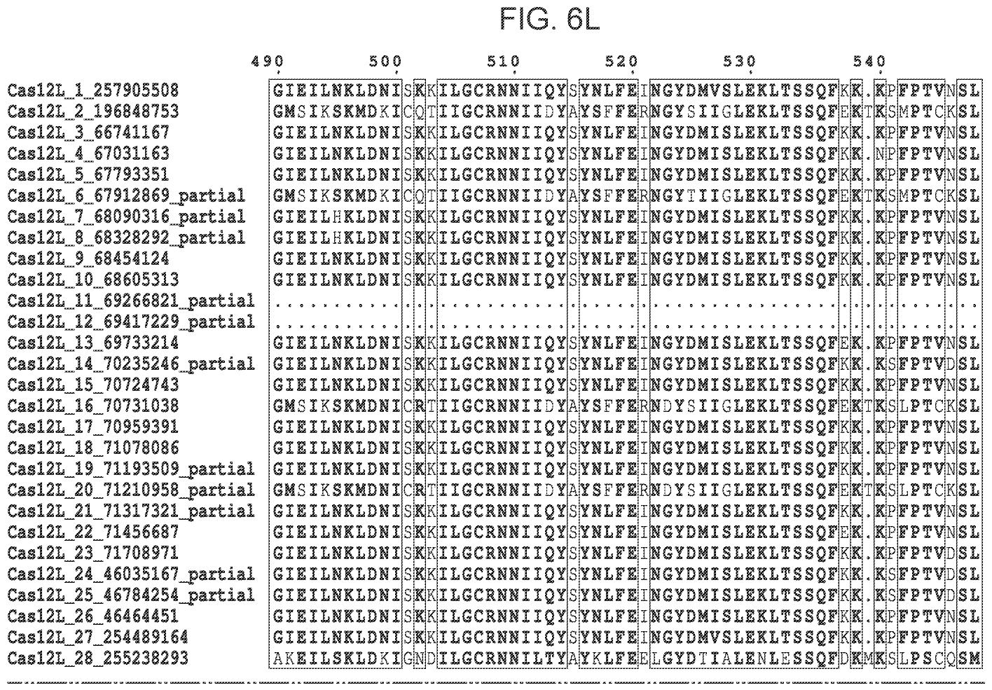

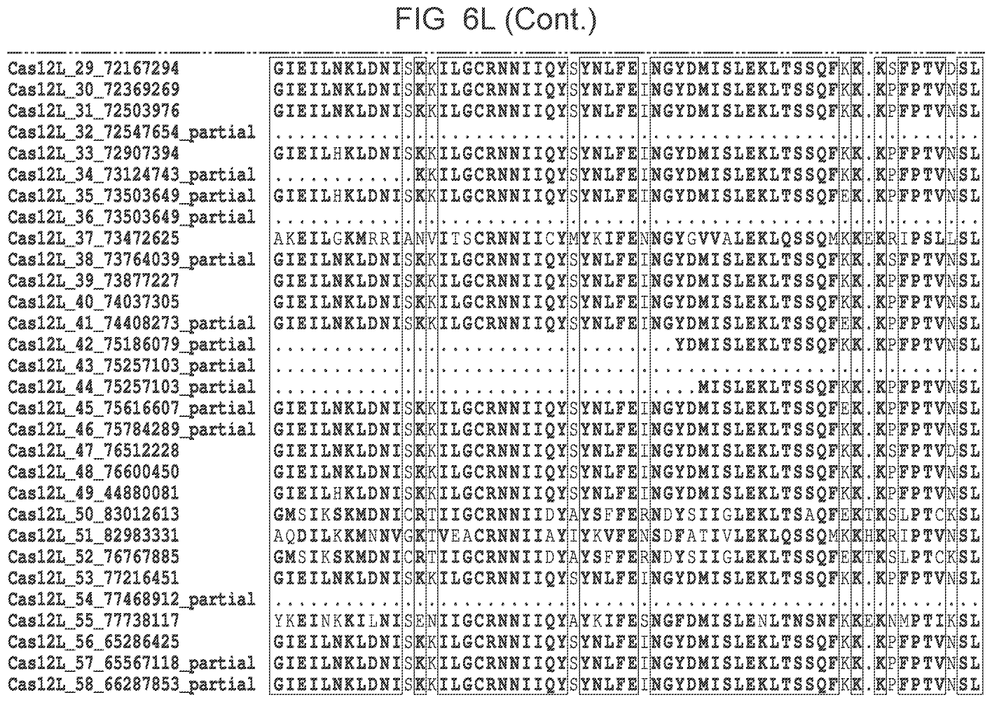

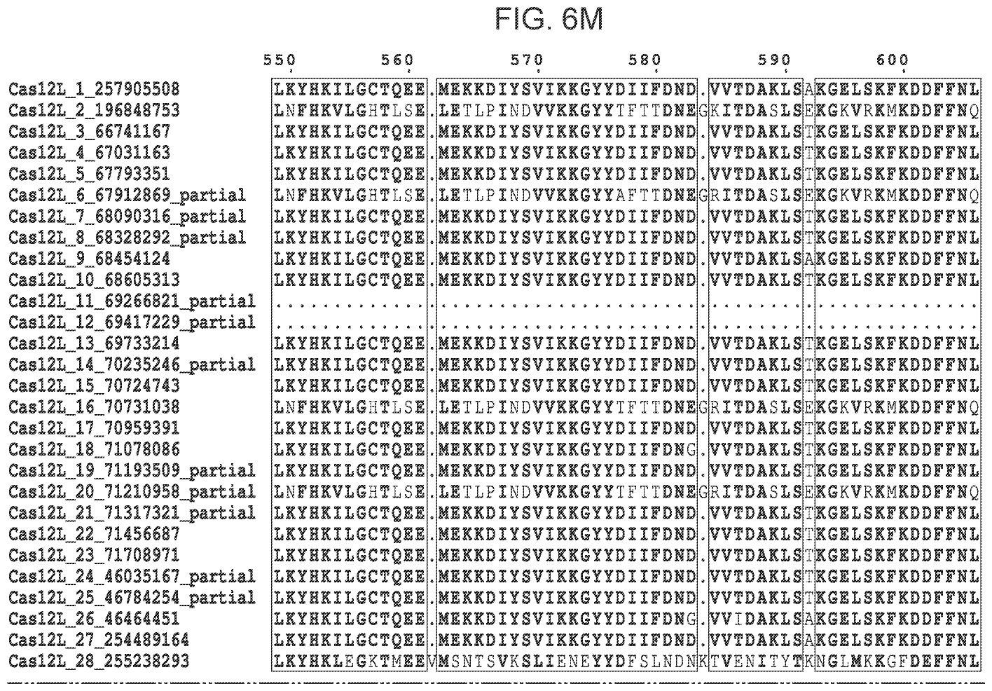

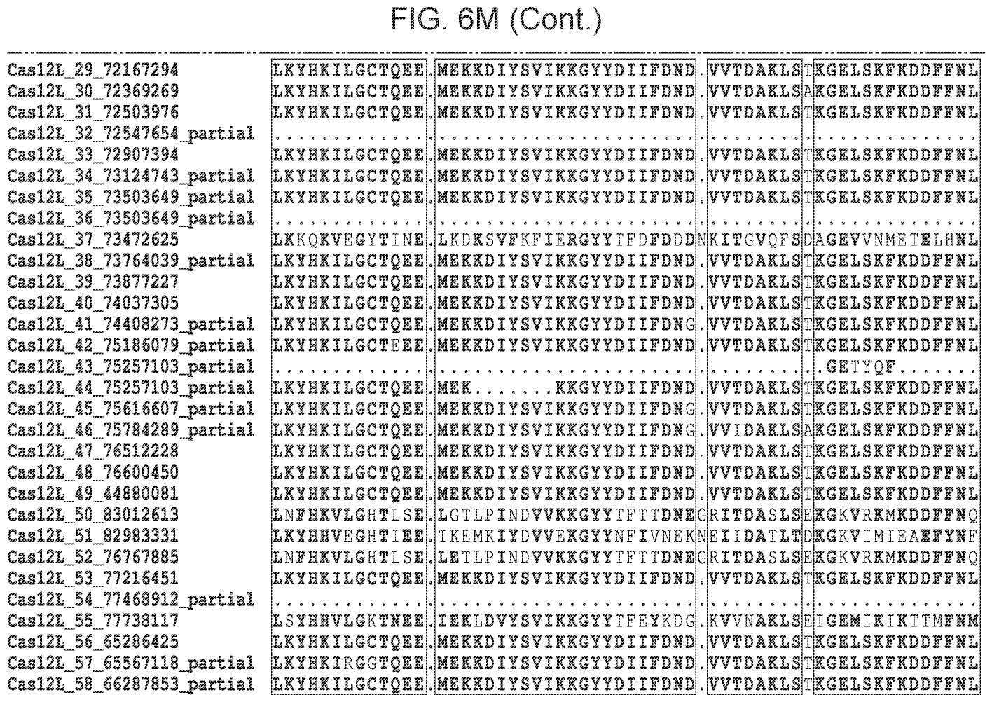

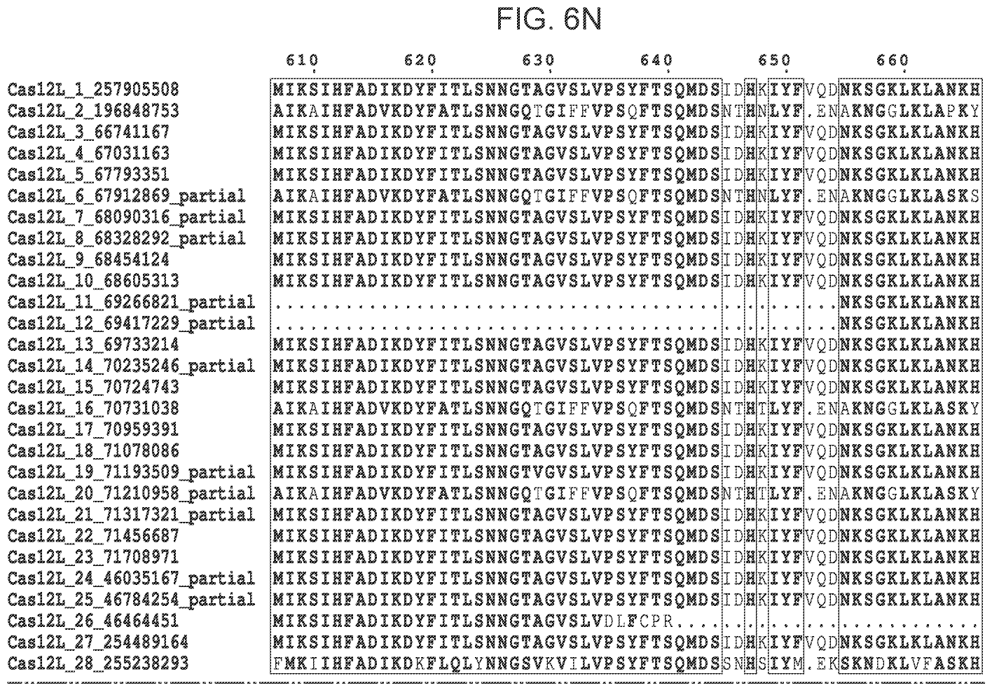

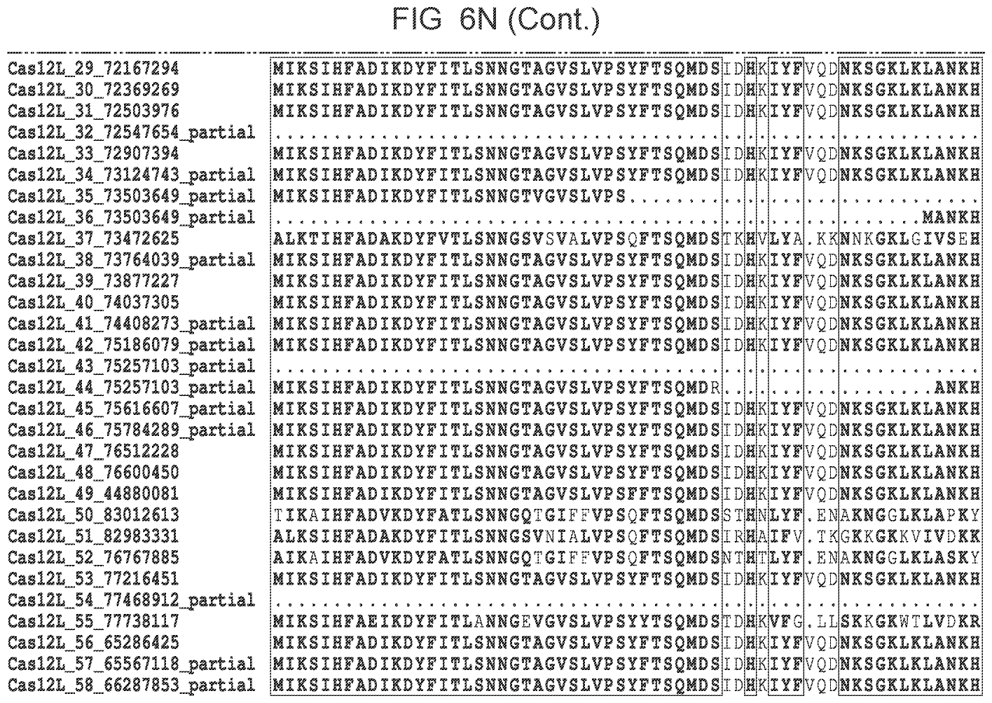

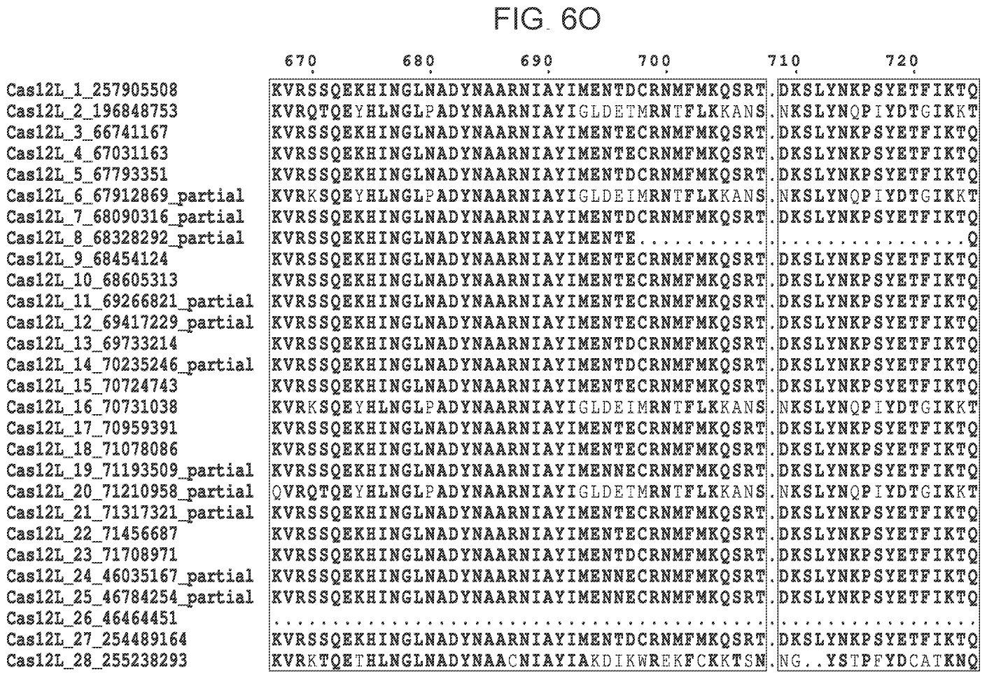

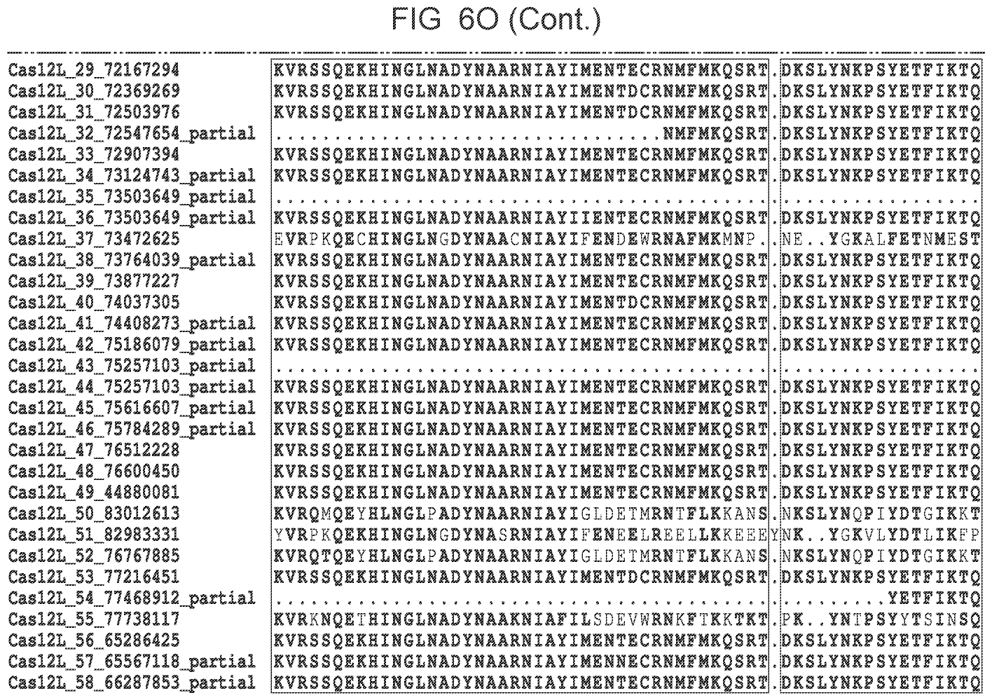

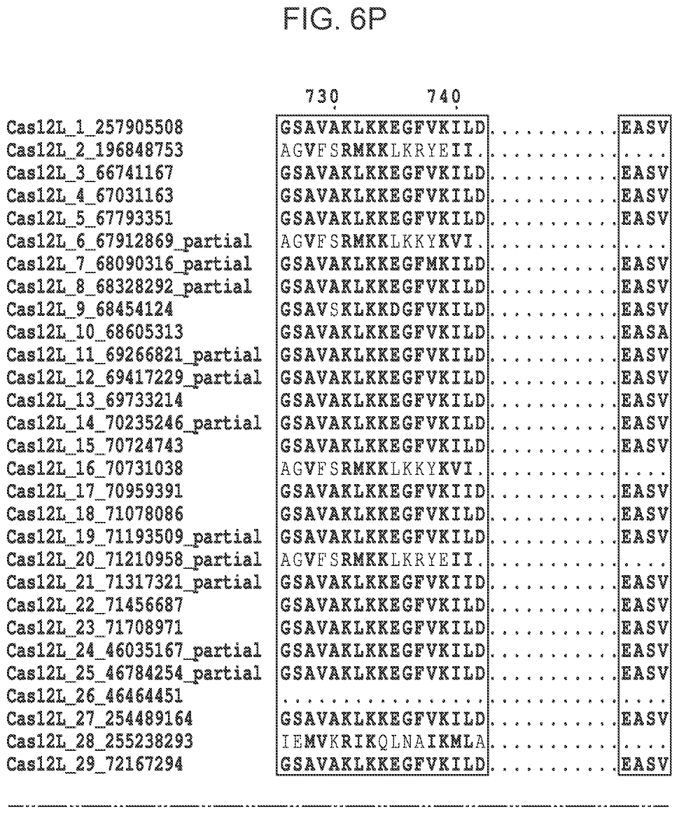

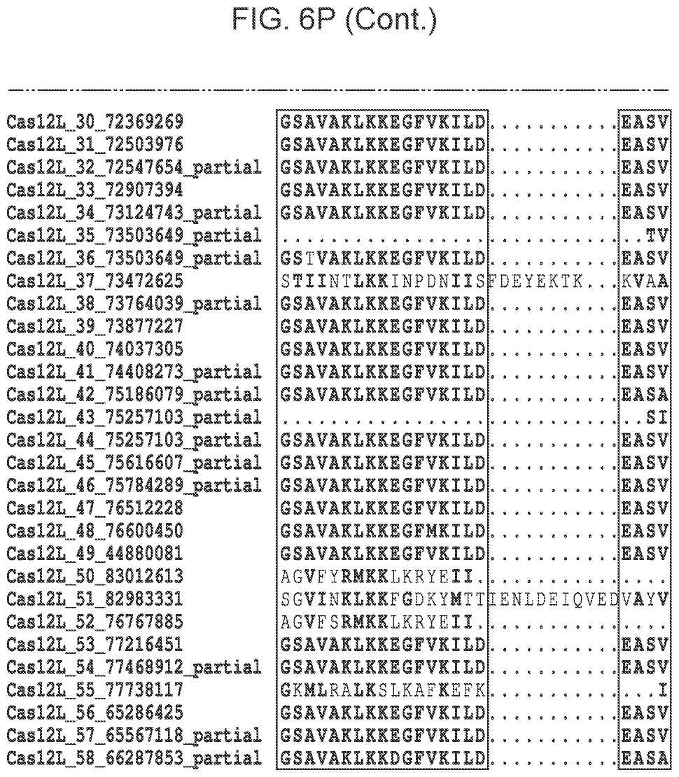

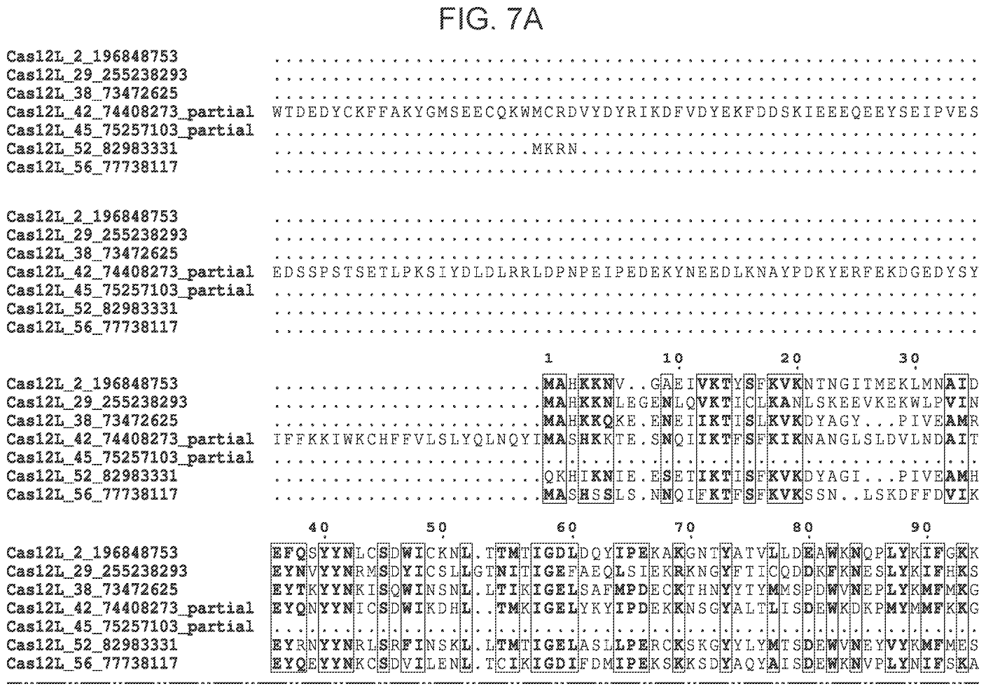

FIG. 6A-6P provide an amino acid sequence alignment of the 58 Cas12L polypeptides depicted in FIG. 4A-4Z, FIG. 4AA-4ZZ, and FIG. 4AAA-4FFF. As shown in FIG. 6A-6P, a number of amino acids are conserved among the 58 Cas12L polypeptides. For example, K14, T15, E37, Y41, Y42, N43, S46, 149, 157, Y73, Y88, F91, N119, L121, N140, Y141, Q169, 1171, E173, W185, Y191, Y205, L209, F212, Y213, F235, G236, G237, C238, R240, G281, N309, K324, 1335, T346, Y357, N359, 1360, Y361, V364, F370, F391, P393, L394, E395, L399, S401, R402, Q406, E417, V423, L424, Y442, K448, L449, R450, K454, A455, V459, K460, Y470, D471, E476, E477, and G594, based on the amino acid numbering of Cas12L_1_257905508 (FIG. 4A) are conserved. Thus, e.g., a Cas12L polypeptide can comprise one or more of K14, T15, E37, Y41, Y42, N43, S46, 149, 157, Y73, Y88, F91, N119, L121, N140, Y141, Q169, 1171, E173, W185, Y191, Y205, L209, F212, Y213, F235, G236, G237, C238, R240, G281, N309, K324, 1335, T346, Y357, N359, 1360, Y361, V364, F370, F391, P393, L394, E395, L399, S401, R402, Q406, E417, V423, L424, Y442, K448, L449, R450, K454, A455, V459, K460, Y470, D471, E476, E477, and G594, based on the amino acid numbering of Cas12L_1_257905508 (FIG. 4A), or the same amino acid at a corresponding position in another Cas12L polypeptide (e.g., a Cas12L polypeptide depicted in FIG. 4A-4Z, FIG. 4AA-4ZZ, and FIG. 4AAA-4FFF), where those skilled in the art would understand, based on the alignment provided in FIG. 6A-6P, what a “corresponding position” would be. A Cas12L polypeptide can comprise a conservative amino acid substitution at one or more of K14, T15, E37, Y41, Y42, N43, S46, 149, 157, Y73, Y88, F91, N119, L121, N140, Y141, Q169, 1171, E173, W185, Y191, Y205, L209, F212, Y213, F235, G236, G237, C238, R240, G281, N309, K324, 1335, T346, Y357, N359, 1360, Y361, V364, F370, F391, P393, L394, E395, L399, S401, R402, Q406, E417, V423, L424, Y442, K448, L449, R450, K454, A455, V459, K460, Y470, D471, E476, E477, and G594, based on the amino acid numbering of Cas12L_1_257905508 (FIG. 4A), or at a corresponding position in another Cas12L polypeptide (e.g., a Cas12L polypeptide depicted in FIG. 4A-4Z, FIG. 4AA-4ZZ, and FIG. 4AAA-4FFF).

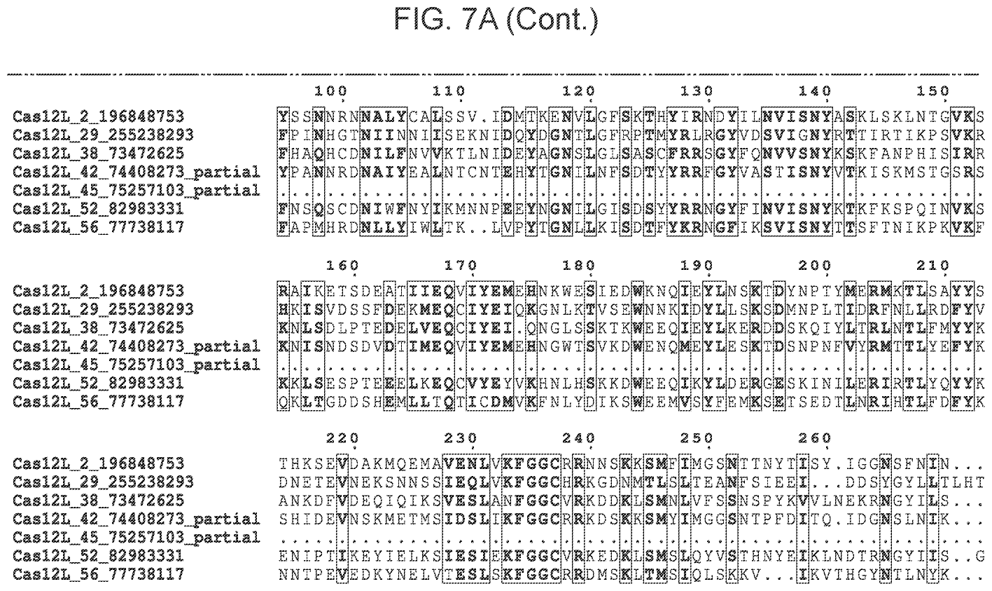

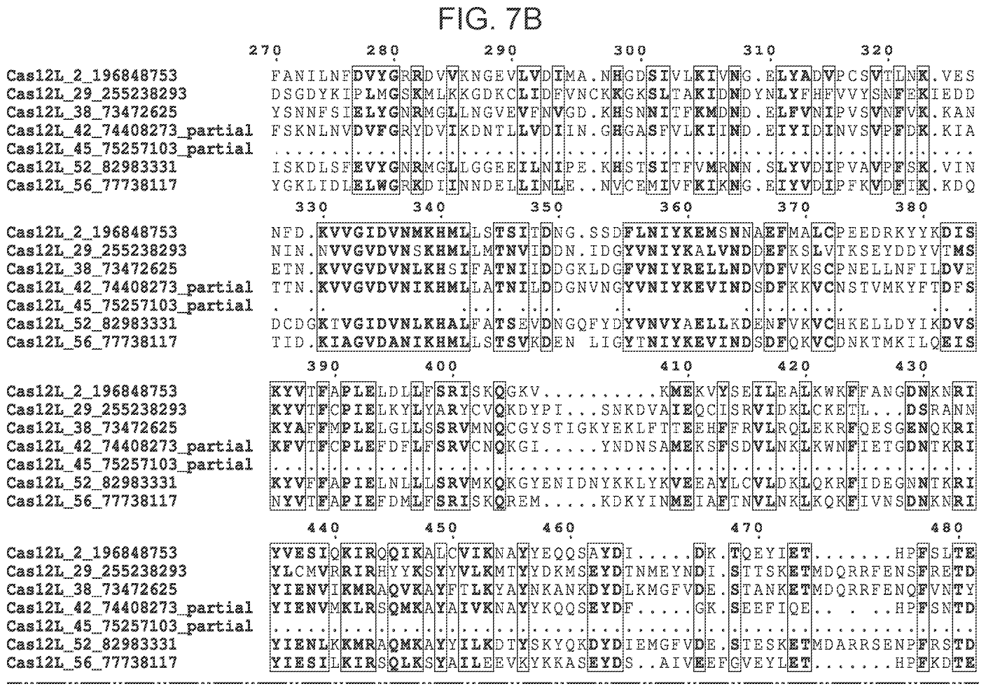

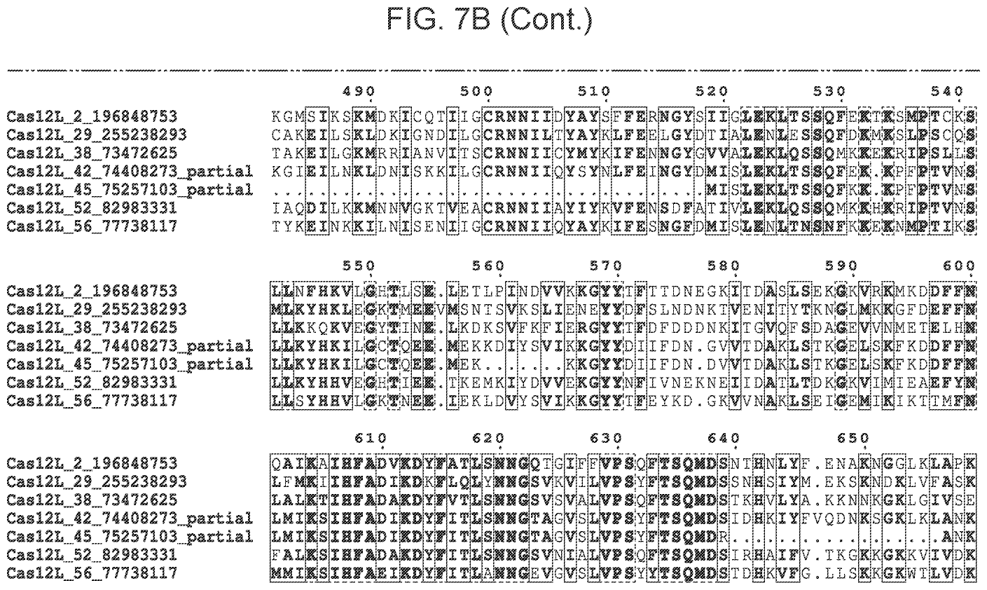

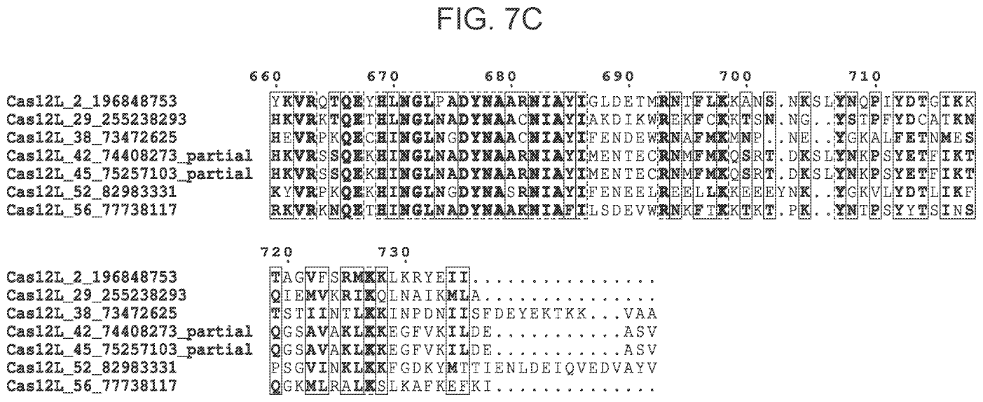

FIG. 7A-7C provide an alignment of the amino acid sequences of Cas12L_2_196848753 (FIG. 4B), Cas12L_29_255238293 (FIG. 4BB; referred to as Cas12L_28_255238293 in FIG. 4BB), Cas12L_38_73472625 (FIG. 4KK; referred to as Cas12L_37_73472625 in FIG. 4KK), Cas12L_42_74408273_partial (FIG. 4NN; referred to as Cas12L_41_74408273_partial in FIG. 4NN), Cas12L_54_75257103_partial (FIG. 4PP; referred to as Cas12L_43_75257103_partial in FIG. 4PP), Cas12L_52_82983331 (FIG. 4XX; referred to as Cas12L_51_82983331 in FIG. 4XX), and Cas12L_56_77738117 (FIG. 4BBB; referred to as Cas12L_55_77738117 in FIG. 4BBB. Amino acids in boxes are conserved among the Cas12L polypeptides in FIG. 7A-7C. A Cas12L polypeptide can comprise the amino acids set out in the boxes in FIG. 7A-7C, or a conservative amino acid substitution of such amino acids, at the positions depicted in FIG. 7A-7C.

In some cases, a Cas12L protein (of the subject compositions and/or methods) includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the RuvC domain (which includes the RuvC-I, RuvC-II, and RuvC-III domains) of any one of the Cas12L amino acid sequences depicted in FIG. 2 (e.g., any one of FIG. 4A-4Z, FIG. 4AA-4ZZ, and FIG. 4AAA-4FFF). In some cases, a Cas12L protein (of the subject compositions and/or methods) includes an amino acid sequence having 70% or more sequence identity (e.g., 75% or more, 80% or more, 85% or more, 90% or more, 95% or more, 97% or more, 98% or more, 99% or more, or 100% sequence identity) with the RuvC domain (which includes the RuvC-I, RuvC-II, and RuvC-III domains) of any one of the Cas12L amino acid sequences depicted in FIG. 4 (e.g., any one of FIG. 4A-4Z, FIG. 4AA-4ZZ, and FIG. 4AAA-4FFF). In some cases, a Cas12L protein (of the subject compositions and/or methods) includes the RuvC domain (which includes the RuvC-I, RuvC-II, and RuvC-III domains) of any one of the Cas12L amino acid sequences depicted in FIG. 4 (e.g., any one of FIG. 4A-4Z, FIG. 4AA-4ZZ, and FIG. 4AAA-4FFF).

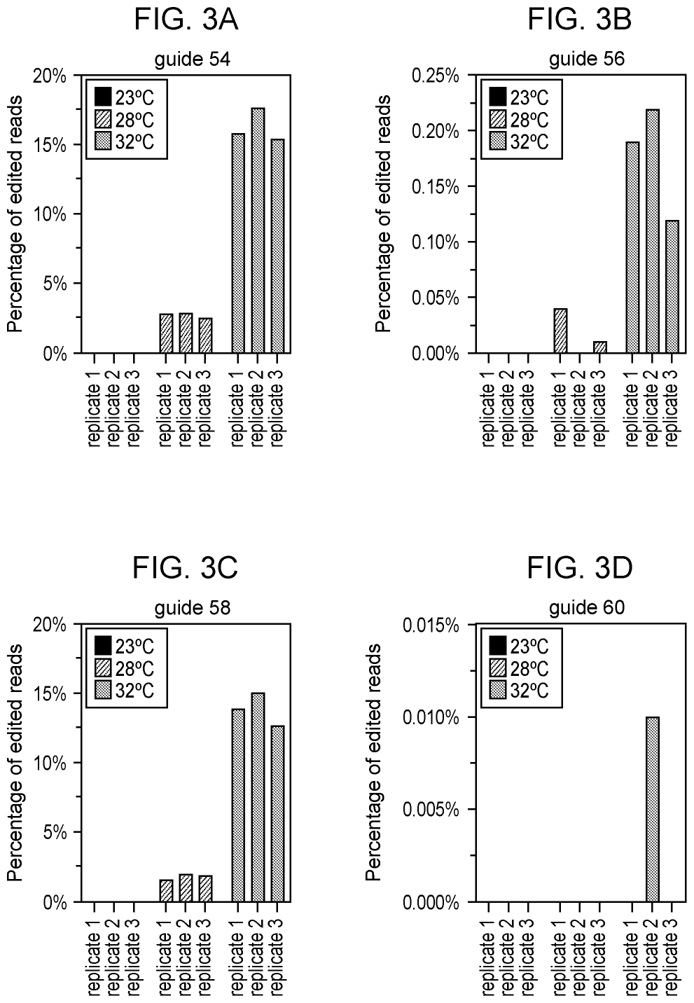

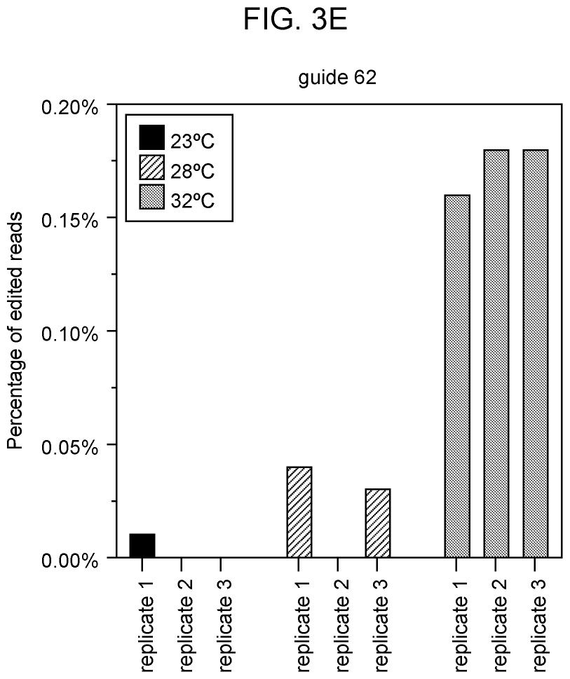

In some cases, a guide RNA that binds a Cas12L polypeptide includes a nucleotide sequence depicted in FIG. 4 (e.g., any one of FIG. 4A-4Z, FIG. 4AA-4ZZ, and FIG. 4AAA-4FFF; where “T” is replaced with “U”) (or in some cases the reverse complement of same). In some cases, the guide RNA comprises the nucleotide sequence (N)nX or the reverse complement of same, where N is any nucleotide, n is an integer from 15 to 30 (e.g., from 15 to 20, from 17 to 25, from 17 to 22, from 18 to 22, from 18 to 20, from 20 to 25, or from 25 to 30), and X is any one of the nucleotide sequences depicted in FIG. 3 (e.g., any one of FIG. 4A-4Z, FIG. 4AA-4ZZ, and FIG. 4AAA-4FFF) (or in some cases the reverse complement of same).

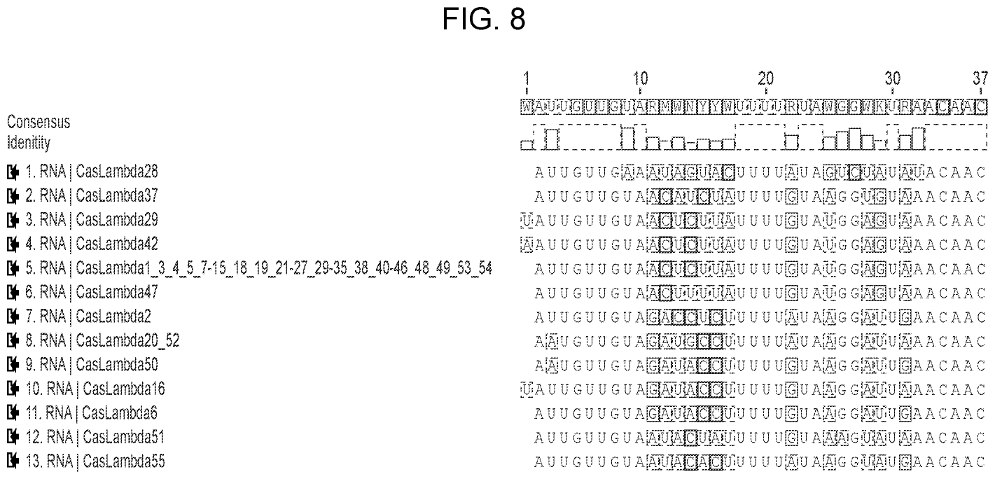

An alignment of nucleotide sequences of the repeat region of various CasLambda (Casλ; Cas12L) guide RNAs is provided in FIG. 8. As shown in FIG. 8, CRISPR repeat sequences contain conserved sequence motifs across homologs. In some cases, a guide RNA that binds a Cas12L polypeptide includes a nucleotide sequence (a repeat sequence; or protein-binding sequence) of the following consensus sequence: WAUUGUUGUARMWNYYWUUUURUAWGGWKURAACAAC (SEQ ID NO:71), where W is A or U; R is G or A; M is A or C; N is A, G, C, or U; Y is U or C; and K is G or U. For example, a guide RNA that binds a CasLambda28 polypeptide (Cas12L 28; FIG. 4BB) can comprise a protein-binding segment comprising the nucleotide sequence: AUUGUUGAAAUAGUACUUUUAUAGUCUAUAUACAAC (SEQ ID NO:92). As another example a guide RNA that binds a CasLambda37 polypeptide (Cas12L 37; FIG. 4KK) can comprise a protein-binding segment comprising the nucleotide sequence: AUUGUUGUAACAUCUAUUUUGUAAGGUGUAAACAAC (SEQ ID NO:93). As another example a guide RNA that binds a CasLambda29 polypeptide (Cas12L 29; FIG. 4CC) can comprise a protein-binding segment comprising the nucleotide sequence: UAUUGUUGUAACUCUUAUUUUGUAUGGAGUAAACAAC (SEQ ID NO:94). As another example a guide RNA that binds a CasLambda42 polypeptide (Cas12L 42; FIG. 4OO) can comprise a protein-binding segment comprising the nucleotide sequence: AAUUGUUGUAACUCUUAUUUUGUAUGGAGUAAACAAC (SEQ ID NO:95). As another example a guide RNA that binds any one of CasLambda polypeptides 1, 3, 4, 5, 7-15, 18, 19, 21-27, 29-35, 38, 40-46, 48, 49, 53, and 54 (any one of Cas12L 1, 3, 4, 5, 7-15, 18, 19, 21-27, 29-35, 38, 40-46, 48, 49, 53, and 54; FIG. 4A, 4C, 4D, 4E, 4G-40, 4R, 4S, 4U-4AA, 4CC-4II, 4LL, 4MM-4SS, 4UU, 4VV, 4ZZ, and 4AAA, respectively) can comprise a protein-binding segment comprising the nucleotide sequence: AUUGUUGUAACUCUUAUUUUGUAUGGAGUAAACAAC (SEQ ID NO:96). As another example a guide RNA that binds a CasLambda47 polypeptide (Cas12L 47; FIG. 4TT) can comprise a protein-binding segment comprising the nucleotide sequence: AUUGUUGUAACUUUUAUUUUGUAUGGAGUAAACAAC (SEQ ID NO:97). As another example a guide RNA that binds a CasLambda2 polypeptide (Cas12L 2; FIG. 4B) can comprise a protein-binding segment comprising the nucleotide sequence: AUUGUUGUAGACCUCUUUUUAUAAGGAUUGAACAAC (SEQ ID NO:98). A Cas12L polypeptide of the present disclosure can form a complex (a ribonucleoprotein (RNP) complex) with a guide RNA comprising a protein-binding segment depicted in FIG. 8. In some cases, a Cas12L polypeptide of the present disclosure can form an RNP complex with different guide RNAs comprising a protein-binding segment of different sequences depicted in FIG. 8.

As another example a guide RNA that binds a CasLambda20 polypeptide or a CasLambda52 polypeptide (Cas12L 20 or Cas12L 52; FIG. 4T and 4YY, respectively) can comprise a protein-binding segment comprising the nucleotide sequence: AAUGUUGUAGAUGCCUUUUUAUAAGGAUUAAACAAC (SEQ ID NO:99). As another example a guide RNA that binds a CasLambda50 polypeptide (Cas12L 50; FIG. 4WW) can comprise a protein-binding segment comprising the nucleotide sequence: AAUGUUGUAGAUACCUUUUUGUAAGGAUUGAACAAC (SEQ ID NO:100). As another example a guide RNA that binds a CasLambda16 polypeptide (Cas12L 16; FIG. 4P) can comprise a protein-binding segment comprising the nucleotide sequence: UAUUGUUGUAGAUACCUUUUUGUAAGGAUUAAACAAC (SEQ ID NO:101). As another example a guide RNA that binds a CasLambda6 polypeptide (Cas12L 6; FIG. 4F) can comprise a protein-binding segment comprising the nucleotide sequence: AUUGUUGUAGAUACCUUUUUGUAAGGAUUGAACAAC (SEQ ID NO:102). As another example a guide RNA that binds a CasLambda51 polypeptide (Cas12L 51; FIG. 4XX) can comprise a protein-binding segment comprising the nucleotide sequence: AUUGUUGUAAUACUAUUUUUGUAAAGUAUAAACAAC (SEQ ID NO:103). As another example a guide RNA that binds a CasLambda55 polypeptide (Cas12L 55; FIG. 4BBB) can comprise a protein-binding segment comprising the nucleotide sequence:

(SEQ ID NO: 104)
AUUGUUGUAAUACACUUUUUAUAAGGUAUGAACAAC.

In addition to containing conserved sequence motifs in the repeat (protein-binding) regions, the repeat region of a CasLambda guide RNA share conserved secondary structures across homologs. For example, the repeat region can include palindromic regions that can form stem and stem-loop structures.

In some cases, a guide RNA that binds a Cas12L polypeptide includes a nucleotide sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with any one of the sequences depicted in FIG. 4 (e.g., any one of FIG. 4A-4Z, FIG. 4AA-4ZZ, and FIG. 4AAA-4FFF) (or in some cases the reverse complement of same). In some cases, the guide RNA comprises the nucleotide sequence (N)nX or the reverse complement of same, where N is any nucleotide, n is an integer from 15 to 30 (e.g., from 15 to 20, from 17 to 25, from 17 to 22, from 18 to 22, from 18 to 20, from 20 to 25, or from 25 to 30), and X is a nucleotide sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with any one of the sequences depicted in FIG. 4 (e.g., any one of FIG. 4A-4Z, FIG. 4AA-4ZZ, and FIG. 4AAA-4FFF).

In some cases, a guide RNA that binds a Cas12L polypeptide includes a nucleotide sequence having 85% or more sequence identity (e.g., 90% or more, 95% or more, 97% or more, 98% or more, 99% or more, or 100% sequence identity) with any one of the sequences depicted in FIG. 4 (e.g., any one of FIG. 4A-4Z, FIG. 4AA-4ZZ, and FIG. 4AAA-4FFF) (or in some cases the reverse complement of same). In some cases, the guide RNA comprises the nucleotide sequence (N)nX or the reverse complement of same, where N is any nucleotide, n is an integer from 15 to 30 (e.g., from 15 to 20, from 17 to 25, from 17 to 22, from 18 to 22, from 18 to 20, from 20 to 25, or from 25 to 30), and X is a nucleotide sequence having 85% or more sequence identity (e.g., 90% or more, 95% or more, 97% or more, 98% or more, 99% or more, or 100% sequence identity) with any one of the sequences depicted in FIG. 4 (e.g., any one of FIG. 4A-4Z, FIG. 4AA-4ZZ, and FIG. 4AAA-4FFF).

In some cases, a guide RNA that binds a Cas12L polypeptide includes a nucleotide sequence depicted in FIG. 4 (e.g., any one of FIG. 4A-4Z, FIG. 4AA-4ZZ, and FIG. 4AAA-4FFF) (or in some cases the reverse complement of same). In some cases, the guide RNA comprises the nucleotide sequence X(N)n, where N is any nucleotide, n is an integer from 15 to 30 (e.g., from 15 to 20, from 17 to 25, from 17 to 22, from 18 to 22, from 18 to 20, from 20 to 25, or from 25 to 30), and X is any one of the nucleotide sequences depicted in FIG. 4 (e.g., any one of FIG. 4A-4Z, FIG. 4AA-4ZZ, and FIG. 4AAA-4FFF) (or in some cases the reverse complement of same).

In some cases, a guide RNA that binds a Cas12L polypeptide includes a nucleotide sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with any one of the sequences depicted in FIG. 4 (e.g., any one of FIG. 4A-4Z, FIG. 4AA-4ZZ, and FIG. 4AAA-4FFF) (or in some cases the reverse complement of same). In some cases, the guide RNA comprises the nucleotide sequence X(N)n, where N is any nucleotide, n is an integer from 15 to 30 (e.g., from 15 to 20, from 17 to 25, from 17 to 22, from 18 to 22, from 18 to 20, from 20 to 25, or from 25 to 30), and X is a nucleotide sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with any one of the sequences depicted in FIG. 4 (e.g., any one of FIG. 4A-4Z, FIG. 4AA-4ZZ, and FIG. 4AAA-4FFF).

In some cases, a guide RNA that binds a Cas12L polypeptide includes a nucleotide sequence having 85% or more sequence identity (e.g., 90% or more, 95% or more, 97% or more, 98% or more, 99% or more, or 100% sequence identity) with any one of the sequences depicted in FIG. 4 (e.g., any one of FIG. 4A-4Z, FIG. 4AA-4ZZ, and FIG. 4AAA-4FFF) (or in some cases the reverse complement of same). In some cases, the guide RNA comprises the nucleotide sequence X(N)n, where N is any nucleotide, n is an integer from 15 to 30 (e.g., from 15 to 20, from 17 to 25, from 17 to 22, from 18 to 22, from 18 to 20, from 20 to 25, or from 25 to 30), and X is a nucleotide sequence having 85% or more sequence identity (e.g., 90% or more, 95% or more, 97% or more, 98% or more, 99% or more, or 100% sequence identity) with any one of the sequences depicted in FIG. 4 (e.g., any one of FIG. 4A-4Z, FIG. 4AA-4ZZ, and FIG. 4AAA-4FFF).

### FIG. 4A

In some cases, a Cas12L protein (of the subject compositions and/or methods) includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4A and designated “Cas12L_1_257905508.” For example, in some cases, a Cas12L protein includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4A. In some cases, a Cas12L protein includes an amino acid sequence having 80% or more sequence identity (e.g., 85% or more, 90% or more, 95% or more, 97% or more, 98% or more, 99% or more, or 100% sequence identity) with the Cas12L amino acid sequence depicted in FIG. 4A. In some cases, a Cas12L protein includes an amino acid sequence having 90% or more sequence identity (e.g., 95% or more, 97% or more, 98% or more, 99% or more, or 100% sequence identity) with the Cas12L amino acid sequence depicted in FIG. 4A. In some cases, a Cas12L protein includes an amino acid sequence having the Cas12L amino acid sequence depicted in FIG. 4A. In some cases, a Cas12L protein includes an amino acid sequence having the Cas12L protein sequence depicted in FIG. 4A, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 746 aa, from 746 aa to 775 aa, or from 775 aa to 800 aa). In some cases, the Cas12L polypeptide has a length of 746 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4A) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4B

In some cases, a Cas12L protein (of the subject compositions and/or methods) includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4B and designated “Cas12L_2_196848753.” For example, in some cases, a Cas12L protein includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4B. In some cases, a Cas12L protein includes an amino acid sequence having 80% or more sequence identity (e.g., 85% or more, 90% or more, 95% or more, 97% or more, 98% or more, 99% or more, or 100% sequence identity) with the Cas12L amino acid sequence depicted in FIG. 4B. In some cases, a Cas12L protein includes an amino acid sequence having 90% or more sequence identity (e.g., 95% or more, 97% or more, 98% or more, 99% or more, or 100% sequence identity) with the Cas12L amino acid sequence depicted in FIG. 4B. In some cases, a Cas12L protein includes an amino acid sequence having the Cas12L amino acid sequence depicted in FIG. 4B. In some cases, a Cas12L protein includes an amino acid sequence having the Cas12L protein sequence depicted in FIG. 4B, with the exception that the sequence includes an amino acid substitution (e.g., 1, 2, or 3 amino acid substitutions) that reduces the naturally occurring catalytic activity of the protein. In some cases, the Cas12L polypeptide has a length of from 675 amino acids (aa) to 800 aa, e.g., from 675 aa to 700 aa, from 700 aa to 725 aa, from 725 aa to 735 aa, from 735 aa to 775 aa, or from 775 aa to 800 aa). In some cases, the Cas12L polypeptide has a length of 735 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4B) includes the following nucleotide sequence: ATTGTTGTAGACCTCTTTTTATAAGGATTGAACAAC (SEQ ID NO:58) or the reverse complement of same. In some cases, the guide RNA comprises the nucleotide sequence (N)nATTGTTGTAGACCTCTTTTTATAAGGATTGAACAAC (SEQ ID NO:106) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4C

In some cases, a Cas12L protein (of the subject compositions and/or methods) includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4C and designated “Cas12L_3_66741167.” For example, in some cases, a Cas12L protein includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4C. In some cases, a Cas12L protein includes an amino acid sequence having 80% or more sequence identity (e.g., 85% or more, 90% or more, 95% or more, 97% or more, 98% or more, 99% or more, or 100% sequence identity) with the Cas12L amino acid sequence depicted in FIG. 4C. In some cases, a Cas12L protein includes an amino acid sequence having 90% or more sequence identity (e.g., 95% or more, 97% or more, 98% or more, 99% or more, or 100% sequence identity) with the Cas12L amino acid sequence depicted in FIG. 4C. In some cases, a Cas12L protein includes an amino acid sequence having the Cas12L amino acid sequence depicted in FIG. 4C. In some cases, a Cas12L protein includes an amino acid sequence having the Cas12L protein sequence depicted in FIG. 4C, with the exception that the sequence includes an amino acid substitution (e.g., 1, 2, or 3 amino acid substitutions) that reduces the naturally occurring catalytic activity of the protein. In some cases, the Cas12L polypeptide has a length of from 725 amino acids (aa) to 875 aa, e.g., from 725 aa to 750 aa, from 750 aa to 778 aa, from 778 aa to 800 aa, from 800 aa to 825 aa, from 825 aa to 850 aa, or from 850 aa to 875 aa). In some cases, the Cas12L polypeptide has a length of 778 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4C) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4D

In some cases, a Cas12L protein (of the subject compositions and/or methods) includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4D and designated “Cas12L_4_67031163.” For example, in some cases, a Cas12L protein includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4D. In some cases, a Cas12L protein includes an amino acid sequence having 80% or more sequence identity (e.g., 85% or more, 90% or more, 95% or more, 97% or more, 98% or more, 99% or more, or 100% sequence identity) with the Cas12L amino acid sequence depicted in FIG. 4D. In some cases, a Cas12L protein includes an amino acid sequence having 90% or more sequence identity (e.g., 95% or more, 97% or more, 98% or more, 99% or more, or 100% sequence identity) with the Cas12L amino acid sequence depicted in FIG. 4D. In some cases, a Cas12L protein includes an amino acid sequence having the Cas12L amino acid sequence depicted in FIG. 4D. In some cases, a Cas12L protein includes an amino acid sequence having the Cas12L protein sequence depicted in FIG. 4D, with the exception that the sequence includes an amino acid substitution (e.g., 1, 2, or 3 amino acid substitutions) that reduces the naturally occurring catalytic activity of the protein. In some cases, the Cas12L polypeptide has a length of from 725 amino acids (aa) to 875 aa, e.g., from 725 aa to 750 aa, from 750 aa to 778 aa, from 778 aa to 800 aa, from 800 aa to 825 aa, from 825 aa to 850 aa, or from 850 aa to 875 aa). In some cases, the Cas12L polypeptide has a length of 778 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4D) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4E

In some cases, a Cas12L protein (of the subject compositions and/or methods) includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4E and designated “Cas12L_5_67793351.” For example, in some cases, a Cas12L protein includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4E. In some cases, a Cas12L protein includes an amino acid sequence having 80% or more sequence identity (e.g., 85% or more, 90% or more, 95% or more, 97% or more, 98% or more, 99% or more, or 100% sequence identity) with the Cas12L amino acid sequence depicted in FIG. 4E. In some cases, a Cas12L protein includes an amino acid sequence having 90% or more sequence identity (e.g., 95% or more, 97% or more, 98% or more, 99% or more, or 100% sequence identity) with the Cas12L amino acid sequence depicted in FIG. 4E. In some cases, a Cas12L protein includes an amino acid sequence having the Cas12L amino acid sequence depicted in FIG. 4E. In some cases, a Cas12L protein includes an amino acid sequence having the Cas12L protein sequence depicted in FIG. 4E, with the exception that the sequence includes an amino acid substitution (e.g., 1, 2, or 3 amino acid substitutions) that reduces the naturally occurring catalytic activity of the protein. In some cases, the Cas12L polypeptide has a length of from 725 amino acids (aa) to 875 aa, e.g., from 725 aa to 750 aa, from 750 aa to 778 aa, from 778 aa to 800 aa, from 800 aa to 825 aa, from 825 aa to 850 aa, or from 850 aa to 875 aa). In some cases, the Cas12L polypeptide has a length of 778 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4E) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4F

In some cases, a Cas12L protein (of the subject compositions and/or methods) includes a contiguous stretch of about 350 amino acids having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4F and designated “Cas12L_6_67912869_partial.” For example, in some cases, a Cas12L protein includes a contiguous stretch of about 350 amino acids having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4F. In some cases, a Cas12L protein includes a contiguous stretch of about 350 amino acids having 80% or more sequence identity (e.g., 85% or more, 90% or more, 95% or more, 97% or more, 98% or more, 99% or more, or 100% sequence identity) with the Cas12L amino acid sequence depicted in FIG. 4F. In some cases, a Cas12L protein includes a contiguous stretch of about 350 amino acids having 90% or more sequence identity (e.g., 95% or more, 97% or more, 98% or more, 99% or more, or 100% sequence identity) with the Cas12L amino acid sequence depicted in FIG. 4F. In some cases, a Cas12L protein includes a contiguous stretch of about 350 amino acids comprising the Cas12L amino acid sequence depicted in FIG. 4F. In some cases, a Cas12L protein includes a contiguous stretch of about 350 amino acids having the Cas12L protein sequence depicted in FIG. 4F, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 750 aa, or from 750 aa to 800 aa). In some cases, the Cas12L polypeptide has a length of from about 725 amino acids to about 775 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4F) includes the following nucleotide sequence: ATTGTTGTAGATACCTTTTTGTAAGGATTGAACAAC (SEQ ID NO:59) or the reverse complement of same. In some cases, the guide RNA comprises the nucleotide sequence (N)nATTGTTGTAGATACCTTTTTGTAAGGATTGAACAAC (SEQ ID NO:107) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4G

In some cases, a Cas12L protein (of the subject compositions and/or methods) includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4G and designated “Cas12L_7_68090316_partial.” For example, in some cases, a Cas12L protein includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4G. In some cases, a Cas12L protein includes an amino acid sequence having 80% or more sequence identity (e.g., 85% or more, 90% or more, 95% or more, 97% or more, 98% or more, 99% or more, or 100% sequence identity) with the Cas12L amino acid sequence depicted in FIG. 4G. In some cases, a Cas12L protein includes an amino acid sequence having 90% or more sequence identity (e.g., 95% or more, 97% or more, 98% or more, 99% or more, or 100% sequence identity) with the Cas12L amino acid sequence depicted in FIG. 4G. In some cases, a Cas12L protein includes an amino acid sequence having the Cas12L amino acid sequence depicted in FIG. 4G. In some cases, a Cas12L protein includes an amino acid sequence having the Cas12L protein sequence depicted in FIG. 4G, with the exception that the sequence includes an amino acid substitution (e.g., 1, 2, or 3 amino acid substitutions) that reduces the naturally occurring catalytic activity of the protein. In some cases, the Cas12L polypeptide has a length of from 650 amino acids (aa) to 800 aa, e.g., from 650 aa to 700 aa, from 700 aa to 717 aa, from 717 aa to 725 aa, from 725 aa to 750 aa, or from 750 aa to 800 aa). In some cases, the Cas12L polypeptide has a length of from about 717 amino acids to about 775 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4G) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4H

In some cases, a Cas12L protein (of the subject compositions and/or methods) includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4H and designated “Cas12L_8_68328292_partial.” For example, in some cases, a Cas12L protein includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4H. In some cases, a Cas12L protein includes an amino acid sequence having 80% or more sequence identity (e.g., 85% or more, 90% or more, 95% or more, 97% or more, 98% or more, 99% or more, or 100% sequence identity) with the Cas12L amino acid sequence depicted in FIG. 4H. In some cases, a Cas12L protein includes an amino acid sequence having 90% or more sequence identity (e.g., 95% or more, 97% or more, 98% or more, 99% or more, or 100% sequence identity) with the Cas12L amino acid sequence depicted in FIG. 4H. In some cases, a Cas12L protein includes an amino acid sequence having the Cas12L amino acid sequence depicted in FIG. 4H. In some cases, a Cas12L protein includes an amino acid sequence having the Cas12L protein sequence depicted in FIG. 4H, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 742 aa, from 742 aa to 750 aa, from 750 aa to 775 aa, or from 775 aa to 800 aa). In some cases, the Cas12L polypeptide has a length of from about 742 amino acids to about 775 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4H) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4I

In some cases, a Cas12L protein (of the subject compositions and/or methods) includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4I and designated “Cas12L_9_68454124.” For example, in some cases, a Cas12L protein includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4I. In some cases, a Cas12L protein includes an amino acid sequence having 80% or more sequence identity (e.g., 85% or more, 90% or more, 95% or more, 97% or more, 98% or more, 99% or more, or 100% sequence identity) with the Cas12L amino acid sequence depicted in FIG. 4I. In some cases, a Cas12L protein includes an amino acid sequence having 90% or more sequence identity (e.g., 95% or more, 97% or more, 98% or more, 99% or more, or 100% sequence identity) with the Cas12L amino acid sequence depicted in FIG. 4I. In some cases, a Cas12L protein includes an amino acid sequence having the Cas12L amino acid sequence depicted in FIG. 4I. In some cases, a Cas12L protein includes an amino acid sequence having the Cas12L protein sequence depicted in FIG. 4I, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 746 aa, from 746 aa to 750 aa, from 750 aa to 775 aa, or from 775 aa to 800 aa). In some cases, the Cas12L polypeptide has a length of 746 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4I) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4J

In some cases, a Cas12L protein (of the subject compositions and/or methods) includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4J and designated “Cas12L_10_68605313.” For example, in some cases, a Cas12L protein includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4J. In some cases, a Cas12L protein includes an amino acid sequence having 80% or more sequence identity (e.g., 85% or more, 90% or more, 95% or more, 97% or more, 98% or more, 99% or more, or 100% sequence identity) with the Cas12L amino acid sequence depicted in FIG. 4J. In some cases, a Cas12L protein includes an amino acid sequence having 90% or more sequence identity (e.g., 95% or more, 97% or more, 98% or more, 99% or more, or 100% sequence identity) with the Cas12L amino acid sequence depicted in FIG. 4J. In some cases, a Cas12L protein includes an amino acid sequence having the Cas12L amino acid sequence depicted in FIG. 4J. In some cases, a Cas12L protein includes an amino acid sequence having the Cas12L protein sequence depicted in FIG. 4J, with the exception that the sequence includes an amino acid substitution (e.g., 1, 2, or 3 amino acid substitutions) that reduces the naturally occurring catalytic activity of the protein. In some cases, the Cas12L polypeptide has a length of from 725 amino acids (aa) to 825 aa, e.g., from 725 aa to 750 aa, from 750 aa to 775 aa, from 775 aa to 782 aa, from 782 aa to 800 aa, or from 800 aa to 825 aa). In some cases, the Cas12L polypeptide has a length of 782 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4J) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4K

In some cases, a Cas12L protein (of the subject compositions and/or methods) includes a contiguous stretch of about 92 amino acids having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4K and designated “Cas12L_11_69266821_partial.” For example, in some cases, a Cas12L protein includes a contiguous stretch of about 92 amino acids having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4K. In some cases, a Cas12L protein includes a contiguous stretch of about 92 amino acids having 80% or more sequence identity (e.g., 85% or more, 90% or more, 95% or more, 97% or more, 98% or more, 99% or more, or 100% sequence identity) with the Cas12L amino acid sequence depicted in FIG. 4K. In some cases, a Cas12L protein includes a contiguous stretch of about 92 amino acids having 90% or more sequence identity (e.g., 95% or more, 97% or more, 98% or more, 99% or more, or 100% sequence identity) with the Cas12L amino acid sequence depicted in FIG. 4K. In some cases, a Cas12L protein includes a contiguous stretch of about 92 amino acids having the Cas12L amino acid sequence depicted in FIG. 4K. In some cases, a Cas12L protein includes a contiguous stretch of about 92 amino acids having the Cas12L protein sequence depicted in FIG. 4K, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 750 aa, from 750 aa to 775 aa, or from 775 aa to 800 aa). In some cases, the Cas12L polypeptide has a length of from 725 amino acids to 775 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4K) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4L

In some cases, a Cas12L protein (of the subject compositions and/or methods) includes a contiguous stretch of about 92 amino acids having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4L and designated “Cas12L_12_69417229_partial.” For example, in some cases, a Cas12L protein includes a contiguous stretch of about 92 amino acids having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4L. In some cases, a Cas12L protein includes a contiguous stretch of about 92 amino acids having 80% or more sequence identity (e.g., 85% or more, 90% or more, 95% or more, 97% or more, 98% or more, 99% or more, or 100% sequence identity) with the Cas12L amino acid sequence depicted in FIG. 4L. In some cases, a Cas12L protein includes a contiguous stretch of about 92 amino acids having 90% or more sequence identity (e.g., 95% or more, 97% or more, 98% or more, 99% or more, or 100% sequence identity) with the Cas12L amino acid sequence depicted in FIG. 4L. In some cases, a Cas12L protein includes a contiguous stretch of about 92 amino acids having the Cas12L amino acid sequence depicted in FIG. 4L. In some cases, a Cas12L protein includes a contiguous stretch of about 92 amino acids having the Cas12L protein sequence depicted in FIG. 4L, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 750 aa, from 750 aa to 775 aa, or from 775 aa to 800 aa). In some cases, the Cas12L polypeptide has a length of from 725 amino acids to 775 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4L) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4M

In some cases, a Cas12L protein (of the subject compositions and/or methods) includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4M and designated “Cas12L_13_69733214.” For example, in some cases, a Cas12L protein includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4M. In some cases, a Cas12L protein includes an amino acid sequence having 80% or more sequence identity (e.g., 85% or more, 90% or more, 95% or more, 97% or more, 98% or more, 99% or more, or 100% sequence identity) with the Cas12L amino acid sequence depicted in FIG. 4M. In some cases, a Cas12L protein includes an amino acid sequence having 90% or more sequence identity (e.g., 95% or more, 97% or more, 98% or more, 99% or more, or 100% sequence identity) with the Cas12L amino acid sequence depicted in FIG. 4M. In some cases, a Cas12L protein includes an amino acid sequence having the Cas12L amino acid sequence depicted in FIG. 4M. In some cases, a Cas12L protein includes an amino acid sequence having the Cas12L protein sequence depicted in FIG. 4M, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 750 aa, from 750 aa to 775 aa, or from 775 aa to 800 aa). In some cases, the Cas12L polypeptide has a length of 746 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4M) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4N

In some cases, a Cas12L protein (of the subject compositions and/or methods) includes a contiguous stretch of about 427 amino acids having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4N and designated “Cas12L_14_70235246_partial.” For example, in some cases, a Cas12L protein includes a contiguous stretch of about 427 amino acids having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4N. In some cases, a Cas12L protein includes a contiguous stretch of about 427 amino acids having 80% or more sequence identity (e.g., 85% or more, 90% or more, 95% or more, 97% or more, 98% or more, 99% or more, or 100% sequence identity) with the Cas12L amino acid sequence depicted in FIG. 4N. In some cases, a Cas12L protein includes a contiguous stretch of about 427 amino acids having 90% or more sequence identity (e.g., 95% or more, 97% or more, 98% or more, 99% or more, or 100% sequence identity) with the Cas12L amino acid sequence depicted in FIG. 4N. In some cases, a Cas12L protein includes a contiguous stretch of about 427 amino acids having the Cas12L amino acid sequence depicted in FIG. 4N. In some cases, a Cas12L protein includes a contiguous stretch of about 427 amino acids having the Cas12L protein sequence depicted in FIG. 4N, with the exception that the sequence includes an amino acid substitution (e.g., 1, 2, or 3 amino acid substitutions) that reduces the naturally occurring catalytic activity of the protein. In some cases, the Cas12L polypeptide has a length of from 425 amino acids (aa) to 800 aa, e.g., from 425 aa to 500 aa, from 500 aa to 600 aa, from 600 aa to 700 aa, from 700 aa to 725 aa, from 725 aa to 750 aa, from 750 aa to 775 aa, or from 775 aa to 800 aa). In some cases, the Cas12L polypeptide has a length of from 725 amino acids to 775 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4N) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4O

In some cases, a Cas12L protein (of the subject compositions and/or methods) includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4O and designated “Cas12L_15_70724743.” For example, in some cases, a Cas12L protein includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4O. In some cases, a Cas12L protein includes an amino acid sequence having 80% or more sequence identity (e.g., 85% or more, 90% or more, 95% or more, 97% or more, 98% or more, 99% or more, or 100% sequence identity) with the Cas12L amino acid sequence depicted in FIG. 4O. In some cases, a Cas12L protein includes an amino acid sequence having 90% or more sequence identity (e.g., 95% or more, 97% or more, 98% or more, 99% or more, or 100% sequence identity) with the Cas12L amino acid sequence depicted in FIG. 4O. In some cases, a Cas12L protein includes an amino acid sequence having the Cas12L amino acid sequence depicted in FIG. 4O. In some cases, a Cas12L protein includes an amino acid sequence having the Cas12L protein sequence depicted in FIG. 4O, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 746 aa, from 746 aa to 775 aa, or from 775 aa to 800 aa). In some cases, the Cas12L polypeptide has a length of 746 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4O) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4P

In some cases, a Cas12L protein (of the subject compositions and/or methods) includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4P and designated “Cas12L_16_70731038.” For example, in some cases, a Cas12L protein includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4P. In some cases, a Cas12L protein includes an amino acid sequence having 80% or more sequence identity (e.g., 85% or more, 90% or more, 95% or more, 97% or more, 98% or more, 99% or more, or 100% sequence identity) with the Cas12L amino acid sequence depicted in FIG. 4P. In some cases, a Cas12L protein includes an amino acid sequence having 90% or more sequence identity (e.g., 95% or more, 97% or more, 98% or more, 99% or more, or 100% sequence identity) with the Cas12L amino acid sequence depicted in FIG. 4P. In some cases, a Cas12L protein includes an amino acid sequence having the Cas12L amino acid sequence depicted in FIG. 4P. In some cases, a Cas12L protein includes an amino acid sequence having the Cas12L protein sequence depicted in FIG. 4P, with the exception that the sequence includes an amino acid substitution (e.g., 1, 2, or 3 amino acid substitutions) that reduces the naturally occurring catalytic activity of the protein. In some cases, the Cas12L polypeptide has a length of from 675 amino acids (aa) to 800 aa, e.g., from 675 aa to 700 aa, from 700 aa to 725 aa, from 725 aa to 735 aa, from 735 aa to 750 aa, from 750 aa to 775 aa, or from 775 aa to 800 aa). In some cases, the Cas12L polypeptide has a length of 735 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4P) includes the following nucleotide sequence: TATTGTTGTAGATACCTTTTTGTAAGGATTAAACAAC (SEQ ID NO:60) or the reverse complement of same. In some cases, the guide RNA comprises the nucleotide sequence (N)nTATTGTTGTAGATACCTTTTTGTAAGGATTAAACAAC (SEQ ID NO:108) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4Q

In some cases, a Cas12L protein (of the subject compositions and/or methods) includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4Q and designated “Cas12L_17_70959391.” For example, in some cases, a Cas12L protein includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4Q. In some cases, a Cas12L protein includes an amino acid sequence having 80% or more sequence identity (e.g., 85% or more, 90% or more, 95% or more, 97% or more, 98% or more, 99% or more, or 100% sequence identity) with the Cas12L amino acid sequence depicted in FIG. 4Q. In some cases, a Cas12L protein includes an amino acid sequence having 90% or more sequence identity (e.g., 95% or more, 97% or more, 98% or more, 99% or more, or 100% sequence identity) with the Cas12L amino acid sequence depicted in FIG. 4Q. In some cases, a Cas12L protein includes an amino acid sequence having the Cas12L amino acid sequence depicted in FIG. 4Q. In some cases, a Cas12L protein includes an amino acid sequence having the Cas12L protein sequence depicted in FIG. 4Q, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 746 aa, from 746 aa to 775 aa, or from 775 aa to 800 aa). In some cases, the Cas12L polypeptide has a length of 746 amino acids.

### FIG. 4R

In some cases, a Cas12L protein (of the subject compositions and/or methods) includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4R and designated “Cas12L_18_71078086.” For example, in some cases, a Cas12L protein includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4R. In some cases, a Cas12L protein includes an amino acid sequence having 80% or more sequence identity (e.g., 85% or more, 90% or more, 95% or more, 97% or more, 98% or more, 99% or more, or 100% sequence identity) with the Cas12L amino acid sequence depicted in FIG. 4R. In some cases, a Cas12L protein includes an amino acid sequence having 90% or more sequence identity (e.g., 95% or more, 97% or more, 98% or more, 99% or more, or 100% sequence identity) with the Cas12L amino acid sequence depicted in FIG. 4R. In some cases, a Cas12L protein includes an amino acid sequence having the Cas12L amino acid sequence depicted in FIG. 4R. In some cases, a Cas12L protein includes an amino acid sequence having the Cas12L protein sequence depicted in FIG. 4R, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 746 aa, from 746 aa to 775 aa, or from 775 aa to 800 aa). In some cases, the Cas12L polypeptide has a length of 746 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4R) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4S

In some cases, a Cas12L protein (of the subject compositions and/or methods) includes a contiguous stretch of about 680 amino acids having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4S and designated “Cas12L_19_71193509_partial.” For example, in some cases, a Cas12L protein includes a contiguous stretch of about 680 amino acids having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4S. In some cases, a Cas12L protein includes a contiguous stretch of about 680 amino acids having 80% or more sequence identity (e.g., 85% or more, 90% or more, 95% or more, 97% or more, 98% or more, 99% or more, or 100% sequence identity) with the Cas12L amino acid sequence depicted in FIG. 4S. In some cases, a Cas12L protein includes a contiguous stretch of about 680 amino acids having 90% or more sequence identity (e.g., 95% or more, 97% or more, 98% or more, 99% or more, or 100% sequence identity) with the Cas12L amino acid sequence depicted in FIG. 4S. In some cases, a Cas12L protein includes a contiguous stretch of about 680 amino acids having the Cas12L amino acid sequence depicted in FIG. 4S. In some cases, a Cas12L protein includes a contiguous stretch of about 680 amino acids having the Cas12L protein sequence depicted in FIG. 4S, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 750 aa, or from 750 aa to 800 aa). In some cases, the Cas12L polypeptide has a length of from about 725 amino acids to about 775 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4S) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4T

In some cases, a Cas12L protein (of the subject compositions and/or methods) includes a contiguous stretch of about 516 amino acids having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4T and designated “Cas12L_20_71210958_partial.” For example, in some cases, a Cas12L protein includes a contiguous stretch of about 516 amino acids having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4T. In some cases, a Cas12L protein includes a contiguous stretch of about 516 amino acids having 80% or more sequence identity (e.g., 85% or more, 90% or more, 95% or more, 97% or more, 98% or more, 99% or more, or 100% sequence identity) with the Cas12L amino acid sequence depicted in FIG. 4T. In some cases, a Cas12L protein includes a contiguous stretch of about 516 amino acids having 90% or more sequence identity (e.g., 95% or more, 97% or more, 98% or more, 99% or more, or 100% sequence identity) with the Cas12L amino acid sequence depicted in FIG. 4T. In some cases, a Cas12L protein includes a contiguous stretch of about 516 amino acids having the Cas12L amino acid sequence depicted in FIG. 4T. In some cases, a Cas12L protein includes a contiguous stretch of about 516 amino acids having the Cas12L protein sequence depicted in FIG. 4T, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 750 aa, or from 750 aa to 800 aa). In some cases, the Cas12L polypeptide has a length of from about 725 amino acids to about 775 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4T) includes the following nucleotide sequence: AATGTTGTAGATGCCTTTTTATAAGGATTAAACAACTTG (SEQ ID NO:61) or the reverse complement of same. In some cases, the guide RNA comprises the nucleotide sequence (N)nAATGTTGTAGATGCCTTTTTATAAGGATTAAACAACTTG (SEQ ID NO:109) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4U

In some cases, a Cas12L protein (of the subject compositions and/or methods) includes a contiguous stretch of about 585 amino acids having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4U and designated “Cas12L_21_71317321_partial.” For example, in some cases, a Cas12L protein includes a contiguous stretch of about 585 amino acids having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4U. In some cases, a Cas12L protein includes a contiguous stretch of about 585 amino acids having 80% or more sequence identity (e.g., 85% or more, 90% or more, 95% or more, 97% or more, 98% or more, 99% or more, or 100% sequence identity) with the Cas12L amino acid sequence depicted in FIG. 4U. In some cases, a Cas12L protein includes a contiguous stretch of about 585 amino acids having 90% or more sequence identity (e.g., 95% or more, 97% or more, 98% or more, 99% or more, or 100% sequence identity) with the Cas12L amino acid sequence depicted in FIG. 4U. In some cases, a Cas12L protein includes a contiguous stretch of about 585 amino acids having the Cas12L amino acid sequence depicted in FIG. 4U. In some cases, a Cas12L protein includes a contiguous stretch of about 585 amino acids having the Cas12L protein sequence depicted in FIG. 4U, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 750 aa, or from 750 aa to 800 aa). In some cases, the Cas12L polypeptide has a length of from about 725 amino acids to about 775 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4U) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4V

In some cases, a Cas12L protein (of the subject compositions and/or methods) includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4V and designated “Cas12L_22_71456687.” For example, in some cases, a Cas12L protein includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4V. In some cases, a Cas12L protein includes an amino acid sequence having 80% or more sequence identity (e.g., 85% or more, 90% or more, 95% or more, 97% or more, 98% or more, 99% or more, or 100% sequence identity) with the Cas12L amino acid sequence depicted in FIG. 4V. In some cases, a Cas12L protein includes an amino acid sequence having 90% or more sequence identity (e.g., 95% or more, 97% or more, 98% or more, 99% or more, or 100% sequence identity) with the Cas12L amino acid sequence depicted in FIG. 4V. In some cases, a Cas12L protein includes an amino acid sequence having the Cas12L amino acid sequence depicted in FIG. 4V. In some cases, a Cas12L protein includes an amino acid sequence having the Cas12L protein sequence depicted in FIG. 4V, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 746 aa, from 746 aa to 775 aa, or from 775 aa to 800 aa). In some cases, the Cas12L polypeptide has a length of 746 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4V) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4W

In some cases, a Cas12L protein (of the subject compositions and/or methods) includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4W and designated “Cas12L_23_71708971.” For example, in some cases, a Cas12L protein includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4W. In some cases, a Cas12L protein includes an amino acid sequence having 80% or more sequence identity (e.g., 85% or more, 90% or more, 95% or more, 97% or more, 98% or more, 99% or more, or 100% sequence identity) with the Cas12L amino acid sequence depicted in FIG. 4W. In some cases, a Cas12L protein includes an amino acid sequence having 90% or more sequence identity (e.g., 95% or more, 97% or more, 98% or more, 99% or more, or 100% sequence identity) with the Cas12L amino acid sequence depicted in FIG. 4W. In some cases, a Cas12L protein includes an amino acid sequence having the Cas12L amino acid sequence depicted in FIG. 4W. In some cases, a Cas12L protein includes an amino acid sequence having the Cas12L protein sequence depicted in FIG. 4W, with the exception that the sequence includes an amino acid substitution (e.g., 1, 2, or 3 amino acid substitutions) that reduces the naturally occurring catalytic activity of the protein. In some cases, the Cas12L polypeptide has a length of from 500 amino acids (aa) to 800 aa, e.g., from 500 aa to 550 aa, from 550 aa to 585 aa, from 585 aa to 600 aa, from 600 aa to 650 aa, from 650 aa to 700 aa, from 700 aa to 725 aa, from 725 aa to 750 aa, from 750 aa to 775 aa, or from 775 aa to 800 aa). In some cases, the Cas12L polypeptide has a length of 585 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4W) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4X

In some cases, a Cas12L protein (of the subject compositions and/or methods) includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4X and designated “Cas12L_24_46035167_partial.” For example, in some cases, a Cas12L protein includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4X. In some cases, a Cas12L protein includes an amino acid sequence having 80% or more sequence identity (e.g., 85% or more, 90% or more, 95% or more, 97% or more, 98% or more, 99% or more, or 100% sequence identity) with the Cas12L amino acid sequence depicted in FIG. 4X. In some cases, a Cas12L protein includes an amino acid sequence having 90% or more sequence identity (e.g., 95% or more, 97% or more, 98% or more, 99% or more, or 100% sequence identity) with the Cas12L amino acid sequence depicted in FIG. 4X. In some cases, a Cas12L protein includes an amino acid sequence having the Cas12L amino acid sequence depicted in FIG. 4X. In some cases, a Cas12L protein includes an amino acid sequence having the Cas12L protein sequence depicted in FIG. 4X, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 758 aa, from 758 aa to 775 aa, or from 775 aa to 800 aa). In some cases, the Cas12L polypeptide has a length of 758 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4X) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4Y

In some cases, a Cas12L protein (of the subject compositions and/or methods) includes a contiguous stretch of about 596 amino acids having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4Y and designated “Cas12L_25_46784254_partial.” For example, in some cases, a Cas12L protein includes a contiguous stretch of about 596 amino acids having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4Y. In some cases, a Cas12L protein includes a contiguous stretch of about 596 amino acids having 80% or more sequence identity (e.g., 85% or more, 90% or more, 95% or more, 97% or more, 98% or more, 99% or more, or 100% sequence identity) with the Cas12L amino acid sequence depicted in FIG. 4Y. In some cases, a Cas12L protein includes a contiguous stretch of about 596 amino acids having 90% or more sequence identity (e.g., 95% or more, 97% or more, 98% or more, 99% or more, or 100% sequence identity) with the Cas12L amino acid sequence depicted in FIG. 4Y. In some cases, a Cas12L protein includes a contiguous stretch of about 596 amino acids having the Cas12L amino acid sequence depicted in FIG. 4Y. In some cases, a Cas12L protein includes a contiguous stretch of about 596 amino acids having the Cas12L protein sequence depicted in FIG. 4Y, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 750 aa, or from 750 aa to 800 aa). In some cases, the Cas12L polypeptide has a length of from about 725 amino acids to about 775 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4Y) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4Z

In some cases, a Cas12L protein (of the subject compositions and/or methods) includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4Z and designated “Cas12L_26_46464451.” For example, in some cases, a Cas12L protein includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4Z. In some cases, a Cas12L protein includes an amino acid sequence having 80% or more sequence identity (e.g., 85% or more, 90% or more, 95% or more, 97% or more, 98% or more, 99% or more, or 100% sequence identity) with the Cas12L amino acid sequence depicted in FIG. 4Z. In some cases, a Cas12L protein includes an amino acid sequence having 90% or more sequence identity (e.g., 95% or more, 97% or more, 98% or more, 99% or more, or 100% sequence identity) with the Cas12L amino acid sequence depicted in FIG. 4Z. In some cases, a Cas12L protein includes an amino acid sequence having the Cas12L amino acid sequence depicted in FIG. 4Z. In some cases, a Cas12L protein includes an amino acid sequence having the Cas12L protein sequence depicted in FIG. 4Z, with the exception that the sequence includes an amino acid substitution (e.g., 1, 2, or 3 amino acid substitutions) that reduces the naturally occurring catalytic activity of the protein. In some cases, the Cas12L polypeptide has a length of from 625 amino acids (aa) to 800 aa, e.g., from 625 aa to 640 aa, from 640 aa to 650 aa, from 650 aa to 700 aa, from 700 aa to 725 aa, from 725 aa to 750 aa, from 750 aa to 775 aa, or from 775 aa to 800 aa). In some cases, the Cas12L polypeptide has a length of 640 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4Z) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4AA

In some cases, a Cas12L protein (of the subject compositions and/or methods) includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4AA and designated “Cas12L_27_254489164.” For example, in some cases, a Cas12L protein includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4AA. In some cases, a Cas12L protein includes an amino acid sequence having 80% or more sequence identity (e.g., 85% or more, 90% or more, 95% or more, 97% or more, 98% or more, 99% or more, or 100% sequence identity) with the Cas12L amino acid sequence depicted in FIG. 4AA. In some cases, a Cas12L protein includes an amino acid sequence having 90% or more sequence identity (e.g., 95% or more, 97% or more, 98% or more, 99% or more, or 100% sequence identity) with the Cas12L amino acid sequence depicted in FIG. 4AA. In some cases, a Cas12L protein includes an amino acid sequence having the Cas12L amino acid sequence depicted in FIG. 4AA. In some cases, a Cas12L protein includes an amino acid sequence having the Cas12L protein sequence depicted in FIG. 4AA, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 746 aa, from 746 aa to 775 aa, or from 775 aa to 800 aa). In some cases, the Cas12L polypeptide has a length of 746 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4AA) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4BB

In some cases, a Cas12L protein (of the subject compositions and/or methods) includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4BB and designated “Cas12L_28_255238293.” For example, in some cases, a Cas12L protein includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4BB. In some cases, a Cas12L protein includes an amino acid sequence having 80% or more sequence identity (e.g., 85% or more, 90% or more, 95% or more, 97% or more, 98% or more, 99% or more, or 100% sequence identity) with the Cas12L amino acid sequence depicted in FIG. 4BB. In some cases, a Cas12L protein includes an amino acid sequence having 90% or more sequence identity (e.g., 95% or more, 97% or more, 98% or more, 99% or more, or 100% sequence identity) with the Cas12L amino acid sequence depicted in FIG. 4BB. In some cases, a Cas12L protein includes an amino acid sequence having the Cas12L amino acid sequence depicted in FIG. 4BB. In some cases, a Cas12L protein includes an amino acid sequence having the Cas12L protein sequence depicted in FIG. 4BB, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 759 aa, from 759 aa to 775 aa, or from 775 aa to 800 aa). In some cases, the Cas12L polypeptide has a length of 759 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4BB) includes the following nucleotide sequence: ATTGTTGAAATAGTACTTTTATAGTCTATATACAAC (SEQ ID NO:62) or the reverse complement of same. In some cases, the guide RNA comprises the nucleotide sequence (N)nATTGTTGAAATAGTACTTTTATAGTCTATATACAAC (SEQ ID NO:110) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4CC

In some cases, a Cas12L protein (of the subject compositions and/or methods) includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4CC and designated “Cas12L_29_72167294.” For example, in some cases, a Cas12L protein includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4CC. In some cases, a Cas12L protein includes an amino acid sequence having 80% or more sequence identity (e.g., 85% or more, 90% or more, 95% or more, 97% or more, 98% or more, 99% or more, or 100% sequence identity) with the Cas12L amino acid sequence depicted in FIG. 4CC. In some cases, a Cas12L protein includes an amino acid sequence having 90% or more sequence identity (e.g., 95% or more, 97% or more, 98% or more, 99% or more, or 100% sequence identity) with the Cas12L amino acid sequence depicted in FIG. 4CC. In some cases, a Cas12L protein includes an amino acid sequence having the Cas12L amino acid sequence depicted in FIG. 4CC. In some cases, a Cas12L protein includes an amino acid sequence having the Cas12L protein sequence depicted in FIG. 4CC, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 746 aa, from 746 aa to 775 aa, or from 775 aa to 800 aa). In some cases, the Cas12L polypeptide has a length of 746 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4CC) includes the following nucleotide sequence: TATTGTTGTAACTCTTATTTTGTATGGAGTAAACAAC (SEQ ID NO:63) or the reverse complement of same. In some cases, the guide RNA comprises the nucleotide sequence (N)nTATTGTTGTAACTCTTATTTTGTATGGAGTAAACAAC (SEQ ID NO:111) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4DD

In some cases, a Cas12L protein (of the subject compositions and/or methods) includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4DD and designated “Cas12L_30_72369269.” For example, in some cases, a Cas12L protein includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4DD. In some cases, a Cas12L protein includes an amino acid sequence having 80% or more sequence identity (e.g., 85% or more, 90% or more, 95% or more, 97% or more, 98% or more, 99% or more, or 100% sequence identity) with the Cas12L amino acid sequence depicted in FIG. 4DD. In some cases, a Cas12L protein includes an amino acid sequence having 90% or more sequence identity (e.g., 95% or more, 97% or more, 98% or more, 99% or more, or 100% sequence identity) with the Cas12L amino acid sequence depicted in FIG. 4DD. In some cases, a Cas12L protein includes an amino acid sequence having the Cas12L amino acid sequence depicted in FIG. 4DD. In some cases, a Cas12L protein includes an amino acid sequence having the Cas12L protein sequence depicted in FIG. 4DD, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 746 aa, from 746 aa to 775 aa, or from 775 aa to 800 aa). In some cases, the Cas12L polypeptide has a length of 746 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4DD) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4EE

In some cases, a Cas12L protein (of the subject compositions and/or methods) includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4EE and designated “Cas12L_31_72503976.” For example, in some cases, a Cas12L protein includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4EE. In some cases, a Cas12L protein includes an amino acid sequence having 80% or more sequence identity (e.g., 85% or more, 90% or more, 95% or more, 97% or more, 98% or more, 99% or more, or 100% sequence identity) with the Cas12L amino acid sequence depicted in FIG. 4EE. In some cases, a Cas12L protein includes an amino acid sequence having 90% or more sequence identity (e.g., 95% or more, 97% or more, 98% or more, 99% or more, or 100% sequence identity) with the Cas12L amino acid sequence depicted in FIG. 4EE. In some cases, a Cas12L protein includes an amino acid sequence having the Cas12L amino acid sequence depicted in FIG. 4EE. In some cases, a Cas12L protein includes an amino acid sequence having the Cas12L protein sequence depicted in FIG. 4EE, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 746 aa, from 746 aa to 775 aa, or from 775 aa to 800 aa). In some cases, the Cas12L polypeptide has a length of 746 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4EE) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4FF

In some cases, a Cas12L protein (of the subject compositions and/or methods) includes a contiguous stretch of about 47 amino acids having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4FF and designated “Cas12L_32_72547654_partial.” For example, in some cases, a Cas12L protein includes a contiguous stretch of about 47 amino acids having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4FF. In some cases, a Cas12L protein includes a contiguous stretch of about 47 amino acids having 80% or more sequence identity (e.g., 85% or more, 90% or more, 95% or more, 97% or more, 98% or more, 99% or more, or 100% sequence identity) with the Cas12L amino acid sequence depicted in FIG. 4FF. In some cases, a Cas12L protein includes a contiguous stretch of about 47 amino acids having 90% or more sequence identity (e.g., 95% or more, 97% or more, 98% or more, 99% or more, or 100% sequence identity) with the Cas12L amino acid sequence depicted in FIG. 4FF. In some cases, a Cas12L protein includes a contiguous stretch of about 47 amino acids having the Cas12L amino acid sequence depicted in FIG. 4FF. In some cases, a Cas12L protein includes a contiguous stretch of about 47 amino acids having the Cas12L protein sequence depicted in FIG. 4FF, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 750 aa, from 750 aa to 775 aa, or from 775 aa to 800 aa). In some cases, the Cas12L polypeptide has a length of from 725 amino acids to 775 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4FF) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4GG

In some cases, a Cas12L protein (of the subject compositions and/or methods) includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4GG and designated “Cas12L_33_72907394.” For example, in some cases, a Cas12L protein includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4GG. In some cases, a Cas12L protein includes an amino acid sequence having 80% or more sequence identity (e.g., 85% or more, 90% or more, 95% or more, 97% or more, 98% or more, 99% or more, or 100% sequence identity) with the Cas12L amino acid sequence depicted in FIG. 4GG. In some cases, a Cas12L protein includes an amino acid sequence having 90% or more sequence identity (e.g., 95% or more, 97% or more, 98% or more, 99% or more, or 100% sequence identity) with the Cas12L amino acid sequence depicted in FIG. 4GG. In some cases, a Cas12L protein includes an amino acid sequence having the Cas12L amino acid sequence depicted in FIG. 4GG. In some cases, a Cas12L protein includes an amino acid sequence having the Cas12L protein sequence depicted in FIG. 4GG, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 746 aa, from 746 aa to 775 aa, or from 775 aa to 800 aa). In some cases, the Cas12L polypeptide has a length of 746 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4GG) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4HH

In some cases, a Cas12L protein (of the subject compositions and/or methods) includes a contiguous stretch of about 245 amino acids having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4HH and designated “Cas12L_34_73124743_partial.” For example, in some cases, a Cas12L protein includes a contiguous stretch of about 245 amino acids having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4HH. In some cases, a Cas12L protein includes a contiguous stretch of about 245 amino acids having 80% or more sequence identity (e.g., 85% or more, 90% or more, 95% or more, 97% or more, 98% or more, 99% or more, or 100% sequence identity) with the Cas12L amino acid sequence depicted in FIG. 4HH. In some cases, a Cas12L protein includes a contiguous stretch of about 245 amino acids having 90% or more sequence identity (e.g., 95% or more, 97% or more, 98% or more, 99% or more, or 100% sequence identity) with the Cas12L amino acid sequence depicted in FIG. 4HH. In some cases, a Cas12L protein includes a contiguous stretch of about 245 amino acids having the Cas12L amino acid sequence depicted in FIG. 4HH. In some cases, a Cas12L protein includes a contiguous stretch of about 245 amino acids having the Cas12L protein sequence depicted in FIG. 4HH, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 750 aa, from 750 aa to 775 aa, or from 775 aa to 800 aa). In some cases, the Cas12L polypeptide has a length of from 725 amino acids to 775 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4HH) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4II

In some cases, a Cas12L protein (of the subject compositions and/or methods) includes a contiguous stretch of about 178 amino acids having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4II and designated “Cas12L_35_73503649_partial.” For example, in some cases, a Cas12L protein includes a contiguous stretch of about 178 amino acids having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4II. In some cases, a Cas12L protein includes a contiguous stretch of about 178 amino acids having 80% or more sequence identity (e.g., 85% or more, 90% or more, 95% or more, 97% or more, 98% or more, 99% or more, or 100% sequence identity) with the Cas12L amino acid sequence depicted in FIG. 4II. In some cases, a Cas12L protein includes a contiguous stretch of about 178 amino acids having 90% or more sequence identity (e.g., 95% or more, 97% or more, 98% or more, 99% or more, or 100% sequence identity) with the Cas12L amino acid sequence depicted in FIG. 4II. In some cases, a Cas12L protein includes a contiguous stretch of about 178 amino acids having the Cas12L amino acid sequence depicted in FIG. 4II. In some cases, a Cas12L protein includes a contiguous stretch of about 178 amino acids having the Cas12L protein sequence depicted in FIG. 4II, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 750 aa, from 750 aa to 775 aa, or from 775 aa to 800 aa). In some cases, the Cas12L polypeptide has a length of from 725 amino acids to 775 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4II) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4JJ

In some cases, a Cas12L protein (of the subject compositions and/or methods) includes a contiguous stretch of about 85 amino acids having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4JJ and designated “Cas12L_36_73503649_partial.” For example, in some cases, a Cas12L protein includes a contiguous stretch of about 85 amino acids having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4JJ. In some cases, a Cas12L protein includes a contiguous stretch of about 85 amino acids having 80% or more sequence identity (e.g., 85% or more, 90% or more, 95% or more, 97% or more, 98% or more, 99% or more, or 100% sequence identity) with the Cas12L amino acid sequence depicted in FIG. 4JJ. In some cases, a Cas12L protein includes a contiguous stretch of about 85 amino acids having 90% or more sequence identity (e.g., 95% or more, 97% or more, 98% or more, 99% or more, or 100% sequence identity) with the Cas12L amino acid sequence depicted in FIG. 4JJ. In some cases, a Cas12L protein includes a contiguous stretch of about 85 amino acids having the Cas12L amino acid sequence depicted in FIG. 4JJ. In some cases, a Cas12L protein includes a contiguous stretch of about 85 amino acids having the Cas12L protein sequence depicted in FIG. 4JJ, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 750 aa, from 750 aa to 775 aa, or from 775 aa to 800 aa). In some cases, the Cas12L polypeptide has a length of from 725 amino acids to 775 amino acids.

### FIG. 4KK

In some cases, a Cas12L protein (of the subject compositions and/or methods) includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4KK and designated “Cas12L_37_73472625.” For example, in some cases, a Cas12L protein includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4KK. In some cases, a Cas12L protein includes an amino acid sequence having 80% or more sequence identity (e.g., 85% or more, 90% or more, 95% or more, 97% or more, 98% or more, 99% or more, or 100% sequence identity) with the Cas12L amino acid sequence depicted in FIG. 4KK. In some cases, a Cas12L protein includes an amino acid sequence having 90% or more sequence identity (e.g., 95% or more, 97% or more, 98% or more, 99% or more, or 100% sequence identity) with the Cas12L amino acid sequence depicted in FIG. 4KK. In some cases, a Cas12L protein includes an amino acid sequence having the Cas12L amino acid sequence depicted in FIG. 4KK. In some cases, a Cas12L protein includes an amino acid sequence having the Cas12L protein sequence depicted in FIG. 4KK, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 750 aa, from 750 aa to 767 aa, from 767 aa to 775 aa, or from 775 aa to 800 aa). In some cases, the Cas12L polypeptide has a length of 767 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4KK) includes the following nucleotide sequence: ATTGTTGTAACATCTATTTTGTAAGGTGTAAACAAC (SEQ ID NO:64) or the reverse complement of same. In some cases, the guide RNA comprises the nucleotide sequence (N)nATTGTTGTAACATCTATTTTGTAAGGTGTAAACAAC (SEQ ID NO:112) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4LL

In some cases, a Cas12L protein (of the subject compositions and/or methods) includes a contiguous stretch of about 652 amino acids having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4LL and designated “Cas12L_38_73764039_partial.” For example, in some cases, a Cas12L protein includes a contiguous stretch of about 652 amino acids having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4LL. In some cases, a Cas12L protein includes a contiguous stretch of about 652 amino acids having 80% or more sequence identity (e.g., 85% or more, 90% or more, 95% or more, 97% or more, 98% or more, 99% or more, or 100% sequence identity) with the Cas12L amino acid sequence depicted in FIG. 4LL. In some cases, a Cas12L protein includes a contiguous stretch of about 652 amino acids having 90% or more sequence identity (e.g., 95% or more, 97% or more, 98% or more, 99% or more, or 100% sequence identity) with the Cas12L amino acid sequence depicted in FIG. 4LL. In some cases, a Cas12L protein includes a contiguous stretch of about 652 amino acids having the Cas12L amino acid sequence depicted in FIG. 4LL. In some cases, a Cas12L protein includes a contiguous stretch of about 652 amino acids having the Cas12L protein sequence depicted in FIG. 4LL, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 750 aa, from 750 aa to 775 aa, or from 775 aa to 800 aa). In some cases, the Cas12L polypeptide has a length of from 725 amino acids to 775 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4LL) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4MM

In some cases, a Cas12L protein (of the subject compositions and/or methods) includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4MM and designated “Cas12L_40_74037305.” For example, in some cases, a Cas12L protein includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4MM. In some cases, a Cas12L protein includes an amino acid sequence having 80% or more sequence identity (e.g., 85% or more, 90% or more, 95% or more, 97% or more, 98% or more, 99% or more, or 100% sequence identity) with the Cas12L amino acid sequence depicted in FIG. 4MM. In some cases, a Cas12L protein includes an amino acid sequence having 90% or more sequence identity (e.g., 95% or more, 97% or more, 98% or more, 99% or more, or 100% sequence identity) with the Cas12L amino acid sequence depicted in FIG. 4MM. In some cases, a Cas12L protein includes an amino acid sequence having the Cas12L amino acid sequence depicted in FIG. 4MM. In some cases, a Cas12L protein includes an amino acid sequence having the Cas12L protein sequence depicted in FIG. 4MM, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 750 aa, from 750 aa to 782 aa, or from 782 aa to 800 aa). In some cases, the Cas12L polypeptide has a length of 782 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4MM) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4NN

In some cases, a Cas12L protein (of the subject compositions and/or methods) includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4NN and designated “Cas12L_41_74408273_partial.” For example, in some cases, a Cas12L protein includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4NN. In some cases, a Cas12L protein includes an amino acid sequence having 80% or more sequence identity (e.g., 85% or more, 90% or more, 95% or more, 97% or more, 98% or more, 99% or more, or 100% sequence identity) with the Cas12L amino acid sequence depicted in FIG. 4NN. In some cases, a Cas12L protein includes an amino acid sequence having 90% or more sequence identity (e.g., 95% or more, 97% or more, 98% or more, 99% or more, or 100% sequence identity) with the Cas12L amino acid sequence depicted in FIG. 4NN. In some cases, a Cas12L protein includes an amino acid sequence having the Cas12L amino acid sequence depicted in FIG. 4NN. In some cases, a Cas12L protein includes an amino acid sequence having the Cas12L protein sequence depicted in FIG. 4NN, with the exception that the sequence includes an amino acid substitution (e.g., 1, 2, or 3 amino acid substitutions) that reduces the naturally occurring catalytic activity of the protein. In some cases, the Cas12L polypeptide has a length of from 850 amino acids (aa) to 950 aa, e.g., from 850 aa to 875 aa, from 875 aa to 889 aa, from 889 aa to 900 aa, or from 900 aa to 950 aa). In some cases, the Cas12L polypeptide has a length of 889 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4NN) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4OO

In some cases, a Cas12L protein (of the subject compositions and/or methods) includes a contiguous stretch of about 223 amino acids having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4OO and designated “Cas12L_42_75186079_partial.” For example, in some cases, a Cas12L protein includes a contiguous stretch of about 223 amino acids having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4OO. In some cases, a Cas12L protein includes a contiguous stretch of about 223 amino acids having 80% or more sequence identity (e.g., 85% or more, 90% or more, 95% or more, 97% or more, 98% or more, 99% or more, or 100% sequence identity) with the Cas12L amino acid sequence depicted in FIG. 4OO. In some cases, a Cas12L protein includes a contiguous stretch of about 223 amino acids having 90% or more sequence identity (e.g., 95% or more, 97% or more, 98% or more, 99% or more, or 100% sequence identity) with the Cas12L amino acid sequence depicted in FIG. 4OO. In some cases, a Cas12L protein includes a contiguous stretch of about 223 amino acids having the Cas12L amino acid sequence depicted in FIG. 4OO. In some cases, a Cas12L protein includes a contiguous stretch of about 223 amino acids having the Cas12L protein sequence depicted in FIG. 4OO, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 750 aa, from 750 aa to 775 aa, or from 775 aa to 800 aa). In some cases, the Cas12L polypeptide has a length of from 725 amino acids to 775 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4OO) includes the following nucleotide sequence: AATTGTTGTAACTCTTATTTTGTATGGAGTAAACAAC (SEQ ID NO:65) or the reverse complement of same. In some cases, the guide RNA comprises the nucleotide sequence (N)nAATTGTTGTAACTCTTATTTTGTATGGAGTAAACAAC (SEQ ID NO:113) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4PP

In some cases, a Cas12L protein (of the subject compositions and/or methods) includes a contiguous stretch of about 439 amino acids having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4PP and designated “Cas12L_43_75257103_partial.” For example, in some cases, a Cas12L protein includes a contiguous stretch of about 439 amino acids having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4PP. In some cases, a Cas12L protein includes a contiguous stretch of about 439 amino acids having 80% or more sequence identity (e.g., 85% or more, 90% or more, 95% or more, 97% or more, 98% or more, 99% or more, or 100% sequence identity) with the Cas12L amino acid sequence depicted in FIG. 4PP. In some cases, a Cas12L protein includes a contiguous stretch of about 439 amino acids having 90% or more sequence identity (e.g., 95% or more, 97% or more, 98% or more, 99% or more, or 100% sequence identity) with the Cas12L amino acid sequence depicted in FIG. 4PP. In some cases, a Cas12L protein includes a contiguous stretch of about 439 amino acids having the Cas12L amino acid sequence depicted in FIG. 4PP. In some cases, a Cas12L protein includes a contiguous stretch of about 439 amino acids having the Cas12L protein sequence depicted in FIG. 4PP, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 750 aa, from 750 aa to 775 aa, or from 775 aa to 800 aa). In some cases, the Cas12L polypeptide has a length of from 725 amino acids to 775 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4PP) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4QQ

In some cases, a Cas12L protein (of the subject compositions and/or methods) includes a contiguous stretch of about 196 amino acids having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4QQ and designated “Cas12L_44_75257103_partial.” For example, in some cases, a Cas12L protein includes a contiguous stretch of about 196 amino acids having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4QQ. In some cases, a Cas12L protein includes a contiguous stretch of about 196 amino acids having 80% or more sequence identity (e.g., 85% or more, 90% or more, 95% or more, 97% or more, 98% or more, 99% or more, or 100% sequence identity) with the Cas12L amino acid sequence depicted in FIG. 4QQ. In some cases, a Cas12L protein includes a contiguous stretch of about 196 amino acids having 90% or more sequence identity (e.g., 95% or more, 97% or more, 98% or more, 99% or more, or 100% sequence identity) with the Cas12L amino acid sequence depicted in FIG. 4QQ. In some cases, a Cas12L protein includes a contiguous stretch of about 196 amino acids having the Cas12L amino acid sequence depicted in FIG. 4QQ. In some cases, a Cas12L protein includes a contiguous stretch of about 196 amino acids having the Cas12L protein sequence depicted in FIG. 4QQ, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 750 aa, from 750 aa to 775 aa, or from 775 aa to 800 aa). In some cases, the Cas12L polypeptide has a length of from 725 amino acids to 775 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4QQ) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4RR

In some cases, a Cas12L protein (of the subject compositions and/or methods) includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4RR and designated “Cas12L_45_75616607_partial.” For example, in some cases, a Cas12L protein includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4RR. In some cases, a Cas12L protein includes an amino acid sequence having 80% or more sequence identity (e.g., 85% or more, 90% or more, 95% or more, 97% or more, 98% or more, 99% or more, or 100% sequence identity) with the Cas12L amino acid sequence depicted in FIG. 4RR. In some cases, a Cas12L protein includes an amino acid sequence having 90% or more sequence identity (e.g., 95% or more, 97% or more, 98% or more, 99% or more, or 100% sequence identity) with the Cas12L amino acid sequence depicted in FIG. 4RR. In some cases, a Cas12L protein includes an amino acid sequence having the Cas12L amino acid sequence depicted in FIG. 4RR. In some cases, a Cas12L protein includes an amino acid sequence having the Cas12L protein sequence depicted in FIG. 4RR, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 748 aa, from 748 aa to 775 aa, or from 775 aa to 800 aa). In some cases, the Cas12L polypeptide has a length of 748 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4RR) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4SS

In some cases, a Cas12L protein (of the subject compositions and/or methods) includes a contiguous stretch of about 481 amino acids having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4SS and designated “Cas12L_46_75784289_partial.” For example, in some cases, a Cas12L) includes a contiguous stretch of about 481 amino acids having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4SS. In some cases, a Cas12L protein) includes a contiguous stretch of about 481 amino acids having 80% or more sequence identity (e.g., 85% or more, 90% or more, 95% or more, 97% or more, 98% or more, 99% or more, or 100% sequence identity) with the Cas12L amino acid sequence depicted in FIG. 4SS. In some cases, a Cas12L protein) includes a contiguous stretch of about 481 amino acids having 90% or more sequence identity (e.g., 95% or more, 97% or more, 98% or more, 99% or more, or 100% sequence identity) with the Cas12L amino acid sequence depicted in FIG. 4SS. In some cases, a Cas12L protein) includes a contiguous stretch of about 481 amino acids having the Cas12L amino acid sequence depicted in FIG. 4SS. In some cases, a Cas12L protein) includes a contiguous stretch of about 481 amino acids having the Cas12L protein sequence depicted in FIG. 4SS, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 750 aa, from 750 aa to 775 aa, or from 775 aa to 800 aa). In some cases, the Cas12L polypeptide has a length of from 725 amino acids to 775 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4SS) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4TT

In some cases, a Cas12L protein (of the subject compositions and/or methods) includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4TT and designated “Cas12L_47_76512228.” For example, in some cases, a Cas12L protein includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4TT. In some cases, a Cas12L protein includes an amino acid sequence having 80% or more sequence identity (e.g., 85% or more, 90% or more, 95% or more, 97% or more, 98% or more, 99% or more, or 100% sequence identity) with the Cas12L amino acid sequence depicted in FIG. 4TT. In some cases, a Cas12L protein includes an amino acid sequence having 90% or more sequence identity (e.g., 95% or more, 97% or more, 98% or more, 99% or more, or 100% sequence identity) with the Cas12L amino acid sequence depicted in FIG. 4TT. In some cases, a Cas12L protein includes an amino acid sequence having the Cas12L amino acid sequence depicted in FIG. 4TT. In some cases, a Cas12L protein includes an amino acid sequence having the Cas12L protein sequence depicted in FIG. 4TT, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 746 aa, from 746 aa to 775 aa, or from 775 aa to 800 aa). In some cases, the Cas12L polypeptide has a length of 746 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4TT) includes the following nucleotide sequence: ATTGTTGTAACTTTTATTTTGTATGGAGTAAACAAC (SEQ ID NO:66) or the reverse complement of same. In some cases, the guide RNA comprises the nucleotide sequence (N)nATTGTTGTAACTTTTATTTTGTATGGAGTAAACAAC (SEQ ID NO:114) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4UU

In some cases, a Cas12L protein (of the subject compositions and/or methods) includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4UU and designated “Cas12L_48_76600450.” For example, in some cases, a Cas12L protein includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4UU. In some cases, a Cas12L protein includes an amino acid sequence having 80% or more sequence identity (e.g., 85% or more, 90% or more, 95% or more, 97% or more, 98% or more, 99% or more, or 100% sequence identity) with the Cas12L amino acid sequence depicted in FIG. 4UU. In some cases, a Cas12L protein includes an amino acid sequence having 90% or more sequence identity (e.g., 95% or more, 97% or more, 98% or more, 99% or more, or 100% sequence identity) with the Cas12L amino acid sequence depicted in FIG. 4UU. In some cases, a Cas12L protein includes an amino acid sequence having the Cas12L amino acid sequence depicted in FIG. 4UU. In some cases, a Cas12L protein includes an amino acid sequence having the Cas12L protein sequence depicted in FIG. 4UU, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 746 aa, from 746 aa to 775 aa, or from 775 aa to 800 aa). In some cases, the Cas12L polypeptide has a length of 746 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4UU) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4VV

In some cases, a Cas12L protein (of the subject compositions and/or methods) includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4VV and designated “Cas12L_49_44880081.” For example, in some cases, a Cas12L protein includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4VV. In some cases, a Cas12L protein includes an amino acid sequence having 80% or more sequence identity (e.g., 85% or more, 90% or more, 95% or more, 97% or more, 98% or more, 99% or more, or 100% sequence identity) with the Cas12L amino acid sequence depicted in FIG. 4VV. In some cases, a Cas12L protein includes an amino acid sequence having 90% or more sequence identity (e.g., 95% or more, 97% or more, 98% or more, 99% or more, or 100% sequence identity) with the Cas12L amino acid sequence depicted in FIG. 4VV. In some cases, a Cas12L protein includes an amino acid sequence having the Cas12L amino acid sequence depicted in FIG. 4VV. In some cases, a Cas12L protein includes an amino acid sequence having the Cas12L protein sequence depicted in FIG. 4VV, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 746 aa, from 746 aa to 775 aa, or from 775 aa to 800 aa). In some cases, the Cas12L polypeptide has a length of 746 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4VV) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4WW

In some cases, a Cas12L protein (of the subject compositions and/or methods) includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4WW and designated “Cas12L_50_83012613.” For example, in some cases, a Cas12L protein includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4WW. In some cases, a Cas12L protein includes an amino acid sequence having 80% or more sequence identity (e.g., 85% or more, 90% or more, 95% or more, 97% or more, 98% or more, 99% or more, or 100% sequence identity) with the Cas12L amino acid sequence depicted in FIG. 4WW. In some cases, a Cas12L protein includes an amino acid sequence having 90% or more sequence identity (e.g., 95% or more, 97% or more, 98% or more, 99% or more, or 100% sequence identity) with the Cas12L amino acid sequence depicted in FIG. 4WW. In some cases, a Cas12L protein includes an amino acid sequence having the Cas12L amino acid sequence depicted in FIG. 4WW. In some cases, a Cas12L protein includes an amino acid sequence having the Cas12L protein sequence depicted in FIG. 4WW, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 735 aa, from 735 aa to 750 aa, from 750 aa to 775 aa, or from 775 aa to 800 aa). In some cases, the Cas12L polypeptide has a length of 735 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4WW) includes the following nucleotide sequence: AATGTTGTAGATACCTTTTTGTAAGGATTGAACAAC (SEQ ID NO:67) or the reverse complement of same. In some cases, the guide RNA comprises the nucleotide sequence (N)nAATGTTGTAGATACCTTTTTGTAAGGATTGAACAAC (SEQ ID NO:115) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4XX

In some cases, a Cas12L protein (of the subject compositions and/or methods) includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4XX and designated “Cas12L_51_82983331.” For example, in some cases, a Cas12L protein includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4XX. In some cases, a Cas12L protein includes an amino acid sequence having 80% or more sequence identity (e.g., 85% or more, 90% or more, 95% or more, 97% or more, 98% or more, 99% or more, or 100% sequence identity) with the Cas12L amino acid sequence depicted in FIG. 4XX. In some cases, a Cas12L protein includes an amino acid sequence having 90% or more sequence identity (e.g., 95% or more, 97% or more, 98% or more, 99% or more, or 100% sequence identity) with the Cas12L amino acid sequence depicted in FIG. 4XX. In some cases, a Cas12L protein includes an amino acid sequence having the Cas12L amino acid sequence depicted in FIG. 4XX. In some cases, a Cas12L protein includes an amino acid sequence having the Cas12L protein sequence depicted in FIG. 4XX, with the exception that the sequence includes an amino acid substitution (e.g., 1, 2, or 3 amino acid substitutions) that reduces the naturally occurring catalytic activity of the protein. In some cases, the Cas12L polypeptide has a length of from 750 amino acids (aa) to 850 aa, e.g., from 750 aa to 779 aa, from 779 aa to 800 aa, or from 800 aa to 850 aa). In some cases, the Cas12L polypeptide has a length of 779 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4XX) includes the following nucleotide sequence: ATTGTTGTAATACTATTTTTGTAAAGTATAAACAAC (SEQ ID NO:68) or the reverse complement of same. In some cases, the guide RNA comprises the nucleotide sequence (N)nATTGTTGTAATACTATTTTTGTAAAGTATAAACAAC (SEQ ID NO:116) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4YY

In some cases, a Cas12L protein (of the subject compositions and/or methods) includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4YY and designated “Cas12L_52_76767885.” For example, in some cases, a Cas12L protein includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4YY. In some cases, a Cas12L protein includes an amino acid sequence having 80% or more sequence identity (e.g., 85% or more, 90% or more, 95% or more, 97% or more, 98% or more, 99% or more, or 100% sequence identity) with the Cas12L amino acid sequence depicted in FIG. 4YY. In some cases, a Cas12L protein includes an amino acid sequence having 90% or more sequence identity (e.g., 95% or more, 97% or more, 98% or more, 99% or more, or 100% sequence identity) with the Cas12L amino acid sequence depicted in FIG. 4YY. In some cases, a Cas12L protein includes an amino acid sequence having the Cas12L amino acid sequence depicted in FIG. 4YY. In some cases, a Cas12L protein includes an amino acid sequence having the Cas12L protein sequence depicted in FIG. 4YY, with the exception that the sequence includes an amino acid substitution (e.g., 1, 2, or 3 amino acid substitutions) that reduces the naturally occurring catalytic activity of the protein. In some cases, the Cas12L polypeptide has a length of from 550 amino acids (aa) to 700 aa, e.g., from 550 aa to 592 aa, from 592 aa to 625 aa, from 625 aa to 650 aa, or from 650 aa to 700 aa). In some cases, the Cas12L polypeptide has a length of 592 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4YY) includes the following nucleotide sequence: AATGTTGTAGATGCCTTTTTATAAGGATTAAACAAC (SEQ ID NO:69) or the reverse complement of same. In some cases, the guide RNA comprises the nucleotide sequence (N)nAATGTTGTAGATGCCTTTTTATAAGGATTAAACAAC (SEQ ID NO:117) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4ZZ

In some cases, a Cas12L protein (of the subject compositions and/or methods) includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4ZZ and designated “Cas12L_53_77216451.” For example, in some cases, a Cas12L protein includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4ZZ. In some cases, a Cas12L protein includes an amino acid sequence having 80% or more sequence identity (e.g., 85% or more, 90% or more, 95% or more, 97% or more, 98% or more, 99% or more, or 100% sequence identity) with the Cas12L amino acid sequence depicted in FIG. 4ZZ. In some cases, a Cas12L protein includes an amino acid sequence having 90% or more sequence identity (e.g., 95% or more, 97% or more, 98% or more, 99% or more, or 100% sequence identity) with the Cas12L amino acid sequence depicted in FIG. 4ZZ. In some cases, a Cas12L protein includes an amino acid sequence having the Cas12L amino acid sequence depicted in FIG. 4ZZ. In some cases, a Cas12L protein includes an amino acid sequence having the Cas12L protein sequence depicted in FIG. 4ZZ, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 746 aa, from 746 aa to 775 aa, or from 775 aa to 800 aa). In some cases, the Cas12L polypeptide has a length of 746 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4ZZ) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4AAA

In some cases, a Cas12L protein (of the subject compositions and/or methods) includes a contiguous stretch of about 29 amino acids having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4AAA and designated “Cas12L_54_77468912_partial.” For example, in some cases, a Cas12L protein includes a contiguous stretch of about 29 amino acids having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4AAA. In some cases, a Cas12L protein includes a contiguous stretch of about 29 amino acids having 80% or more sequence identity (e.g., 85% or more, 90% or more, 95% or more, 97% or more, 98% or more, 99% or more, or 100% sequence identity) with the Cas12L amino acid sequence depicted in FIG. 4AAA. In some cases, a Cas12L protein includes a contiguous stretch of about 29 amino acids having 90% or more sequence identity (e.g., 95% or more, 97% or more, 98% or more, 99% or more, or 100% sequence identity) with the Cas12L amino acid sequence depicted in FIG. 4AAA. In some cases, a Cas12L protein includes a contiguous stretch of about 29 amino acids having the Cas12L amino acid sequence depicted in FIG. 4AAA. In some cases, a Cas12L protein includes a contiguous stretch of about 29 amino acids having the Cas12L protein sequence depicted in FIG. 4AAA, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 750 aa, from 750 aa to 775 aa, or from 775 aa to 800 aa). In some cases, the Cas12L polypeptide has a length of from 725 amino acids to 775 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4AAA) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4BBB

In some cases, a Cas12L protein (of the subject compositions and/or methods) includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4BBB and designated “Cas12L_55_77738117.” For example, in some cases, a Cas12L protein includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4BBB. In some cases, a Cas12L protein includes an amino acid sequence having 80% or more sequence identity (e.g., 85% or more, 90% or more, 95% or more, 97% or more, 98% or more, 99% or more, or 100% sequence identity) with the Cas12L amino acid sequence depicted in FIG. 4BBB. In some cases, a Cas12L protein includes an amino acid sequence having 90% or more sequence identity (e.g., 95% or more, 97% or more, 98% or more, 99% or more, or 100% sequence identity) with the Cas12L amino acid sequence depicted in FIG. 4BBB. In some cases, a Cas12L protein includes an amino acid sequence having the Cas12L amino acid sequence depicted in FIG. 4BBB. In some cases, a Cas12L protein includes an amino acid sequence having the Cas12L protein sequence depicted in FIG. 4BBB, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 750 aa, from 750 aa to 775 aa, or from 775 aa to 800 aa). In some cases, the Cas12L polypeptide has a length of from 725 amino acids to 775 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4BBB) includes the following nucleotide sequence: ATTGTTGTAATACACTTTTTATAAGGTATGAACAAC (SEQ ID NO:70) or the reverse complement of same. In some cases, the guide RNA comprises the nucleotide sequence
(N)nATTGTTGTAATACACTTTTTATAAGGTATGAACAAC (SEQ ID NO:118) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4CCC

In some cases, a Cas12L protein (of the subject compositions and/or methods) includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4CCC and designated “Cas12L_56_65286425.” For example, in some cases, a Cas12L protein includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4CCC. In some cases, a Cas12L protein includes an amino acid sequence having 80% or more sequence identity (e.g., 85% or more, 90% or more, 95% or more, 97% or more, 98% or more, 99% or more, or 100% sequence identity) with the Cas12L amino acid sequence depicted in FIG. 4CCC. In some cases, a Cas12L protein includes an amino acid sequence having 90% or more sequence identity (e.g., 95% or more, 97% or more, 98% or more, 99% or more, or 100% sequence identity) with the Cas12L amino acid sequence depicted in FIG. 4CCC. In some cases, a Cas12L protein includes an amino acid sequence having the Cas12L amino acid sequence depicted in FIG. 4CCC. In some cases, a Cas12L protein includes an amino acid sequence having the Cas12L protein sequence depicted in FIG. 4CCC, with the exception that the sequence includes an amino acid substitution (e.g., 1, 2, or 3 amino acid substitutions) that reduces the naturally occurring catalytic activity of the protein. In some cases, the Cas12L polypeptide has a length of from 650 amino acids (aa) to 750 aa, e.g., from 650 aa to 692 aa, from 692 aa to 725 aa, or from 725 aa to 750 aa). In some cases, the Cas12L polypeptide has a length of 692 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4CCC) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4DDD

In some cases, a Cas12L protein (of the subject compositions and/or methods) includes a contiguous stretch of about 441 amino acids having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4DDD and designated “Cas12L_57_65567118_partial.” For example, in some cases, a Cas12L protein) includes a contiguous stretch of about 441 amino acids having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4DDD. In some cases, a Cas12L protein) includes a contiguous stretch of about 441 amino acids having 80% or more sequence identity (e.g., 85% or more, 90% or more, 95% or more, 97% or more, 98% or more, 99% or more, or 100% sequence identity) with the Cas12L amino acid sequence depicted in FIG. 4DDD. In some cases, a Cas12L protein) includes a contiguous stretch of about 441 amino acids having 90% or more sequence identity (e.g., 95% or more, 97% or more, 98% or more, 99% or more, or 100% sequence identity) with the Cas12L amino acid sequence depicted in FIG. 4DDD. In some cases, a Cas12L protein) includes a contiguous stretch of about 441 amino acids having the Cas12L amino acid sequence depicted in FIG. 4DDD. In some cases, a Cas12L protein) includes a contiguous stretch of about 441 amino acids having the Cas12L protein sequence depicted in FIG. 4DDD, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 750 aa, from 750 aa to 775 aa, or from 775 aa to 800 aa). In some cases, the Cas12L polypeptide has a length of from 725 amino acids to 775 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4DDD) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4EEE

In some cases, a Cas12L protein (of the subject compositions and/or methods) includes a contiguous stretch of about 397 amino acids having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4EEE and designated “Cas12L_58_66287853_partial.” For example, in some cases, a Cas12L protein includes a contiguous stretch of about 397 amino acids having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4EEE. In some cases, a Cas12L protein includes a contiguous stretch of about 397 amino acids having 80% or more sequence identity (e.g., 85% or more, 90% or more, 95% or more, 97% or more, 98% or more, 99% or more, or 100% sequence identity) with the Cas12L amino acid sequence depicted in FIG. 4EEE. In some cases, a Cas12L protein includes a contiguous stretch of about 397 amino acids having 90% or more sequence identity (e.g., 95% or more, 97% or more, 98% or more, 99% or more, or 100% sequence identity) with the Cas12L amino acid sequence depicted in FIG. 4EEE. In some cases, a Cas12L protein includes a contiguous stretch of about 397 amino acids having the Cas12L amino acid sequence depicted in FIG. 4EEE. In some cases, a Cas12L protein includes a contiguous stretch of about 397 amino acids having the Cas12L protein sequence depicted in FIG. 4EEE, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 750 aa, from 750 aa to 775 aa, or from 775 aa to 800 aa). In some cases, the Cas12L polypeptide has a length of from 725 amino acids to 775 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4EEE) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### FIG. 4FFF

In some cases, a Cas12L protein (of the subject compositions and/or methods) includes an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12L amino acid sequence depicted in FIG. 4FFF and designated “Cas12L_39_73877227.” For example, in some cases, a Cas12L protein includes an amino acid sequence having 50% or more sequence identity (e.g., 60% or more, 70% or more, 80% or more, 85% or more, 90% or more, 95% or more, 97% or more, 98% or more, 99% or more, or 100% sequence identity) with the Cas12L amino acid sequence depicted in FIG. 4FFF. In some cases, a Cas12L protein includes an amino acid sequence having 80% or more sequence identity (e.g., 85% or more, 90% or more, 95% or more, 97% or more, 98% or more, 99% or more, or 100% sequence identity) with the Cas12L amino acid sequence depicted in FIG. 4FFF. In some cases, a Cas12L protein includes an amino acid sequence having 90% or more sequence identity (e.g., 95% or more, 97% or more, 98% or more, 99% or more, or 100% sequence identity) with the Cas12L amino acid sequence depicted in FIG. 4FFF. In some cases, a Cas12L protein includes an amino acid sequence having the Cas12L amino acid sequence depicted in FIG. 4FFF. In some cases, a Cas12L protein includes an amino acid sequence having the Cas12L protein sequence depicted in FIG. 4FFF, with the exception that the sequence includes an amino acid substitution (e.g., 1, 2, or 3 amino acid substitutions) that reduces the naturally occurring catalytic activity of the protein. In some cases, the Cas12L polypeptide has a length of from 700 amino acids (aa) to 800 aa, e.g., from 700 aa to 725 aa, from 725 aa to 746 aa, from 746 aa to 775 aa, or from 775 aa to 800 aa). In some cases, the Cas12L polypeptide has a length of 746 amino acids. In some cases, a guide RNA that binds a Cas12L polypeptide (e.g., a Cas12L polypeptide comprising an amino acid sequence having 20% or more, 30% or more, 40% or more, 50% or more, 60% or more, 70% or more, 80% or more, 85% or more, 90% or more, 95% or more, 97% or more, 98% or more, 99% or more, or 100%, amino acid sequence identity to the Cas12L amino acid sequence depicted in FIG. 4FFF) includes the following nucleotide sequence: ATTGTTGTAACTCTTATTTTGTATGGAGTAAACAAC (SEQ ID NO:57) or the reverse complement of same. In some cases, the guide RNA comprises the nucleotide sequence (N)nATTGTTGTAACTCTTATTTTGTATGGAGTAAACAAC (SEQ ID NO:105) or the reverse complement of same, where N is any nucleotide and n is an integer from 15 to 30, e.g., from 15 to 20, from 17 to 25, from 17 to 22, from 18 to 22, from 18 to 20, from 20 to 25, or from 25 to 30).

### Cas12L Variants

A variant Cas12L protein has an amino acid sequence that is different by at least one amino acid (e.g., has a deletion, insertion, substitution, fusion) when compared to the amino acid sequence of the corresponding wild type Cas12L protein, e.g., when compared to the Cas12L amino acid sequence depicted in any one of FIG. 4A-4Z, FIG. 4AA-4ZZ, and FIG. 4AAA-4FFF. In some cases, a Cas12L variant comprises from 1 amino acid substitution to 10 amino acid substitutions compared to the Cas12L amino acid sequence depicted in any one of FIG. 4A-4Z, FIG. 4AA-4ZZ, and FIG. 4AAA-4FFF. In some cases, a Cas12L variant comprises from 1 amino acid substitution to 10 amino acid substitutions in the RuvC domain, compared to the Cas12L amino acid sequence depicted in any one of FIG. 4A-4Z, FIG. 4AA-4ZZ, and FIG. 4AAA-4FFF.

### Variants—Catalytic Activity

In some cases, the Cas12L protein is a variant Cas12L protein, e.g., mutated relative to the naturally occurring catalytically active sequence, and exhibits reduced cleavage activity (e.g., exhibits 90%, or less, 80% or less, 70% or less, 60% or less, 50% or less, 40% or less, or 30% or less cleavage activity) when compared to the corresponding naturally occurring sequence. In some cases, such a variant Cas12L protein is a catalytically ‘dead’ protein (has substantially no cleavage activity) and can be referred to as a ‘dCas12L.’ In some cases, the variant Cas12L protein is a nickase (cleaves only one strand of a double stranded target nucleic acid, e.g., a double stranded target DNA). As described in more detail herein, in some cases, a Cas12L protein (in some case a Cas12L protein with wild type cleavage activity and in some cases a variant Cas12L with reduced cleavage activity, e.g., a dCas12L or a nickase Cas12L) is fused (conjugated) to a heterologous polypeptide that has an activity of interest (e.g., a catalytic activity of interest) to form a fusion protein (a fusion Cas12L protein).

In some cases, a variant Cas12L polypeptide comprises a substitution of one, two, or three amino acids of the active site residues indicated in FIG. 3A-3B, where the variant Cas12L polypeptide exhibits reduced catalytic activity compared to a control Cas12L polypeptide that does not include the one, two, or three substitutions.

### Variants—Fusion Cas12L Polypeptides

As noted above, in some cases, a Cas12L protein (in some cases a Cas12L protein with wild type cleavage activity and in some cases a variant Cas12L with reduced cleavage activity, e.g., a dCas12L or a nickase Cas12L) is fused (conjugated) to a heterologous polypeptide that has an activity of interest (e.g., a catalytic activity of interest) to form a fusion protein. A heterologous polypeptide to which a Cas12L protein can be fused is referred to herein as a ‘fusion partner.’

In some cases, the fusion partner can modulate transcription (e.g., inhibit transcription, increase transcription) of a target DNA. For example, in some cases the fusion partner is a protein (or a domain from a protein) that inhibits transcription (e.g., a transcriptional repressor, a protein that functions via recruitment of transcription inhibitor proteins, modification of target DNA such as methylation, recruitment of a DNA modifier, modulation of histones associated with target DNA, recruitment of a histone modifier such as those that modify acetylation and/or methylation of histones, and the like). In some cases the fusion partner is a protein (or a domain from a protein) that increases transcription (e.g., a transcription activator, a protein that acts via recruitment of transcription activator proteins, modification of target DNA such as demethylation, recruitment of a DNA modifier, modulation of histones associated with target DNA, recruitment of a histone modifier such as those that modify acetylation and/or methylation of histones, and the like).

In some cases, the fusion partner (heterologous polypeptide) is a reverse transcriptase. In some cases, the fusion partner is a base editor. In some cases, the fusion partner (heterologous polypeptide) is a deaminase.

In some cases, a fusion Cas12L protein includes a heterologous polypeptide that has enzymatic activity that modifies a target nucleic acid (e.g., nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity).

In some cases, a fusion Cas12L protein includes a heterologous polypeptide that has enzymatic activity that modifies a polypeptide (e.g., a histone) associated with a target nucleic acid (e.g., methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity).

Examples of proteins (or fragments thereof) that can be used in increase transcription include but are not limited to: transcriptional activators such as VP16, VP64, VP48, VP160, p65 subdomain (e.g., from NFkB), and activation domain of EDLL and/or TAL activation domain (e.g., for activity in plants); histone lysine methyltransferases such as SET1A, SET1B, MLL1 to 5, ASHI, SYMD2, NSD1, and the like; histone lysine demethylases such as JHDM2a/b, UTX, JMJD3, and the like; histone acetyltransferases such as GCN5, PCAF, CBP, p300, TAF1, TIP60/PLIP, MOZ/MYST3, MORF/MYST4, SRC1, ACTR, P160, CLOCK, and the like; and DNA demethylases such as Ten-Eleven Translocation (TET) dioxygenase 1 (TET1CD), TET1, DME, DML1, DML2, ROS1, and the like.

Examples of proteins (or fragments thereof) that can be used in decrease transcription include but are not limited to: transcriptional repressors such as the Kruppel associated box (KRAB or SKD); KOX1 repression domain; the Mad mSIN3 interaction domain (SID); the ERF repressor domain (ERD), the SRDX repression domain (e.g., for repression in plants), and the like; histone lysine methyltransferases such as Pr-SET7/8, SUV4-20H1, RIZ1, and the like; histone lysine demethylases such as JMJD2A/JHDM3A, JMJD2B, JMJD2C/GASC1, JMJD2D, JARID1A/RBP2, JARID1B/PLU-1, JARID1C/SMCX, JARID1D/SMCY, and the like; histone lysine deacetylases such as HDAC1, HDAC2, HDAC3, HDAC8, HDAC4, HDAC5, HDAC7, HDAC9, SIRT1, SIRT2, HDAC11, and the like; DNA methylases such as HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants), and the like; and periphery recruitment elements such as Lamin A, Lamin B, and the like.

In some cases, the fusion partner has enzymatic activity that modifies the target nucleic acid (e.g., ssRNA, dsRNA, ssDNA, dsDNA). Examples of enzymatic activity that can be provided by the fusion partner include but are not limited to: nuclease activity such as that provided by a restriction enzyme (e.g., FokI nuclease), methyltransferase activity such as that provided by a methyltransferase (e.g., HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants), and the like); demethylase activity such as that provided by a demethylase (e.g., Ten-Eleven Translocation (TET) dioxygenase 1 (TET1CD), TET1, DME, DML1, DML2, ROS1, and the like), DNA repair activity, DNA damage activity, deamination activity such as that provided by a deaminase (e.g., a cytosine deaminase enzyme such as rat APOBEC1), dismutase activity, alkylation activity, depurination activity, oxidation activity, pyrimidine dimer forming activity, integrase activity such as that provided by an integrase and/or resolvase (e.g., Gin invertase such as the hyperactive mutant of the Gin invertase, GinH106Y; human immunodeficiency virus type 1 integrase (IN); Tn3 resolvase; and the like), transposase activity, recombinase activity such as that provided by a recombinase (e.g., catalytic domain of Gin recombinase), polymerase activity, ligase activity, helicase activity, photolyase activity, and glycosylase activity).

In some cases, the fusion partner has enzymatic activity that modifies a protein associated with the target nucleic acid (e.g., ssRNA, dsRNA, ssDNA, dsDNA) (e.g., a histone, an RNA binding protein, a DNA binding protein, and the like). Examples of enzymatic activity (that modifyies a protein associated with a target nucleic acid) that can be provided by the fusion partner include but are not limited to: methyltransferase activity such as that provided by a histone methyltransferase (HMT) (e.g., suppressor of variegation 3-9 homolog 1 (SUV39H1, also known as KMT1A), euchromatic histone lysine methyltransferase 2 (G9A, also known as KMT1C and EHMT2), SUV39H2, ESET/SETDB1, and the like, SET1A, SET1B, MLL1 to 5, ASHI, SYMD2, NSD1, DOT1L, Pr-SET7/8, SUV4-20H1, EZH2, RIZ1), demethylase activity such as that provided by a histone demethylase (e.g., Lysine Demethylase 1A (KDM1A also known as LSD1), JHDM2a/b, JMJD2A/JHDM3A, JMJD2B, JMJD2C/GASC1, JMJD2D, JARID1A/RBP2, JARID1B/PLU-1, JARID1C/SMCX, JARID1D/SMCY, UTX, JMJD3, and the like), acetyltransferase activity such as that provided by a histone acetylase transferase (e.g., catalytic core/fragement of the human acetyltransferase p300, GCN5, PCAF, CBP, TAFI, TIP60/PLIP, MOZ/MYST3, MORF/MYST4, HBO1/MYST2, HMOF/MYST1, SRC1, ACTR, P160, CLOCK, and the like), deacetylase activity such as that provided by a histone deacetylase (e.g., HDAC1, HDAC2, HDAC3, HDAC8, HDAC4, HDAC5, HDAC7, HDAC9, SIRT1, SIRT2, HDAC11, and the like), kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, and demyristoylation activity.

Additional examples of a suitable fusion partners are dihydrofolate reductase (DHFR) destabilization domain (e.g., to generate a chemically controllable fusion Cas12L protein), and a chloro last transit peptide. Suitable chloro last transit peptides include but are not limited to:

(SEQ ID NO: 119)
MASMISSSAVTTVSRASRGQSAAMAPFGGLKSMTGFPVRKVNTDITSITSNGGRV
KCMQVWPPIGKKKFETLSYLPPLTRDSRA;
(SEQ ID NO: 120)
MASMISSSAVTTVSRASRGQSAAMAPFGGLKSMTGFPVRKVNTDITSITSNGGRVKS;
(SEQ ID NO: 121)
MASSMLSSATMVASPAQATMVAPFNGLKSSAAFPATRKANNDITSITSNGGRVNCMQVWPPIEK
KKFETLSYLPDLTDSGGRVNC;
(SEQ ID NO: 122)
MAQVSRICNGVQNPSLISNLSKSSQRKSPLSVSLKTQQHPRAYPISSSWGLKKSGMTLIGSELRPL
KVMSSVSTAC;
(SEQ ID NO: 123)
MAQVSRICNGVWNPSLISNLSKSSQRKSPLSVSLKTQQHPRAYPISSSWGLKKSGMTLIGSELRPL
KVMSSVSTAC;
(SEQ ID NO: 124)
MAQINNMAQGIQTLNPNSNFHKPQVPKSSSFLVFGSKKLKNSANSMLVLKKDSIFMQLFCSFRIS
ASVATAC;
(SEQ ID NO: 125)
MAALVTSQLATSGTVLSVTDRFRRPGFQGLRPRNPADAALGMRTVGASAAPKQSRKPHRFDRR
CLSMVV;
(SEQ ID NO: 126)
MAALTTSQLATSATGFGIADRSAPSSLLRHGFQGLKPRSPAGGDATSLSVTTSARATPKQQRSVQ
RGSRRFPSVVVC;
(SEQ ID NO: 127)
MASSVLSSAAVATRSNVAQANMVAPFTGLKSAASFPVSRKQNLDITSIASNGGRVQC;
(SEQ ID NO: 128)
MESLAATSVFAPSRVAVPAARALVRAGTVVPTRRTSSTSGTSGVKCSAAVTPQASPVISRSAAAA;
and
(SEQ ID NO: 129)
MGAAATSMQSLKFSNRLVPPSRRLSPVPNNVTCNNLPKSAAPVRTVKCCASSWNSTINGAAATT
NGASAASS.

In some case, a Cas12L fusion polypeptide of the present disclosure comprises: a) a Cas12L polypeptide of the present disclosure; and b) a chloroplast transit peptide. Thus, for example, a Cas12L polypeptide/guide RNA complex can be targeted to the chloroplast. In some cases, this targeting may be achieved by the presence of an N-terminal extension, called a chloroplast transit peptide (CTP) or plastid transit peptide. Chromosomal transgenes from bacterial sources must have a sequence encoding a CTP sequence fused to a sequence encoding an expressed polypeptide if the expressed polypeptide is to be compartmentalized in the plant plastid (e.g. chloroplast). Accordingly, localization of an exogenous polypeptide to a chloroplast is often 1 accomplished by means of operably linking a polynucleotide sequence encoding a CTP sequence to the 5′ region of a polynucleotide encoding the exogenous polypeptide. The CTP is removed in a processing step during translocation into the plastid. Processing efficiency may, however, be affected by the amino acid sequence of the CTP and nearby sequences at the amino terminus (NH₂ terminus) of the peptide. Other options for targeting to the chloroplast which have been described are the maize cab-m7 signal sequence (U.S. Pat. No. 7,022,896, WO 97/41228) a pea glutathione reductase signal sequence (WO 97/41228) and the CTP described in US2009029861.

In some cases, a Cas12L fusion polypeptide of the present disclosure can comprise: a) a Cas12L polypeptide of the present disclosure; and b) an endosomal escape peptide. In some cases, an endosomal escape polypeptide comprises the amino acid sequence GLFXALLXLLXSLWXLLLXA (SEQ ID NO:130), wherein each X is independently selected from lysine, histidine, and arginine. In some cases, an endosomal escape polypeptide comprises the amino acid sequence

(SEQ ID NO: 131)
GLFHALLHLLHSLWHLLLHA.

For examples of some of the above fusion partners (and more) used in the context of fusions with Cas9, Zinc Finger, and/or TALE proteins (for site specific target nucleic modification, modulation of transcription, and/or target protein modification, e.g., histone modification), see, e.g.: Nomura et al, J Am Chem Soc. 2007 Jul. 18; 129(28):8676-7; Rivenbark et al., Epigenetics. 2012 April; 7(4):350-60; Nucleic Acids Res. 2016 Jul. 8; 44(12):5615-28; Gilbert et al., Cell. 2013 Jul. 18; 154(2):442-51; Kearns et al., Nat Methods. 2015 May; 12(5):401-3; Mendenhall et al., Nat Biotechnol. 2013 December; 31(12):1133-6; Hilton et al., Nat Biotechnol. 2015 May; 33(5):510-7; Gordley et al., Proc Natl Acad Sci USA. 2009 Mar. 31; 106(13):5053-8; Akopian et al., Proc Natl Acad Sci USA. 2003 Jul. 22; 100(15):8688-91; Tan et., al., J Virol. 2006 February; 80(4):1939-48; Tan et al., Proc Natl Acad Sci USA. 2003 Oct. 14; 100(21):11997-2002; Papworth et al., Proc Natl Acad Sci USA. 2003 Feb. 18; 100(4):1621-6; Sanjana et al., Nat Protoc. 2012 Jan. 5; 7(1):171-92; Beerli et al., Proc Natl Acad Sci USA. 1998 Dec. 8; 95(25):14628-33; Snowden et al., Curr Biol. 2002 Dec. 23; 12(24):2159-66; Xu et. al., Xu et al., Cell Discov. 2016 May 3; 2:16009; Komor et al., Nature. 2016 Apr. 20; 533(7603):420-4; Chaikind et al., Nucleic Acids Res. 2016 Aug. 11; Choudhury at. al., Oncotarget. 2016 Jun. 23; Du et al., Cold Spring Harb Protoc. 2016 Jan. 4; Pham et al., Methods Mol Biol. 2016; 1358:43-57; Balboa et al., Stem Cell Reports. 2015 Sep. 8; 5(3):448-59; Hara et al., Sci Rep. 2015 Jun. 9; 5:11221; Piatek et al., Plant Biotechnol J. 2015 May; 13(4):578-89; Hu et al., Nucleic Acids Res. 2014 April; 42(7):4375-90; Cheng et al., Cell Res. 2013 October; 23(10):1163-71; and Maeder et al., Nat Methods. 2013 October; 10(10):977-9.

Additional suitable heterologous polypeptides include, but are not limited to, a polypeptide that directly and/or indirectly provides for increased transcription and/or translation of a target nucleic acid (e.g., a transcription activator or a fragment thereof, a protein or fragment thereof that recruits a transcription activator, a small molecule/drug-responsive transcription and/or translation regulator, a translation-regulating protein, etc.). Non-limiting examples of heterologous polypeptides to accomplish increased or decreased transcription include transcription activator and transcription repressor domains. In some such cases, a fusion Cas12L polypeptide is targeted by the guide nucleic acid (guide RNA) to a specific location (i.e., sequence) in the target nucleic acid and exerts locus-specific regulation such as blocking RNA polymerase binding to a promoter (which selectively inhibits transcription activator function), and/or modifying the local chromatin status (e.g., when a fusion sequence is used that modifies the target nucleic acid or modifies a polypeptide associated with the target nucleic acid). In some cases, the changes are transient (e.g., transcription repression or activation). In some cases, the changes are inheritable (e.g., when epigenetic modifications are made to the target nucleic acid or to proteins associated with the target nucleic acid, e.g., nucleosomal histones).

Non-limiting examples of heterologous polypeptides for use when targeting ssRNA target nucleic acids include (but are not limited to): splicing factors (e.g., RS domains); protein translation components (e.g., translation initiation, elongation, and/or release factors; e.g., eIF4G); RNA methylases; RNA editing enzymes (e.g., RNA deaminases, e.g., adenosine deaminase acting on RNA (ADAR), including A to I and/or C to U editing enzymes); helicases; RNA-binding proteins; and the like. It is understood that a heterologous polypeptide can include the entire protein or in some cases can include a fragment of the protein (e.g., a functional domain).

The heterologous polypeptide of a subject fusion Cas12L polypeptide can be any domain capable of interacting with ssRNA (which, for the purposes of this disclosure, includes intramolecular and/or intermolecular secondary structures, e.g., double-stranded RNA duplexes such as hairpins, stem-loops, etc.), whether transiently or irreversibly, directly or indirectly, including but not limited to an effector domain selected from the group comprising; Endonucleases (for example RNase III, the CRR22 DYW domain, Dicer, and PIN (PilT N-terminus) domains from proteins such as SMG5 and SMG6); proteins and protein domains responsible for stimulating RNA cleavage (for example CPSF, CstF, CFIm and CFIIm); Exonucleases (for example XRN-1 or Exonuclease T); Deadenylases (for example HNT3); proteins and protein domains responsible for nonsense mediated RNA decay (for example UPF1, UPF2, UPF3, UPF3b, RNP Si, Y14, DEK, REF2, and SRm160); proteins and protein domains responsible for stabilizing RNA (for example PABP); proteins and protein domains responsible for repressing translation (for example Ago2 and Ago4); proteins and protein domains responsible for stimulating translation (for example Staufen); proteins and protein domains responsible for (e.g., capable of) modulating translation (e.g., translation factors such as initiation factors, elongation factors, release factors, etc., e.g., eIF4G); proteins and protein domains responsible for polyadenylation of RNA (for example PAP1, GLD-2, and Star- PAP); proteins and protein domains responsible for polyuridinylation of RNA (for example CI D1 and terminal uridylate transferase); proteins and protein domains responsible for RNA localization (for example from IMP1, ZBP1, She2p, She3p, and Bicaudal-D); proteins and protein domains responsible for nuclear retention of RNA (for example Rrp6); proteins and protein domains responsible for nuclear export of RNA (for example TAP, NXF1, THO, TREX, REF, and Aly); proteins and protein domains responsible for repression of RNA splicing (for example PTB, Sam68, and hnRNP A1); proteins and protein domains responsible for stimulation of RNA splicing (for example Serine/Arginine-rich (SR) domains); proteins and protein domains responsible for reducing the efficiency of transcription (for example FUS (TLS)); and proteins and protein domains responsible for stimulating transcription (for example CDK7 and HIV Tat). Alternatively, the effector domain may be selected from the group comprising Endonucleases; proteins and protein domains capable of stimulating RNA cleavage; Exonucleases; Deadenylases; proteins and protein domains having nonsense mediated RNA decay activity; proteins and protein domains capable of stabilizing RNA; proteins and protein domains capable of repressing translation; proteins and protein domains capable of stimulating translation; proteins and protein domains capable of modulating translation (e.g., translation factors such as initiation factors, elongation factors, release factors, etc., e.g., eIF4G); proteins and protein domains capable of polyadenylation of RNA; proteins and protein domains capable of polyuridinylation of RNA; proteins and protein domains having RNA localization activity; proteins and protein domains capable of nuclear retention of RNA; proteins and protein domains having RNA nuclear export activity; proteins and protein domains capable of repression of RNA splicing; proteins and protein domains capable of stimulation of RNA splicing; proteins and protein domains capable of reducing the efficiency of transcription; and proteins and protein domains capable of stimulating transcription. Another suitable heterologous polypeptide is a PUF RNA-binding domain, which is described in more detail in WO2012068627, which is hereby incorporated by reference in its entirety.

Some RNA splicing factors that can be used (in whole or as fragments thereof) as heterologous polypeptides for a fusion Cas12L polypeptide have modular organization, with separate sequence-specific RNA binding modules and splicing effector domains. For example, members of the Serine/Arginine-rich (SR) protein family contain N-terminal RNA recognition motifs (RRMs) that bind to exonic splicing enhancers (ESEs) in pre-mRNAs and C-terminal RS domains that promote exon inclusion. As another example, the hnRNP protein hnRNP Al binds to exonic splicing silencers (ESSs) through its RRM domains and inhibits exon inclusion through a C-terminal Glycine-rich domain. Some splicing factors can regulate alternative use of splice site (ss) by binding to regulatory sequences between the two alternative sites. For example, ASF/SF2 can recognize ESEs and promote the use of intron proximal sites, whereas hnRNP Al can bind to ESSs and shift splicing towards the use of intron distal sites. One application for such factors is to generate ESFs that modulate alternative splicing of endogenous genes, particularly disease associated genes. For example, Bcl-x pre-mRNA produces two splicing isoforms with two alternative 5′ splice sites to encode proteins of opposite functions. The long splicing isoform Bcl-xL is a potent apoptosis inhibitor expressed in long-lived postmitotic cells and is up-regulated in many cancer cells, protecting cells against apoptotic signals. The short isoform Bcl-xS is a pro-apoptotic isoform and expressed at high levels in cells with a high turnover rate (e.g., developing lymphocytes). The ratio of the two Bcl-x splicing isoforms is regulated by multiple cω-elements that are located in either the core exon region or the exon extension region (i.e., between the two alternative 5′ splice sites). For more examples, see WO2010075303, which is hereby incorporated by reference in its entirety.

Further suitable fusion partners include, but are not limited to, proteins (or fragments thereof) that are boundary elements (e.g., CTCF), proteins and fragments thereof that provide periphery recruitment (e.g., Lamin A, Lamin B, etc.), protein docking elements (e.g., FKBP/FRB, Pil1/Aby1, etc.).

### Nucleases

In some cases, a subject fusion Cas12L polypeptide comprises: i) a Cas12L polypeptide of the present disclosure; and ii) a heterologous polypeptide (a “fusion partner”), where the heterologous polypeptide is a nuclease. Suitable nucleases include, but are not limited to, a homing nuclease polypeptide; a FokI polypeptide; a transcription activator-like effector nuclease (TALEN) polypeptide; a MegaTAL polypeptide; a meganuclease polypeptide; a zinc finger nuclease (ZFN); an ARCUS nuclease; and the like. The meganuclease can be engineered from an LADLIDADG homing endonuclease (LHE). A megaTAL polypeptide can comprise a TALE DNA binding domain and an engineered meganuclease. See, e.g., WO 2004/067736 (homing endonuclease); Urnov et al. (2005) Nature 435:646 (ZFN); Mussolino et al. (2011) Nucle. Acids Res. 39:9283 (TALE nuclease); Boissel et al. (2013) Nucl. Acids Res. 42:2591 (MegaTAL).

### Reverse Transcriptases

In some cases, a subject fusion Cas12L polypeptide comprises: i) a Cas12L polypeptide of the present disclosure; and ii) a heterologous polypeptide (a “fusion partner”), where the heterologous polypeptide is a reverse transcriptase polypeptide. In some cases, the Cas12L polypeptide is catalytically inactive. Suitable reverse transcriptases include, e.g., a murine leukemia virus reverse transcriptase; a Rous sarcoma virus reverse transcriptase; a human immunodeficiency virus type I reverse transcriptase; a Moloney murine leukemia virus reverse transcriptase; and the like.

### Base Editors

In some cases, a Cas12L fusion polypeptide of the present disclosure comprises: i) a Cas12L polypeptide of the present disclosure; and ii) a heterologous polypeptide (a “fusion partner”), where the heterologous polypeptide is a base editor. Suitable base editors include, e.g., an adenosine deaminase; a cytidine deaminase (e.g., an activation-induced cytidine deaminase (AID)); APOBEC3G; and the like); and the like.

A suitable adenosine deaminase is any enzyme that is capable of deaminating adenosine in DNA. In some cases, the deaminase is a TadA deaminase.

In some cases, a suitable adenosine deaminase comprises an amino acid sequence having at least 80%, at least 85%, at least 90%, at least 95%, at least 98%, at least 99%, or 100%, amino acid sequence identity to the following amino acid sequence:

(SEQ ID NO: 132)
MSEVEFSHEYWMRHALTLAKRAWDEREVPVGAVLVHNNRVIGEGWNRPIG
RHDPTAHAEIMALRQGGLVMQNYRLIDATLYVTLEPCVMCAGAMIHSRIG
RVVFGARDAKTGAAGSLMDVLHHPGMNHRVEITEGILADECAALLSDFFR
MRRQEIKAQKKAQSSTD

In some cases, a suitable adenosine deaminase comprises an amino acid sequence having at least 80%, at least 85%, at least 90%, at least 95%, at least 98%, at least 99%, or 100%, amino acid sequence identity to the following amino acid sequence:

(SEQ ID NO: 133)
MRRAFITGVFFLSEVEFSHEYWMRHALTLAKRAWDEREVPVGAVLVHNNR
VIGEGWNRPIGRHDPTAHAEIMALRQGGLVMQNYRLIDATLYVTLEPCVM
CAGAMIHSRIGRVVFGARDAKTGAAGSLMDVLHHPGMNHRVEITEGILAD
ECAALLSDFFRMRRQEIKAQKKAQSSTD.

In some cases, a suitable adenosine deaminase comprises an amino acid sequence having at least 80%, at least 85%, at least 90%, at least 95%, at least 98%, at least 99%, or 100%, amino acid sequence identity to the following Staphylococcus aureus TadA amino acid sequence:

(SEQ ID NO: 134)
MGSHMTNDIYFMTLAIEEAKKAAQLGEVPIGAIITKDDEVIARAHNLRET
LQQPTAHAEHIAIERAAKVLGSWRLEGCTLYVTLEPCVMCAGTIVMSRIP
RVVYGADDPKGGCSGSLMNLLQQSNFNHRAIVDKGVLKEACSTLLTTFFK
NLRANKKSTN:

In some cases, a suitable adenosine deaminase comprises an amino acid sequence having at least 80%, at least 85%, at least 90%, at least 95%, at least 98%, at least 99%, or 100%, amino acid sequence identity to the following Bacillus subtilis TadA amino acid sequence:

(SEQ ID NO: 135)
MTQDELYMKEAIKEAKKAEEKGEVPIGAVLVINGEIIARAHNLRETEQRS
IAHAEMLVIDEACKALGTWRLEGATLYVTLEPCPMCAGAVVLSRVEKVVF
GAFDPKGGCSGTLMNLLQEERFNHQAEVVSGVLEEECGGMLSAFFRELRK
KKKAARKNLSE

In some cases, a suitable adenosine deaminase comprises an amino acid sequence having at least 80%, at least 85%, at least 90%, at least 95%, at least 98%, at least 99%, or 100%, amino acid sequence identity to the following Salmonella typhimurium TadA:

(SEQ ID NO: 136)
MPPAFITGVTSLSDVELDHEYWMRHALTLAKRAWDEREVPVGAVLVHNHR
VIGEGWNRPIGRHDPTAHAEIMALRQGGLVLQNYRLLDTTLYVTLEPCVM
CAGAMVHSRIGRVVFGARDAKTGAAGSLIDVLHHPGMNHRVEIIEGVLRD
ECATLLSDFFRMRRQEIKALKKADRAEGAGPAV

In some cases, a suitable adenosine deaminase comprises an amino acid sequence having at least 80%, at least 85%, at least 90%, at least 95%, at least 98%, at least 99%, or 100%, amino acid sequence identity to the following Shewanella putrefaciens TadA amino acid sequence:

(SEQ ID NO: 137)
MDEYWMQVAMQMAEKAEAAGEVPVGAVLVKDGQQIATGYNLSISQHDPTA
HAEILCLRSAGKKLENYRLLDATLYITLEPCAMCAGAMVHSRIARVVYGA
RDEKTGAAGTVVNLLQHPAFNHQVEVTSGVLAEACSAQLSRFFKRRRDEK
KALKLAQRAQQGIE

In some cases, a suitable adenosine deaminase comprises an amino acid sequence having at least 80%, at least 85%, at least 90%, at least 95%, at least 98%, at least 99%, or 100%, amino acid sequence identity to the following Haemophilus influenzae F3031 TadA amino acid sequence:

(SEQ ID NO: 138)
MDAAKVRSEFDEKMMRYALELADKAEALGEIPVGAVLVDDARNIIGEGWN
LSIVQSDPTAHAEIIALRNGAKNIQNYRLLNSTLYVTLEPCTMCAGAILH
SRIKRLVFGASDYKTGAIGSRFHFFDDYKMNHTLEITSGVLAEECSQKLS
TFFQKRREEKKIEKALLKSLSDK

In some cases, a suitable adenosine deaminase comprises an amino acid sequence having at least 80%, at least 85%, at least 90%, at least 95%, at least 98%, at least 99%, or 100%, amino acid sequence identity to the following Caulobacter crescentus TadA amino acid sequence:

(SEQ ID NO: 139)
MRTDESEDQDHRMMRLALDAARAAAEAGETPVGAVILDPSTGEVIATAG
NGPIAAHDPTAHAEIAAMRAAAAKLGNYRLTDLTLVVTLEPCAMCAGAI
SHARIGRVVFGADDPKGGAVVHGPKFFAQPTCHWRPEVTGGVLADESAD
LLRGFFRARRKAKI

In some cases, a suitable adenosine deaminase comprises an amino acid sequence having at least 80%, at least 85%, at least 90%, at least 95%, at least 98%, at least 99%, or 100%, amino acid sequence identity to the following Geobacter sulfurreducens TadA amino acid sequence:

(SEQ ID NO: 140)
MSSLKKTPIRDDAYWMGKAIREAAKAAARDEVPIGAVIVRDGAVIGRGH
NLREGSNDPSAHAEMIAIRQAARRSANWRLTGATLYVTLEPCLMCMGAI
ILARLERVVFGCYDPKGGAAGSLYDLSADPRLNHQVRLSPGVCQEECGT
MLSDFFRDLRRRKKAKATPALFIDERKVPPEP

Cytidine deaminases suitable for inclusion in a Cas12L fusion polypeptide include any enzyme that is capable of deaminating cytidine in DNA.

In some cases, the cytidine deaminase is a deaminase from the apolipoprotein B mRNA-editing complex (APOBEC) family of deaminases. In some cases, the APOBEC family deaminase is selected from the group consisting of APOBEC1 deaminase, APOBEC2 deaminase, APOBEC3A deaminase, APOBEC3B deaminase, APOBEC3C deaminase, APOBEC3D deaminase, APOBEC3F deaminase, APOBEC3G deaminase, and APOBEC3H deaminase. In some cases, the cytidine deaminase is an activation induced deaminase (AID).

In some cases, a suitable cytidine deaminase comprises an amino acid sequence having at least 80%, at least 85%, at least 90%, at least 95%, at least 98%, at least 99%, or 100%, amino acid sequence identity to the following amino acid sequence:

(SEQ ID NO: 141)
MDSLLMNRRKFLYQFKNVRWAKGRRETYLCYVVKRRDSATSFSLDFGYL
RNKNGCHVELLFLRYISDWDLDPGRCYRVTWFTSWSPCYDCARHVADFL
RGNPNLSLRIFTARLYFCEDRKAEPEGLRRLHRAGVQIAIMTFKDYFYC
WNTFVENHERTFKAWEGLHENSVRLSRQLRRILLPLYEVDDLRDAFRTL
GL

In some cases, a suitable cytidine deaminase is an AID and comprises an amino acid sequence having at least 80%, at least 85%, at least 90%, at least 95%, at least 98%, at least 99%, or 100%, amino acid sequence identity to the following amino acid sequence: MDSLLMNRRK FLYQFKNVRW AKGRRETYLC YVVKRRDSAT SFSLDFGYLR NKNGCHVELL FLRYISDWDL DPGRCYRVTW FTSWSPCYDC ARHVADFLRG NPNLSLRIFT ARLYFCEDRK AEPEGLRRLH RAGVQIAIMT FKENHERTFK AWEGLHENSV RLSRQLRRIL LPLYEVDDLR DAFRTLGL (SEQ ID NO:142).

In some cases, a suitable cytidine deaminase is an AID and comprises an amino acid sequence having at least 80%, at least 85%, at least 90%, at least 95%, at least 98%, at least 99%, or 100%, amino acid sequence identity to the following amino acid sequence: MDSLLMNRRK FLYQFKNVRW AKGRRETYLC YVVKRRDSAT SFSLDFGYLR NKNGCHVELL FLRYISDWDL DPGRCYRVTW FTSWSPCYDC ARHVADFLRG NPNLSLRIFT ARLYFCEDRK AEPEGLRRLH RAGVQIAIMT FKDYFYCWNT FVENHERTFK AWEGLHENSV RLSRQLRRIL LPLYEVDDLR DAFRTLGL (SEQ ID NO:141).

### Transcription Factors

In some cases, a Cas12L fusion polypeptide of the present disclosure comprises: i) a Cas12L polypeptide of the present disclosure; and ii) a heterologous polypeptide (a “fusion partner”), where the heterologous polypeptide is a transcription factor. A transcription factor can include: i) a DNA binding domain; and ii) a transcription activator. A transcription factor can include: i) a DNA binding domain; and ii) a transcription repressor. Suitable transcription factors include polypeptides that include a transcription activator or a transcription repressor domain (e.g., the Kruppel associated box (KRAB or SKD); the Mad mSIN3 interaction domain (SID); the ERF repressor domain (ERD), etc.); zinc-finger-based artificial transcription factors (see, e.g., Sera (2009) Adv. Drug Deliv. 61:513); TALE-based artificial transcription factors (see, e.g., Liu et al. (2013) Nat. Rev. Genetics 14:781); and the like. In some cases, the transcription factor comprises a VP64 polypeptide (transcriptional activation). In some cases, the transcription factor comprises a Kruppel-associated box (KRAB) polypeptide (transcriptional repression). In some cases, the transcription factor comprises a Mad mSIN3 interaction domain (SID) polypeptide (transcriptional repression). In some cases, the transcription factor comprises an ERF repressor domain (ERD) polypeptide (transcriptional repression). For example, in some cases, the transcription factor is a transcriptional activator, where the transcriptional activator is GAL4-VP16.

### Recombinases

In some cases, a Cas12L fusion polypeptide of the present disclosure comprises: i) a Cas12L polypeptide of the present disclosure; and ii) a heterologous polypeptide (a “fusion partner”), where the heterologous polypeptide is a recombinase. Suitable recombinases include, e.g., a Cre recombinase; a Hin recombinase; a Tre recombinase; a FLP recombinase; and the like.

Examples of various additional suitable heterologous polypeptide (or fragments thereof) for a subject fusion Cas12L polypeptide include, but are not limited to, those described in the following applications (which publications are related to other CRISPR endonucleases such as Cas9, but the described fusion partners can also be used with Cas12L instead): PCT patent applications: WO2010075303, WO2012068627, and WO2013155555, and can be found, for example, in U.S. patents and patent applications: U.S. Pat. Nos. 8,906,616; 8,895,308; 8,889,418; 8,889,356; 8,871,445; 8,865,406; 8,795,965; 8,771,945; 8,697,359; 20140068797; 20140170753; 20140179006; 20140179770; 20140186843; 20140186919; 20140186958; 20140189896; 20140227787; 20140234972; 20140242664; 20140242699; 20140242700; 20140242702; 20140248702; 20140256046; 20140273037; 20140273226; 20140273230; 20140273231; 20140273232; 20140273233; 20140273234; 20140273235; 20140287938; 20140295556; 20140295557; 20140298547; 20140304853; 20140309487; 20140310828; 20140310830; 20140315985; 20140335063; 20140335620; 20140342456; 20140342457; 20140342458; 20140349400; 20140349405; 20140356867; 20140356956; 20140356958; 20140356959; 20140357523; 20140357530; 20140364333; and 20140377868; all of which are hereby incorporated by reference in their entirety.

In some cases, a heterologous polypeptide (a fusion partner) provides for subcellular localization, i.e., the heterologous polypeptide contains a subcellular localization sequence (e.g., a nuclear localization signal (NLS) for targeting to the nucleus, a sequence to keep the fusion protein out of the nucleus, e.g., a nuclear export sequence (NES), a sequence to keep the fusion protein retained in the cytoplasm, a mitochondrial localization signal for targeting to the mitochondria, a chloroplast localization signal for targeting to a chloroplast, an ER retention signal, and the like). In some embodiments, a Cas12L fusion polypeptide does not include an NLS so that the protein is not targeted to the nucleus (which can be advantageous, e.g., when the target nucleic acid is an RNA that is present in the cytosol). In some embodiments, the heterologous polypeptide can provide a tag (i.e., the heterologous polypeptide is a detectable label) for ease of tracking and/or purification (e.g., a fluorescent protein, e.g., green fluorescent protein (GFP), yellow fluorescent protein (YFP), red fluorescent protein (RFP), cyan fluorescent protein (CFP), mCherry, tdTomato, and the like; a histidine tag, e.g., a 6×His tag; a hemagglutinin (HA) tag; a FLAG tag; a Myc tag; and the like).

In some cases, a Cas12L protein (e.g., a wild type Cas12L protein, a variant Cas12L protein, a fusion Cas12L protein, a dCas12L protein, and the like) includes (is fused to) a nuclear localization signal (NLS) (e.g., in some cases 2 or more, 3 or more, 4 or more, or 5 or more NLSs). Thus, in some cases, a Cas12L polypeptide includes one or more NLSs (e.g., 2 or more, 3 or more, 4 or more, or 5 or more NLSs). In some cases, one or more NLSs (2 or more, 3 or more, 4 or more, or 5 or more NLSs) are positioned at or near (e.g., within 50 amino acids of) the N-terminus and/or the C-terminus. In some cases, one or more NLSs (2 or more, 3 or more, 4 or more, or 5 or more NLSs) are positioned at or near (e.g., within 50 amino acids of) the N-terminus. In some cases, one or more NLSs (2 or more, 3 or more, 4 or more, or 5 or more NLSs) are positioned at or near (e.g., within 50 amino acids of) the C-terminus. In some cases, one or more NLSs (3 or more, 4 or more, or 5 or more NLSs) are positioned at or near (e.g., within 50 amino acids of) both the N-terminus and the C-terminus. In some cases, an NLS is positioned at the N-terminus and an NLS is positioned at the C-terminus.

In some cases, a Cas12L protein (e.g., a wild type Cas12L protein, a variant Cas12L protein, a fusion Cas12L protein, a dCas12L protein, and the like) includes (is fused to) between 1 and 10 NLSs (e.g., 1-9, 1-8, 1-7, 1-6, 1-5, 2-10, 2-9, 2-8, 2-7, 2-6, or 2-5 NLSs). In some cases, a Cas12L protein (e.g., a wild type Cas12L protein, a variant Cas12L protein, a fusion Cas12L protein, a dCas12L protein, and the like) includes (is fused to) between 2 and 5 NLSs (e.g., 2-4, or 2-3 NLSs).

Non-limiting examples of NLSs include an NLS sequence derived from: the NLS of the SV40 virus large T-antigen, having the amino acid sequence PKKKRKV (SEQ ID NO:143); the NLS from nucleoplasmin (e.g., the nucleoplasmin bipartite NLS with the sequence KRPAATKKAGQAKKKK (SEQ ID NO:144)); the c-myc NLS having the amino acid sequence PAAKRVKLD (SEQ ID NO:145) or RQRRNELKRSP (SEQ ID NO:146); the hRNPA1 M9 NLS having the sequence NQSSNFGPMKGGNFGGRSSGPYGGGGQYFAKPRNQGGY (SEQ ID NO:147); the sequence RMRIZFKNKGKDTAELRRRRVEVSVELRKAKKDEQILKRRNV (SEQ ID NO:148) of the IBB domain from importin-alpha; the sequences VSRKRPRP (SEQ ID NO:149) and PPKKARED (SEQ ID NO:150) of the myoma T protein; the sequence PQPKKKPL (SEQ ID NO:151) of human p53; the sequence SALIKKKKKMAP (SEQ ID NO:152) of mouse c-abl IV; the sequences DRLRR (SEQ ID NO:153) and PKQKKRK (SEQ ID NO:154) of the influenza virus NS1; the sequence RKLKKKIKKL (SEQ ID NO:155) of the Hepatitis virus delta antigen; the sequence REKKKFLKRR (SEQ ID NO:156) of the mouse Mx1 protein; the sequence KRKGDEVDGVDEVAKKKSKK (SEQ ID NO:157) of the human poly(ADP-ribose) polymerase; and the sequence RKCLQAGMNLEARKTKK (SEQ ID NO:158) of the steroid hormone receptors (human) glucocorticoid. In general, NLS (or multiple NLSs) are of sufficient strength to drive accumulation of the Cas12L protein in a detectable amount in the nucleus of a eukaryotic cell. Detection of accumulation in the nucleus may be performed by any suitable technique. For example, a detectable marker may be fused to the Cas12L protein such that location within a cell may be visualized. Cell nuclei may also be isolated from cells, the contents of which may then be analyzed by any suitable process for detecting protein, such as immunohistochemistry, Western blot, or enzyme activity assay. Accumulation in the nucleus may also be determined indirectly.

In some cases, a Cas12L fusion polypeptide includes a “Protein Transduction Domain” or PTD (also known as a CPP—cell penetrating peptide), which refers to a polypeptide, polynucleotide, carbohydrate, or organic or inorganic compound that facilitates traversing a lipid bilayer, micelle, cell membrane, organelle membrane, or vesicle membrane. A PTD attached to another molecule, which can range from a small polar molecule to a large macromolecule and/or a nanoparticle, facilitates the molecule traversing a membrane, for example going from extracellular space to intracellular space, or cytosol to within an organelle. In some embodiments, a PTD is covalently linked to the amino terminus a polypeptide (e.g., linked to a wild type Cas12L polypeptide to generate a fusion protein, or linked to a variant Cas12L protein such as a dCas12L, nickase Cas12L, or fusion Cas12L protein, to generate a fusion protein). In some cases, a PTD is covalently linked to the carboxyl terminus of a polypeptide (e.g., linked to a wild type Cas12L to generate a fusion protein, or linked to a variant Cas12L protein such as a dCas12L, nickase Cas12L, or fusion Cas12L protein to generate a fusion protein). In some cases, the PTD is inserted internally in the Cas12L fusion polypeptide (i.e., is not at the N- or C-terminus of the Cas12L fusion polypeptide) at a suitable insertion site. In some cases, a subject Cas12L fusion polypeptide includes (is conjugated to, is fused to) one or more PTDs (e.g., two or more, three or more, four or more PTDs). In some cases, a PTD includes a nuclear localization signal (NLS) (e.g., in some cases 2 or more, 3 or more, 4 or more, or 5 or more NLSs). Thus, in some cases, a Cas12L fusion polypeptide includes one or more NLSs (e.g., 2 or more, 3 or more, 4 or more, or 5 or more NLSs). In some embodiments, a PTD is covalently linked to a nucleic acid (e.g., a Cas12L guide nucleic acid, a polynucleotide encoding a Cas12L guide nucleic acid, a polynucleotide encoding a Cas12L fusion polypeptide, a donor polynucleotide, etc.). Examples of PTDs include but are not limited to a minimal undecapeptide protein transduction domain (corresponding to residues 47-57 of HIV-1 TAT comprising YGRKKRRQRRR; SEQ ID NO:159); a polyarginine sequence comprising a number of arginines sufficient to direct entry into a cell (e.g., 3, 4, 5, 6, 7, 8, 9, 10, or 10-50 arginines); a VP22 domain (Zender et al. (2002) Cancer Gene Ther. 9(6):489-96); a Drosophila Antennapedia protein transduction domain (Noguchi et al. (2003) Diabetes 52(7):1732-1737); a truncated human calcitonin peptide (Trehin et al. (2004) Pharm. Research 21:1248-1256); polylysine (Wender et al. (2000) Proc. Natl. Acad. Sci. USA 97:13003-13008); RRQRRTSKLMKR (SEQ ID NO:160); Transportan GWTLNSAGYLLGKINLKALAALAKKIL (SEQ ID NO:161); KALAWEAKLAKALAKALAKHLAKALAKALKCEA (SEQ ID NO:162); and RQIKIWFQNRRMKWKK (SEQ ID NO:163). Exemplary PTDs include but are not limited to, YGRKKRRQRRR (SEQ ID NO:159), RKKRRQRRR (SEQ ID NO:164); an arginine homopolymer of from 3 arginine residues to 50 arginine residues; Exemplary PTD domain amino acid sequences include, but are not limited to, any of the following: YGRKKRRQRRR (SEQ ID NO:159); RKKRRQRR (SEQ ID NO:165); YARAAARQARA (SEQ ID NO:166); THRLPRRRRRR (SEQ ID NO:167); and GGRRARRRRRR (SEQ ID NO:168). In some cases, the PTD is an activatable CPP (ACPP) (Aguilera et al. (2009) Integr Biol (Camb) June; 1(5-6): 371-381). ACPPs comprise a polycationic CPP (e.g., Arg9 or “R9”) connected via a cleavable linker to a matching polyanion (e.g., Glu9 or “E9”), which reduces the net charge to nearly zero and thereby inhibits adhesion and uptake into cells. Upon cleavage of the linker, the polyanion is released, locally unmasking the polyarginine and its inherent adhesiveness, thus “activating” the ACPP to traverse the membrane.

### Linkers (e.g., for Fusion Partners)

In some embodiments, a subject Cas12L protein can fused to a fusion partner via a linker polypeptide (e.g., one or more linker polypeptides). The linker polypeptide may have any of a variety of amino acid sequences. Proteins can be joined by a spacer peptide, generally of a flexible nature, although other chemical linkages are not excluded. Suitable linkers include polypeptides of between 4 amino acids and 40 amino acids in length, or between 4 amino acids and 25 amino acids in length. These linkers can be produced by using synthetic, linker-encoding oligonucleotides to couple the proteins, or can be encoded by a nucleic acid sequence encoding the fusion protein. Peptide linkers with a degree of flexibility can be used. The linking peptides may have virtually any amino acid sequence, bearing in mind that the preferred linkers will have a sequence that results in a generally flexible peptide. The use of small amino acids, such as glycine and alanine, are of use in creating a flexible peptide. The creation of such sequences is routine to those of skill in the art. A variety of different linkers are commercially available and are considered suitable for use.

Examples of linker polypeptides include glycine polymers (G)ₙ, glycine-serine polymers (including, for example, (GS)ₙ, and (GGGGS)n (SEQ ID NO:169), where n is an integer from 1 to 10), glycine-alanine polymers, alanine-serine polymers. Exemplary linkers can comprise amino acid sequences including, but not limited to, GGSG (SEQ ID NO:170), GGSGG (SEQ ID NO:171), GSGSG (SEQ ID NO:172), GSGGG (SEQ ID NO:173), GGGSG (SEQ ID NO:174), GSSSG (SEQ ID NO:175), and the like. The ordinarily skilled artisan will recognize that design of a peptide conjugated to any desired element can include linkers that are all or partially flexible, such that the linker can include a flexible linker as well as one or more portions that confer less flexible structure.

A variety of shorter or longer linker regions are known in the art, for example corresponding to a series of glycine residues, a series of adjacent glycine-serine dipeptides, a series of adjacent glycine-glycine-serine tripeptides, or known linkers from other proteins. A flexible linker may include, for example, the amino acid sequence: SSGPPPGTG (SEQ ID NO:176) and variants thereof. A rigid linker may include, for example, the amino acid sequence: AEAAAKEAAAKA (SEQ ID NO:177) and variants thereof. The XTEN linker, SGSETPGTSESATPES (SEQ ID NO:178) and variants thereof, described in Guilinget et al, 2014 (Nature Biotechnology 32, 577-582), may also be used.

### Tags, Reporters, and Other Features

A Cas12L polypeptide may contain one or more tags that allow for e.g. purification and/or detection of the recombinant polypeptide. Various tags may be used herein and are well-known to those of skill in the art. Exemplary tags may include HA, GST, FLAG, MBP, etc., and multiple copies of one or more tags may be present in a Cas12L polypeptide.

A Cas12L polypeptide may contain one or more reporters that allow for e.g. visualization and/or detection of the Cas12L polypeptide. A reporter polypeptide encodes a protein that may be readily detectable due to its biochemical characteristics such as, for example, enzymatic activity or chemifluorescent features. Reporter polypeptides may be detected in a number of ways depending on the characteristics of the particular reporter. For example, a reporter polypeptide may be detected by its ability to generate a detectable signal (e.g. fluorescence), by its ability to form a detectable product, etc. Various reporters may be used herein and are well-known to those of skill in the art. Exemplary reporters may include GFP, GUS, mCherry, luciferase, etc., and multiple copies of one or more tags may be present in a recombinant polypeptide.

A Cas12L polypeptide may contain one or more polypeptide domains that serve a particular purpose depending on the particular goal/need. For example, a Cas12L polypeptide may contain a GB1 polypeptide. A Cas12L polypeptide may contain translocation sequences that target the polypeptide to a particular cellular compartment or area. Suitable features will be readily apparent to those of skill in the art.

### Protospacer Adjacent Motif (PAM)

A Cas12L protein binds to target DNA at a target sequence defined by the region of complementarity between the DNA-targeting RNA and the target DNA. As is the case for many CRISPR endonucleases, site-specific binding (and/or cleavage) of a double stranded target DNA occurs at locations determined by both (i) base-pairing complementarity between the guide RNA and the target DNA; and (ii) a short motif [referred to as the protospacer adjacent motif (PAM)] in the target DNA.

In some cases, the PAM for a Cas12L protein is immediately 5′ of the target sequence of the non-complementary strand of the target DNA (the complementary strand: (i) hybridizes to the guide sequence of the guide RNA, while the non-complementary strand does not directly hybridize with the guide RNA; and (ii) is the reverse complement of the non-complementary strand).

In some cases, different Cas12L proteins (i.e., Cas12L proteins from various species) may be advantageous to use in the various provided methods in order to capitalize on various enzymatic characteristics of the different Cas12L proteins (e.g., for different PAM sequence preferences; for increased or decreased enzymatic activity; for an increased or decreased level of cellular toxicity; to change the balance between NHEJ, homology-directed repair, single strand breaks, double strand breaks, etc.; to take advantage of a short total sequence; and the like). Cas12L proteins from different species may require different PAM sequences in the target DNA. Various methods (including in silico and/or wet lab methods) for identification of the appropriate PAM sequence are known in the art and are routine, and any convenient method can be used.

A Cal12L polypeptide of the present disclosure can be reprogrammed (by complexing with a guide RNA) to cleave any sequence of a target nucleic acid (e.g., a target DNA) that is complementary to the targeting segment of the guide RNA, where the PAM is present on the 5′ end of the target (e.g., a T-rich PAM for Casλ1); additional RNA components are not required for the formation of functional effectors in vivo. In some cases, a PAM sequence is a T-rich sequence (e.g., TTR, where R is a purine). In some cases, a PAM sequence is TTA. In some cases, a PAM sequence is TTG.

### Cas12L Guide RNA

A nucleic acid that binds to a Cas12L protein, forming a ribonucleoprotein complex (RNP), and targets the complex to a specific location within a target nucleic acid (e.g., a target DNA) is referred to herein as a “Cas12L guide RNA” or simply as a “guide RNA.” It is to be understood that in some cases, a hybrid DNA/RNA can be made such that a Cas12L guide RNA includes DNA bases in addition to RNA bases, but the term “Cas12L guide RNA” is still used to encompass such a molecule herein.

A Cas12L guide RNA can be said to include two segments, a targeting segment and a protein-binding segment. The protein-binding segment is also referred to herein as the “constant region” of the guide RNA. The targeting segment of a Cas12L guide RNA includes a nucleotide sequence (a guide sequence) that is complementary to (and therefore hybridizes with) a specific sequence (a target site) within a target nucleic acid (e.g., a target dsDNA, a target ssRNA, a target ssDNA, the complementary strand of a double stranded target DNA, etc.). The protein-binding segment (or “protein-binding sequence”) interacts with (binds to) a Cas12L polypeptide. The protein-binding segment of a subject Cas12L guide RNA can include two complementary stretches of nucleotides that hybridize to one another to form a double stranded RNA duplex (dsRNA duplex). Site-specific binding and/or cleavage of a target nucleic acid (e.g., genomic DNA, ds DNA, RNA, etc.) can occur at locations (e.g., target sequence of a target locus) determined by base-pairing complementarity between the Cas12L guide RNA (the guide sequence of the Cas12L guide RNA) and the target nucleic acid.

A Cas12L guide RNA and a Cas12L protein (e.g., a wild-type Cas12L protein; a variant Cas12L protein; a fusion Cas12L polypeptide; etc.) form a complex (e.g., bind via non-covalent interactions). The Cas12L guide RNA provides target specificity to the complex by including a targeting segment, which includes a guide sequence (a nucleotide sequence that is complementary to a sequence of a target nucleic acid). The Cas12L protein of the complex provides the site-specific activity (e.g., cleavage activity provided by the Cas12L protein and/or an activity provided by the fusion partner in the case of a fusion Cas12L protein). In other words, the Cas12L protein is guided to a target nucleic acid sequence (e.g. a target sequence) by virtue of its association with the Cas12L guide RNA.

The “guide sequence” also referred to as the “targeting sequence” of a Cas12L guide RNA can be modified so that the Cas12L guide RNA can target a Cas12L protein (e.g., a naturally occurring Cas12L protein, a fusion Cas12L polypeptide, and the like) to any desired sequence of any desired target nucleic acid, with the exception (e.g., as described herein) that the PAM sequence can be taken into account. Thus, for example, a Cas12L guide RNA can have a guide sequence with complementarity to (e.g., can hybridize to) a sequence in a nucleic acid in a eukaryotic cell, e.g., a viral nucleic acid, a eukaryotic nucleic acid (e.g., a eukaryotic chromosome, chromosomal sequence, a eukaryotic RNA, etc.), and the like.

### Guide Sequence of a Cas12L Guide RNA

A subject Cas12L guide RNA includes a guide sequence (i.e., a targeting sequence), which is a nucleotide sequence that is complementary to a sequence (a target site) in a target nucleic acid. In other words, the guide sequence of a Cas12L guide RNA can interact with a target nucleic acid (e.g., double stranded DNA (dsDNA), single stranded DNA (ssDNA), single stranded RNA (ssRNA), or double stranded RNA (dsRNA)) in a sequence-specific manner via hybridization (i.e., base pairing). The guide sequence of a Cas12L guide RNA can be modified (e.g., by genetic engineering)/designed to hybridize to any desired target sequence (e.g., while taking the PAM into account, e.g., when targeting a dsDNA target) within a target nucleic acid (e.g., a eukaryotic target nucleic acid such as genomic DNA).

In some cases, the percent complementarity between the guide sequence and the target site of the target nucleic acid is 60% or more (e.g., 65% or more, 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid is 80% or more (e.g., 85% or more, 90% or more, 95% or more, 97% or more, 98% or more, 99% or more, or 100%). In some cases, the percent complementarity between the guide sequence and the target site of the target nucleic acid is 90% or more (e.g., 95% or more, 97% or more, 98% or more, 99% or more, or 100%). In some cases, the percent complementarity between the guide sequence and the target site of the target nucleic acid is 100%.

In some cases, the percent complementarity between the guide sequence and the target site of the target nucleic acid is 100% over the seven contiguous 3′-most nucleotides of the target site of the target nucleic acid.

In some cases, the percent complementarity between the guide sequence and the target site of the target nucleic acid is 60% or more (e.g., 70% or more, 75% or more, 80% or more, 85% or more, 90% or more, 95% or more, 97% or more, 98% or more, 99% or more, or 100%) over 17 or more (e.g., 18 or more, 19 or more, 20 or more, 21 or more, 22 or more) contiguous nucleotides. In some cases, the percent complementarity between the guide sequence and the target site of the target nucleic acid is 80% or more (e.g., 85% or more, 90% or more, 95% or more, 97% or more, 98% or more, 99% or more, or 100%) over 17 or more (e.g., 18 or more, 19 or more, 20 or more, 21 or more, 22 or more) contiguous nucleotides. In some cases, the percent complementarity between the guide sequence and the target site of the target nucleic acid is 90% or more (e.g., 95% or more, 97% or more, 98% or more, 99% or more, or 100%) over 17 or more (e.g., 18 or more, 19 or more, 20 or more, 21 or more, 22 or more) contiguous nucleotides. In some cases, the percent complementarity between the guide sequence and the target site of the target nucleic acid is 100% over 17 or more (e.g., 18 or more, 19 or more, 20 or more, 21 or more, 22 or more) contiguous nucleotides.

In some cases, the percent complementarity between the guide sequence and the target site of the target nucleic acid is 60% or more (e.g., 70% or more, 75% or more, 80% or more, 85% or more, 90% or more, 95% or more, 97% or more, 98% or more, 99% or more, or 100%) over 19 or more (e.g., 20 or more, 21 or more, 22 or more) contiguous nucleotides. In some cases, the percent complementarity between the guide sequence and the target site of the target nucleic acid is 80% or more (e.g., 85% or more, 90% or more, 95% or more, 97% or more, 98% or more, 99% or more, or 100%) over 19 or more (e.g., 20 or more, 21 or more, 22 or more) contiguous nucleotides. In some cases, the percent complementarity between the guide sequence and the target site of the target nucleic acid is 90% or more (e.g., 95% or more, 97% or more, 98% or more, 99% or more, or 100%) over 19 or more (e.g., 20 or more, 21 or more, 22 or more) contiguous nucleotides. In some cases, the percent complementarity between the guide sequence and the target site of the target nucleic acid is 100% over 19 or more (e.g., 20 or more, 21 or more, 22 or more) contiguous nucleotides.

In some cases, the percent complementarity between the guide sequence and the target site of the target nucleic acid is 60% or more (e.g., 70% or more, 75% or more, 80% or more, 85% or more, 90% or more, 95% or more, 97% or more, 98% or more, 99% or more, or 100%) over 17-25 contiguous nucleotides. In some cases, the percent complementarity between the guide sequence and the target site of the target nucleic acid is 80% or more (e.g., 85% or more, 90% or more, 95% or more, 97% or more, 98% or more, 99% or more, or 100%) over 17-25 contiguous nucleotides. In some cases, the percent complementarity between the guide sequence and the target site of the target nucleic acid is 90% or more (e.g., 95% or more, 97% or more, 98% or more, 99% or more, or 100%) over 17-25 contiguous nucleotides. In some cases, the percent complementarity between the guide sequence and the target site of the target nucleic acid is 100% over 17-25 contiguous nucleotides.

In some cases, the percent complementarity between the guide sequence and the target site of the target nucleic acid is 60% or more (e.g., 70% or more, 75% or more, 80% or more, 85% or more, 90% or more, 95% or more, 97% or more, 98% or more, 99% or more, or 100%) over 19-25 contiguous nucleotides. In some cases, the percent complementarity between the guide sequence and the target site of the target nucleic acid is 80% or more (e.g., 85% or more, 90% or more, 95% or more, 97% or more, 98% or more, 99% or more, or 100%) over 19-25 contiguous nucleotides. In some cases, the percent complementarity between the guide sequence and the target site of the target nucleic acid is 90% or more (e.g., 95% or more, 97% or more, 98% or more, 99% or more, or 100%) over 19-25 contiguous nucleotides. In some cases, the percent complementarity between the guide sequence and the target site of the target nucleic acid is 100% over 19-25 contiguous nucleotides.

In some cases, the guide sequence has a length in a range of from 17-30 nucleotides (nt) (e.g., from 17-25, 17-22, 17-20, 19-30, 19-25, 19-22, 19-20, 20-30, 20-25, or 20-22 nt). In some cases, the guide sequence has a length in a range of from 17-25 nucleotides (nt) (e.g., from 17-22, 17-20, 19-25, 19-22, 19-20, 20-25, or 20-22 nt). In some cases, the guide sequence has a length of 17 or more nt (e.g., 18 or more, 19 or more, 20 or more, 21 or more, or 22 or more nt; 19 nt, 20 nt, 21 nt, 22 nt, 23 nt, 24 nt, 25 nt, etc.). In some cases, the guide sequence has a length of 19 or more nt (e.g., 20 or more, 21 or more, or 22 or more nt; 19 nt, 20 nt, 21 nt, 22 nt, 23 nt, 24 nt, 25 nt, etc.). In some cases, the guide sequence has a length of 17 nt. In some cases, the guide sequence has a length of 18 nt. In some cases, the guide sequence has a length of 19 nt. In some cases, the guide sequence has a length of 20 nt. In some cases, the guide sequence has a length of 21 nt. In some cases, the guide sequence has a length of 22 nt. In some cases, the guide sequence has a length of 23 nt.

In some cases, the guide sequence (also referred to as a “spacer sequence”) has a length of from 15 to 50 nucleotides (e.g., from 15 nucleotides (nt) to 20 nt, from 20 nt to 25 nt, from 25 nt to 30 nt, from 30 nt to 35 nt, from 35 nt to 40 nt, from 40 nt to 45 nt, or from 45 nt to 50 nt).

### Protein-Binding Segment of a Cas12L Guide RNA

The protein-binding segment (the “constant region”) of a subject Cas12L guide RNA interacts with a Cas12L protein. The Cas12L guide RNA guides the bound Cas12L protein to a specific nucleotide sequence within target nucleic acid via the above-mentioned guide sequence. The protein-binding segment of a Cas12L guide RNA can include two stretches of nucleotides that are complementary to one another and hybridize to form a double stranded RNA duplex (dsRNA duplex). Thus, in some cases, the protein-binding segment includes a dsRNA duplex.

In some cases, the dsRNA duplex region includes a range of from 5-25 base pairs (bp) (e.g., from 5-22, 5-20, 5-18, 5-15, 5-12, 5-10, 5-8, 8-25, 8-22, 8-18, 8-15, 8-12, 12-25, 12-22, 12-18, 12-15, 13-25, 13-22, 13-18, 13-15, 14-25, 14-22, 14-18, 14-15, 15-25, 15-22, 15-18, 17-25, 17-22, or 17-18 bp, e.g., 5 bp, 6 bp, 7 bp, 8 bp, 9 bp, 10 bp, etc.). In some cases, the dsRNA duplex region includes a range of from 6-15 base pairs (bp) (e.g., from 6-12, 6-10, or 6-8 bp, e.g., 6 bp, 7 bp, 8 bp, 9 bp, 10 bp, etc.). In some cases, the duplex region includes 5 or more bp (e.g., 6 or more, 7 or more, or 8 or more bp). In some cases, the duplex region includes 6 or more bp (e.g., 7 or more, or 8 or more bp). In some cases, not all nucleotides of the duplex region are paired, and therefore the duplex forming region can include a bulge. The term “bulge” herein is used to mean a stretch of nucleotides (which can be one nucleotide) that do not contribute to a double stranded duplex, but which are surround 5′ and 3′ by nucleotides that do contribute, and as such a bulge is considered part of the duplex region. In some cases, the dsRNA includes 1 or more bulges (e.g., 2 or more, 3 or more, 4 or more bulges). In some cases, the dsRNA duplex includes 2 or more bulges (e.g., 3 or more, 4 or more bulges). In some cases, the dsRNA duplex includes 1-5 bulges (e.g., 1-4, 1-3, 2-5, 2-4, or 2-3 bulges).

Thus, in some cases, the stretches of nucleotides that hybridize to one another to form the dsRNA duplex have 70%-100% complementarity (e.g., 75%-100%, 80%-10%, 85%-100%, 90%-100%, 95%-100% complementarity) with one another. In some cases, the stretches of nucleotides that hybridize to one another to form the dsRNA duplex have 70%-100% complementarity (e.g., 75%-100%, 80%-10%, 85%-100%, 90%-100%, 95%-100% complementarity) with one another. In some cases, the stretches of nucleotides that hybridize to one another to form the dsRNA duplex have 85%-100% complementarity (e.g., 90%-100%, 95%-100% complementarity) with one another. In some cases, the stretches of nucleotides that hybridize to one another to form the dsRNA duplex have 70%-95% complementarity (e.g., 75%-95%, 80%-95%, 85%-95%, 90%-95% complementarity) with one another.

In other words, in some embodiments, the dsRNA duplex includes two stretches of nucleotides that have 70%-100% complementarity (e.g., 75%-100%, 80%-10%, 85%-100%, 90%-100%, 95%-100% complementarity) with one another. In some cases, the dsRNA duplex includes two stretches of nucleotides that have 85%-100% complementarity (e.g., 90%-100%, 95%-100% complementarity) with one another. In some cases, the dsRNA duplex includes two stretches of nucleotides that have 70%-95% complementarity (e.g., 75%-95%, 80%-95%, 85%-95%, 90%-95% complementarity) with one another.

The duplex region of a subject Cas12L guide RNA can include one or more (1, 2, 3, 4, 5, etc) mutations relative to a naturally occurring duplex region. For example, in some cases a base pair can be maintained while the nucleotides contributing to the base pair from each segment can be different. In some cases, the duplex region of a subject Cas12L guide RNA includes more paired bases, less paired bases, a smaller bulge, a larger bulge, fewer bulges, more bulges, or any convenient combination thereof, as compared to a naturally occurring duplex region (of a naturally occurring Cas12L guide RNA).

Examples of various Cas9 guide RNAs can be found in the art, and in some cases variations similar to those introduced into Cas9 guide RNAs can also be introduced into Cas12L guide RNAs of the present disclosure (e.g., mutations to the dsRNA duplex region, extension of the 5′ or 3′ end for added stability for to provide for interaction with another protein, and the like). For example, see Jinek et al., Science. 2012 Aug. 17; 337(6096):816-21; Chylinski et al., RNA Biol. 2013 May; 10(5):726-37; Ma et al., Biomed Res Int. 2013; 2013:270805; Hou et al., Proc Natl Acad Sci USA. 2013 Sep. 24; 110(39):15644-9; Jinek et al., Elife. 2013; 2:e00471; Pattanayak et al., Nat Biotechnol. 2013 September; 31(9):839-43; Qi et al, Cell. 2013 Feb. 28; 152(5):1173-83; Wang et al., Cell. 2013 May 9; 153(4):910-8; Auer et al., Genome Res. 2013 Oct. 31; Chen et al., Nucleic Acids Res. 2013 Nov. 1; 41(20):e19; Cheng et al., Cell Res. 2013 October; 23(10):1163-71; Cho et al., Genetics. 2013 November; 195(3):1177-80; DiCarlo et al., Nucleic Acids Res. 2013 April; 41(7):4336-43; Dickinson et al., Nat Methods. 2013 October; 10(10):1028-34; Ebina et al., Sci Rep. 2013; 3:2510; Fujii et. al, Nucleic Acids Res. 2013 Nov. 1; 41(20):e187; Hu et al., Cell Res. 2013 November; 23(11):1322-5; Jiang et al., Nucleic Acids Res. 2013 Nov. 1; 41(20):e188; Larson et al., Nat Protoc. 2013 November; 8(11):2180-96; Mali et. at., Nat Methods. 2013 October; 10(10):957-63; Nakayama et al., Genesis. 2013 December; 51(12):835-43; Ran et al., Nat Protoc. 2013 November; 8(11):2281-308; Ran et al., Cell. 2013 Sep. 12; 154(6):1380-9; Upadhyay et al., G3 (Bethesda). 2013 Dec. 9; 3(12):2233-8; Walsh et al., Proc Natl Acad Sci USA. 2013 Sep. 24; 110(39):15514-5; Xie et al., Mol Plant. 2013 Oct. 9; Yang et al., Cell. 2013 Sep. 12; 154(6):1370-9; Briner et al., Mol Cell. 2014 Oct. 23; 56(2):333-9; and U.S. patents and patent applications: U.S. Pat. Nos. 8,906,616; 8,895,308; 8,889,418; 8,889,356; 8,871,445; 8,865,406; 8,795,965; 8,771,945; 8,697,359; 20140068797; 20140170753; 20140179006; 20140179770; 20140186843; 20140186919; 20140186958; 20140189896; 20140227787; 20140234972; 20140242664; 20140242699; 20140242700; 20140242702; 20140248702; 20140256046; 20140273037; 20140273226; 20140273230; 20140273231; 20140273232; 20140273233; 20140273234; 20140273235; 20140287938; 20140295556; 20140295557; 20140298547; 20140304853; 20140309487; 20140310828; 20140310830; 20140315985; 20140335063; 20140335620; 20140342456; 20140342457; 20140342458; 20140349400; 20140349405; 20140356867; 20140356956; 20140356958; 20140356959; 20140357523; 20140357530; 20140364333; and 20140377868; all of which are hereby incorporated by reference in their entirety.

Examples of constant regions suitable for inclusion in a Cas12L guide RNA are provided in FIG. 4A-4Z, FIG. 4AA-4ZZ, and FIG. 4AAA-4FFF (e.g., where T is substituted with U). A Cas12L guide RNA can include a constant region having from 1 to 5 nucleotide substitutions compared to any one of the nucleotide sequences depicted in FIG. 4A-4Z, FIG. 4AA-4ZZ, and FIG. 4AAA-4FFF.

The nucleotide sequences (with T substituted with U) can be combined with a spacer sequence (where the spacer sequence comprises a target nucleic acid-binding sequence (“guide sequence”)) of choice that is from 15 to 50 nucleotides (e.g., from 15 nucleotides (nt) to 20 nt, from 20 nt to 25 nt, from 25 nt to 30 nt, from 30 nt to 35 nt, from 35 nt to 40 nt, from 40 nt to 45 nt, or from 45 nt to 50 nt in length). In some cases, the spacer sequence is 35-38 nucleotides in length. For example, any one of the nucleotide sequences (with T substituted with U) depicted in FIG. 4A-4Z, FIG. 4AA-4ZZ, and FIG. 4AAA-4FFF can be included in a guide RNA comprising (N)n-constant region, where N is any nucleotide and n is an integer from 15 to 50 (e.g., from 15 to 20, from 20 to 25, from 25 to 30, from 30 to 35, from 35 to 38, from 35 to 40, from 40 to 45, or from 45 to 50).

As one example, the constant region of a Cas12L guide RNA can comprise the nucleotide sequence: AUUGUUGUAACUCUUAUUUUGUAUGGAGUAAACAAC (SEQ ID NO:96). As another example, the constant region of a Cas12L guide RNA can comprise the nucleotide sequence: AUUGUUGUAGACCUCUUUUUAUAAGGAUUGAACAAC (SEQ ID NO:98; see FIG. 4B). As another example, the constant region of a Cas12L guide RNA can comprise the nucleotide sequence: UAUUGUUGUAGAUACCUUUUUGUAAGGAUUAAACAAC (SEQ ID NO:101; see FIG. 4P). As another example, the constant region of a Cas12L guide RNA can comprise the nucleotide sequence: AAUGUUGUAGAUGCCUUUUUAUAAGGAUUAAACAACUUG (SEQ ID NO:180; see FIG. 4T). As another example, the constant region of a Cas12L guide RNA can comprise the nucleotide sequence: AUUGUUGAAAUAGUACUUUUAUAGUCUAUAUACAAC (SEQ ID NO:92; see FIG. 4BB). As another example, the constant region of a Cas12L guide RNA can comprise the nucleotide sequence: UAUUGUUGUAACUCUUAUUUUGUAUGGAGUAAACAAC (SEQ ID NO:94; see FIG. 4CC). As another example, the constant region of a Cas12L guide RNA can comprise the nucleotide sequence: AUUGUUGUAACUUUUAUUUUGUAUGGAGUAAACAAC (SEQ ID NO:97; see FIG. 4TT). As another example, the constant region of a Cas12L guide RNA can comprise the nucleotide sequence: AAUGUUGUAGAUACCUUUUUGUAAGGAUUGAACAAC (SEQ ID NO:100; see FIG. 4WW). As another example, the constant region of a Cas12L guide RNA can comprise the nucleotide sequence: AUUGUUGUAAUACUAUUUUUGUAAAGUAUAAACAAC (SEQ ID NO:103; see FIG. 4XX). As another example, the constant region of a Cas12L guide RNA can comprise the nucleotide sequence: AAUGUUGUAGAUGCCUUUUUAUAAGGAUUAAACAAC (SEQ ID NO:99; see FIG. 4YY). As another example, the constant region of a Cas12L guide RNA can comprise the nucleotide sequence: AUUGUUGUAAUACACUUUUUAUAAGGUAUGAACAAC (SEQ ID NO:104; see FIG. 4BBB). As another example, the constant region of a Cas12L guide RNA can comprise the nucleotide sequence: AUUGUUGUAACAUCUAUUUUGUAAGGUGUAAACAAC (SEQ ID NO:93; see FIG. 4KK).

The reverse complement of any one of the nucleotide sequences depicted in FIG. 4A-4Z, FIG. 4AA-4ZZ, and FIG. 4AAA-4FFF (but with T substituted with U) can be included in a guide RNA comprising constant region-(N)n, where N is any nucleotide and n is an integer from 15 to 50 (e.g., from 15 to 20, from 20 to 25, from 25 to 30, from 30 to 35, from 35 to 38, from 35 to 40, from 40 to 45, or from 45 to 50). As one example, a guide RNA can have the following nucleotide sequence: NNNNNNNNNNNNNNNNNNNNNNNNNNNNNNNNNNNAUUGUUGUAACUCUUAUUUUGUAU GGAGUAAACAAC (SEQ ID NO:179) or in some cases the reverse complement, where N is any nucleotide, e.g., where the stretch of Ns includes a target nucleic acid-binding sequence.

### Cas12L Guide Polynucleotides

In some cases, a nucleic acid that binds to a Cas12L protein, forming a nucleic acid/Cas12L polypeptide complex, and that targets the complex to a specific location within a target nucleic acid (e.g., a target DNA) comprises ribonucleotides only, deoxyribonucleotides only, or a mixture of ribonucleotides and deoxyribonucleotides. In some cases, a guide polynucleotide comprises ribonucleotides only, and is referred to herein as a “guide RNA.” In some cases, a guide polynucleotide comprises deoxyribonucleotides only, and is referred to herein as a “guide DNA.” In some cases, a guide polynucleotide comprises both ribonucleotides and deoxyribonucleotides. A guide polynucleotide can comprise combinations of ribonucleotide bases, deoxyribonucleotide bases, nucleotide analogs, modified nucleotides, and the like; and may further include naturally-occurring backbone residues and/or linkages and/or non-naturally-occurring backbone residues and/or linkages.

### Recombinant Nucleic Acids

Certain aspects of the present disclosure relate to recombinant nucleic acids. In some embodiments, recombinant nucleic acids encode recombinant polypeptides of the present disclosure.

As used herein, the terms “polynucleotide,” “nucleic acid,” and variations thereof shall be generic to polydeoxyribonucleotides (containing 2-deoxy-D-ribose), to polyribonucleotides (containing D-ribose), to any other type of polynucleotide that is an N-glycoside of a purine or pyrimidine base, and to other polymers containing non-nucleotidic backbones, provided that the polymers contain nucleobases in a configuration that allows for base pairing and base stacking, as found in DNA and RNA. Thus, these terms include known types of nucleic acid sequence modifications, for example, substitution of one or more of the naturally occurring nucleotides with an analog, and inter-nucleotide modifications. As used herein, the symbols for nucleotides and polynucleotides are those recommended by the IUPAC-IUB Commission of Biochemical Nomenclature.

“Recombinant nucleic acid” or “heterologous nucleic acid” or “recombinant polynucleotide” as used herein refers to a polymer of nucleic acids wherein at least one of the following is true: (a) the sequence of nucleic acids is foreign to (i.e., not naturally found in) a given host cell; (b) the sequence may be naturally found in a given host cell, but in an unnatural (e.g., greater than expected) amount; or (c) the sequence of nucleic acids contains two or more subsequences that are not found in the same relationship to each other in nature. For example, regarding instance (c), a recombinant nucleic acid sequence will have two or more sequences from unrelated genes arranged to make a new functional nucleic acid. In some embodiments, the present disclosure describes the introduction of an expression vector into a plant cell, where the expression vector contains a nucleic acid sequence coding for a protein that is not normally found in a plant cell or contains a nucleic acid coding for a protein that is normally found in a plant cell but is under the control of different regulatory sequences. With reference to the plant cell's genome, then, the nucleic acid sequence that codes for the protein is recombinant. A protein that is referred to as recombinant may be encoded by a recombinant nucleic acid sequence which may be present in the plant cell. Recombinant proteins of the present disclosure may also be exogenously supplied directly to host cells (e.g. plant cells).

### Nucleic Acids

The present disclosure provides one or more nucleic acids comprising one or more of: a donor polynucleotide sequence, a nucleotide sequence encoding a Cas12L polypeptide (e.g., a wild type Cas12L protein, a nickase Cas12L protein, a dCas12L protein, fusion Cas12L protein, and the like), a Cas12L guide RNA, and a nucleotide sequence encoding a Cas12L guide RNA. The present disclosure provides a nucleic acid comprising a nucleotide sequence encoding a Cas12L fusion polypeptide. The present disclosure provides a recombinant expression vector that comprises a nucleotide sequence encoding a Cas12L polypeptide. The present disclosure provides a recombinant expression vector that comprises a nucleotide sequence encoding a Cas12L fusion polypeptide. The present disclosure provides a recombinant expression vector that comprises: a) a nucleotide sequence encoding a Cas12L polypeptide; and b) a nucleotide sequence encoding a Cas12L guide RNA(s). The present disclosure provides a recombinant expression vector that comprises: a) a nucleotide sequence encoding a Cas12L fusion polypeptide; and b) a nucleotide sequence encoding a Cas12L guide RNA(s). In some cases, the nucleotide sequence encoding the Cas12L protein and/or the nucleotide sequence encoding the Cas12L guide RNA is operably linked to a promoter that is operable in a cell type of choice (e.g., a prokaryotic cell, a eukaryotic cell, a plant cell, an animal cell, a mammalian cell, a primate cell, a rodent cell, a human cell, etc.).

In some cases, a nucleotide sequence encoding a Cas12L polypeptide of the present disclosure is codon optimized. This type of optimization can entail a mutation of a Cas12L-encoding nucleotide sequence to mimic the codon preferences of the intended host organism or cell while encoding the same protein. Thus, the codons can be changed, but the encoded protein remains unchanged. For example, if the intended target cell was a human cell, a human codon-optimized Cas12L-encoding nucleotide sequence could be used. As another non-limiting example, if the intended host cell were a mouse cell, then a mouse codon-optimized Cas12L-encoding nucleotide sequence could be generated. As another non-limiting example, if the intended host cell were a plant cell, then a plant codon-optimized Cas12L-encoding nucleotide sequence could be generated. As another non-limiting example, if the intended host cell were an insect cell, then an insect codon-optimized Cas12L-encoding nucleotide sequence could be generated.

Codon usage tables are readily available, for example, at the “Codon Usage Database” available at www[dot]kazusa[dot]or[dot]jp[forwardslash]codon. In some cases, a nucleic acid of the present disclosure comprises a Cas12L polypeptide-encoding nucleotide sequence that is codon optimized for expression in a eukaryotic cell. In some cases, a nucleic acid of the present disclosure comprises a Cas12L polypeptide-encoding nucleotide sequence that is codon optimized for expression in an animal cell. In some cases, a nucleic acid of the present disclosure comprises a Cas12L polypeptide-encoding nucleotide sequence that is codon optimized for expression in a fungus cell. In some cases, a nucleic acid of the present disclosure comprises a Cas12L polypeptide-encoding nucleotide sequence that is codon optimized for expression in a plant cell. In some cases, a nucleic acid of the present disclosure comprises a Cas12L polypeptide-encoding nucleotide sequence that is codon optimized for expression in a monocotyledonous plant species. In some cases, a nucleic acid of the present disclosure comprises a Cas12L polypeptide-encoding nucleotide sequence that is codon optimized for expression in a dicotyledonous plant species. In some cases, a nucleic acid of the present disclosure comprises a Cas12L polypeptide-encoding nucleotide sequence that is codon optimized for expression in a gymnosperm plant species. In some cases, a nucleic acid of the present disclosure comprises a Cas12L polypeptide-encoding nucleotide sequence that is codon optimized for expression in an angiosperm plant species. In some cases, a nucleic acid of the present disclosure comprises a Cas12L polypeptide-encoding nucleotide sequence that is codon optimized for expression in a corn cell. In some cases, a nucleic acid of the present disclosure comprises a Cas12L polypeptide-encoding nucleotide sequence that is codon optimized for expression in a soybean cell. In some cases, a nucleic acid of the present disclosure comprises a Cas12L polypeptide-encoding nucleotide sequence that is codon optimized for expression in a rice cell. In some cases, a nucleic acid of the present disclosure comprises a Cas12L polypeptide-encoding nucleotide sequence that is codon optimized for expression in a wheat cell. In some cases, a nucleic acid of the present disclosure comprises a Cas12L polypeptide-encoding nucleotide sequence that is codon optimized for expression in a cotton cell. In some cases, a nucleic acid of the present disclosure comprises a Cas12L polypeptide-encoding nucleotide sequence that is codon optimized for expression in a sorghum cell. In some cases, a nucleic acid of the present disclosure comprises a Cas12L polypeptide-encoding nucleotide sequence that is codon optimized for expression in an alfalfa cell. In some cases, a nucleic acid of the present disclosure comprises a Cas12L polypeptide-encoding nucleotide sequence that is codon optimized for expression in a sugar cane cell. In some cases, a nucleic acid of the present disclosure comprises a Cas12L polypeptide-encoding nucleotide sequence that is codon optimized for expression in an Arabidopsis cell. In some cases, a nucleic acid of the present disclosure comprises a Cas12L polypeptide-encoding nucleotide sequence that is codon optimized for expression in a tomato cell. In some cases, a nucleic acid of the present disclosure comprises a Cas12L polypeptide-encoding nucleotide sequence that is codon optimized for expression in a cucumber cell. In some cases, a nucleic acid of the present disclosure comprises a Cas12L polypeptide-encoding nucleotide sequence that is codon optimized for expression in a potato cell. In some cases, a nucleic acid of the present disclosure comprises a Cas12L polypeptide-encoding nucleotide sequence that is codon optimized for expression in an algae cell.

The present disclosure provides one or more recombinant expression vectors that include (in different recombinant expression vectors in some cases, and in the same recombinant expression vector in some cases): (i) a nucleotide sequence of a donor template nucleic acid (where the donor template comprises a nucleotide sequence having homology to a target sequence of a target nucleic acid (e.g., a target genome)); (ii) a nucleotide sequence that encodes a Cas12L guide RNA that hybridizes to a target sequence of the target locus of the targeted genome (e.g., operably linked to a promoter that is operable in a target cell such as a eukaryotic cell); and (iii) a nucleotide sequence encoding a Cas12L protein (e.g., operably linked to a promoter that is operable in a target cell such as a eukaryotic cell). The present disclosure provides one or more recombinant expression vectors that include (in different recombinant expression vectors in some cases, and in the same recombinant expression vector in some cases): (i) a nucleotide sequence of a donor template nucleic acid (where the donor template comprises a nucleotide sequence having homology to a target sequence of a target nucleic acid (e.g., a target genome)); and (ii) a nucleotide sequence that encodes a Cas12L guide RNA that hybridizes to a target sequence of the target locus of the targeted genome (e.g., operably linked to a promoter that is operable in a target cell such as a eukaryotic cell). The present disclosure provides one or more recombinant expression vectors that include (in different recombinant expression vectors in some cases, and in the same recombinant expression vector in some cases): (i) a nucleotide sequence that encodes a Cas12L guide RNA that hybridizes to a target sequence of the target locus of the targeted genome (e.g., operably linked to a promoter that is operable in a target cell such as a eukaryotic cell); and (ii) a nucleotide sequence encoding a Cas12L protein (e.g., operably linked to a promoter that is operable in a target cell such as a eukaryotic cell).

Suitable expression vectors include viral expression vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (AAV) (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. In some cases, a recombinant expression vector of the present disclosure is a recombinant adeno-associated virus (AAV) vector. In some cases, a recombinant expression vector of the present disclosure is a recombinant lentivirus vector. In some cases, a recombinant expression vector of the present disclosure is a recombinant retroviral vector.

For plant applications, viral vectors based on Tobamoviruses, Potexviruses, Potyviruses, Tobraviruses, Tombusviruses, Geminiviruses, Bromoviruses, Carmoviruses, Alfamoviruses, or Cucumoviruses can be used. See, e.g., Peyret and Lomonossoff (2015) Plant Biotechnol. J. 13:1121. Suitable Tobamovirus vectors include, for example, a tomato mosaic virus (ToMV) vector, a tobacco mosaic virus (TMV) vector, a tobacco mild green mosaic virus (TMGMV) vector, a pepper mild mottle virus (PMMoV) vector, a paprika mild mottle virus (PaMMV) vector, a cucumber green mottle mosaic virus (CGMMV) vector, a kyuri green mottle mosaic virus (KGMMV) vector, a hibiscus latent fort pierce virus (HLFPV) vector, an odontoglossum ringspot virus (ORSV) vector, a rehmannia mosaic virus (ReMV) vector, a Sammon's opuntia virus (SOV) vector, a wasabi mottle virus (WMoV) vector, a youcai mosaic virus (YoMV) vector, a sunn-hemp mosaic virus (SHMV) vector, and the like. Suitable Potexvirus vectors include, for example, a potato virus X (PVX) vector, a potato aucubamosaicvirus (PAMV) vector, an Alstroemeria virus X (AlsVX) vector, a cactus virus X (CVX) vector, a Cymbidium mosaic virus (CymMV) vector, a hosta virus X (HVX) vector, a lily virus X (LVX) vector, a Narcissus mosaic virus (NMV) vector, a Nerine virus X (NVX) vector, a Plantago asiatica mosaic virus (PlAMV) vector, a strawberry mild yellow edge virus (SMYEV) vector, a tulip virus X (TVX) vector, a white clover mosaic virus (WClMV) vector, a bamboo mosaic virus (BaMV) vector, and the like. Suitable Potyvirus vectors include, for example, a potato virus Y (PVY) vector, a bean common mosaic virus (BCMV) vector, a clover yellow vein virus (ClYVV) vector, an East Asian Passiflora virus (EAPV) vector, a Freesia mosaic virus (FreMV) vector, a Japanese yam mosaic virus (JYMV) vector, a lettuce mosaic virus (LMV) vector, a Maize dwarf mosaic virus (MDMV) vector, an onion yellow dwarf virus (OYDV) vector, a papaya ringspot virus (PRSV) vector, a pepper mottle virus (PepMoV) vector, a Perilla mottle virus (PerMoV) vector, a plum pox virus (PPV) vector, a potato virus A (PVA) vector, a sorghum mosaic virus (SrMV) vector, a soybean mosaic virus (SMV) vector, a sugarcane mosaic virus (SCMV) vector, a tulip mosaic virus (TulMV) vector, a turnip mosaic virus (TuMV) vector, a watermelon mosaic virus (WMV) vector, a zucchini yellow mosaic virus (ZYMV) vector, a tobacco etch virus (TEV) vector, and the like. Suitable Tobravirus vectors include, for example, a tobacco rattle virus (TRV) vector and the like. Suitable Tombusvirus vectors include, for example, a tomato bushy stunt virus (TBSV) vector, an eggplant mottled crinkle virus (EMCV) vector, a grapevine Algerian latent virus (GALV) vector, and the like. Suitable Cucumovirus vectors include, for example, a cucumber mosaic virus (CMV) vector, a peanut stunt virus (PSV) vector, a tomato aspermy virus (TAV) vector, and the like. Suitable Bromovirus vectors include, for example, a brome mosaic virus (BMV) vector, a cowpea chlorotic mottle virus (CCMV) vector, and the like. Suitable Carmovirus vectors include, for example, a carnation mottle virus (CarMV) vector, a melon necrotic spot virus (MNSV) vector, a pea stem necrotic virus (PSNV) vector, a turnip crinkle virus (TCV) vector, and the like. Suitable Alfamovirus vectors include, for example, an alfalfa mosaic virus (AMV) vector, and the like.

Depending on the host/vector system utilized, any of a number of suitable transcription and translation control elements, including constitutive and inducible promoters, transcription enhancer elements, transcription terminators, etc. may be used in the expression vector.

In some embodiments, a nucleotide sequence encoding a Cas12L guide RNA is operably linked to a control element, e.g., a transcriptional control element, such as a promoter. In some embodiments, a nucleotide sequence encoding a Cas12L protein or a Cas12L fusion polypeptide is operably linked to a control element, e.g., a transcriptional control element, such as a promoter.

The transcriptional control element can be a promoter. In some cases, the promoter is a constitutively active promoter. In some cases, the promoter is a regulatable promoter. In some cases, the promoter is an inducible promoter. In some cases, the promoter is a tissue-specific promoter. In some cases, the promoter is a cell type-specific promoter. In some cases, the transcriptional control element (e.g., the promoter) is functional in a targeted cell type or targeted cell population. For example, in some cases, the transcriptional control element can be functional in eukaryotic cells, e.g., hematopoietic stem cells (e.g., mobilized peripheral blood (mPB) CD34(+) cell, bone marrow (BM) CD34(+) cell, etc.).

Non-limiting examples of eukaryotic promoters (promoters functional in a eukaryotic cell) include EF1α, those from cytomegalovirus (CMV) immediate early, herpes simplex virus (HSV) thymidine kinase, early and late SV40, long terminal repeats (LTRs) from retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression. The expression vector may also include nucleotide sequences encoding protein tags (e.g., 6×His tag, hemagglutinin tag, fluorescent protein, etc.) that can be fused to the Cas12L protein, thus resulting in a fusion Cas12L polypeptide.

In some embodiments, a nucleotide sequence encoding a Cas12L guide RNA and/or a Cas12L fusion polypeptide is operably linked to an inducible promoter. In some embodiments, a nucleotide sequence encoding a Cas12L guide RNA and/or a Cas12L fusion protein is operably linked to a constitutive promoter.

A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular temperature, compound, or protein.), it may be a spatially restricted promoter (i.e., transcriptional control element, enhancer, etc.)(e.g., tissue specific promoter, cell type specific promoter, etc.), and it may be a temporally restricted promoter (i.e., the promoter is in the “ON” state or “OFF” state during specific stages of embryonic development or during specific stages of a biological process, e.g., hair follicle cycle in mice).

Suitable promoters can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6) (Miyagishi et al., Nature Biotechnology 20, 497-500 (2002)), an enhanced U6 promoter (e.g., Xia et al., Nucleic Acids Res. 2003 Sep. 1; 31(17)), a human H1 promoter (H1), and the like.

In some cases, a nucleotide sequence encoding a Cas12L guide RNA is operably linked to (under the control of) a promoter operable in a eukaryotic cell (e.g., a U6 promoter, an enhanced U6 promoter, an H1 promoter, and the like). As would be understood by one of ordinary skill in the art, when expressing an RNA (e.g., a guide RNA) from a nucleic acid (e.g., an expression vector) using a U6 promoter (e.g., in a eukaryotic cell), or another PolIII promoter, the RNA may need to be mutated if there are several Ts in a row (coding for Us in the RNA). This is because a string of Ts (e.g., 5 Ts) in DNA can act as a terminator for polymerase III (PolIII). Thus, in order to ensure transcription of a guide RNA in a eukaryotic cell it may sometimes be necessary to modify the sequence encoding the guide RNA to eliminate runs of Ts. In some cases, a nucleotide sequence encoding a Cas12L protein (e.g., a wild type Cas12L protein, a nickase Cas12L protein, a dCas12L protein, a fusion Cas12L protein and the like) is operably linked to a promoter operable in a eukaryotic cell (e.g., a CMV promoter, an EF1α promoter, an estrogen receptor-regulated promoter, and the like).

Examples of inducible promoters include, but are not limited toT7 RNA polymerase promoter, T3 RNA polymerase promoter, Isopropyl-beta-D-thiogalactopyranoside (IPTG)-regulated promoter, lactose induced promoter, heat shock promoter, Tetracycline-regulated promoter, Steroid-regulated promoter, Metal-regulated promoter, estrogen receptor-regulated promoter, etc. Inducible promoters can therefore be regulated by molecules including, but not limited to, doxycycline; estrogen and/or an estrogen analog; IPTG; etc.

Inducible promoters suitable for use include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

In some cases, the promoter is a spatially restricted promoter (i.e., cell type specific promoter, tissue specific promoter, etc.) such that in a multi-cellular organism, the promoter is active (i.e., “ON”) in a subset of specific cells. Spatially restricted promoters may also be referred to as enhancers, transcriptional control elements, control sequences, etc. Any convenient spatially restricted promoter may be used as long as the promoter is functional in the targeted host cell (e.g., eukaryotic cell; prokaryotic cell).

In some cases, the promoter is a reversible promoter. Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

RNA polymerase III (Pol III) promoters can be used to drive the expression of non-protein coding RNA molecules (e.g., guide RNAs). In some cases, a suitable promoter is a Pol III promoter. In some cases, a Pol III promoter is operably linked to a nucleotide sequence encoding a guide RNA (gRNA). In some cases, a Pol III promoter is operably linked to a nucleotide sequence encoding a single-guide RNA (sgRNA). In some cases, a Pol III promoter is operably linked to a nucleotide sequence encoding a CRISPR RNA (crRNA). In some cases, a Pol III promoter is operably linked to a nucleotide sequence encoding a encoding a tracrRNA.

Non-limiting examples of Pol III promoters include a U6 promoter, an Hl promoter, a 5S promoter, an Adenovirus 2 (Ad2) VAI promoter, a tRNA promoter, and a 7SK promoter. See, for example, Schramm and Hernandez (2002) Genes & Development 16:2593-2620. In some cases, a Pol III promoter is selected from the group consisting of a U6 promoter, an Hl promoter, a 5S promoter, an Adenovirus 2 (Ad2) VAI promoter, a tRNA promoter, and a 7SK promoter. In some cases, a guide RNA-encoding nucleotide sequence is operably linked to a promoter selected from the group consisting of a U6 promoter, an Hl promoter, a 5S promoter, an Adenovirus 2 (Ad2) VAI promoter, a tRNA promoter, and a 7SK promoter. In some cases, a single-guide RNA-encoding nucleotide sequence is operably linked to a promoter selected from the group consisting of a U6 promoter, an Hl promoter, a 5S promoter, an Adenovirus 2 (Ad2) VAI promoter, a tRNA promoter, and a 7SK promoter.

Examples describing a promoter that can be used herein in connection with expression in plants, plant tissues, and plant cells include, but are not limited to, promoters described in: U.S. Pat. No. 6,437,217 (maize RS81 promoter), U.S. Pat. No. 5,641,876 (rice actin promoter), U.S. Pat. No. 6,426,446 (maize RS324 promoter), U.S. Pat. No. 6,429,362 (maize PR-1 promoter), U.S. Pat. No. 6,232,526 (maize A3 promoter), U.S. Pat. No. 6,177,611 (constitutive maize promoters), U.S. Pat. Nos. 5,322,938, 5,352,605, 5,359,142 and 5,530,196 (35S promoter), U.S. Pat. No. 6,433,252 (maize L3 oleosin promoter), U.S. Pat. No. 6,429,357 (rice actin 2 promoter as well as a rice actin 2 intron), U.S. Pat. No. 5,837,848 (root specific promoter), U.S. Pat. No. 6,294,714 (light inducible promoters), U.S. Pat. No. 6,140,078 (salt inducible promoters), U.S. Pat. No. 6,252,138 (pathogen inducible promoters), U.S. Pat. No. 6,175,060 (phosphorus deficiency inducible promoters), U.S. Pat. No. 6,635,806 (gamma-coixin promoter), and U.S. patent application Ser. No. 09/757,089 (maize chloroplast aldolase promoter). Additional promoters that can find use include a nopaline synthase (NOS) promoter (Ebert et al., 1987), the octopine synthase (OCS) promoter (which is carried on tumor-inducing plasmids of Agrobacterium tumefaciens), the caulimovirus promoters such as the cauliflower mosaic virus (CaMV) 19S promoter (Lawton et al. Plant Molecular Biology (1987) 9: 315-324), the CaMV 35S promoter (Odell et al., Nature (1985) 313: 810-812), the figwort mosaic virus 35S-promoter (U.S. Pat. Nos. 6,051,753; 5,378,619), the sucrose synthase promoter (Yang and Russell, Proceedings of the National Academy of Sciences, USA (1990) 87: 4144-4148), the R gene complex promoter (Chandler et al., Plant Cell (1989) 1: 1175-1183), and the chlorophyll a/b binding protein gene promoter, PC1SV (U.S. Pat. No. 5,850,019), and AGRtu.nos (GenBank Accession V00087; Depicker et al., Journal of Molecular and Applied Genetics (1982) 1: 561-573; Bevan et al., 1983) promoters.

Methods of introducing a nucleic acid (e.g., a nucleic acid comprising a donor polynucleotide sequence, one or more nucleic acids encoding a Cas12L protein and/or a Cas12L guide RNA, and the like) into a host cell are known in the art, and any convenient method can be used to introduce a nucleic acid (e.g., an expression construct) into a cell. Suitable methods include e.g., viral infection, transfection, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery, and the like.

Introducing the recombinant expression vector into cells can occur in any culture media and under any culture conditions that promote the survival of the cells. Introducing the recombinant expression vector into a target cell can be carried out in vivo or ex vivo. Introducing the recombinant expression vector into a target cell can be carried out in vitro.

In some embodiments, a Cas12L protein can be provided as RNA. The RNA can be provided by direct chemical synthesis or may be transcribed in vitro from a DNA (e.g., encoding the Cas12L protein). Once synthesized, the RNA may be introduced into a cell by any of the well-known techniques for introducing nucleic acids into cells (e.g., microinjection, electroporation, transfection, etc.).

Nucleic acids may be provided to the cells using well-developed transfection techniques; see, e.g. Angel and Yanik (2010) PLoS ONE 5(7): e11756, and the commercially available TransMessenger® reagents from Qiagen, Stemfect™ RNA Transfection Kit from Stemgent, and TransIT®-mRNA Transfection Kit from Mirus Bio LLC. See also Beumer et al. (2008) PNAS 105(50):19821-19826.

Vectors may be provided directly to a target host cell. In other words, the cells are contacted with vectors comprising the subject nucleic acids (e.g., recombinant expression vectors having the donor template sequence and encoding the Cas12L guide RNA; recombinant expression vectors encoding the Cas12L protein; etc.) such that the vectors are taken up by the cells. Methods for contacting cells with nucleic acid vectors that are plasmids, include electroporation, calcium chloride transfection, microinjection, and lipofection are well known in the art. For viral vector delivery, cells can be contacted with viral particles comprising the subject viral expression vectors.

Retroviruses, for example, lentiviruses, are suitable for use in methods of the present disclosure. Commonly used retroviral vectors are “defective”, i.e. unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein (ecotropic, amphotropic or xenotropic) to be incorporated into the capsid, this envelope protein determining the specificity of the viral particle for the cells (ecotropic for murine and rat; amphotropic for most mammalian cell types including human, dog and mouse; and xenotropic for most mammalian cell types except murine cells). The appropriate packaging cell line may be used to ensure that the cells are targeted by the packaged viral particles. Methods of introducing subject vector expression vectors into packaging cell lines and of collecting the viral particles that are generated by the packaging lines are well known in the art. Nucleic acids can also introduced by direct micro-injection (e.g., injection of RNA).

Vectors used for providing the nucleic acids encoding Cas12L guide RNA and/or a Cas12L polypeptide to a target host cell can include suitable promoters for driving the expression, that is, transcriptional activation, of the nucleic acid of interest. In other words, in some cases, the nucleic acid of interest will be operably linked to a promoter. This may include ubiquitously acting promoters, for example, the CMV-3-actin promoter, or inducible promoters, such as promoters that are active in particular cell populations or that respond to the presence of drugs such as tetracycline. By transcriptional activation, it is intended that transcription will be increased above basal levels in the target cell by 10 fold, by 100 fold, more usually by 1000 fold. In addition, vectors used for providing a nucleic acid encoding a Cas12L guide RNA and/or a Cas12L protein to a cell may include nucleic acid sequences that encode for selectable markers in the target cells, so as to identify cells that have taken up the Cas12L guide RNA and/or Cas12L protein.

A nucleic acid comprising a nucleotide sequence encoding a Cas12L polypeptide, or a Cas12L fusion polypeptide, is in some cases an RNA. Thus, a Cas12L fusion protein can be introduced into cells as RNA. Methods of introducing RNA into cells are known in the art and may include, for example, direct injection, transfection, or any other method used for the introduction of DNA. A Cas12L protein may instead be provided to cells as a polypeptide. Such a polypeptide may optionally be fused to a polypeptide domain that increases solubility of the product. The domain may be linked to the polypeptide through a defined protease cleavage site, e.g. a TEV sequence, which is cleaved by TEV protease. The linker may also include one or more flexible sequences, e.g. from 1 to 10 glycine residues. In some embodiments, the cleavage of the fusion protein is performed in a buffer that maintains solubility of the product, e.g. in the presence of from 0.5 to 2 M urea, in the presence of polypeptides and/or polynucleotides that increase solubility, and the like. Domains of interest include endosomolytic domains, e.g. influenza HA domain; and other polypeptides that aid in production, e.g. IF2 domain, GST domain, GRPE domain, and the like. The polypeptide may be formulated for improved stability. For example, the peptides may be PEGylated, where the polyethyleneoxy group provides for enhanced lifetime in the blood stream.

Additionally or alternatively, a Cas12L polypeptide of the present disclosure may be fused to a polypeptide permeant domain to promote uptake by the cell. A number of permeant domains are known in the art and may be used in the non-integrating polypeptides of the present disclosure, including peptides, peptidomimetics, and non-peptide carriers. For example, a permeant peptide may be derived from the third alpha helix of Drosophila melanogaster transcription factor Antennapaedia, referred to as penetratin, which comprises the amino acid sequence RQIKIWFQNRRMKWKK (SEQ ID NO:163). As another example, the permeant peptide comprises the HIV-1 tat basic region amino acid sequence, which may include, for example, amino acids 49-57 of naturally-occurring tat protein. Other permeant domains include poly-arginine motifs, for example, the region of amino acids 34-56 of HIV-1 rev protein, nona-arginine, octa-arginine, and the like. (See, for example, Futaki et al. (2003) Curr Protein Pept Sci. 2003 April; 4(2): 87-9 and 446; and Wender et al. (2000) Proc. Natl. Acad. Sci. U.S.A 2000 Nov. 21; 97(24):13003-8; published U.S. Patent applications 20030220334; 20030083256; 20030032593; and 20030022831, herein specifically incorporated by reference for the teachings of translocation peptides and peptoids). The nona-arginine (R9) sequence is one of the more efficient PTDs that have been characterized (Wender et al. 2000; Uemura et al. 2002). The site at which the fusion is made may be selected in order to optimize the biological activity, secretion or binding characteristics of the polypeptide. The optimal site will be determined by routine experimentation.

As noted above, in some cases, the target cell is a plant cell. Numerous methods for transforming chromosomes or plastids in a plant cell with a recombinant nucleic acid are known in the art, which can be used according to methods of the present application to produce a transgenic plant cell and/or a transgenic plant. Any suitable method or technique for transformation of a plant cell known in the art can be used. Effective methods for transformation of plants include bacterially mediated transformation, such as Agrobacterium-mediated or Rhizobium-mediated transformation and microprojectile bombardment-mediated transformation. A variety of methods are known in the art for transforming explants with a transformation vector via bacterially mediated transformation or microprojectile bombardment and then subsequently culturing, etc., those explants to regenerate or develop transgenic plants. Other methods for plant transformation, such as microinjection, electroporation, vacuum infiltration, pressure, sonication, silicon carbide fiber agitation, PEG-mediated transformation, etc., are also known in the art. Transgenic plants produced by these transformation methods can be chimeric or non-chimeric for the transformation event depending on the methods and explants used.

Methods of transforming plant cells are well known by persons of ordinary skill in the art. For instance, specific instructions for transforming plant cells by microprojectile bombardment with particles coated with recombinant DNA (e.g., biolistic transformation) are found in U.S. Pat. Nos. 5,550,318; 5,538,880 6,160,208; 6,399,861; and 6,153,812 and Agrobacterium-mediated transformation is described in U.S. Pat. Nos. 5,159,135; 5,824,877; 5,591,616; 6,384,301; 5,750,871; 5,463,174; and 5,188,958. Additional methods for transforming plants can be found in, for example, Compendium of Transgenic Crop Plants (2009) Blackwell Publishing. Any appropriate method known to those skilled in the art can be used to transform a plant cell with any of the nucleic acids provided herein.

A Cas12L polypeptide of the present disclosure may be produced in vitro or by eukaryotic cells or by prokaryotic cells, and it may be further processed by unfolding, e.g. heat denaturation, dithiothreitol reduction, etc. and may be further refolded, using methods known in the art.

Modifications of interest that do not alter primary sequence include chemical derivatization of polypeptides, e.g., acylation, acetylation, carboxylation, amidation, etc. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine.

Also suitable for inclusion in embodiments of the present disclosure are nucleic acids (e.g., encoding a Cas12L guide RNA, encoding a Cas12L fusion protein, etc.) and proteins (e.g., a Cas12L fusion protein derived from a wild type protein or a variant protein) that have been modified using ordinary molecular biological techniques and synthetic chemistry so as to improve their resistance to proteolytic degradation, to change the target sequence specificity, to optimize solubility properties, to alter protein activity (e.g., transcription modulatory activity, enzymatic activity, etc.) or to render them more suitable. Analogs of such polypeptides include those containing residues other than naturally occurring L-amino acids, e.g. D-amino acids or non-naturally occurring synthetic amino acids. D-amino acids may be substituted for some or all of the amino acid residues.

A Cas12L polypeptide of the present disclosure may be prepared by in vitro synthesis, using conventional methods as known in the art. Various commercial synthetic apparatuses are available, for example, automated synthesizers by Applied Biosystems, Inc., Beckman, etc. By using synthesizers, naturally occurring amino acids may be substituted with unnatural amino acids. The particular sequence and the manner of preparation will be determined by convenience, economics, purity required, and the like.

If desired, various groups may be introduced into the peptide during synthesis or during expression, which allow for linking to other molecules or to a surface. Thus, cysteines can be used to make thioethers, histidines for linking to a metal ion complex, carboxyl groups for forming amides or esters, amino groups for forming amides, and the like.

A Cas12L polypeptide of the present disclosure may also be isolated and purified in accordance with conventional methods of recombinant synthesis. A lysate may be prepared of the expression host and the lysate purified using high performance liquid chromatography (HPLC), exclusion chromatography, gel electrophoresis, affinity chromatography, or other purification technique. For the most part, the compositions which are used will comprise 20% or more by weight of the desired product, more usually 75% or more by weight, preferably 95% or more by weight, and for therapeutic purposes, usually 99.5% or more by weight, in relation to contaminants related to the method of preparation of the product and its purification. Usually, the percentages will be based upon total protein. Thus, in some cases, a Cas12L polypeptide, or a Cas12L fusion polypeptide, of the present disclosure is at least 80% pure, at least 85% pure, at least 90% pure, at least 95% pure, at least 98% pure, or at least 99% pure (e.g., free of contaminants, non-Cas12L proteins or other macromolecules, etc.).

To induce cleavage or any desired modification to a target nucleic acid (e.g., genomic DNA), or any desired modification to a polypeptide associated with target nucleic acid, the Cas12L guide RNA and/or the Cas12L polypeptide of the present disclosure and/or the donor template sequence, whether they be introduced as nucleic acids or polypeptides, are provided to the cells for about 30 minutes to about 24 hours, e.g., 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which may be repeated with a frequency of about every day to about every 4 days, e.g., every 1.5 days, every 2 days, every 3 days, or any other frequency from about every day to about every four days. The agent(s) may be provided to the subject cells one or more times, e.g. one time, twice, three times, or more than three times, and the cells allowed to incubate with the agent(s) for some amount of time following each contacting event e.g. 16-24 hours, after which time the media is replaced with fresh media and the cells are cultured further.

In cases in which two or more different targeting complexes are provided to the cell (e.g., two different Cas12L guide RNAs that are complementary to different sequences within the same or different target nucleic acid), the complexes may be provided simultaneously (e.g. as two polypeptides and/or nucleic acids), or delivered simultaneously. Alternatively, they may be provided consecutively, e.g. the targeting complex being provided first, followed by the second targeting complex, etc. or vice versa.

To improve the delivery of a DNA vector into a target cell, the DNA can be protected from damage and its entry into the cell facilitated, for example, by using lipoplexes and polyplexes. Thus, in some cases, a nucleic acid of the present disclosure (e.g., a recombinant expression vector of the present disclosure) can be covered with lipids in an organized structure like a micelle or a liposome. When the organized structure is complexed with DNA it is called a lipoplex. There are three types of lipids, anionic (negatively-charged), neutral, or cationic (positively-charged). Lipoplexes that utilize cationic lipids have proven utility for gene transfer. Cationic lipids, due to their positive charge, naturally complex with the negatively charged DNA. Also as a result of their charge, they interact with the cell membrane. Endocytosis of the lipoplex then occurs, and the DNA is released into the cytoplasm. The cationic lipids also protect against degradation of the DNA by the cell.

Complexes of polymers with DNA are called polyplexes. Most polyplexes consist of cationic polymers and their production is regulated by ionic interactions. One large difference between the methods of action of polyplexes and lipoplexes is that polyplexes cannot release their DNA load into the cytoplasm, so to this end, co-transfection with endosome-lytic agents (to lyse the endosome that is made during endocytosis) such as inactivated adenovirus must occur. However, this is not always the case; polymers such as polyethylenimine have their own method of endosome disruption as does chitosan and trimethylchitosan.

Dendrimers, a highly branched macromolecule with a spherical shape, may be also be used to genetically modify stem cells. The surface of the dendrimer particle may be functionalized to alter its properties. In particular, it is possible to construct a cationic dendrimer (i.e., one with a positive surface charge). When in the presence of genetic material such as a DNA plasmid, charge complementarity leads to a temporary association of the nucleic acid with the cationic dendrimer. On reaching its destination, the dendrimer-nucleic acid complex can be taken up into a cell by endocytosis.

In some cases, a nucleic acid of the disclosure (e.g., an expression vector) includes an insertion site for a guide sequence of interest. For example, a nucleic acid can include an insertion site for a guide sequence of interest, where the insertion site is immediately adjacent to a nucleotide sequence encoding the portion of a Cas12L guide RNA that does not change when the guide sequence is changed to hybridized to a desired target sequence (e.g., sequences that contribute to the Cas12L binding aspect of the guide RNA, e.g., the sequences that contribute to the dsRNA duplex(es) of the Cas12L guide RNA—this portion of the guide RNA can also be referred to as the ‘scaffold’ or ‘constant region’ of the guide RNA). Thus, in some cases, a subject nucleic acid (e.g., an expression vector) includes a nucleotide sequence encoding a Cas12L guide RNA, except that the portion encoding the guide sequence portion of the guide RNA is an insertion sequence (an insertion site). An insertion site is any nucleotide sequence used for the insertion of the desired sequence. “Insertion sites” for use with various technologies are known to those of ordinary skill in the art and any convenient insertion site can be used. An insertion site can be for any method for manipulating nucleic acid sequences. For example, in some cases the insertion site is a multiple cloning site (MCS) (e.g., a site including one or more restriction enzyme recognition sequences), a site for ligation independent cloning, a site for recombination based cloning (e.g., recombination based on att sites), a nucleotide sequence recognized by a CRISPR/Cas (e.g. Cas9) based technology, and the like.

An insertion site can be any desirable length, and can depend on the type of insertion site (e.g., can depend on whether (and how many) the site includes one or more restriction enzyme recognition sequences, whether the site includes a target site for a CRISPR/Cas protein, etc.). In some cases, an insertion site of a subject nucleic acid is 3 or more nucleotides (nt) in length (e.g., 5 or more, 8 or more, 10 or more, 15 or more, 17 or more, 18 or more, 19 or more, 20 or more or 25 or more, or 30 or more nt in length). In some cases, the length of an insertion site of a subject nucleic acid has a length in a range of from 2 to 50 nucleotides (nt) (e.g., from 2 to 40 nt, from 2 to 30 nt, from 2 to 25 nt, from 2 to 20 nt, from 5 to 50 nt, from 5 to 40 nt, from 5 to 30 nt, from 5 to 25 nt, from 5 to 20 nt, from 10 to 50 nt, from 10 to 40 nt, from 10 to 30 nt, from 10 to 25 nt, from 10 to 20 nt, from 17 to 50 nt, from 17 to 40 nt, from 17 to 30 nt, from 17 to 25 nt). In some cases, the length of an insertion site of a subject nucleic acid has a length in a range of from 5 to 40 nt.

### Nucleic Acid Modifications

In some embodiments, a subject nucleic acid (e.g., a Cas12L guide RNA) has one or more modifications, e.g., a base modification, a backbone modification, etc., to provide the nucleic acid with a new or enhanced feature (e.g., improved stability). A nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to the 2′, the 3′, or the 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound, however, linear compounds are suitable. In addition, linear compounds may have internal nucleotide base complementarity and may therefore fold in a manner as to produce a fully or partially double-stranded compound. Within oligonucleotides, the phosphate groups are commonly referred to as forming the internucleoside backbone of the oligonucleotide. The normal linkage or backbone of RNA and DNA is a 3′ to 5′ phosphodiester linkage.

Suitable nucleic acid modifications include, but are not limited to: 2′Omethyl modified nucleotides, 2′ Fluoro modified nucleotides, locked nucleic acid (LNA) modified nucleotides, peptide nucleic acid (PNA) modified nucleotides, nucleotides with phosphorothioate linkages, and a 5′ cap (e.g., a 7-methylguanylate cap (m7G)). Additional details and additional modifications are described below.

A 2′-O-Methyl modified nucleotide (also referred to as 2′-O-Methyl RNA) is a naturally occurring modification of RNA found in tRNA and other small RNAs that arises as a post-transcriptional modification. Oligonucleotides can be directly synthesized that contain 2′-O-Methyl RNA. This modification increases Tm of RNA:RNA duplexes but results in only small changes in RNA:DNA stability. It is stabile with respect to attack by single-stranded ribonucleases and is typically 5 to 10-fold less susceptible to DNases than DNA. It is commonly used in antisense oligos as a means to increase stability and binding affinity to the target message.

2′ Fluoro modified nucleotides (e.g., 2′ Fluoro bases) have a fluorine modified ribose which increases binding affinity (Tm) and also confers some relative nuclease resistance when compared to native RNA. These modifications are commonly employed in ribozymes and siRNAs to improve stability in serum or other biological fluids.

LNA bases have a modification to the ribose backbone that locks the base in the C3′-endo position, which favors RNA A-type helix duplex geometry. This modification significantly increases Tm and is also very nuclease resistant. Multiple LNA insertions can be placed in an oligo at any position except the 3′-end. Applications have been described ranging from antisense oligos to hybridization probes to SNP detection and allele specific PCR. Due to the large increase in Tm conferred by LNAs, they also can cause an increase in primer dimer formation as well as self-hairpin formation. In some cases, the number of LNAs incorporated into a single oligo is 10 bases or less.

The phosphorothioate (PS) bond (i.e., a phosphorothioate linkage) substitutes a sulfur atom for a non-bridging oxygen in the phosphate backbone of a nucleic acid (e.g., an oligo). This modification renders the internucleotide linkage resistant to nuclease degradation. Phosphorothioate bonds can be introduced between the last 3-5 nucleotides at the 5′- or 3′-end of the oligo to inhibit exonuclease degradation. Including phosphorothioate bonds within the oligo (e.g., throughout the entire oligo) can help reduce attack by endonucleases as well.

In some cases, a subject nucleic acid has one or more nucleotides that are 2′-O-Methyl modified nucleotides. In some embodiments, a subject nucleic acid (e.g., a dsRNA, a siNA, etc.) has one or more 2′ Fluoro modified nucleotides. In some embodiments, a subject nucleic acid (e.g., a dsRNA, a siNA, etc.) has one or more LNA bases. In some embodiments, a subject nucleic acid (e.g., a dsRNA, a siNA, etc.) has one or more nucleotides that are linked by a phosphorothioate bond (i.e., the subject nucleic acid has one or more phosphorothioate linkages). In some embodiments, a subject nucleic acid (e.g., a dsRNA, a siNA, etc.) has a 5′ cap (e.g., a 7-methylguanylate cap (m7G)). In some embodiments, a subject nucleic acid (e.g., a dsRNA, a siNA, etc.) has a combination of modified nucleotides. For example, a subject nucleic acid (e.g., a dsRNA, a siNA, etc.) can have a 5′ cap (e.g., a 7-methylguanylate cap (m7G)) in addition to having one or more nucleotides with other modifications (e.g., a 2′-O-Methyl nucleotide and/or a 2′ Fluoro modified nucleotide and/or a LNA base and/or a phosphorothioate linkage).

### Modified Backbones and Modified Internucleoside Linkages

Examples of suitable nucleic acids (e.g., a Cas12L guide RNA) containing modifications include nucleic acids containing modified backbones or non-natural internucleoside linkages. Nucleic acids having modified backbones include those that retain a phosphorus atom in the backbone and those that do not have a phosphorus atom in the backbone.

Suitable modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Suitable oligonucleotides having inverted polarity comprise a single 3′ to 3′ linkage at the 3′-most internucleotide linkage i.e. a single inverted nucleoside residue which may be a basic (the nucleobase is missing or has a hydroxyl group in place thereof). Various salts (such as, for example, potassium or sodium), mixed salts and free acid forms are also included.

In some embodiments, a subject nucleic acid comprises one or more phosphorothioate and/or heteroatom internucleoside linkages, in particular —CH₂—NHO—CH₂—, —CH₂—N(CH₃)—O—CH₂-(known as a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(═O)(OH)—O—CH₂—). MMI type internucleoside linkages are disclosed in the above referenced U.S. Pat. No. 5,489,677, the disclosure of which is incorporated herein by reference in its entirety. Suitable amide internucleoside linkages are disclosed in U.S. Pat. No. 5,602,240, the disclosure of which is incorporated herein by reference in its entirety.

Also suitable are nucleic acids having morpholino backbone structures as described in, e.g., U.S. Pat. No. 5,034,506. For example, in some embodiments, a subject nucleic acid comprises a 6-membered morpholino ring in place of a ribose ring. In some of these embodiments, a phosphorodiamidate or other non-phosphodiester internucleoside linkage replaces a phosphodiester linkage.

Suitable modified polynucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

### Mimetics

A subject nucleic acid can be a nucleic acid mimetic. The term “mimetic” as it is applied to polynucleotides is intended to include polynucleotides wherein only the furanose ring or both the furanose ring and the internucleotide linkage are replaced with non-furanose groups, replacement of only the furanose ring is also referred to in the art as being a sugar surrogate. The heterocyclic base moiety or a modified heterocyclic base moiety is maintained for hybridization with an appropriate target nucleic acid. One such nucleic acid, a polynucleotide mimetic that has been shown to have excellent hybridization properties, is referred to as a peptide nucleic acid (PNA). In PNA, the sugar-backbone of a polynucleotide is replaced with an amide containing backbone, in particular an aminoethylglycine backbone. The nucleotides are retained and are bound directly or indirectly to aza nitrogen atoms of the amide portion of the backbone.

One polynucleotide mimetic that has been reported to have excellent hybridization properties is a peptide nucleic acid (PNA). The backbone in PNA compounds is two or more linked aminoethylglycine units which gives PNA an amide containing backbone. The heterocyclic base moieties are bound directly or indirectly to aza nitrogen atoms of the amide portion of the backbone. Representative U.S. patents that describe the preparation of PNA compounds include, but are not limited to: U.S. Pat. Nos. 5,539,082; 5,714,331; and 5,719,262, the disclosures of which are incorporated herein by reference in their entirety.

Another class of polynucleotide mimetic that has been studied is based on linked morpholino units (morpholino nucleic acid) having heterocyclic bases attached to the morpholino ring. A number of linking groups have been reported that link the morpholino monomeric units in a morpholino nucleic acid. One class of linking groups has been selected to give a non-ionic oligomeric compound. The non-ionic morpholino-based oligomeric compounds are less likely to have undesired interactions with cellular proteins. Morpholino-based polynucleotides are non-ionic mimics of oligonucleotides which are less likely to form undesired interactions with cellular proteins (Dwaine A. Braasch and David R. Corey, Biochemistry, 2002, 41(14), 4503-4510). Morpholino-based polynucleotides are disclosed in U.S. Pat. No. 5,034,506, the disclosure of which is incorporated herein by reference in its entirety. A variety of compounds within the morpholino class of polynucleotides have been prepared, having a variety of different linking groups joining the monomeric subunits.

A further class of polynucleotide mimetic is referred to as cyclohexenyl nucleic acids (CeNA). The furanose ring normally present in a DNA/RNA molecule is replaced with a cyclohexenyl ring. CeNA DMT protected phosphoramidite monomers have been prepared and used for oligomeric compound synthesis following classical phosphoramidite chemistry. Fully modified CeNA oligomeric compounds and oligonucleotides having specific positions modified with CeNA have been prepared and studied (see Wang et al., J. Am. Chem. Soc., 2000, 122, 8595-8602, the disclosure of which is incorporated herein by reference in its entirety). In general the incorporation of CeNA monomers into a DNA chain increases its stability of a DNA/RNA hybrid. CeNA oligoadenylates formed complexes with RNA and DNA complements with similar stability to the native complexes. The study of incorporating CeNA structures into natural nucleic acid structures was shown by NMR and circular dichroism to proceed with easy conformational adaptation.

A further modification includes Locked Nucleic Acids (LNAs) in which the 2′-hydroxyl group is linked to the 4′ carbon atom of the sugar ring thereby forming a 2′-C,4′-C-oxymethylene linkage thereby forming a bicyclic sugar moiety. The linkage cfan be a methylene (—CH₂—), group bridging the 2′ oxygen atom and the 4′ carbon atom wherein n is 1 or 2 (Singh et al., Chem. Commun., 1998, 4, 455-456, the disclosure of which is incorporated herein by reference in its entirety). LNA and LNA analogs display very high duplex thermal stabilities with complementary DNA and RNA (Tm=+3 to +100 C), stability towards 3′-exonucleolytic degradation and good solubility properties. Potent and nontoxic antisense oligonucleotides containing LNAs have been described (e.g., Wahlestedt et al., Proc. Natl. Acad. Sci. U.S.A., 2000, 97, 5633-5638, the disclosure of which is incorporated herein by reference in its entirety).

The synthesis and preparation of the LNA monomers adenine, cytosine, guanine, 5-methyl-cytosine, thymine and uracil, along with their oligomerization, and nucleic acid recognition properties have been described (e.g., Koshkin et al., Tetrahedron, 1998, 54, 3607-3630, the disclosure of which is incorporated herein by reference in its entirety). LNAs and preparation thereof are also described in WO 98/39352 and WO 99/14226, as well as U.S. applications 20120165514, 20100216983, 20090041809, 20060117410, 20040014959, 20020094555, and 20020086998, the disclosures of which are incorporated herein by reference in their entirety.

### Modified Sugar Moieties

A subject nucleic acid can also include one or more substituted sugar moieties. Suitable polynucleotides comprise a sugar substituent group selected from: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly suitable are O((CH₂)ₙO)ₘCH₃, O(CH₂)ₙOCH₃, O(CH₂)ₙNH₂, O(CH₂)ₙCH₃, O(CH₂)ₙONH₂, and O(CH₂)ₙON((CH₂)ₙCH₃)₂, where n and m are from 1 to about 10. Other suitable polynucleotides comprise a sugar substituent group selected from: C₁ to C₁₀ lower alkyl, substituted lower alkyl, alkenyl, alkynyl, alkaryl, aralkyl, O-alkaryl or O-aralkyl, SH, SCH₃, OCN, Cl Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A suitable modification includes 2′-methoxyethoxy (2′-O—CH₂ CH₂OCH₃, also known as 2′-O-(2-methoxyethyl) or 2′-MOE) (Martin et al., Helv. Chim. Acta, 1995, 78, 486-504, the disclosure of which is incorporated herein by reference in its entirety) i.e., an alkoxyalkoxy group. A further suitable modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Other suitable sugar substituent groups include methoxy (—O—CH₃), aminopropoxy (—O CH₂ CH₂ CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. A suitable 2′-arabino modification is 2′-F. Similar modifications may also be made at other positions on the oligomeric compound, particularly the 3′ position of the sugar on the 3′ terminal nucleoside or in 2′-5′ linked oligonucleotides and the 5′ position of 5′ terminal nucleotide. Oligomeric compounds may also have sugar mimetics such as cyclobutyl moieties in place of the pentofuranosyl sugar.

### Base Modifications and Substitutions

A subject nucleic acid may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (H-pyrido(3′,2′:4,5)pyrrolo(2,3-d)pyrimidin-2-one).

Heterocyclic base moieties may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993; the disclosures of which are incorporated herein by reference in their entirety. Certain of these nucleobases are useful for increasing the binding affinity of an oligomeric compound. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi et al., eds., Antisense Research and Applications, CRC Press, Boca Raton, 1993, pp. 276-278; the disclosure of which is incorporated herein by reference in its entirety) and are suitable base substitutions, e.g., when combined with 2′-O-methoxyethyl sugar modifications.

### Conjugates

Another possible modification of a subject nucleic acid involves chemically linking to the polynucleotide one or more moieties or conjugates which enhance the activity, cellular distribution or cellular uptake of the oligonucleotide. These moieties or conjugates can include conjugate groups covalently bound to functional groups such as primary or secondary hydroxyl groups. Conjugate groups include, but are not limited to, intercalators, reporter molecules, polyamines, polyamides, polyethylene glycols, polyethers, groups that enhance the pharmacodynamic properties of oligomers, and groups that enhance the pharmacokinetic properties of oligomers. Suitable conjugate groups include, but are not limited to, cholesterols, lipids, phospholipids, biotin, phenazine, folate, phenanthridine, anthraquinone, acridine, fluoresceins, rhodamines, coumarins, and dyes. Groups that enhance the pharmacodynamic properties include groups that improve uptake, enhance resistance to degradation, and/or strengthen sequence-specific hybridization with the target nucleic acid. Groups that enhance the pharmacokinetic properties include groups that improve uptake, distribution, metabolism or excretion of a subject nucleic acid.

Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N. Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937).

A conjugate may include a “Protein Transduction Domain” or PTD (also known as a CPP—cell penetrating peptide), which may refer to a polypeptide, polynucleotide, carbohydrate, or organic or inorganic compound that facilitates traversing a lipid bilayer, micelle, cell membrane, organelle membrane, or vesicle membrane. A PTD attached to another molecule, which can range from a small polar molecule to a large macromolecule and/or a nanoparticle, facilitates the molecule traversing a membrane, for example going from extracellular space to intracellular space, or cytosol to within an organelle (e.g., the nucleus). In some cases, a PTD is covalently linked to the 3′ end of an exogenous polynucleotide. In some cases, a PTD is covalently linked to the 5′ end of an exogenous polynucleotide. Exemplary PTDs include but are not limited to a minimal undecapeptide protein transduction domain (corresponding to residues 47-57 of HIV-1 TAT comprising YGRKKRRQRRR; SEQ ID NO:159); a polyarginine sequence comprising a number of arginines sufficient to direct entry into a cell (e.g., 3, 4, 5, 6, 7, 8, 9, 10, or 10-50 arginines); a VP22 domain (Zender et al. (2002) Cancer Gene Ther. 9(6):489-96); a Drosophila Antennapedia protein transduction domain (Noguchi et al. (2003) Diabetes 52(7):1732-1737); a truncated human calcitonin peptide (Trehin et al. (2004) Pharm. Research 21:1248-1256); polylysine (Wender et al. (2000) Proc. Natl. Acad. Sci. USA 97:13003-13008); RRQRRTSKLMKR (SEQ ID NO:160); Transportan GWTLNSAGYLLGKINLKALAALAKKIL (SEQ ID NO:161); KALAWEAKLAKALAKALAKHLAKALAKALKCEA (SEQ ID NO:162); and RQIKIWFQNRRMKWKK (SEQ ID NO:163). Exemplary PTDs include but are not limited to, YGRKKRRQRRR (SEQ ID NO:159), RKKRRQRRR (SEQ ID NO:164); an arginine homopolymer of from 3 arginine residues to 50 arginine residues; Exemplary PTD domain amino acid sequences include, but are not limited to, any of the following: YGRKKRRQRRR (SEQ ID NO:159); RKKRRQRR (SEQ ID NO:165); YARAAARQARA (SEQ ID NO:166); THRLPRRRRRR (SEQ ID NO:167); and GGRRARRRRRR (SEQ ID NO:168). In some cases, the PTD is an activatable CPP (ACPP) (Aguilera et al. (2009) Integr Biol (Camb) June; 1(5-6): 371-381). ACPPs comprise a polycationic CPP (e.g., Arg9 or “R9”) connected via a cleavable linker to a matching polyanion (e.g., Glu9 or “E9”), which reduces the net charge to nearly zero and thereby inhibits adhesion and uptake into cells. Upon cleavage of the linker, the polyanion is released, locally unmasking the polyarginine and its inherent adhesiveness, thus “activating” the ACPP to traverse the membrane.

Sequences of the polynucleotides of the present disclosure may be prepared by various suitable methods known in the art, including, for example, direct chemical synthesis or cloning. For direct chemical synthesis, formation of a polymer of nucleic acids typically involves sequential addition of 3′-blocked and 5′-blocked nucleotide monomers to the terminal 5′-hydroxyl group of a growing nucleotide chain, wherein each addition is effected by nucleophilic attack of the terminal 5′-hydroxyl group of the growing chain on the 3′-position of the added monomer, which is typically a phosphorus derivative, such as a phosphotriester, phosphoramidite, or the like. Such methodology is known to those of ordinary skill in the art and is described in the pertinent texts and literature (e.g., in Matteucci et al., (1980) Tetrahedron Lett 21:719-722; U.S. Pat. Nos. 4,500,707; 5,436,327; and 5,700,637). In addition, the desired sequences may be isolated from natural sources by splitting DNA using appropriate restriction enzymes, separating the fragments using gel electrophoresis, and thereafter, recovering the desired polynucleotide sequence from the gel via techniques known to those of ordinary skill in the art, such as utilization of a polymerase chain reaction (PCR; e.g., U.S. Pat. No. 4,683,195).

The nucleic acids employed in the methods and compositions described herein may be codon optimized relative to a parental template for expression in a particular host cell. Cells differ in their usage of particular codons, and codon bias corresponds to relative abundance of particular tRNAs in a given cell type. By altering codons in a sequence so that they are tailored to match with the relative abundance of corresponding tRNAs, it is possible to increase expression of a product (e.g. a polypeptide) from a nucleic acid. Similarly, it is possible to decrease expression by deliberately choosing codons corresponding to rare tRNAs. Thus, codon optimization/deoptimization can provide control over nucleic acid expression in a particular cell type (e.g. bacterial cell, plant cell, mammalian cell, etc.). Methods of codon optimizing a nucleic acid for tailored expression in a particular cell type are well-known to those of skill in the art.

### Guide RNAs

Certain aspects of the present disclosure relate to guide RNAs and their use in CRISPR-based targeting of a target nucleic acid. Guide RNAs of the present disclosure are capable of binding or otherwise interacting with a Cas12L polypeptide to facilitate targeting of the Cas12L polypeptide to a target nucleic acid. Suitable and exemplary guide RNAs are provided herein and design of such to target a particular nucleic acid will be readily apparent to one of skill in the art. Guide RNAs may also be modified to improve the efficiency of their function in guiding Cas12L to a target nucleic acid.

Guide RNAs of the present disclosure contain a CRISPR RNA (crRNA) sequence, and the sequence of the crRNA is involved in conferring specificity to targeting a specific nucleic acid sequence.

In some embodiments, guide RNA molecules may be extended to include sites for the binding of RNA binding proteins. In some embodiments, multiple guide RNAs can be assembled into a pre-crRNA array that can be processed by the RuvC domain of Cas12L. This will allow for multiplex editing to enable simultaneous targeting to several sites.

In some embodiments, a guide RNA contains both RNA and a repeat sequence that is composed of DNA. In this sense, a guide RNA may be an RNA-DNA hybrid molecule.

A guide RNA (gRNA) may be expressed in a variety of ways as will be apparent to one of skill in the art. For example, a gRNA may be expressed from a recombinant nucleic acid in vivo, from a recombinant nucleic acid in vitro, from a recombinant nucleic acid ex vivo, or can be synthetically synthesized.

A guide RNA of the present disclosure may have various nucleotide lengths. A guide RNA may contain, for example, at least 5, at least 10, at least 15, at least 20, at least 25, at least 30, at least 35, at least 40, at least 50, at least 60, at least 70, at least 80, at least 90, at least 100, at least 110, at least 120, at least 130, at least 140, at least 150, at least 160, at least 170, at least 180 nucleotides, at least 190 nucleotides, or at least 200 nucleotides or more. Longer guide RNAs may result in increased editing efficiency by Cas12L polypeptides.

A guide RNA of the present disclosure may hybridize with a particular nucleotide sequence on a target nucleic acid. This hybridization may be 100% complimentary or it may be less than 100% complimentary so long as the hybridization is sufficient to allow Cas12L to bind to or interact with the target nucleic acid. A guide RNA may contain a nucleotide sequence that is, for example, at least 80%, at least 85%, at least 90%, at least 91%, at least 92%, at least 93%, at least 94%, at least 95%, at least 96%, at least 97%, at least 98%, at least 99%, or 100% identical or complimentary to the target nucleotide sequence in the target nucleic acid that is targeted by/to be hybridized with the guide RNA.

In some cases, increasing expression of a guide RNA may increase the editing efficiency of a target nucleic acid according to the methods of the present disclosure. In some cases, use of a Pol II promoter (e.g. a CmYLCV promoter) to drive gRNA expression may result in increased expression of the guide RNA as compared to a corresponding control promoter (e.g. a Pol III promoter, such as a U6 promoter for example). Use of a Pol II promoter to drive gRNA expression may increase the expression of the guide RNA by, for example, at least about 1%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 125%, at least about 150%, at least about 175%, at least about 200%, at least about 225%, at least about 250%, at least about 275%, or at least about 300% or more as compared to a corresponding control (e.g. a U6 promoter).

In some embodiments, a guide RNA of the present disclosure may be recombinantly fused with a ribozyme sequence to assist in gRNA processing. Exemplary ribozymes for use herein will be readily apparent to one of skill in the art. Exemplary ribozymes may include, for example, a Hammerhead-type ribozyme and a hepatitis delta virus ribyzome. Use of a ribozyme to assist in processing of guide RNAs may increase efficiency of editing of a target nucleic acid sequence by a Cas12L polypeptide of the present disclosure. Use of a ribozyme fused to a gRNA may increase relative editing efficiency by, for example, at least about 1%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 125%, at least about 150%, at least about 175%, at least about 200%, at least about 225%, at least about 250%, at least about 275%, or at least about 300% or more as compared to a corresponding control (e.g. a guide RNA that is expressed without the assistance of any additional processing machinery).

### Methods of Identifying Sequence Similarity

Various methods are known to those of skill in the art for identifying similar (e.g. homologs, orthologs, paralogs, etc.) polypeptide and/or polynucleotide sequences, including phylogenetic methods, sequence similarity analysis, and hybridization methods.

Phylogenetic trees may be created for a gene family by using a program such as CLUSTAL (Thompson et al. Nucleic Acids Res. 22: 4673-4680 (1994); Higgins et al. Methods Enzymol 266: 383-402 (1996)) or MEGA (Tamura et al. Mol. Biol. & Evo. 24:1596-1599 (2007)). Once an initial tree for genes from one species is created, potential orthologous sequences can be placed in the phylogenetic tree and their relationships to genes from the species of interest can be determined. Evolutionary relationships may also be inferred using the Neighbor-Joining method (Saitou and Nei, Mol. Biol. & Evo. 4:406-425 (1987)). Homologous sequences may also be identified by a reciprocal BLAST strategy. Evolutionary distances may be computed using the Poisson correction method (Zuckerkandl and Pauling, pp. 97-166 in Evolving Genes and Proteins, edited by V. Bryson and H. J. Vogel. Academic Press, New York (1965)).

In addition, evolutionary information may be used to predict gene function. Functional predictions of genes can be greatly improved by focusing on how genes became similar in sequence (i.e. by evolutionary processes) rather than on the sequence similarity itself (Eisen, Genome Res. 8: 163-167 (1998)). Many specific examples exist in which gene function has been shown to correlate well with gene phylogeny (Eisen, Genome Res. 8: 163-167 (1998)). By using a phylogenetic analysis, one skilled in the art would recognize that the ability to deduce similar functions conferred by closely-related polypeptides is predictable.

When a group of related sequences are analyzed using a phylogenetic program such as CLUSTAL, closely related sequences typically cluster together or in the same clade (a group of similar genes). Groups of similar genes can also be identified with pair-wise BLAST analysis (Feng and Doolittle, J. Mol. Evol. 25: 351-360 (1987)). Analysis of groups of similar genes with similar function that fall within one clade can yield sub-sequences that are particular to the clade. These sub-sequences, known as consensus sequences, can not only be used to define the sequences within each clade, but define the functions of these genes; genes within a clade may contain paralogous sequences, or orthologous sequences that share the same function (see also, for example, Mount, Bioinformatics: Sequence and Genome Analysis Cold Spring Harbor Laboratory Press, Cold Spring Harbor, N.Y., page 543 (2001)).

To find sequences that are homologous to a reference sequence, BLAST nucleotide searches can be performed with the BLASTN program, score=100, wordlength=12, to obtain nucleotide sequences homologous to a nucleotide sequence encoding a protein of the disclosure. BLAST protein searches can be performed with the BLASTX program, score=50, wordlength=3, to obtain amino acid sequences homologous to a protein or polypeptide of the disclosure. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, or PSI-BLAST, the default parameters of the respective programs (e.g., BLASTN for nucleotide sequences, BLASTX for proteins) can be used.

Methods for the alignment of sequences and for the analysis of similarity and identity of polypeptide and polynucleotide sequences are well-known in the art.

As used herein “sequence identity” refers to the percentage of residues that are identical in the same positions in the sequences being analyzed. As used herein “sequence similarity” refers to the percentage of residues that have similar biophysical/biochemical characteristics in the same positions (e.g. charge, size, hydrophobicity) in the sequences being analyzed.

Methods of alignment of sequences for comparison are well-known in the art, including manual alignment and computer assisted sequence alignment and analysis. This latter approach is a preferred approach in the present disclosure, due to the increased throughput afforded by computer assisted methods. As noted below, a variety of computer programs for performing sequence alignment are available, or can be produced by one of skill.

The determination of percent sequence identity and/or similarity between any two sequences can be accomplished using a mathematical algorithm. Examples of such mathematical algorithms are the algorithm of Myers and Miller, CABIOS 4:11-17 (1988); the local homology algorithm of Smith et al., Adv. Appl. Math. 2:482 (1981); the homology alignment algorithm of Needleman and Wunsch, J. Mol. Biol. 48:443-453 (1970); the search-for-similarity-method of Pearson and Lipman, Proc. Natl. Acad. Sci. 85:2444-2448 (1988); the algorithm of Karlin and Altschul, Proc. Natl. Acad. Sci. USA 87:2264-2268 (1990), modified as in Karlin and Altschul, Proc. Natl. Acad. Sci. USA 90:5873-5877 (1993).

Computer implementations of these mathematical algorithms can be utilized for comparison of sequences to determine sequence identity and/or similarity. Such implementations include, for example: CLUSTAL in the PC/Gene program (available from Intelligenetics, Mountain View, Calif.); the AlignX program, version10.3.0 (Invitrogen, Carlsbad, CA) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. Gene 73:237-244 (1988); Higgins et al. CABIOS 5:151-153 (1989); Corpet et al., Nucleic Acids Res. 16:10881-90 (1988); Huang et al. CABIOS 8:155-65 (1992); and Pearson et al., Meth. Mol. Biol. 24:307-331 (1994). The BLAST programs of Altschul et al. J. Mol. Biol. 215:403-410 (1990) are based on the algorithm of Karlin and Altschul (1990) supra.

Polynucleotides homologous to a reference sequence can be identified by hybridization to each other under stringent or under highly stringent conditions. Single stranded polynucleotides hybridize when they associate based on a variety of well characterized physical-chemical forces, such as hydrogen bonding, solvent exclusion, base stacking and the like. The stringency of a hybridization reflects the degree of sequence identity of the nucleic acids involved, such that the higher the stringency, the more similar are the two polynucleotide strands. Stringency is influenced by a variety of factors, including temperature, salt concentration and composition, organic and non-organic additives, solvents, etc. present in both the hybridization and wash solutions and incubations (and number thereof), as described in more detail in references cited below (e.g., Sambrook et al., Molecular Cloning: A Laboratory Manual, 2nd Ed., Vol. 1-3, Cold Spring Harbor Laboratory, Cold Spring Harbor, N.Y. (“Sambrook”) (1989); Berger and Kimmel, Guide to Molecular Cloning Techniques, Methods in Enzymology, vol. 152 Academic Press, Inc., San Diego, Calif. (“Berger and Kimmel”) (1987); and Anderson and Young, “Quantitative Filter Hybridisation.” In: Hames and Higgins, ed., Nucleic Acid Hybridisation, A Practical Approach. Oxford, TRL Press, 73-111 (1985)).

Encompassed by the disclosure are polynucleotide sequences that are capable of hybridizing to the disclosed polynucleotide sequences and fragments thereof under various conditions of stringency (see, for example, Wahl and Berger, Methods Enzymol. 152: 399-407 (1987); and Kimmel, Methods Enzymo. 152: 507-511, (1987)). Full length cDNA, homologs, orthologs, and paralogs of polynucleotides of the present disclosure may be identified and isolated using well-known polynucleotide hybridization methods.

With regard to hybridization, conditions that are highly stringent, and means for achieving them, are well known in the art. See, for example, Sambrook et al. (1989) (supra); Berger and Kimmel (1987) pp. 467-469 (supra); and Anderson and Young (1985)(supra).

Hybridization experiments are generally conducted in a buffer of pH between 6.8 to 7.4, although the rate of hybridization is nearly independent of pH at ionic strengths likely to be used in the hybridization buffer (Anderson and Young (1985)(supra)). In addition, one or more of the following may be used to reduce non-specific hybridization: sonicated salmon sperm DNA or another non-complementary DNA, bovine serum albumin, sodium pyrophosphate, sodium dodecylsulfate (SDS), polyvinyl-pyrrolidone, ficoll and Denhardt's solution. Dextran sulfate and polyethylene glycol 6000 act to exclude DNA from solution, thus raising the effective probe DNA concentration and the hybridization signal within a given unit of time. In some instances, conditions of even greater stringency may be desirable or required to reduce non-specific and/or background hybridization. These conditions may be created with the use of higher temperature, lower ionic strength and higher concentration of a denaturing agent such as formamide.

Stringency conditions can be adjusted to screen for moderately similar fragments such as homologous sequences from distantly related organisms, or to highly similar fragments such as genes that duplicate functional enzymes from closely related organisms. The stringency can be adjusted either during the hybridization step or in the post-hybridization washes. Salt concentration, formamide concentration, hybridization temperature and probe lengths are variables that can be used to alter stringency. As a general guideline, high stringency is typically performed at Tm−5° C. to Tm−20° C., moderate stringency at Tm−20° C. to Tm−35° C. and low stringency at Tm−35° C. to Tm−50° C. for duplex >150 base pairs. Hybridization may be performed at low to moderate stringency (25-50° C. below Tm), followed by post-hybridization washes at increasing stringencies. Maximum rates of hybridization in solution are determined empirically to occur at Tm−25° C. for DNA-DNA duplex and Tm−15° C. for RNA-DNA duplex. Optionally, the degree of dissociation may be assessed after each wash step to determine the need for subsequent, higher stringency wash steps.

High stringency conditions may be used to select for nucleic acid sequences with high degrees of identity to the disclosed sequences. An example of stringent hybridization conditions obtained in a filter-based method such as a Southern or northern blot for hybridization of complementary nucleic acids that have more than 100 complementary residues is about 5° C. to 20° C. lower than the thermal melting point (Tm) for the specific sequence at a defined ionic strength and pH.

Hybridization and wash conditions that may be used to bind and remove polynucleotides with less than the desired homology to the nucleic acid sequences or their complements of the present disclosure include, for example: 6×SSC and 1% SDS at 65° C.; 50% formamide, 4×SSC at 42° C.; 0.5×SSC to 2.0×SSC, 0.1% SDS at 50° C. to 65° C.; or 0.1×SSC to 2×SSC, 0.1% SDS at 50° C.-65° C.; with a first wash step of, for example, 10 minutes at about 42° C. with about 20% (v/v) formamide in 0.1×SSC, and with, for example, a subsequent wash step with 0.2×SSC and 0.1% SDS at 65° C. for 10, 20 or 30 minutes.

For identification of less closely related homologs, wash steps may be performed at a lower temperature, e.g., 50o C. An example of a low stringency wash step employs a solution and conditions of at least 25° C. in 30 mM NaCl, 3 mM trisodium citrate, and 0.1% SDS over 30 min. Greater stringency may be obtained at 42° C. in 15 mM NaCl, with 1.5 mM trisodium citrate, and 0.1% SDS over 30 min. Wash procedures will generally employ at least two final wash steps. Additional variations on these conditions will be readily apparent to those skilled in the art (see, for example, US Patent Application No. 20010010913).

If desired, one may employ wash steps of even greater stringency, including conditions of 65° C.-68° C. in a solution of 15 mM NaCl, 1.5 mM trisodium citrate, and 0.1% SDS, or about 0.2×SSC, 0.1% SDS at 65° C. and washing twice, each wash step of 10, 20 or 30 min in duration, or about 0.1×SSC, 0.1% SDS at 65° C. and washing twice for 10, 20 or 30 min. Hybridization stringency may be increased further by using the same conditions as in the hybridization steps, with the wash temperature raised about 3° C. to about 5° C., and stringency may be increased even further by using the same conditions except the wash temperature is raised about 6° C. to about 9° C.

### Recombinant Expression

Recombinant nucleic acids and/or recombinant polypeptides of the present disclosure may be present in host cells (e.g. plant cells). In some embodiments, recombinant nucleic acids are present in an expression vector and may encode a recombinant polypeptide, and the expression vector may be present in host cells (e.g. plant cells). In some embodiments, recombinant nucleic acids and/or recombinant polypeptides are present in host cells (e.g. plant cells) via direct introduction into the cell (e.g. via RNPs).

In some embodiments, the genes encoding the recombinant polypeptides in the plant cell may be heterologous to the plant cell. In certain embodiments, the plant cell does not naturally produce one or more polypeptides of the present disclosure, and contains heterologous nucleic acid constructs capable of expressing one or more genes necessary for producing those molecules. In certain embodiments, the plant cell does not naturally produce one or more polypeptides of the present disclosure, and is provided the one or more polypeptides through exogenous delivery of the polypeptides directly to the plant cell without the need to express a recombinant nucleic acid encoding the recombinant polypeptide in the plant cell.

Recombinant polypeptides of the present disclosure may be introduced into host cells (e.g. plant cells) via any suitable methods known in the art. For example, a Cas12L polypeptide can be exogenously added to plant cells and the plant cells are maintained under conditions such that the recombinant polypeptide is targeted (via a guide RNA) to one or more target nucleic acids to edit/modify the target nucleic acids in the plant cells. Alternatively, a recombinant nucleic acid encoding a Cas12L polypeptide of the present disclosure can be expressed in plant cells and the plant cells are maintained under conditions such that the Cas12L polypeptide is targeted (via a guide RNA) to one or more target nucleic acids to edit/modify the target nucleic acids in the plant cells. Additionally, in some embodiments, a Cas12L polypeptide of the present disclosure may be transiently expressed in a plant via viral infection of the plant, or by introducing a Cas12L polypeptide-encoding RNA into a plant to facilitate editing/modification of a target nucleic acid of interest. This approach may be particularly well-suited for Cas12L-based editing given that the small size of Cas12L proteins may make them more amenable to delivery via virus. Methods of introducing proteins via viral infection or via the introduction of RNAs into plants are well known in the art. For example, Tobacco rattle virus (TRV) has been successfully used to introduce zinc finger nucleases in plants to cause genome modification (“Nontransgenic Genome Modification in Plant Cells”, Plant Physiology 154:1079-1087 (2010)). TRV and other appropriate viruses may be used herein to facilitate editing in plants cells.

In some embodiments, a Cas12L polypeptide and a guide RNA may be exogenously and directly supplied to a plant cell as a ribonucleoprotein (RNP) complex. This particular form of delivery is useful for facilitating transgene-free editing in plants. Modified guide RNAs which are resistant to nuclease digestion could also be used in this approach. Transgene-free callus from plants cells provided with an RNP could be used to regenerate whole edited plants.

A recombinant nucleic acid encoding a recombinant polypeptide of the present disclosure can be expressed in a plant with any suitable plant expression vector. Typical vectors useful for expression of recombinant nucleic acids in higher plants are well known in the art and include, for example, vectors derived from the tumor-inducing (Ti) plasmid of Agrobacterium tumefaciens (e.g., see Rogers et al., Meth. in Enzymol. (1987) 153:253-277). These vectors are plant integrating vectors in that on transformation, the vectors integrate a portion of vector DNA into the genome of the host plant. Exemplary A. tumefaciens vectors useful herein are plasmids pKYLX6 and pKYLX7 (e.g., see of Schardl et al., Gene (1987) 61:1-11; and Berger et al., Proc. Natl. Acad. Sci. USA (1989) 86:8402-8406); and plasmid pBI 101.2 that is available from Clontech Laboratories, Inc. (Palo Alto, CA).

In addition to regulatory domains, recombinant polypeptides of the present disclosure can be expressed as a fusion protein that is coupled to, for example, a maltose binding protein (“MBP”), glutathione S transferase (GST), hexahistidine, c-myc, or the FLAG epitope for ease of purification, monitoring expression, or monitoring cellular and subcellular localization.

Moreover, a recombinant nucleic acid encoding a recombinant polypeptide of the present disclosure can be modified to improve expression of the recombinant protein in plants by using codon preference/codon optimization to target preferential expression in plant cells. When the recombinant nucleic acid is prepared or altered synthetically, advantage can be taken of known codon preferences of the intended plant host where the nucleic acid is to be expressed. For example, recombinant nucleic acids of the present disclosure can be modified to account for the specific codon preferences and GC content preferences of monocotyledons and dicotyledons, as these preferences have been shown to differ (Murray et al., Nucl. Acids Res. (1989) 17: 477-498).

The present disclosure further provides expression vectors encoding recombinant polypeptides of the present disclosure. A nucleic acid sequence coding for the desired recombinant nucleic acid of the present disclosure can be used to construct a recombinant expression vector which can be introduced into the desired host cell. A recombinant expression vector will typically contain a nucleic acid encoding a recombinant protein of the present disclosure, operably linked to transcriptional initiation regulatory sequences which will direct the transcription of the nucleic acid in the intended host cell, such as tissues of a transformed plant.

Recombinant nucleic acids e.g. encoding recombinant polypeptides of the present disclosure may be expressed on multiple expression vectors or they may be expressed on a single expression vector. For example, plant expression vectors may include (1) a cloned gene under the transcriptional control of 5′ and 3′ regulatory sequences and (2) a dominant selectable marker. Such plant expression vectors may also contain, if desired, a promoter regulatory region (e.g., one conferring inducible or constitutive, environmentally-regulated or developmentally-regulated expression, or cell- or tissue-specific/selective expression), a transcription initiation start site, a ribosome binding site, an RNA processing signal, a transcription termination site, and/or a polyadenylation signal.

In some embodiments, expression of a nucleic acid of the present disclosure may be driven (in operable linkage) with a promoter (e.g. a promoter functional in plants or a plant-specific promoter). A promoter generally refers to a DNA sequence that contains an RNA polymerase binding site, transcription start site, and/or TATA box and assists or promotes the transcription and expression of an associated transcribable polynucleotide sequence such as, for example, a gene. A plant promoter, or functional fragment thereof, can be employed to e.g. control the expression of a recombinant nucleic acid of the present disclosure in regenerated plants. The selection of the promoter used in expression vectors will determine the spatial and temporal expression pattern of the recombinant nucleic acid in the modified plant, e.g., the nucleic acid encoding the recombinant polypeptide of the present disclosure is only expressed in the desired tissue or at a certain time in plant development or growth. Certain promoters will express recombinant nucleic acids in all plant tissues and are active under most environmental conditions and states of development or cell differentiation (i.e., constitutive promoters). Other promoters will express recombinant nucleic acids in specific cell types (such as leaf epidermal cells, mesophyll cells, root cortex cells) or in specific tissues or organs (roots, leaves or flowers, for example) and the selection will reflect the desired location of accumulation of the gene product. Alternatively, the selected promoter may drive expression of the recombinant nucleic acid under various inducing conditions.

Examples of suitable constitutive promoters may include, for example, the core promoter of the Rsyn7, the core CaMV 35S promoter (Odell et al., Nature (1985) 313:810-812), CaMV 19S (Lawton et al., 1987), rice actin (Wang et al., 1992; U.S. Pat. No. 5,641,876; and McElroy et al., Plant Cell (1985) 2:163-171); ubiquitin (Christensen et al., Plant Mol. Biol. (1989)_{12:619}-632; and Christensen et al., Plant Mol. Biol. (1992) 18:675-689), pEMU (Last et al., Theor. Appl. Genet. (1991) 81:581-588), MAS (Velten et al., EMBO J. (1984) 3:2723-2730), nos (Ebert et al., 1987), Adh (Walker et al., 1987), the P- or 2′-promoter derived from T-DNA of Agrobacterium tumefaciens, the Smas promoter, the cinnamyl alcohol dehydrogenase promoter (U.S. Pat. No. 5,683,439), the Nos promoter, the pEmu promoter, the rubisco promoter, the GRP 1-8 promoter, and other transcription initiation regions from various plant genes known to those of skilled artisans, and constitutive promoters described in, for example, U.S. Pat. Nos. 5,608,149; 5,608,144; 5,604,121; 5,569,597; 5,466,785; 5,399,680; 5,268,463; and 5, 608,142.

In some embodiments, expression of a nucleic acid of the present disclosure may be driven (in operable linkage) with a UBQ10 promoter. In some embodiments, expression of a nucleic acid of the present disclosure may be driven (in operable linkage) with a promoter having a nucleic acid sequence with at least about 20%, at least about 25%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% nucleic acid sequence identity to the following UBQ10 promoter sequence:

(SEQ ID NO: 85)
CGACGAGTCAGTAATAAACGGCGTCAAAGTGGTTGCAGCCGGCACACACGAGTCGTGTTTA
TCAACTCAAAGCACAAATACTTTTCCTCAACCTAAAAATAAGGCAATTAGCCAAAAACAACT
TTGCGTGTAAACAACGCTCAATACACGTGTCATTTTATTATTAGCTATTGCTTCACCGCCTTA
GCTTTCTCGTGACCTAGTCGTCCTCGTCTTTTCTTCTTCTTCTTCTATAAAACAATACCCAAAG
AGCTCTTCTTCTTCACAATTCAGATTTCAATTTCTCAAAATCTTAAAAACTTTCTCTCAATTCT
CTCTACCGTGATCAAGGTAAATTTCTGTGTTCCTTATTCTCTCAAAATCTTCGATTTTGTTTTC
GTTCGATCCCAATTTCGTATATGTTCTTTGGTTTAGATTCTGTTAATCTTAGATCGAAGACGA
TTTTCTGGGTTTGATCGTTAGATATCATCTTAATTCTCGATTAGGGTTTCATAGATATCATCC
GATTTGTTCAAATAATTTGAGTTTTGTCGAATAATTACTCTTCGATTTGTGATTTCTATCTAGA
TCTGGTGTTAGTTTCTAGTTTGTGCGATCGAATTTGTAGATTAATCTGAGTTTTTCTGATTAAC
A

In some cases, a UBQ10 promoter comprises the following nucleotide sequence:

(SEQ ID NO: 85)
CGACGAGTCAGTAATAAACGGCGTCAAAGTGGTTGCAGCCGGCACACACGAGTCGTGTTTA
TCAACTCAAAGCACAAATACTTTTCCTCAACCTAAAAATAAGGCAATTAGCCAAAAACAACT
TTGCGTGTAAACAACGCTCAATACACGTGTCATTTTATTATTAGCTATTGCTTCACCGCCTTA
GCTTTCTCGTGACCTAGTCGTCCTCGTCTTTTCTTCTTCTTCTTCTATAAAACAATACCCAAAG
AGCTCTTCTTCTTCACAATTCAGATTTCAATTTCTCAAAATCTTAAAAACTTTCTCTCAATTCT
CTCTACCGTGATCAAGGTAAATTTCTGTGTTCCTTATTCTCTCAAAATCTTCGATTTTGTTTTC
GTTCGATCCCAATTTCGTATATGTTCTTTGGTTTAGATTCTGTTAATCTTAGATCGAAGACGA
TTTTCTGGGTTTGATCGTTAGATATCATCTTAATTCTCGATTAGGGTTTCATAGATATCATCC
GATTTGTTCAAATAATTTGAGTTTTGTCGAATAATTACTCTTCGATTTGTGATTTCTATCTAGA
TCTGGTGTTAGTTTCTAGTTTGTGCGATCGAATTTGTAGATTAATCTGAGTTTTTCTGATTAAC
A

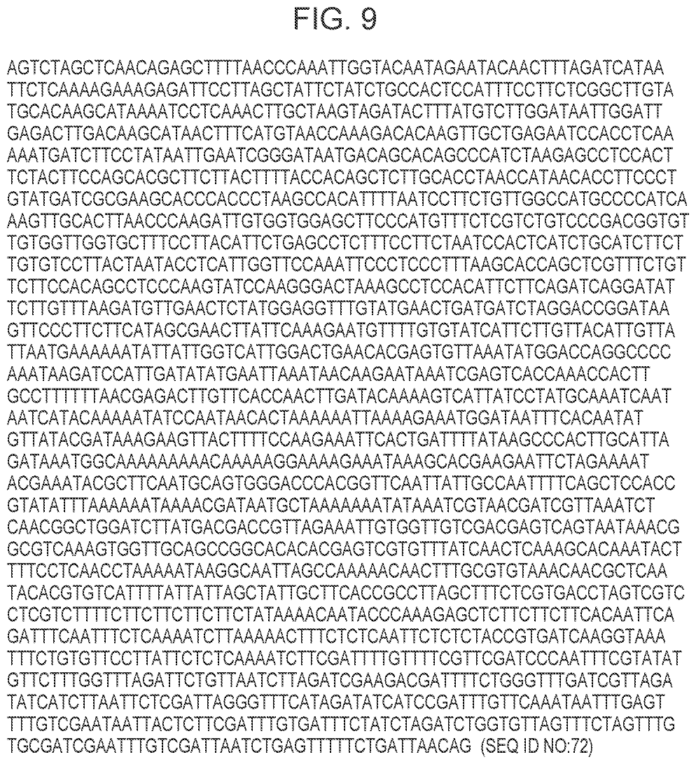

In some cases, expression of a nucleic acid of the present disclosure may be driven with a UBQ10 promoter (i.e., the nucleic acid is operably linked to a UBQ10 promoter) having a nucleic acid sequence with at least about 20%, at least about 25%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% nucleic acid sequence identity to the UBQ10 promoter sequence depicted in FIG. 9 (SEQ ID NO:72).

Recombinant nucleic acids of the present disclosure may be expressed using an RNA Polymerase III (Pol III) promoter such as, for example, the U6 promoter or the H1 promoter (eLife 2013 2:e00471). For example, an approach in plants has been described using three different Pol III promoters from three different Arabidopsis U6 genes, and their corresponding gene terminators (BMC Plant Biology 2014 14:327). One skilled in the art would readily understand that many additional Pol III promoters could be utilized to, for example, simultaneously express many guide RNAs to many different locations in the genome simultaneously. The use of different Pol III promoters for each gRNA expression cassette may be desirable to reduce the chances of natural gene silencing that can occur when multiple copies of identical sequences are expressed in plants.

In some cases, expression of a nucleic acid of the present disclosure may be driven (in operable linkage) with a U6 promoter. In some embodiments, expression of a nucleic acid of the present disclosure may be driven (in operable linkage) with a promoter having a nucleic acid sequence with at least about 20%, at least about 25%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or at least about 100% nucleic acid sequence identity to the following U6 promoter sequence:

(SEQ ID NO: 89)
AAGGTCGGGCAGGAAGAGGGCCTATTTCCCATGATTCCTTCATATTTGCATATACGATACAA
GGCTGTTAGAGAGATAATTAGAATTAATTTGACTGTAAACACAAAGATATTAGTACAAAATA
CGTGACGTAGAAAGTAATAATTTCTTGGGTAGTTTGCAGTTTTAAAATTATGTTTTAAAATGG
ACTATCATATGCTTACCGTAACTTGAAAGTATTTCGATTTCTTGGCTTTATATATCTTGTGGA
AAGGACG

A U6 promoter can have the following nucleotide sequence:

(SEQ ID NO: 89)
AAGGTCGGGCAGGAAGAGGGCCTATTTCCCATGATTCCTTCATATTTGCATATACGATACAA
GGCTGTTAGAGAGATAATTAGAATTAATTTGACTGTAAACACAAAGATATTAGTACAAAATA
CGTGACGTAGAAAGTAATAATTTCTTGGGTAGTTTGCAGTTTTAAAATTATGTTTTAAAATGG
ACTATCATATGCTTACCGTAACTTGAAAGTATTTCGATTTCTTGGCTTTATATATCTTGTGGA
AAGGACG

In some cases, a nucleic acid comprises a nucleotide sequence that is operably linked to a U6 promoter having a nucleic acid sequence with at least about 20%, at least about 25%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% nucleic acid sequence identity to the following AtU626 promoter sequence:

(SEQ ID NO: 91)
AAGCTTCGTTGAACAACGGAAACTCGACTTGCCTTCCGCACAATACATCATTTCTTCTTAGCT
TTTTTTCTTCTTCTTCGTTCATACAGTTTTTTTTTGTTTATCAGCTTACATTTTCTTGAACCGTA
GCTTTCGTTTTCTTCTTTTTAACTTTCCATTCGGAGTTTTTGTATCTTGTTTCATAGTTTGTCCC
AGGATTAGAATGATTAGGCATCGAACCTTCAAGAATTTGATTGAATAAAACATCTTCATTCT
TAAGATATGAAGATAATCTTCAAAAGGCCCCTGGGAATCTGAAAGAAGAGAAGCAGGCCCA
TTTATATGGGAAAGAACAATAGTATTTCTTATATAGGCCCATTTAAGTTGAAAACAATCTTC
AAAAGTCCCACATCGCTTAGATAAGAAAACGAAGCTGAGTTTATATACAGCTAGAGTCGAA
GTAGTGATT

Recombinant nucleic acids of the present disclosure may be expressed using an RNA Polymerase II (Pol 11) promoter such as, for example, the CmYLCV promoter and the 35S promoter. See, e.g., Sahoo et al. (2014) Planta 240:855. Use of a Pol II promoter to drive expression of nucleic acids (e.g. guide RNA expression) may provide additional flexibility for controlling the strength/degree of expression and may provide the possibility of tissue-specific expression. One skilled in the art would recognize appropriate Pol II promoters for use in the methods and compositions of the present disclosure.

In some cases, expression of a nucleic acid of the present disclosure may be driven (in operable linkage) with a CmYLCV promoter. CmYLCV promoters are described in, e.g., WO 2001/073087; and Sahoo et al. (2016) Methods Mol. Biol. 1482:111. In some cases, expression of a nucleic acid of the present disclosure may be driven (in operable linkage) with a promoter having a nucleic acid sequence with at least about 20%, at least about 25%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%, nucleic acid sequence identity to the following CmYLCV promoter nucleotide sequence:

(SEQ ID NO: 87)
TGGCAGACATACTGTCCCACAAATGAAGATGGAATCTGTAAAAGAAAACGCGTGAAATAAT
GCGTCTGACAAAGGTTAGGTCGGCTGCCTTTAATCAATACCAAAGTGGTCCCTACCACGATG
GAAAAACTGTGCAGTCGGTTTGGCTTTTTCTGACGAACAAATAAGATTCGTGGCCGACAGGT
GGGGGTCCACCATGTGAAGGCATCTTCAGACTCCAATAATGGAGCAATGACGTAAGGGCTT
ACGAAATAAGTAAGGGTAGTTTGGGAAATGTCCACTCACCCGTCAGTCTATAAATACTTAGC
CCCTCCCTCATTGTTAAGGGAGCAAAATCTCAGAGAGATAGTCCTAGAGAGAGAAAGAGAG
CAAGTAGCCTAGAAGTAGTCAAGGCGGCGAAGTATTCAGGCACGTGGCCAGGAAGAAGAA
AAGCCAAGACGACGAAAACAGGTAAGAGCTAAGCTT.

In some cases, expression of a nucleic acid of the present disclosure may be driven (in operable linkage) with a Cauliflower mosaic virus 35S promoter (CaMV 35S promoter). In some cases, a nucleic acid of the present disclosure comprises a nucleotide sequence operably linked to a promoter having a nucleic acid sequence with at least about 20%, at least about 25%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%, nucleic acid sequence identity to the following CaMV 35S promoter nucleotide sequence:

(SEQ ID NO: 88)
GGTCAACATGGTGGAGCACGACACACTTGTCTACTCCAAAAATATCAAAGATACAGTCTCAG
AAGACCAAAGGGCAATTGAGACTTTTCAACAAAGGGTAATATCCGGAAACCTCCTCGGATT
CCATTGCCCAGCTATCTGTCACTTTATTGTGAAGATAGTGGAAAAGGAAGGTGGCTCCTACA
AATGCCATCATTGCGATAAAGGAAAGGCCATCGTTGAAGATGCCTCTGCCGACAGTGGTCCC
AAAGATGGACCCCCACCCACGAGGAGCATCGTGGAAAAAGAAGACGTTCCAACCACGTCTT
CAAAGCAAGTGGATTGATGTGATAACATGGTGGAGCACGACACACTTGTCTACTCCAAAAA
TATCAAAGATACAGTCTCAGAAGACCAAAGGGCAATTGAGACTTTTCAACAAAGGGTAATA
TCCGGAAACCTCCTCGGATTCCATTGCCCAGCTATCTGTCACTTTATTGTGAAGATAGTGGAA
AAGGAAGGTGGCTCCTACAAATGCCATCATTGCGATAAAGGAAAGGCCATCGTTGAAGATG
CCTCTGCCGACAGTGGTCCCAAAGATGGACCCCCACCCACGAGGAGCATCGTGGAAAAAGA
AGACGTTCCAACCACGTCTTCAAAGCAAGTGGATTGATGTGATATCTCCACTGACGTAAGGG
ATGACGCACAATCCCACTATCCTTCGCAAGACCCTTCCTCTATATAAGGAAGTTCATTTCATT
TGGAGAGGACCTCGACTCTAGAGGATCCC. This promoter sequence is referred to
as “2x35S” promoter.

In some cases, a CaMV 35S promoter has the following nucleotide sequence:

(SEQ ID NO: 88)
GGTCAACATGGTGGAGCACGACACACTTGTCTACTCCAAAAATATCAAAGATACAGTCTCAG
AAGACCAAAGGGCAATTGAGACTTTTCAACAAAGGGTAATATCCGGAAACCTCCTCGGATT
CCATTGCCCAGCTATCTGTCACTTTATTGTGAAGATAGTGGAAAAGGAAGGTGGCTCCTACA
AATGCCATCATTGCGATAAAGGAAAGGCCATCGTTGAAGATGCCTCTGCCGACAGTGGTCCC
AAAGATGGACCCCCACCCACGAGGAGCATCGTGGAAAAAGAAGACGTTCCAACCACGTCTT
CAAAGCAAGTGGATTGATGTGATAACATGGTGGAGCACGACACACTTGTCTACTCCAAAAA
TATCAAAGATACAGTCTCAGAAGACCAAAGGGCAATTGAGACTTTTCAACAAAGGGTAATA
TCCGGAAACCTCCTCGGATTCCATTGCCCAGCTATCTGTCACTTTATTGTGAAGATAGTGGAA
AAGGAAGGTGGCTCCTACAAATGCCATCATTGCGATAAAGGAAAGGCCATCGTTGAAGATG
CCTCTGCCGACAGTGGTCCCAAAGATGGACCCCCACCCACGAGGAGCATCGTGGAAAAAGA
AGACGTTCCAACCACGTCTTCAAAGCAAGTGGATTGATGTGATATCTCCACTGACGTAAGGG
ATGACGCACAATCCCACTATCCTTCGCAAGACCCTTCCTCTATATAAGGAAGTTCATTTCATT
TGGAGAGGACCTCGACTCTAGAGGATCCC.

In some embodiments, expression of a nucleic acid of the present disclosure may be driven (in operable linkage) with a 2×35S promoter. In some embodiments, expression of a nucleic acid of the present disclosure may be driven (in operable linkage) with a promoter having a nucleic acid sequence with at least about 20%, at least about 25%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% nucleic acid sequence identity to the nucleic acid sequence of SEQ ID NO:88.

Examples of suitable tissue specific promoters may include, for example, the lectin promoter (Vodkin et al., 1983; Lindstrom et al., 1990), the corn alcohol dehydrogenase 1 promoter (Vogel et al., 1989; Dennis et al., 1984), the corn light harvesting complex promoter (Simpson, 1986; Bansal et al., 1992), the corn heat shock protein promoter (Odell et al., Nature (1985) 313:810-812; Rochester et al., 1986), the pea small subunit RuBP carboxylase promoter (Poulsen et al., 1986; Cashmore et al., 1983), the Ti plasmid mannopine synthase promoter (Langridge et al., 1989), the Ti plasmid nopaline synthase promoter (Langridge et al., 1989), the petunia chalcone isomerase promoter (Van Tunen et al., 1988), the bean glycine rich protein 1 promoter (Keller et al., 1989), the truncated CaMV 35s promoter (Odell et al., Nature (1985) 313:810-812), the potato patatin promoter (Wenzler et al., 1989), the root cell promoter (Conkling et al., 1990), the maize zein promoter (Reina et al., 1990; Kriz et al., 1987; Wandelt and Feix, 1989; Langridge and Feix, 1983; Reina et al., 1990), the globulin-1 promoter (Belanger and Kriz et al., 1991), the a-tubulin promoter, the cab promoter (Sullivan et al., 1989), the PEPCase promoter (Hudspeth & Grula, 1989), the R gene complex-associated promoters (Chandler et al., 1989), and the chalcone synthase promoters (Franken et al., 1991).

Alternatively, the plant promoter can direct expression of a recombinant nucleic acid of the present disclosure in a specific tissue or may be otherwise under more precise environmental or developmental control. Such promoters are referred to here as “inducible” promoters. Environmental conditions that may affect transcription by inducible promoters include, for example, pathogen attack, anaerobic conditions, or the presence of light. Examples of inducible promoters include, for example, the AdhI promoter which is inducible by hypoxia or cold stress, the Hsp70 promoter which is inducible by heat stress, and the PPDK promoter which is inducible by light. Examples of promoters under developmental control include, for example, promoters that initiate transcription only, or preferentially, in certain tissues, such as leaves, roots, fruit, seeds, or flowers. An exemplary promoter is the anther specific promoter 5126 (U.S. Pat. Nos. 5,689,049 and 5,689,051). The operation of a promoter may also vary depending on its location in the genome. Thus, an inducible promoter may become fully or partially constitutive in certain locations.

Moreover, any combination of a constitutive or inducible promoter, and a non-tissue specific or tissue specific promoter may be used to control the expression of various recombinant polypeptides of the present disclosure.

The recombinant nucleic acids of the present disclosure and/or a vector housing a recombinant nucleic acid of the present disclosure, may also contain a regulatory sequence that serves as a 3′ terminator sequence. A terminator sequence generally refers to a nucleic acid sequence that marks the end of a gene or transcribable nucleic acid during transcription. One of skill in the art would readily recognize a variety of terminators that may be used in the recombinant nucleic acids of the present disclosure. For example, a recombinant nucleic acid of the present disclosure may contain a 3′ NOS terminator. In some embodiments, recombinant nucleic acids of the present disclosure contain a transcriptional termination site. Transcription termination sites may include, for example, OCS terminators, rbcS-E9 terminators, NOS terminators, HSP18.2 terminators, and poly-T terminators.

In some embodiments, a nucleic acid of the present disclosure may contain a transcriptional termination site having a nucleic acid sequence with at least about 20%, at least about 25%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% nucleic acid sequence identity to the nucleic acid sequence of an 35S terminator, a HSP18 terminator, and/or an RbcS-E9 terminator.

Recombinant nucleic acids of the present disclosure may include one or more introns. Introns may be included in e.g. recombinant nucleic acids being expressed on a vector in a host cell. The inclusion of one of more introns in a recombinant nucleic acid to be expressed may be particularly helpful to increase expression in plant cells.

Recombinant nucleic acids of the present disclosure may also contain selectable markers. A selectable marker can be used to assist in the selection of transformed cells or tissue due to the presence of a selection agent, such as an antibiotic or herbicide, where the selectable marker gene provides tolerance or resistance to the selection agent. Thus, the selection agent can bias or favor the survival, development, growth, proliferation, etc., of transformed cells expressing the selectable marker gene. Selectable marker genes may include, for example, those conferring tolerance or resistance to antibiotics, such as kanamycin and paromomycin (nptll), hygromycin B (aph IV), streptomycin or spectinomycin (aadA) and gentamycin (aac3 and aacC4), or those conferring tolerance or resistance to herbicides such as glufosinate (bar or pat), dicamba (DMO) and glyphosate (aroA or Cp4-EPSPS). Selectable marker genes which provide an ability to visually screen for transformants may also be used such as, for example, luciferase or green fluorescent protein (GFP), or a gene expressing a beta glucuronidase or uidA gene (GUS) for which various chromogenic substrates are known. In some embodiments, a nucleic acid molecule provided herein contains a selectable marker gene selected from the group consisting of nptll, aph IV, aadA, aac3, aacC4, bar, pat, DMO, EPSPS, aroA, luciferase, GFP, and GUS.

### Plants and Plant Cells

Certain aspects of the present disclosure relate to plants and plant cells that contain Cas12L polypeptides that are targeted to one or more target nucleic acids in the plant/plant cell in order to edit/modify the target nucleic acid.

As used herein, a “plant” refers to any of various photosynthetic, eukaryotic multi-cellular organisms of the kingdom Plantae, characteristically producing embryos, containing chloroplasts, having cellulose cell walls and lacking locomotion. As used herein, a “plant” includes any plant or part of a plant at any stage of development, including seeds, suspension cultures, plant cells, embryos, meristematic regions, callus tissue, leaves, roots, shoots, gametophytes, sporophytes, pollen, microspores, and progeny thereof. Also included are cuttings, and cell or tissue cultures. As used in conjunction with the present disclosure, plant tissue includes, for example, whole plants, plant cells, plant organs, e.g., leafs, stems, roots, meristems, plant seeds, protoplasts, callus, cell cultures, and any groups of plant cells organized into structural and/or functional units.

Various plant cells may be used in the present disclosure so long as they remain viable after being transformed or otherwise modified to express recombinant nucleic acids or house recombinant polypeptides. Preferably, the plant cell is not adversely affected by the transduction of the necessary nucleic acid sequences, the subsequent expression of the proteins or the resulting intermediates.

As disclosed herein, a broad range of plant types may be modified to incorporate recombinant polypeptides and/or polynucleotides of the present disclosure. Suitable plants that may be modified include both monocotyledonous (monocot) plants and dicotyledonous (dicot) plants.

Examples of suitable plants may include, for example, species of the Family Gramineae, including Sorghum bicolor and Zea mays; species of the genera: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Avena, Hordeum, Secale, and Triticum.

In some embodiments, plant cells may include, for example, those from corn (Zea mays), canola (Brassica napus, Brassica rapa ssp.), Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), duckweed (Lemna), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucijra), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia spp.), almond (Prunus amygdalus), sugar beets (Beta vulgaris), sugarcane (Saccharum spp.), oats, barley, vegetables, ornamentals, and conifers.

Examples of suitable vegetables plants may include, for example, tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo).

Examples of suitable ornamental plants may include, for example, azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbiapulcherrima), and chrysanthemum.

Examples of suitable conifer plants may include, for example, loblolly pine (Pinus taeda), slash pine (Pinus elliotii), ponderosa pine (Pinus ponderosa), lodgepole pine (Pinus contorta), Monterey pine (Pinus radiata), Douglas-fir (Pseudotsuga menziesii), Western hemlock (Isuga canadensis), Sitka spruce (Picea glauca), redwood (Sequoia sempervirens), silver fir (Abies amabilis), balsam fir (Abies balsamea), Western red cedar (Thuja plicata), and Alaska yellow-cedar (Chamaecyparis nootkatensis).

Examples of suitable leguminous plants may include, for example, guar, locust bean, fenugreek, soybean, garden beans, cowpea, mungbean, lima bean, fava bean, lentils, chickpea, peanuts (Arachis sp.), crown vetch (Vicia sp.), hairy vetch, adzuki bean, lupine (Lupinus sp.), trifolium, common bean (Phaseolus sp.), field bean (Pisum sp.), clover (Melilotus sp.) Lotus, trefoil, lens, and false indigo.

Examples of suitable forage and turf grass may include, for example, alfalfa (Medicago s sp.), orchard grass, tall fescue, perennial ryegrass, creeping bent grass, and redtop.

Examples of suitable crop plants and model plants may include, for example, Arabidopsis, corn, rice, alfalfa, sunflower, canola, soybean, cotton, peanut, sorghum, wheat, tobacco, and lemna.

The plants and plant cells of the present disclosure may be genetically modified in that recombinant nucleic acids have been introduced into the plants, and as such the genetically modified plants and/or plant cells do not occur in nature. A suitable plant of the present disclosure is e.g. one capable of expressing one or more nucleic acid constructs encoding one or more recombinant proteins. The recombinant proteins encoded by the nucleic acids may be e.g. Cas12L polypeptides.

As used herein, the terms “transgenic plant” and “genetically modified plant” are used interchangeably and refer to a plant which contains within its genome a recombinant nucleic acid. Generally, the recombinant nucleic acid is stably integrated within the genome such that the polynucleotide is passed on to successive generations. However, in certain embodiments, the recombinant nucleic acid is transiently expressed in the plant. The recombinant nucleic acid may be integrated into the genome alone or as part of a recombinant expression cassette. “Transgenic” is used herein to include any cell, cell line, callus, tissue, plant part or plant, the genotype of which has been altered by the presence of exogenous nucleic acid including those transgenics initially so altered as well as those created by sexual crosses or asexual propagation from the initial transgenic.

Plant transformation protocols as well as protocols for introducing recombinant nucleic acids of the present disclosure into plants may vary depending on the type of plant or plant cell, e.g., monocot or dicot, targeted for transformation. Suitable methods of introducing recombinant nucleic acids of the present disclosure into plant cells and subsequent insertion into the plant genome include, for example, microinjection (Crossway et al., Biotechniques (1986) 4:320-334), electroporation (Riggs et al., Proc. Natl. Acad Sci. USA (1986) 83:5602-5606), Agrobacterium-mediated transformation (U.S. Pat. No. 5,563,055), direct gene transfer (Paszkowski et al., EMBO J. (1984) 3:2717-2722), and ballistic particle acceleration (U.S. Pat. No. 4,945,050; Tomes et al. (1995). “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); and McCabe et al., Biotechnology (1988) 6:923-926).

Additionally, recombinant polypeptides of the present disclosure can be targeted to a specific organelle within a plant cell. Targeting can be achieved by providing the recombinant protein with an appropriate targeting peptide sequence. Examples of such targeting peptides include, for example, secretory signal peptides (for secretion or cell wall or membrane targeting), plastid transit peptides, chloroplast transit peptides, mitochondrial target peptides, vacuole targeting peptides, nuclear targeting peptides, and the like (e.g., see Reiss et al., Mol. Gen. Genet. (1987) 209(1):116-121; Settles and Martienssen, Trends Cell Biol (1998) 12:494-501; Scott et al., J Biol Chem (2000) 10:1074; and Luque and Correas, J Cell Sci (2000) 113:2485-2495).

Modified plant may be grown in accordance with conventional methods (e.g., see McCormick et al., Plant Cell. Reports (1986) 81-84.). These plants may then be grown, and pollinated with either the same transformed strain or different strains, with the resulting hybrid having the desired phenotypic characteristic. Two or more generations may be grown to ensure that the subject phenotypic characteristic is stably maintained and inherited and then seeds harvested to ensure the desired phenotype or other property has been achieved.

The present disclosure also provides plants derived from plants having an edited/modified nucleic acid as a consequence of the methods of the present disclosure. A plant having an edited/modified nucleic acid as a consequence of the methods of the present disclosure may be crossed with itself or with another plant to produce an F1 plant. In some embodiments, one or more of the resulting F1 plants may also have an edited/modified nucleic acid. Accordingly, in some embodiments, provided are progeny plants that are the progeny (either directly or indirectly) of plants having an edited/modified nucleic acid as a consequence of the methods of the present disclosure. These progeny plants may also have an edited/modified nucleic acid. Progeny plants may also have an altered or modified phenotype as compared to a corresponding control plant.

Further provided are methods of screening plants derived from plants having an edited/modified nucleic acid as a consequence of the methods of the present disclosure. In some embodiments, the derived plants (e.g. F1 or F2 plants resulting from or derived from crossing the plant having an edited/modified nucleic acid expression as a consequence of the methods of the present disclosure with another plant) can be selected from a population of derived plants. For example, provided are methods of selecting one or more of the derived plants that (i) lack recombinant nucleic acids, and (ii) have an edited/modified nucleic acid. Because the edit/modification of the target nucleic acid may be heritable, progeny plants as described herein do not necessarily need to contain a Cas12L polypeptide and/or a guide RNA in order to maintain the edit/modification to the target nucleic acid.

Plants with genetic backgrounds that are susceptible to transgene silencing may exhibit reduced Cas12L-mediated editing efficiency. It may thus be desireable, in some embodiments, to employ a genetic background that has reduced or eliminated susceptibility to transgene silencing. In some embodiments, employing a genetic background with reduced or eliminated susceptibility to transgene silencing may improve editing efficiency. Exemplary genetic backgrounds with reduced or eliminated susceptibility to transgene silencing will be readily apparent to one of skill in the art and include, for example, plants with mutations in RDR6 that reduce or eliminate RDR6 expression or function.

Conducting the methods of the present disclosure in a plant with a genetic background that reduces or eliminates susceptibility to transgene silencing may increase the relative editing efficiency of a target nucleic acid by, for example, at least about 1%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 125%, at least about 150%, at least about 175%, at least about 200%, at least about 225%, at least about 250%, at least about 275%, or at least about 300% or more as compared to a corresponding control (e.g. a wild-type plant).

### Methods of Modifying a Target Nucleic Acid

Growing and/or cultivation conditions sufficient for the recombinant polypeptides and/or polynucleotides of the present disclosure to be expressed and/or maintained in the plant/plant cell and to be targeted to and edit/modify one or more target nucleic acids of the present disclosure are well known in the art and include any suitable growing conditions disclosed herein. Typically, the plant is grown under conditions sufficient to express a recombinant polypeptide of the present disclosure, and for the expressed recombinant polypeptides to be localized to the nucleus of cells of the plant in order to be targeted to and edit/modify the target nucleic acids (if those target nucleic acids are present in the nucleus). Generally, the conditions sufficient for the expression of the recombinant polypeptide (if being encoded from a recombinant nucleic acid) will depend on the promoter used to control the expression of the recombinant polypeptide. For example, if an inducible promoter is utilized, expression of the recombinant polypeptide in a plant will require that the plant to be grown in the presence of the inducer.

### Growth Conditions

As noted above, growing conditions sufficient for the recombinant polypeptides of the present disclosure to be expressed and/or maintained in the plant and to be targeted to one or more target nucleic acids to edit/modify the one or more target nucleic acids may vary depending on a number of factors (e.g. species of plant, use of inducible promoter, etc.). Suitable growing conditions may include, for example, ambient environmental conditions, standard laboratory conditions, standard greenhouse conditions, growth in long days under standard environmental conditions (e.g. 16 hours of light, 8 hours of dark), growth in 12 hour light: 12 hour dark day/night cycles, etc.

Plants and/or plant cells of the present disclosure housing a Cas12L polypeptide and a guide RNA may be maintained at a variety of temperatures. In general, the temperature should be sufficient for the Cas12L polypeptide and guide RNA to form, maintain, or otherwise be present as a complex that is able to target a target nucleic acid in order to edit/modify the target nucleic acids. Exemplary growth/cultivation temperatures include, for example, at least about 20° C., at least about 21° C., at least about 22° C., at least about 23° C., at least about 24° C., at least about 25° C., at least about 26° C., at least about 27° C., at least about 28° C., at least about 29° C., at least about 30° C., at least about 31° C., at least about 32° C., at least about 33° C., at least about 34° C., at least about 35° C., at least about 36° C., at least about 37° C., at least about 38° C., at least about 39° C., or at least about 40° C. Exemplary growth/cultivation temperatures include, for example, about 20° C. to about 25° C., about 25° C. to about 30° C., about 30° C. to about 35° C., or about 35° C. to about 40° C. Plants and plant cells may be maintained at a constant temperature throughout the duration of the growth and/or incuation period, or the temperature schedule can be adjusted at various points throughout the duration of the growth and/or incuation period as will be readily apparent to one of skill in the art depending on the particular growth and/or incubation purpose.

In some embodiments, plants and plant cells may be maintained at a relative constant temperature with one or more periodic or intermittent exposures to a different temperature. For example, a plant or plant cell may be maintained at e.g. 20° C.-25° C. and then have a brief exposure to a different temperature (e.g. 37° C. for between 5 minutes to 5 hours), and then be returned to the original growth temperature (e.g. 20° C.-25° C.). The exposure to a different temperature may occur once or it may occur on a plurality of occasions over the full growth interval of plants and plant cells according to the methods of the present disclosure.

In some embodiments, plants and plant cells may be exposed to a first temperature and a second temperature for varying amounts of time, where the first and second temperatures are not the same temperature/are different temperatures. In some embodiments, the first temperature may be, for example, at least about 20° C., at least about 21° C., at least about 22° C., at least about 23° C., at least about 24° C., at least about 25° C., at least about 26° C., at least about 27° C., at least about 28° C., at least about 29° C., at least about 30° C., at least about 31° C., at least about 32° C., at least about 33° C., at least about 34° C., at least about 35° C., at least about 36° C., at least about 37° C., at least about 38° C., at least about 39° C., or at least about 40° C. and the duration of exposure to the first temperature may be, for example, about 30 minutes, about 45 minutes, about 1 hour, about 2.5 hours, about 5 hours, about 7.5 hours, about 10 hours, about 15 hours, about 20 hours, about 1 day, about 5 days, about 10 days, about 15 days, about 20 days, about 25 days, about 30 days, about 35 days, about 40 days, about 45 days, about 50 days, or about 55 days or more. In some embodiments, the second temperature may be, for example, at least about 20° C., at least about 21° C., at least about 22° C., at least about 23° C., at least about 24° C., at least about 25° C., at least about 26° C., at least about 27° C., at least about 28° C., at least about 29° C., at least about 30° C., at least about 31° C., at least about 32° C., at least about 33° C., at least about 34° C., at least about 35° C., at least about 36° C., at least about 37° C., at least about 38° C., at least about 39° C., or at least about 40° C. and the duration of exposure to the second temperature may be, for example, about 30 minutes, about 45 minutes, about 1 hour, about 2.5 hours, about 5 hours, about 7.5 hours, about 10 hours, about 15 hours, about 20 hours, about 1 day, about 5 days, about 10 days, about 15 days, about 20 days, about 25 days, about 30 days, about 35 days, about 40 days, about 45 days, about 50 days, or about 55 days or more.

Various time frames may be used to observe editing/modification of a target nucleic acid according to the methods of the present disclosure. Plants and/or plant cells may be observed/assayed for editing/modification of a target nucleic acid after, for example, about 30 minutes, about 45 minutes, about 1 hour, about 2.5 hours, about 5 hours, about 7.5 hours, about 10 hours, about 15 hours, about 20 hours, about 1 day, about 5 days, about 10 days, about 15 days, about 20 days, about 25 days, about 30 days, about 35 days, about 40 days, about 45 days, about 50 days, or about 55 days or more after being cultivated/grown in conditions sufficient for a Cas12L polypeptide to facilitate editing/modification of a target nucleic acid.

### Editing/Modifying a Target Nucleic Acid

Certain aspects of the present disclosure relate to editing or modifying a target nucleic acid using Cas12L polypeptides. In some embodiments, a Cas12L polypeptide is used to create a mutation in a target nucleic acid. Mutation of a nucleic acid generally refers to an insertion, deletion, substitution, duplication, or inversion of one or more nucleotides in the nucleic acid as compared to a reference or control nucleotide sequence.

In some embodiments, a Cas12L polypeptide of the present disclosure may induce a double-stranded break (DSB) at a target site of a nucleic acid sequence that is then repaired by the natural processes of either homologous recombination (HR) or non-homologous end-joining (NHEJ). Sequence modifications, such as for example insertions and deletions, can occur at the DSB locations via NHEJ repair. If two DSBs flanking one target region are created, the breaks can be repaired via NHEJ by reversing the orientation of the targeted DNA (also referred to as an “inversion”). HR can be used to integrate a donor nucleic acid sequence into a target site. In one aspect, a double-stranded break provided herein is repaired by NHEJ. In another aspect, a double-stranded break provided herein is repaired by HR.

In some embodiments, a Cas12L polypeptide of the present disclosure may induce a double-stranded break with 5′ nucleotide overhangs at a target site of a nucleic acid sequence such that an exogenous DNA segment of interest can serve as the donor nucleic acid to be ligated into the target nucleic acid. The presence of 5′ nucleotide overhangs allows the insertion of the exogenous DNA to be directional.

In some embodiments, a nucleic acid that encodes a polypeptide may be targeted and edited such that the modification to the nucleic acid results in a change to one or more codons in the encoded polypeptide. In some embodiments, the modification of the target nucleic acid may result in deletion of one or more codons in the encoded polypeptide.

A target nucleic acid of the present disclosure may be edited or modified in a variety of ways (e.g. deletion of nucleotides in the target nucleic acid) depending on the particular application as will be readily apparent to one of skill in the art. A target nucleic acid subjected to the methods of the present disclosure may have an edit or modification of at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, or at least 25 nucleotides or more.

A target nucleic acid of the present disclosure may have its expression decreased/downregulated as compared to a corresponding control nucleic acid. A target nucleic acid of the present disclosure in a plant cell housing recombinant polypeptides of the present disclosure may have its expression decreased/downregulated by at least about 5%, at least about 10%, at least about 15%, at least about 20%, at least about 25%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% as compared to a corresponding control. Various controls will be readily apparent to one of skill in the art. For example, a control may be a corresponding plant or plant cell that does not contain recombinant polypeptides of the present disclosure (e.g. wild-type plant or plant cell).

A target nucleic acid may have its expression decreased/downregulated at least about 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more, as compared to a corresponding control nucleic acid. As stated above, various controls will be readily apparent to one of skill in the art. For example, a control nucleic acid may be a corresponding nucleic acid from a plant or plant cell that does not contain a nucleic acid encoding a recombinant polypeptide of the present disclosure.

A target nucleic acid of the present disclosure may have its expression increased/upregulated/activated as compared to a corresponding control nucleic acid. A target nucleic acid of the present disclosure in a plant cell housing recombinant polypeptides of the present disclosure may have its expression increased/upregulated/activated by at least about 5%, at least about 10%, at least about 15%, at least about 20%, at least about 25%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, at least about 100% (or two-fold), at least 2.5-fold, at least 5-fold, at least 10-fold, at least 25-fold, at least 50-fold, at least 75-fold, at least 100-fold, or more than 100-fold, as compared to a corresponding control. Various controls will be readily apparent to one of skill in the art. For example, a control may be a corresponding plant or plant cell that does not contain recombinant polypeptides of the present disclosure (e.g. wild-type plant or plant cell).

A target nucleic acid may have its expression increased/upregulated/activated at least about 1-fold, at least about 2-fold, at least about 3-fold, at least about 4-fold, at least about 5-fold, at least about 10-fold, at least about 15-fold, at least about 20-fold, at least about 25-fold, at least about 30-fold, at least about 40-fold, at least about 50-fold, at least about 75-fold, at least about 100-fold, at least about 150-fold, at least about 200-fold, at least about 300-fold, at least about 400-fold, at least about 500-fold, at least about 600-fold, at least about 700-fold, at least about 800-fold, at least about 900-fold, at least about 1,000-fold, at least about 1,250-fold, at least about 1,500-fold, at least about 1,750-fold, at least about 2,000-fold, at least about 2,500-fold, at least about 3,000-fold, at least about 3,500-fold, at least about 4,000-fold, at least about 4,500-fold, at least about 5,000-fold, at least about 5,500-fold, at least about 6,000-fold, at least about 6,500-fold, at least about 7,000-fold, at least about 7,500-fold, at least about 8,000-fold, at least about 8,500-fold, at least about 9,000-fold, at least about 9,500-fold, at least about 10,000-fold, at least about 12,000-fold, at least about 14,000-fold, at least about 16,000-fold, at least about 18,000-fold, or at least about 20,000-fold or more as compared to a corresponding control nucleic acid. As stated above, various controls will be readily apparent to one of skill in the art. For example, a control nucleic acid may be a corresponding nucleic acid from a plant or plant cell that does not contain a nucleic acid encoding a recombinant polypeptide of the present disclosure.

Certain aspects of the present disclosure relate to increasing editing efficiency of a Cas12L polypeptides of the present disclosure. Editing frequency and efficiency, as well as methods of determing such, are well-known in the art. Generally speaking, editing efficiency is evaluated by determining the observed quantity of a given target sequence that experienced an editing event (editing frequency) as compared to the total quantity of the target sequence observed (whether edited or unedited). An increase in editing efficiency generally refers to an increase in the number of sequences experiencing an editing event (editing frequency) as compared to the total quantity of the target sequence observed (whether edited or unedited).

In some embodiments, increases in editing efficiency are compared to corresponding controls in relative terms (relative editing efficiency). For example, if the absolute editing frequency in one condition is 0.5% and the absolute editing frequency in a second condition is 1%, the second condition represents a doubling of the absolute editing frequency relative to the first condition, or in other words, the second condition represents a 100% increase in relative editing efficiency as compared to the first condition.

The frequency or efficiency of editing of a target nucleic acid of the present disclosure may vary. For example, the particular promoter used to drive gRNA expression may influence the editing efficiency of a target nucleic acid. In some embodiments, use of a Pol II promoter (e.g. a CmYLCV promoter) to drive gRNA expression may result in increased editing efficiency as compared to a corresponding control promoter (e.g. a Pol III promoter, such as a U6 promoter for example). Use of a Pol II promoter to drive gRNA expression may increase the relative editing efficiency of a target nucleic acid by, for example, at least about 1%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 125%, at least about 150%, at least about 175%, at least about 200%, at least about 225%, at least about 250%, at least about 275%, or at least about 300% or more as compared to a corresponding control (e.g. a U6 promoter).

Various conditions or variables described herein may improve editing efficiency of a Cas12L polypeptide as described herein (e.g. targeting a region of open chromatin for editing, use of a ribozyme in the gRNA targeting, performing editing in a plant genetic background that exhibits reduced transgene silencing, etc.) as compared to corresponding control conditions or variables. Various conditions or variables described herein may increase the relative editing efficiency of a target nucleic acid by, for example, at least about 1%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 125%, at least about 150%, at least about 175%, at least about 200%, at least about 225%, at least about 250%, at least about 275%, or at least about 300% or more as compared to a corresponding control condition or variable. Applicable control conditions or variables will be readily apparent to one of skill in the art depending on the particular editing context. For example, the corresponding control may be as compared to a region of closed chromatin or heterochromatin, editing without the use of a ribozyme, and/or editing in a plant genetic background that exhibits relatively high transgene silencing.

Comparisons in the present disclosure may also be in reference to corresponding control plants/plant cells. Various control plants will be readily apparent to one of skill in the art. For example, a control plant or plant cell may be a plant or plant cell that does not contain one or more of: (1) a Cas12L polypeptide, (2) a guide RNA, and/or (3) both a Cas12L polypeptide and a guide RNA.

Methods of probing the expression level of a nucleic acid are well-known to those of skill in the art. For example, qRT-PCR analysis may be used to determine the expression level of a population of nucleic acids isolated from a nucleic acid-containing sample (e.g. plants, plant tissues, or plant cells).

### Kits

Certain aspects of the present disclosure relate to an article of manufacture or kit comprising a polynucleotide, vector, cell, and/or composition described herein. In some embodiments, the kit further comprises a packed insert comprising instructions for the use of the polynucleotide, vector, cell, and/or composition. In some embodiments, the article of manufacture or kit further comprises one or more buffer, e.g., for storing, transferring, or otherwise using the polynucleotide, vector, cell, and/or composition. In some embodiments, the kit further comprises one or more containers for storing the polynucleotide, vector, cell, and/or composition.

The foregoing written description is considered to be sufficient to enable one skilled in the art to practice the present disclosure. The following Examples are offered for illustrative purposes only, and are not intended to limit the scope of the present disclosure in any way. Indeed, various modifications of the present disclosure in addition to those shown and described herein will become apparent to those skilled in the art from the foregoing description and fall within the scope of the appended claims.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
Aspect 1. A method for modifying a target nucleic acid in a eukaryotic cell, the method comprising contacting the target nucleic acid with: a) a Cas12L polypeptide; and b) a Cas12L guide nucleic acid, whereby said contacting results in modification of the target nucleic acid, and wherein said contacting is carried out at a temperature of from about 28° C. to about 37° C.
Aspect 2. The method of aspect 1, wherein the Cas12L polypeptide comprises an amino acid sequence having at least 80% amino acid identity to the amino acid sequence depicted in any one of FIG. 4A-4Z, FIG. 4AA-4ZZ, and FIG. 4AAA-4FFF.
Aspect 3. The method of any one of aspects 1-2, wherein the Cas12L polypeptide is a fusion Cas12L polypeptide comprising one or more nuclear localization signals (NLS).
Aspect 4. The method of aspect 3, wherein at least one of the one or more nuclear localization signals is an SV40-type NLS.
Aspect 5. The method of any one of aspects 1-4, wherein the cell comprises one or more nucleic acids comprising a nucleotide sequence encoding the Cas12L polypeptide and/or a nucleotide sequence encoding the Cas12L guide nucleic acid.
Aspect 6. The method of aspect 5, wherein the nucleotide sequence encoding the Cas12L polypeptide and/or the nucleotide sequence encoding the Cas12L guide nucleic acid is operably linked to a transcriptional control element that is functional in the eukaryotic cell.
Aspect 7. The method of aspect 6, wherein the transcriptional control element is a promoter.
Aspect 8. The method of aspect 7, wherein the promoter is a UBQ10 promoter.
Aspect 9. The method of aspect 8, wherein the UBQ10 promoter comprises a nucleic acid sequence that is at least 80% identical to SEQ ID NO:85 or SEQ ID NO:72.
Aspect 10. The method of any one of aspects 5-9, wherein the nucleotide sequence encoding the Cas12L guide nucleic acid is operably linked to an RNA Polymerase II promoter.
Aspect 11. The method of aspect 10, wherein the RNA Polymerase II promoter is a CmYLCV promoter or a 2×35S promoter.
Aspect 12. The method of aspect 11, wherein the promoter comprises a nucleic acid sequence that is at least 80% identical to SEQ ID NO:87 or SEQ ID NO:88.
Aspect 13. The method of any one of aspects 1-12, wherein the eukaryotic cell is maintained at a temperature in the range of about 28° C. to about 32° C.
Aspect 14. The method of any one of aspects 1-12, further comprising maintaining the eukaryotic cell at a temperature of less than 28° C., optionally maintaining the plant cell at a temperature of from about 17° C. to about 25° C.
Aspect 15. The method of any one of aspects 1-14, wherein the modification comprises a deletion of one or more nucleotides in the target nucleic acid.
Aspect 16. The method of aspect 15, wherein the deletion comprises deletion of 3-15 nucleotides in the target nucleic acid.
Aspect 17. The method of aspect 16, wherein the deletion comprises deletion of 9 nucleotides in the target nucleic acid.
Aspect 18. The method of any one of aspects 1-17, wherein the target nucleic acid sequence is located in a region of repressive chromatin.
Aspect 19. The method of any one of aspects 1-18, wherein the target nucleic acid sequence is located in a region of open chromatin.
Aspect 20. The method of any one of aspects 1-19, wherein the guide nucleic acid is fused to a ribozyme.
Aspect 21. The method of any of aspects 1-20, wherein the eukaryotic cell comprises a genetic background that exhibits reduced susceptibility to transgene silencing.
Aspect 22. The method of any one of aspects 1-21, wherein the method comprises:
(I): a) maintaining the eukaryotic cell for a first period of time at a first temperature of from about 17° C. to about 25° C., wherein the target nucleic acid is substantially not modified by the Cas12L polypeptide; and b) maintaining the eukaryotic cell for a second period of time at a second temperature of from about 25° C. to about 37° C., or from about 28° C. to about 37° C., or from about 28° C. to about 40° C., wherein the target nucleic acid is modified by the Cas12L polypeptide; or wherein the method comprises
(II): a) maintaining the eukaryotic cell for a first period of time at a first temperature of from about 25° C. to about 37° C., or from about 28° C. to about 37° C., or from about 28° C. to about 40° C., wherein the target nucleic acid is modified by the Cas12L polypeptide; and b) maintaining the eukaryotic cell for a second period of time at a second temperature of from about 17° C. to about 25° C., wherein the target nucleic acid is substantially not modified by the Cas12L polypeptide.
Aspect 23. The method of any one of aspects 1-22, wherein the eukaryotic cell is a plant cell.
Aspect 24. The method of aspect 23, wherein the target nucleic acid comprises a male reproductive pathway gene.
Aspect 25. The method of aspect 23, wherein the target nucleic acid comprises a nucleic acid in a pigment production pathway.
Aspect 26. The method of aspect 23, wherein the target nucleic acid provides for resistance to a disease caused by a fungus or a bacterium.
Aspect 27. The method of aspect 23, wherein the target nucleic acid provides for resistance to a plant pathogen.
Aspect 28. The method of aspect 23, wherein the target nucleic acid is a nucleic acid in the ethylene pathway.
Aspect 29. The method of any one of aspects 1-22, wherein the eukaryotic cell is an insect cell, an arachnid cell, a mammalian cell, a fish cell, a fungal cell, a yeast cell, an amphibian cell, or an avian cell.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or see, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

The following examples are offered to illustrate provided embodiments and are not intended to limit the scope of the present disclosure. In the Examples provided herein, tables appear beneath the table heading that describes the respective table.

### Example 1: Cas12L Conducts Gene Editing in Plant Cells

This Example demonstrates that Cas12L1, as a member of compact type V-L CRSIPR-Cas system, is able to conduct gene editing in plant cells. The in vivo gene editing in plant cells of this example was achieved by introducing RNPs into cells which are composed of the CAS12L1 protein loaded by guide RNA. Cas12L1 is able to edit the target gene with higher editing efficiency at higher temperature (28° C. and 32° C.). On the contrary, at ambient temperature as 23° C., the editing efficiency from CAS12L-x is very minimal compared to higher temperature. This distinct property of the CAS12L1 protein allows for potential applications involving temperature control of editing activities.

### Materials and Methods

### RNP Reconstitution

Guide RNAs were synthesized (36nt repeat+20nt spacer as shown in Table 1-1) by IDT. Guide RNA was dissolved by adding DEPC-treated H₂O to a concentration of 0.5 mM. 5 μL of the dissolved RNA was incubated at 65° C. for 3 minutes, then cooled to room temperature. For RNP reconstitution, 2.5 μL of heated-and-cooled RNA was added to 218.9 μL 2×CB buffer (2×CB buffer contains: 20 mM Hepes-Na, 300 mM KCl, 10 mM MgCl₂, 20% glycrol, 1 mM TCEP; pH 7.5), vortexed to mix, and spun. Then, 28.6 μL of 35 μM Cas12L1 protein was added and pipetted to mix. The mixture was then incubated at room temperature for 30 minutes. The resulting mixture contains 4 μM RNP in 2×CB buffer. All reagents were maintained as RNase free.

### Protoplast Isolation and Transfection

Protoplast isolation was performed as described in the following publication: PMID: 17585298. Special care was performed for an overall sterile environment when preparing protoplast.

For RNP transfection, 26 μL of 4 μM RNP were first added to a round-bottom 2 mL tube. Then 200 μL of protoplasts (at 2×10⁵ cells/mL) were added to the tube. 2 μL of 5 μg/μL salmon sperm DNA was added and mixed gently by tapping the tube 3-4 times. Then, 228 μL of fresh, sterile and RNase free PEG-CaCl₂ solution (PMID: 17585298) was added to the protoplast-plasmid mixture and mixed well by gently tapping the tubes. The protoplasts with PEG solution were incubated at room temperature for 10 minutes, then 880 μL of W5 solution (PMID: 17585298; Yoo et al. (2007) Nature Protocols 2:1565) was added and mixed with the protoplasts by inverting the tube 2-3 times to stop the transfection. Protoplasts were harvested by centrifugation at 100 rcf for 2 min, resuspended in 1 mL WI solution (PMID: 17585298), and plated into 6-well plates pre-coated with 5% calf serum. The lids of the 6-well plates were closed to begin the incubation of the protoplasts. The plates with protoplasts were incubated for 48 hours at 23° C., 28° C. and 32° C., respectively.

For control samples, 10 μL of HBT-sGFP (S65T) plasmid (ABRC stock CD3-911) were added to 200 μL protoplasts. Then, 220 μL of PEG-CaCl₂ solution (PMID: 17585298) was added to the protoplast-plasmid mixture and mixed well by gently tapping tubes. The protoplasts with PEG solution were incubated at room temperature for 10 minutes, then 880 μL of W5 solution (PMID: 17585298) was added and mixed with the protoplasts by inverting the tube 2-3 times to stop the transfection. Protoplasts were then harvested and plated to the 6-well plates as described above in the RNP transfection sections and incubated together with RNP transfected protoplasts for 48 hours at 23° C., 28° C. and 32° C., respectively.

At the end of the incubations, the protoplasts were harvested by first centrifugation at 100 rcf for 2-3 minutes. Keeping the pellet, the supernatant was moved to another tube and went through another centrifugation at 3000 rcf for 3 minutes to collect any residue protoplasts. Pellets from these two centrifugations were combined and flash frozen for further analysis.

### Amplicon Sequencing

DNAs of protoplast samples were extracted using the Qiagen DNeasy plant mini kit. Amplicons were obtained by two rounds of PCR. Amplification primers for the first round of PCR were designed to have the 3′ part of primer with sequences flanking a 200-300 bp fragment of the AtPDS3 gene around the guide RNA of interest. The 5′ part of the primer contained sequences to be bound by common sequencing primers. The first round of PCR was done with Thermo fusion enzyme. Half of all DNA from a protoplast sample was used as the template, and 25 cycles of amplification were done for the first round. Then the reaction was cleaned by 1× Ampure XP beads. The elution from the cleanup was used as the template for the second round of PCR by fusion enzyme with 12 cycles. The second round of PCR was designed so that indexes were added to each sample. The samples were then purified by 0.8-1× Ampure beads for 1-2 rounds until no primer dimers were seen, with fragments below 200 bp considered primer dimers. Then amplicons were sent next generation sequencing.

### Amplicon Sequencing Result Analysis

Reads were first quality- and adaptor-trimmed with trim-galore, then mapped to the AtPDS3 genomic region by BWA aligner. Sorted and indexed bam files were used as input files for further analysis by the CrispRvariants R package. Each mutation pattern with corresponding reads counts were exported by the CrispRvariants R package. For comparing detected indel frequency of different indel sizes (FIG. 2), indels with >100 reads were counted. For calculating editing efficiency, after assessing all control samples, a criterion to classify reads containing indels was established: only reads with ≥2 bp deletion or insertion of the same pattern (deletion or insertion of same size starting with same location) with ≥100 reads counts per pattern from a sample were included in determining the number of reads having indels. This criterion was established due to the fact that 1-bp indels were observed with reads number ≥100 in control samples. Larger deletions were also observed at very low frequencies (much lower than 100 reads) in control samples. These observations indicate that occasional PCR inaccuracy and low-quality sequencing in a small fraction of reads can result in the deletion patterns with corresponding read number ranges as stated above in control samples. These stringent criteria were employed so that the counted deletion signals were true signal indicating editing events, though it is possible that Cas12L1 might be able to create 1 bp indels at lower frequency compared to the larger sized indels.

### Results

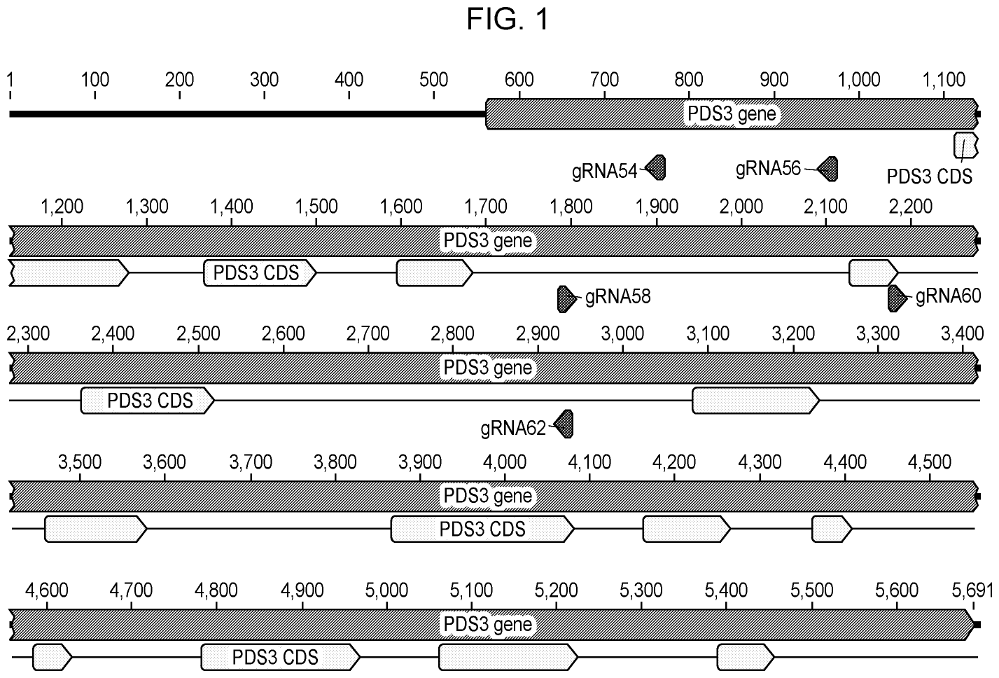

To test if the Cas12L1 protein is able to conduct gene editing in plant cells, Arabidopsis PDS3 gene was used as the target gene and 5 guide RNAs were designed targeting different loci in the AtPDS3 gene. The guide RNA loci are denoted in FIG. 1 and the guide RNA sequences are listed in Table 1-1. Arabidopsis mesophyll protoplasts were transfected with Cas12L1 RNPs reconstituted with the Cas12L1 protein and the guide RNAs designed. After incubating transfected protoplasts for 48 hours at 23° C., 28° C. and 32° C., the protoplasts were harvested and the DNA of protoplasts were extracted. Amplicon sequencing was performed to assess the detailed gene editing products.

TABLE 1-1
Guide RNA sequence as used in RNPs.

| Purpose | Cas12L1 Guide RNA repeat sequence (common to all guides) | | |
|---|---|---|---|
| For RNPs | ATTGTTGTAACTCTTATTTTGTATGGAGTAAACAAC (SEQ ID NO: 57) | | |
| | Guide RNA spacer sequence | | Direction relative |
| Guide # | (Denoted in DNA sequence) | PAM | to AtPDS3 gene |
| 54 | GTTTTCAGACCAAGCTCTTT (SEQ | TTG | reverse |
| | ID NO: 181) | | |
| 56 | TATCTTTAACACATAATTGA (SEQ | TTG | reverse |
| | ID NO: 182) | | |
| 58 | TGAGACTACAAGGTATAACT (SEQ | TTG | forward |
| | ID NO: 183) | | |
| 60 | CATATTTTCTGTAAGTCCAA (SEQ | TTA | forward |
| | ID NO: 184) | | |
| 62 | ATTACCCATCCTAAAGTATG (SEQ | TTG | reverse |
| | ID NO: 185) | | |

Read counts of different indel sizes detected from all protoplast samples transfected with the CasL1 RNPs were compared (FIG. 2A). Deletions of 7 bp to 11 bp in size were the most abundant gene editing products created by the Cas12L1 protein, while insertions were much less abundant compared to deletions (FIG. 2A). 1 bp indels were much less frequently detected compared to deletions of bigger sizes. Occasionally, in the control samples, which were transfected by the HBT-sGFP plasmid, 1 bp indels were also detected (FIG. 2B), indicating that PCR and sequencing errors could contribute to the appearance of 1 bp indels at the depth of amplicon sequencing used for these samples (1-5 million reads per sample). To ensure that the editing efficiencies reveal the true editing signals, 1 bp indels were not included for the calculation of editing efficiencies. These results suggest that the Cas12L1 protein tends to generate deletions of relatively big size in plant cells.

Gene editing products were readily detected with guide RNA 54, 56, 58, and 62, with the guide RNA 54 and guide RNA 58 yielding relatively high editing efficiencies (FIG. 3A-C and FIG. 3E). With guide RNA 60, gene editing was only detected in one replicate sample incubated at 32° C. (FIG. 3D), suggesting that either the guide RNA 60 barely functions to target gene editing or the specific target locus of guide RNA 60 is very difficult to access. The detailed gene editing patterns and corresponding patterns were listed in Table 1-2 to Table 1-10. In Table 1-2 through Table 1-10, the left column of each table shows the editing pattern and the right column of each table shows the number of reads for that editing pattern. Editing patterns are shown as: (position where the editing starts):(number of nucleotides of) D (deletion) or I (insertion). Position 0 is between the 18th and 19th nucleotides of the guide, so that the 18th nucleotide is position −1, the 19th nucleotide is position +1. Editing patterns of indel size >=2 bp and of read counts >=100 in each sample are shown.

TABLE 1-2
Detailed amplicon sequencing results of WT protoplasts
transfected with Cas12L1 RNP and gRNA54 incubated at
23° C. Three replicate transfections were performed.

| | |
|---|---|
| Editing Pattern | Reads |

RNP of Cas12L and gRNA54 at 23° C., replicate 3

| | |
|---|---|
| −4:8D | 203 |
| 2:5D | 146 |
| −2:11D | 129 |
| −2:5D | 106 |
| −8:25D | 103 |
| edited reads | 687 |
| total reads | 1526436 |
| editing efficency | 0.045% |

RNP of Cas12L1 and gRNA54 at 23° C., replicate 1

| | |
|---|---|
| −2:8D | 210 |
| −5:9D | 139 |
| −2:11D | 118 |
| edited reads | 467 |
| total reads | 1384171 |
| editing efficency | 0.03% |

RNP of Cas12L1 and gRNA54 at 23° C., replicate 2

| | |
|---|---|
| −2:11D | 246 |
| −5:10 | 168 |
| −8:16D | 124 |
| −9:37D | 119 |
| 2:5D | 116 |
| edited reads | 773 |
| total reads | 1338510 |
| editing efficency | 0.06% |

TABLE 1-3
Detailed amplicon sequencing results of WT protoplasts
transfected with Cas12L1 RNP and gRNA 54 incubated at
28° C. Three replicate transfections were performed.
RNP of Cas12L1 and gRNA54 at 28° C., replicate 1

| | |
|---|---|
| −4:8D | [symbol] |
| 2:5D | [symbol] |
| −2:7D | [symbol] |
| [symbol] | [symbol] |
| −2:11D | [symbol] |
| [symbol] | [symbol] |
| −6:10D | [symbol] |
| −5:10D | [symbol] |
| −7:15D | [symbol] |
| −2:5D | [symbol] |
| −6:11D | [symbol] |
| 2:9D | 1241 |
| −5:13D | [symbol] |
| [symbol] | [symbol] |
| 1:6D | [symbol] |
| [symbol] | [symbol] |
| −1:7D | [symbol] |
| [symbol] | [symbol] |
| [symbol] | 685 |
| −7:11D | [symbol] |
| [symbol] | [symbol] |
| [symbol] | 524 |
| [symbol] | [symbol] |
| −2:3D | [symbol] |
| 2:2D | [symbol] |
| [symbol] | [symbol] |
| −7:10D | [symbol] |
| [symbol] | [symbol] |
| −17:24D | [symbol] |
| −4:17D | [symbol] |
| −2:12D | 325 |
| [symbol] | 307 |
| −12:18D | [symbol] |
| [symbol] | [symbol] |
| [symbol] | [symbol] |
| [symbol] | [symbol] |
| [symbol] | 299 |
| −10:18D | 297 |
| −10:14D | 295 |
| [symbol] | 289 |
| −6:7D | [symbol] |
| [symbol] | [symbol] |
| [symbol] | [symbol] |
| [symbol] | [symbol] |
| [symbol] | 277 |
| [symbol] | 272 |
| 3:4D | 270 |
| −7:8D | 261 |
| [symbol] | 259 |
| [symbol] | 246 |
| [symbol] | 240 |
| [symbol] | 239 |
| [symbol] | 239 |
| [symbol] | 233 |
| 4:2D | 227 |
| 1:10D | 205 |
| [symbol] | [symbol] |
| −11:19D | 123 |
| edited reads | [symbol] |
| total reads | [symbol] |
| editing efficency | [symbol] |

RNP of Cas12L1 and gRNA54 at 28° C., replicate 2

| | |
|---|---|
| −4:8D | [symbol] |
| [symbol] | [symbol] |
| [symbol] | [symbol] |
| −5:10D | [symbol] |
| −2:11D | [symbol] |
| −6:8D | [symbol] |
| −6:10D | 2915 |
| [symbol] | [symbol] |
| [symbol] | [symbol] |
| −2:7D | 2455 |
| −6:11D | [symbol] |
| [symbol] | [symbol] |
| [symbol] | [symbol] |
| [symbol] | [symbol] |
| [symbol] | [symbol] |
| −6:14D | 1041 |
| −7:11D | [symbol] |
| −7:10D | [symbol] |
| [symbol] | [symbol] |
| [symbol] | 619 |
| 5:3D | [symbol] |
| [symbol] | [symbol] |
| [symbol] | 517 |
| −2:4D | 514 |
| [symbol] | 499 |
| [symbol] | 477 |
| [symbol] | 457 |
| −5:12D | [symbol] |
| −15:25D | [symbol] |
| [symbol] | [symbol] |
| [symbol] | [symbol] |
| [symbol] | [symbol] |
| [symbol] | 342 |
| [symbol] | [symbol] |
| [symbol] | [symbol] |
| [symbol] | [symbol] |
| [symbol] | 301 |
| 1:3D | 299 |
| −7:15D | 256 |
| [symbol] | 243 |
| [symbol] | 221 |
| [symbol] | 210 |
| [symbol] | 198 |
| [symbol] | 198 |
| −5:16D | 197 |
| [symbol] | [symbol] |
| [symbol] | 190 |
| [symbol] | 186 |
| [symbol] | 186 |
| [symbol] | 180 |
| [symbol] | 180 |
| [symbol] | 179 |
| [symbol] | 177 |
| [symbol] | 177 |
| [symbol] | [symbol] |
| [symbol] | 172 |
| [symbol] | 172 |
| [symbol] | 172 |
| [symbol] | 170 |
| [symbol] | 170 |
| [symbol] | 168 |
| [symbol] | 167 |
| [symbol] | 165 |
| [symbol] | [symbol] |
| 2:2D | [symbol] |
| [symbol] | [symbol] |
| [symbol] | [symbol] |
| −14:20D | 101 |
| edited reads | 63588 |
| total reads | [symbol] |
| editing efficency | 2.9% |

RNP of Cas12L1 and gRNA54 at 28° C., replicate 3

| | |
|---|---|
| −4:8D | 7606 |
| −5:9D | 4366 |
| −2:11D | 3423 |
| −6:10D | 3244 |
| 2:5D | 3180 |
| −2:5D | [symbol] |
| −2:8D | 2524 |
| −5:10D | 2259 |
| −6:8D | 2206 |
| −2:7D | 1942 |
| −5:13D | [symbol] |
| −6:11D | 1477 |
| [symbol] | 1114 |
| −6:9D | 1101 |
| −2:6D | 932 |
| [symbol] | 851 |
| −1:7D | 812 |
| −4:9D | [symbol] |
| −3:9D | 596 |
| −5:6D | 555 |
| −2:4D | [symbol] |
| −4:10D | [symbol] |
| −6:17D | 465 |
| −7:8D | 442 |
| [symbol] | [symbol] |
| −6:12D | [symbol] |
| −7:10D | [symbol] |
| −7:15D | 382 |
| −5:12D | 361 |
| −18:25D | 356 |
| −3:15D | 342 |
| −8:18D | 335 |
| −10:17D | 323 |
| −7:11D | [symbol] |
| −1:11D | [symbol] |
| −5:14D | 304 |
| −8:16D | 298 |
| −7:13D | 293 |
| [symbol] | 289 |
| −9:15D | 271 |
| −2:12D | 267 |
| −20:26D | [symbol] |
| [symbol] | 184 |
| [symbol] | 180 |
| −12:15D | 173 |
| [symbol] | 172 |
| −5:33D | 166 |
| −10:30D | 166 |
| −4:6D | 166 |
| −3:14D | 165 |
| −10:15D | 164 |
| −8:11D | 161 |
| [symbol] | 160 |
| −4:24D | 157 |
| −5:7D | 156 |
| [symbol] | 156 |
| [symbol] | 152 |
| −4:5D | 149 |
| [symbol] | 148 |
| [symbol] | 144 |
| [symbol] | 144 |
| −2:10D | 143 |
| [symbol] | 137 |
| 3:8D | 133 |
| [symbol] | 132 |
| −12:13D | 128 |
| −2:2D | [symbol] |
| 2:2D | 124 |
| −12:20D | 119 |
| [symbol] | 116 |
| −22:29D | 114 |
| −8:10D | 111 |
| [symbol] | 111 |
| −8:9D | 102 |
| 3:4D | 100 |
| edited reads | 56266 |
| total reads | 2200775 |
| editing efficency | 2.6% |

[symbol]

indicates data missing or illegible when filed

TABLE 1-4
Detailed amplicon sequencing results of WT protoplasts transfected
with Cas12L1 RNP and gRNA 54 incubated at 32° C. WT protoplasts
were transfected with RNP of Cas12L protein and gRNA54 and incubated
at 32° C. Three replicate transfections were performed.
RNP of Cas12L1 and gRNA54 at 32° C., replicate 1

| | |
|---|---|
| −4:8D | 70221 |
| −5:9D | 43822 |
| −2:8D | 33014 |
| −5:10D | 22171 |
| −2:11D | 21959 |
| −6:8D | 21934 |
| −6:11D | 20245 |
| −2:7D | 18204 |
| −6:10D | 16684 |
| −2:5D | 14662 |
| 2:5D | 13204 |
| −7:11D | 9056 |
| 2:9D | 9016 |
| −6:9D | 8140 |
| −6:7D | 6986 |
| −5:13D | 6019 |
| −3:6D | 5652 |
| −4:9D | 4875 |
| −1:7D | 4540 |
| −7:10D | 4394 |
| −7:13D | 3605 |
| −5:14D | 3513 |
| −15:25D | 3149 |
| −18:25D | 3047 |
| −8:25D | 2836 |
| −7:12D | 2716 |
| −4:10D | 2660 |
| −4:5D | 2592 |
| 1:6D | 2303 |
| −3:9D | 2141 |
| −7:9D | 2003 |
| −9:14D | 1978 |
| 5:3D | 1959 |
| −1:4D | 1884 |
| −5:19D | 1823 |
| −4:6D | 1815 |
| −3:10D | 1720 |
| −5:7D | 1714 |
| 3:4D | 1692 |
| −7:16D | 1676 |
| −5:12D | 1406 |
| −6:14D | 1389 |
| −6:12D | 1363 |
| −10:14D | 1299 |
| −15:46D | 1117 |
| −16:30D | 1094 |
| −20:26D | [symbol] |
| −17:24D | [symbol] |
| −9:27D | 1037 |
| −5:21D | 1027 |
| −1:11D | 991 |
| −25:31D | 975 |
| −5:16D | 971 |
| −8:17D | 951 |
| −4:13D | 950 |
| −4:14D | 927 |
| 3:8D | 914 |
| −10:1D, −5:10D | 905 |
| −4:17D | 890 |
| −9:17D | 874 |
| −31:35D | 873 |
| −4:12D | 851 |
| −5:5D | 827 |
| −6:13D | 814 |
| −5:25D | 800 |
| −2:10D | [symbol] |
| −3:30D | 778 |
| 4:2D | 749 |
| −16:22D | 748 |
| [symbol] | 745 |
| −8:16D | 711 |
| −13:18D | 696 |
| −23:33D | 604 |
| −27:34D | 540 |
| −1:13D | 521 |
| −14:24D | 512 |
| −3:20D | 502 |
| −6:22D | 494 |
| −5:6D | 484 |
| −2:15D | 455 |
| [symbol] | 412 |
| −7:8D | 378 |
| 1:7D | 122 |
| edited reads | 437276 |
| total reads | 2752332 |
| editing efficency | 15.9% |

RNP of Cas12L1 and gRNA54 at 32° C., replicate 2

| | |
|---|---|
| −4:8D | 63653 |
| −2:11D | 30003 |
| −5:9D | 29075 |
| −2:8D | 27122 |
| −6:8D | 24556 |
| −6:11D | 19712 |
| −5:10D | 17469 |
| −6:10D | 15945 |
| 2:5D | 13915 |
| −2:7D | 13806 |
| −2:5D | 11032 |
| −6:9D | 7596 |
| −7:11D | 7077 |
| −7:10D | 6136 |
| −4:9D | 6070 |
| −5:13D | 4652 |
| −1:7D | 4619 |
| 1:3D | 4262 |
| 2:9D | 4246 |
| −7:15D | 3919 |
| −6:7D | 3736 |
| 2:2D | 3126 |
| −18:25D | 3115 |
| −5:12D | 3037 |
| −3:6D | 3033 |
| −7:12D | 3020 |
| −5:16D | 2938 |
| −5:15D | 2803 |
| −6:12D | 2652 |
| −7:13D | 2597 |
| 1:6D | 2458 |
| −2:6D | 2361 |
| −1:11D | 2236 |
| 3:4D | 2234 |
| −3:9D | [symbol] |
| −1:4D | 2152 |
| −5:18D | 1999 |
| −2:12D | 1662 |
| −4:10D | 1638 |
| −2:14D | 1530 |
| −2:3D | 1507 |
| −2:4D | 1425 |
| −4:6D | 1374 |
| −4:17D | 1292 |
| −17:24D | 1250 |
| −18:22D | 1197 |
| −6:13D | 1190 |
| −9:14D | 1090 |
| −6:15D | 1086 |
| −8:16D | 1081 |
| −10:14D | 1076 |
| −5:6D | 1045 |
| −5:20D | 1024 |
| −4:5D | 998 |
| −8:25D | 965 |
| −7:9D | 936 |
| −20:26D | 933 |
| −14:20D | 898 |
| −4:7D | 898 |
| 3:8D | 892 |
| 5:3D | 885 |
| −6:17D | 857 |
| −7:8D | 770 |
| −6:14D | 750 |
| −15:25D | 652 |
| −23:14D, −7:13D | 650 |
| −5:40D | 612 |
| −15:21D | 612 |
| −26:30D | 602 |
| −5:23D | 602 |
| [symbol] | 580 |
| −9:27D | 570 |
| −6:30D | 567 |
| −3:10D | 564 |
| −6:22D | 557 |
| −7:19D | 555 |
| −3:13D | 543 |
| −12:10D, 2:16D | 537 |
| −13:23D | 504 |
| −7:2D, −3:22D | 502 |
| −5:22D | 502 |
| −9:13D | 498 |
| −5:14D | 490 |
| −5:49D | 488 |
| −8:14D | 475 |
| −5:21D | 473 |
| −9:15D | 460 |
| −5:7D | 457 |
| −1:13D | 454 |
| −6:18D | 432 |
| 7:3D | 430 |
| −7:16D, 15:5D | 344 |
| −5:25D | 315 |
| −28:35D | 307 |
| [symbol] | 306 |
| −3:15D | 297 |
| −9:17D | 284 |
| −8:13D | 276 |
| −12:20D | 273 |
| −2:21D | 261 |
| −5:19D | 248 |
| edited reads | 408071 |
| total reads | 2309093 |
| editing efficency | 17.7% |

RNP of Cas12L1 and gRNA54 at 32° C., replicate 3

| | |
|---|---|
| −4:8D | 67339 |
| −5:9D | 32199 |
| [symbol] | [symbol] |
| −2:11D | [symbol] |
| [symbol] | [symbol] |
| [symbol] | [symbol] |
| −5:10D | 17754 |
| −6:11D | [symbol] |
| −2:7D | 16630 |
| 2:5D | 15475 |
| −2:5D | [symbol] |
| [symbol] | [symbol] |
| −7:11D | [symbol] |
| [symbol] | [symbol] |
| [symbol] | [symbol] |
| −5:13D | [symbol] |
| [symbol] | 4241 |
| [symbol] | [symbol] |
| [symbol] | [symbol] |
| [symbol] | [symbol] |
| [symbol] | 3443 |
| [symbol] | [symbol] |
| [symbol] | [symbol] |
| [symbol] | [symbol] |
| [symbol] | [symbol] |
| [symbol] | [symbol] |
| [symbol] | 2611 |
| [symbol] | [symbol] |
| [symbol] | [symbol] |
| [symbol] | [symbol] |
| [symbol] | [symbol] |
| −3:9D | 1990 |
| −4:7D | [symbol] |
| −6:15D | [symbol] |
| −5:14D | 1651 |
| [symbol] | [symbol] |
| [symbol] | [symbol] |
| [symbol] | [symbol] |
| [symbol] | [symbol] |
| [symbol] | [symbol] |
| [symbol] | 1561 |
| [symbol] | [symbol] |
| [symbol] | 1434 |
| [symbol] | 1392 |
| [symbol] | [symbol] |
| [symbol] | 1252 |
| [symbol] | [symbol] |
| [symbol] | [symbol] |
| [symbol] | [symbol] |
| [symbol] | [symbol] |
| [symbol] | 1161 |
| [symbol] | 1127 |
| [symbol] | 1107 |
| [symbol] | [symbol] |
| [symbol] | 1090 |
| [symbol] | [symbol] |
| [symbol] | [symbol] |
| [symbol] | 1016 |
| [symbol] | [symbol] |
| [symbol] | 993 |
| −5:6D | 950 |
| −2:3D | 948 |
| −5:22D | 831 |
| −6:24D | 816 |
| [symbol] | 812 |
| [symbol] | 785 |
| [symbol] | 745 |
| [symbol] | 674 |
| [symbol] | 672 |
| −14:44D | [symbol] |
| [symbol] | [symbol] |
| [symbol] | [symbol] |
| [symbol] | [symbol] |
| [symbol] | [symbol] |
| [symbol] | [symbol] |
| −5:29D | [symbol] |
| [symbol] | [symbol] |
| [symbol] | [symbol] |
| [symbol] | [symbol] |
| [symbol] | [symbol] |
| [symbol] | [symbol] |
| −12:24D | [symbol] |
| [symbol] | 564 |
| −14:24D | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| 3:4D | [symbol] |
| [symbol] | [symbol] |
| −2:6D | [symbol] |
| [symbol] | [symbol] |
| [symbol] | [symbol] |
| [symbol] | [symbol] |
| −11:15D | [symbol] |
| −4:22D | [symbol] |
| −2:15D | [symbol] |
| [symbol] | [symbol] |
| [symbol] | [symbol] |
| −1:11D | [symbol] |
| 4:2D | [symbol] |
| [symbol] | [symbol] |
| −11:24D | 300 |
| −8:20D | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| edited reads | [symbol] |
| total reads | [symbol] |
| editing efficency | 15.5% |

[symbol]

indicates data missing or illegible when filed

TABLE 1-5
Detailed amplicon sequencing results of WT protoplasts
transfected with Cas12L1 RNP and gRNA 56 incubated
at 28° C. and 32° C. Three replicate transfections
were performed for each incubation temperature.
RNP of Cas12L1 and gRNA56 at 28° C., replicate 1

| | |
|---|---|
| −2:6D | 239 |
| −2:5D | 179 |
| −4:9D | 164 |
| −2:10D | 127 |
| 4:2D | 121 |
| edited reads | 830 |
| total reads | [symbol] |
| editing efficency | 0.04% |

RNP of Cas12L1 and gRNA56 at 28° C., replicate 2

| | |
|---|---|
| −2:6D | 105 |
| edited reads | 105 |
| total reads | 2202021 |
| editing efficency | 0.005% |

RNP of Cas12L1 and gRNA56 at 28° C., replicate 3

| | |
|---|---|
| −3:8D | 172 |
| edited reads | 172 |
| total reads | 2412516 |
| editing efficency | 0.007% |

RNP of Cas12L1 and gRNA56 at 32° C., replicate 1

| | |
|---|---|
| −2:6D | 1744 |
| −5:12D | 1621 |
| [symbol] | 725 |
| −7:12D | 718 |
| −5:10D | 421 |
| edited reads | 5229 |
| total reads | 2792089 |
| editing efficency | 0.19% |

RNP of Cas12L1 and gRNA56 at 32° C., replicate 2

| | |
|---|---|
| −2:6D | 1328 |
| −5:12D | 602 |
| −4:9D | 533 |
| −5:10D | 476 |
| −5:9D | 458 |
| −2:7D | 438 |
| [symbol] | 424 |
| [symbol] | 273 |
| −2:13D | 244 |
| −5:18D | 243 |
| −2:10D | 219 |
| −2:9D | 198 |
| −6:11D | 146 |
| −3:8D | 137 |
| −1:6D | 131 |
| −3:17D | 119 |
| −5:22D | 119 |
| edited reads | 6088 |
| total reads | 2714455 |
| editing efficency | 0.22% |

RNP of Cas12L1 and gRNA56 at 32° C., replicate 3

| | |
|---|---|
| −2:7D | 398 |
| −3:8D | 384 |
| −2:8D | 383 |
| 4:2D | 376 |
| −5:8D | 371 |
| −5:10D | 358 |
| −5:12D | 239 |
| −4:9D | 218 |
| −2:9D | 201 |
| −2:6D | 200 |
| −5:9D | 142 |
| edited reads | 3270 |
| total reads | 2702657 |
| editing efficency | 0.12% |

[symbol]

indicates data missing or illegible when filed

TABLE 1-6
Detailed amplicon sequencing results of WT protoplasts
transfected with Cas12L1 RNP and gRNA58 incubated at
23° C. Three replicate transfections were performed.
RNP of Cas12L1 and gRNA58 at 23° C., replicate 1

| | |
|---|---|
| −6:11D | 168 |
| −3:9D | 123 |
| −3:7D | 121 |
| edited reads | 412 |
| total reads | 1389154 |
| editing efficency | 0.03% |

RNP of Cas12L1 and gRNA58 at 23° C., replicate 2

| | |
|---|---|
| −3:8D | 118 |
| −6:11D | 111 |
| [symbol] | 100 |
| edited reads | 329 |
| total reads | 1522269 |
| editing efficency | 0.02% |

RNP of Cas12L1 and gRNA58 at 23° C., replicate 3

| | |
|---|---|
| −6:11D | 211 |
| −4:8D | [symbol] |
| −3:9D | 106 |
| edited reads | 454 |
| total reads | 1706607 |
| editing efficency | 0.03% |

[symbol]

indicates data missing or illegible when filed

TABLE 1-7
Detailed amplicon sequencing results of WT protoplasts
transfected with Cas12L1 RNP and gRNA58 incubated at
28° C. Three replicate transfections were performed.
RNP of Cas12L1 and gRNA58 at 28° C., replicate 1

| | |
|---|---|
| −6:11D | 4335 |
| −3:7D | 3184 |
| −6:9D | 3044 |
| −3:9D | 2879 |
| −3:4D | 2553 |
| −3:11D | 1696 |
| −8:34D | 1691 |
| −3:10D | 1690 |
| −5:6D | 1372 |
| −1:3D | 1204 |
| −5:13D | 895 |
| −3:5D | 858 |
| 1:7D | 647 |
| −2:6D | 578 |
| −6:43D | 555 |
| −12:18D | 533 |
| −7:12D | 458 |
| −1:6D | 454 |
| −13:18D | 441 |
| −8:19D | 343 |
| −7:15D | 327 |
| −17:27D | 303 |
| −10:43D | 294 |
| −19:31D | 287 |
| −4:16D | 279 |
| −23:27D, 8:6D | 274 |
| −9:21D | 268 |
| −12:28D | 268 |
| −9:14D | 263 |
| −1:12D | 259 |
| −6:22D | 258 |
| −5:12D | 257 |
| −5:10D | 252 |
| −24:37D | 250 |
| −8:17D | 249 |
| −17:23D | 247 |
| −4:19D | 247 |
| [symbol] | 245 |
| −1:10D | 241 |
| −14:31D | 238 |
| −1:14D | 235 |
| −1:9D | 228 |
| −10:24D | 219 |
| −4:17D | 217 |
| −6:13D | 210 |
| −1:8D | 209 |
| −3:19D | 208 |
| −2:5D | 207 |
| −2:13D | 206 |
| −1:5D | 206 |
| −8:10D | 203 |
| −2:8D | 187 |
| −3:18D | 186 |
| [symbol] | 169 |
| −10:26D | 156 |
| −12:23D | 142 |
| 1:4D | 113 |
| edited reads | 38017 |
| total reads | 2427511 |
| editing efficency | 1.57% |

RNP of Cas12L1 and gRNA58 at 28° C., replicate 2

| | |
|---|---|
| −6:11D | 4147 |
| −6:9D | 3983 |
| −3:9D | 3945 |
| −3:7D | 3906 |
| −3:11D | 2052 |
| −3:10D | 1992 |
| −3:4D | 1782 |
| −5:13D | 1720 |
| −24:28D | 1694 |
| −5:6D | 1692 |
| −8:34D | 1575 |
| −3:8D | 975 |
| −7:12D | 853 |
| −1:6D | 836 |
| −5:21D | 807 |
| −3:5D | 794 |
| −1:9D | 605 |
| −1:7D | 579 |
| −1:3D | 576 |
| −1:5D | 516 |
| −5:17D | 507 |
| −2:8D | 472 |
| −8:10D | 458 |
| −6:20D | 441 |
| 1:4D | 416 |
| −5:12D | 415 |
| −7:11D | 408 |
| −24:37D | 378 |
| −5:10D | 345 |
| −8:19D | 326 |
| −8:17D | 325 |
| −6:16D | 306 |
| −4:8D | 305 |
| −1:8D | 271 |
| −27:33D | 263 |
| −9:24D | 262 |
| −4:7D | 262 |
| −3:12D | 252 |
| −2:6D | 248 |
| −1:10D | 247 |
| −2:5D | 246 |
| −4:15D | 242 |
| −2:13D | 241 |
| −7:15D | 238 |
| −7:14D | 223 |
| 4:2D | 206 |
| −4:10D | 204 |
| 2:3D | 204 |
| −10:16D | 196 |
| −20:33D | 176 |
| −6:43D | 175 |
| −8:3D, −2:40D | 175 |
| −3:18D | 169 |
| −23:29D | 167 |
| −11:30D | 159 |
| 1:22D | 153 |
| −3:28D | 149 |
| −3:49D | 148 |
| −14:16D | 147 |
| −20:24D | 146 |
| −6:18D | 145 |
| −1:18D | 143 |
| −19:31D | 140 |
| −4:17D | 137 |
| −3:27D | 136 |
| −4:25D | 133 |
| −3:16D | 133 |
| 1:5D | 132 |
| −7:28D | 131 |
| −27:36D | 131 |
| −11:15D | 125 |
| −12:16D | 123 |
| [symbol] | 123 |
| −13:19D | 122 |
| 2:6D | 121 |
| −14:31D | 120 |
| 2:7D | 119 |
| −2:17D | 119 |
| 4:3D | 117 |
| −4:23D | 116 |
| [symbol] | 109 |
| [symbol] | 109 |
| 1:6D | 108 |
| 2:5D | 107 |
| [symbol] | 104 |
| [symbol] | 103 |
| −14:13D | 101 |
| edited reads | 49007 |
| total reads | 2487706 |
| editing efficency | 1.97% |

RNP of Cas12L1 and gRNA58 at 28° C., replicate 3

| | |
|---|---|
| −6:11D | 5514 |
| −3:7D | 4344 |
| −3:10D | 2711 |
| −3:9D | 2625 |
| −6:9D | 2392 |
| −5:6D | 1950 |
| −3:11D | 1766 |
| −8:34D | 1330 |
| −3:8D | 1249 |
| −5:13D | 979 |
| −1:6D | 947 |
| −1:3D | 940 |
| −4:10D | 885 |
| −24:28D | 868 |
| −3:4D | 836 |
| −5:12D | 829 |
| −4:15D | 681 |
| 1:4D | 575 |
| −5:21D | 561 |
| −1:7D | 536 |
| −3:18D | 533 |
| −7:12D | 497 |
| −2:6D | 450 |
| −6:18D | 444 |
| −2:8D | 444 |
| −27:34D | 432 |
| −1:9D | 414 |
| −3:5D | 413 |
| −12:20D | 369 |
| −17:27D | 347 |
| −3:19D | 313 |
| −4:7D | 303 |
| −1:5D | 302 |
| −8:17D | 300 |
| −4:8D | 291 |
| −6:20D | 290 |
| 1:5D | 266 |
| −2:5D | 260 |
| −3:27D | 259 |
| −9:14D | 221 |
| −5:17D | 221 |
| −5:10D | 220 |
| −8:14D | 202 |
| −9:18D | 196 |
| −3:17D | 191 |
| 1:7D | 183 |
| 4:2D | 181 |
| −9:17D | 179 |
| 2:6D | 170 |
| −3:16D | 167 |
| −2:12D | 161 |
| −5:4D | 155 |
| −5:30D | 152 |
| −10:43D | 146 |
| −10:31D | 142 |
| −8:19D | 139 |
| −20:33D | 139 |
| −17:41D | 137 |
| −27:32D | 136 |
| −16:21D | 135 |
| −6:13D | 127 |
| −5:45D | 126 |
| −4:29D | 122 |
| −18:25D | 117 |
| −1:15D | 116 |
| −16:25D | 115 |
| −24:37D | 113 |
| −14:13D | 113 |
| −4:14D | 113 |
| −19:30D, 14:7D | 110 |
| −19:22D | 110 |
| −10:19D | 110 |
| −1:10D | 108 |
| −16:24D | 107 |
| −13:19D | 106 |
| −9:19D | 102 |
| −10:21D | 102 |
| −11:17D | 102 |
| 1:13D | 101 |
| edited reads | 45138 |
| total reads | 2383492 |
| editing efficency | 1.89% |

[symbol]

indicates data missing or illegible when filed

TABLE 1-8
Detailed amplicon sequencing results of WT protoplasts
transfected with Cas12L1 RNP and gRNA58 incubated at
32° C. Three replicate transfections were performed.
RNP of Cas12L1 and gRNA58 at 32° C., replicate 1

| | |
|---|---|
| −6:11D | 51270 |
| −3:7D | 45707 |
| −5:6D | 35964 |
| −6:9D | 25841 |
| −3:9D | 18671 |
| −3:10D | 16584 |
| [symbol] | 11358 |
| −3:8D | 11317 |
| −3:11D | 9649 |
| −5:13D | 8532 |
| −4:7D | 7542 |
| −3:16D | 7124 |
| −3:5D | 6375 |
| −3:4D | 6286 |
| −4:8D | 6286 |
| −1:3D | 5584 |
| −24:28D | 5493 |
| −5:12D | 5061 |
| −14:16D | 4862 |
| 1:4D | 4674 |
| −6:18D | 4612 |
| −8:10D | 4268 |
| −4:10D | 4238 |
| 4:2D | 4017 |
| −1:5D | 3787 |
| −5:10D | 3215 |
| −3:18D | 3187 |
| −6:16D | 3145 |
| −6:20D | 3056 |
| −2:5D | 2970 |
| [symbol] | 2690 |
| −1:10D | 2556 |
| −3:19D | 2462 |
| −1:7D | 2376 |
| −7:12D | 2218 |
| −24:37D | 2105 |
| −4:15D | 2072 |
| 2:4D | 1997 |
| −7:14D | 1909 |
| −2:14D | 1760 |
| −1:9D | 1643 |
| −3:14D | 1612 |
| −10:17D | 1577 |
| [symbol] | 1483 |
| −12:41D | 1270 |
| −7:21D | 1267 |
| −6:43D | 1210 |
| −1:16D | 1189 |
| −27:40D | 1181 |
| −12:25D | 1152 |
| −20:33D | 1121 |
| −19:31D | 1117 |
| −3:25D | 1112 |
| −12:12D, 3:9D | 1103 |
| −4:16D | 1067 |
| −14:13D | 1066 |
| −27:34D | 1052 |
| −14:23D | 1050 |
| −4:17D | 1040 |
| −5:2D | 1023 |
| −7:15D | 1017 |
| −2:15D | 1009 |
| −16:25D | 1001 |
| −24:30D | 1001 |
| −2:6D | 996 |
| −10:25D | 987 |
| −4:14D | 977 |
| −1:15D | 973 |
| −8:15D | 965 |
| 1:8D | 957 |
| 1:13D | 946 |
| [symbol] | 923 |
| −8:17D | 921 |
| 2:5D | 912 |
| −3:10D, 13:2D | 910 |
| 1:5D | 893 |
| −27:33D | 884 |
| 3:9D | 881 |
| [symbol] | 871 |
| 4:5D | 847 |
| [symbol] | 804 |
| [symbol] | 759 |
| 4:11D | 734 |
| [symbol] | 683 |
| −10:34D | 555 |
| −4:19D | 551 |
| −5:27D | 546 |
| −17:27D | 531 |
| −5:21D | 522 |
| −12:28D | 509 |
| −9:14D | 499 |
| −6:13D | 481 |
| 1:7D | 458 |
| −7:11D | 443 |
| −7:9D | 197 |
| edited reads | 404298 |
| total reads | 2897596 |
| editing efficency | 13.95% |

RNP of Cas12L1 and gRNAS8 at 32° C., replicate 2

| | |
|---|---|
| −3:7D | 69161 |
| −6:11D | 50429 |
| −6:9D | 30795 |
| −5:6D | [symbol] |
| −3:9D | 24572 |
| −3:10D | [symbol] |
| −24:28D | 17485 |
| −3:4D | 15363 |
| −3:11D | 14996 |
| −8:34D | 14467 |
| −5:13D | 13964 |
| [symbol] | 10894 |
| −5:12D | 9478 |
| [symbol] | 8783 |
| −4:8D | 7808 |
| −6:18D | [symbol] |
| −4:10D | [symbol] |
| −4:7D | 6978 |
| −3:5D | 6775 |
| −1:5D | 6081 |
| −3:16D | 6022 |
| −7:12D | 5009 |
| 1:4D | [symbol] |
| −3:18D | 4296 |
| −6:20D | [symbol] |
| −1:6D | [symbol] |
| −1:7D | 3454 |
| −3:19D | 3426 |
| 4:2D | [symbol] |
| −2:6D | 3399 |
| −19:22D | 3334 |
| −6:16D | [symbol] |
| [symbol] | [symbol] |
| −7:11D | 2614 |
| −8:10D | [symbol] |
| −2:5D | [symbol] |
| −8:17D | 2389 |
| −4:15D | [symbol] |
| −2:8D | 2102 |
| −20:33D | 2060 |
| [symbol] | 2004 |
| −5:21D | [symbol] |
| −1:9D | 1898 |
| −19:31D | 1882 |
| −1:8D | 1796 |
| −2:34D | 1729 |
| 1:5D | 1518 |
| −8:19D | 1450 |
| 1:7D | 1302 |
| −25:37D | 1187 |
| −4:14D | 1164 |
| −7:13D | 1163 |
| 2:3D | 1162 |
| −18:25D | 1045 |
| −4:16D | 1025 |
| −3:27D | 1022 |
| −3:25D | 1015 |
| 2:7D | 980 |
| −5:15D | 968 |
| [symbol] | 961 |
| −12:19D | 961 |
| −19:25D | 959 |
| [symbol] | 938 |
| −12:18D | 903 |
| −26:38D | 893 |
| [symbol] | 785 |
| −4:19D | 779 |
| −17:27D | [symbol] |
| [symbol] | 704 |
| [symbol] | 686 |
| −5:14D | [symbol] |
| −9:45D | [symbol] |
| −13:35D | 595 |
| −5:30D | 592 |
| −24:33D | 579 |
| −27:34D | 574 |
| [symbol] | 571 |
| [symbol] | [symbol] |
| −11:33D | 557 |
| [symbol] | 557 |
| −24:37D | 556 |
| −7:18D | 550 |
| −28:32D | 545 |
| [symbol] | 526 |
| [symbol] | 521 |
| [symbol] | 521 |
| −8:21D | 516 |
| −16:25D | 501 |
| −12:23D | 501 |
| [symbol] | 493 |
| −4:23D | 491 |
| −16:18D | 488 |
| −3:17D | 486 |
| −26:32D | 486 |
| [symbol] | 485 |
| −27:29D | 480 |
| [symbol] | 477 |
| [symbol] | 475 |
| −2:14D | 474 |
| −13:20D | [symbol] |
| −3:22D | [symbol] |
| −2:12D | 468 |
| −27:33D | 468 |
| −2:13D | 464 |
| −4:17D | 461 |
| −23:26D | 457 |
| [symbol] | 455 |
| −6:15D | 455 |
| [symbol] | 454 |
| −14:16D | 451 |
| [symbol] | 449 |
| [symbol] | 442 |
| −8:25D | 442 |
| [symbol] | 434 |
| [symbol] | 432 |
| [symbol] | 428 |
| [symbol] | 428 |
| −10:16D | 427 |
| [symbol] | 418 |
| −9:11D | 416 |
| [symbol] | 411 |
| [symbol] | 409 |
| 2:4D | 404 |
| [symbol] | 401 |
| [symbol] | 397 |
| [symbol] | 394 |
| [symbol] | 894 |
| −7:14D | 380 |
| [symbol] | 380 |
| 4:3D | 379 |
| [symbol] | 365 |
| [symbol] | 356 |
| −10:12D | 351 |
| −10:18D | 329 |
| −8:12D | 310 |
| [symbol] | 301 |
| −9:23D | 286 |
| [symbol] | [symbol] |
| −22:29D | 272 |
| −5:8D | 270 |
| −10:34D | 270 |
| −5:27D | 257 |
| −28:34D | 258 |
| [symbol] | 250 |
| −19:29D | 248 |
| −13:18D | 244 |
| −21:27D | 243 |
| −12:17D | 241 |
| −11:10D | 240 |
| [symbol] | 232 |
| −3:12D | 231 |
| −10:23D | 221 |
| −11:17D | 218 |
| [symbol] | 218 |
| [symbol] | 206 |
| [symbol] | 202 |
| [symbol] | 201 |
| [symbol] | 175 |
| [symbol] | 146 |
| [symbol] | 119 |
| edited reads | 509527 |
| total reads | 3378837 |
| editing efficency | 15.08% |

RNP of Cas12L1 and gRNA58 at 32° C., replicate 3

| | |
|---|---|
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| edited reads | [symbol] |
| total reads | [symbol] |
| editing efficency | [symbol] |

[symbol]

indicates data missing or illegible when filed

TABLE 1-9
Detailed amplicon sequencing results of WT protoplasts transfected
with Cas12L1 RNP and gRNA 60 incubated at 32° C. Three
replicate transfections were performed and only replicate
2 which editing was detected was shown here.

| Editing Pattern | Reads |
|---|---|
| −10:14D | 345 |
| edited reads | 345 |
| total reads | 3494744 |
| editing efficency | 0.01% |

TABLE 1-10
Detailed amplicon sequencing results of WT protoplasts transfected
with Cas12L1 RNP and gRNA 62 incubated at 28° C. and 32°
C. Three replicate transfections were performed for each incubation
temperature and only the replicates with editing detected are shown.
RNP of Cas12L1 and gRNA62 at 28° C., replicate 1

| | |
|---|---|
| 4:2D | 632 |
| −4:8D | 312 |
| −5:12D | 167 |
| edited reads | 1111 |
| total reads | 2706875 |
| editing efficency | 0.04% |

RNP of Cas12L1 and gRNA62 at 28° C., replicate 3

| | |
|---|---|
| −3:9D | 260 |
| 4:2D | 224 |
| −2:10D | 214 |
| edited reads | 698 |
| total reads | 2623596 |
| editing efficency | 0.03% |

RNP of Cas12L1 and gRNA62 at 32° C., replicate 1

| | |
|---|---|
| 4:2D | 1001 |
| −3:9D | 889 |
| −2:10D | 630 |
| −2:7D | 594 |
| [symbol] | 363 |
| −4:11D | 303 |
| −10:17D | 227 |
| −8:17D | 227 |
| 3:3D | 197 |
| edited reads | 4431 |
| total reads | 2732168 |
| editing efficency | 0.16% |

RNP of Cas12L1 and gRNA62 at 32° C., replicate 2

| | |
|---|---|
| −4:11D | 963 |
| 4:2D | 829 |
| −2:10D | 585 |
| −6:16D | 387 |
| −5:9D | 295 |
| 4:5D | 292 |
| 1:6D | 271 |
| −2:7D | 253 |
| −4:12D | 245 |
| −6:8D | 158 |
| [symbol] | 145 |
| [symbol] | 144 |
| edited reads | 4567 |
| total reads | 2496251 |
| editing efficency | 0.18% |

RNP of Cas12L1 and gRNA62 at 32° C., replicate 3

| | |
|---|---|
| 4:2D | 810 |
| −4:11D | 719 |
| −4:8D | 365 |
| 5:2D | 323 |
| −2:10D | 313 |
| −3:18D | 300 |
| 1:6D | 244 |
| −5:5D, 3:21D | 236 |
| −20:32D | 177 |
| −1:10D | 156 |
| −3:9D | 145 |
| −6:10D | 142 |
| −2:7D | 139 |
| −3:8D | 136 |
| −10:22D | 136 |
| −5:9D | 124 |
| [symbol] | 109 |
| −4:12D | 108 |
| −4:13D | 100 |
| edited reads | 4782 |
| total reads | 2648760 |
| editing efficency | 0.18% |

[symbol]

indicates data missing or illegible when filed

Strikingly, for all the functioning guide RNAs, the editing efficiencies increased dramatically as the incubation temperature of transfected protoplasts increased within the temperature range tested. At 23° C., no editing events or very minimal editing were detected, while at 28° C., gene editing products were more frequently detected (FIG. 3A-C and FIG. 3E). At 32° C., even though it was only 4° C. higher than 28° C., gene editing efficiencies were much higher compared to that at 28° C. For guide RNA 54 and guide RNA 58, it was about 6-7-folds of increase in editing efficiencies when the incubation temperature raised from 28° C. to 32° C. Such drastic changes in editing efficiencies of the Cas12L1 protein at different temperatures exhibit a distinct feature of this Cas protein, which barely functions at ambient room temperature for the guide RNAs tested, but is able to perform very efficient gene editing at higher temperature.

The results of this example suggest that the Cas12L1 protein created large deletions rather than insertions or indels of smaller sizes, with the editing efficiency increased drastically as temperature rises. This strong temperature sensitivity of Cas12L1 function opens interesting possibilities for using this enzyme as a “temperature switch” for applications where incubation temperature can be controlled for control of gene editing and thus, gene function.

### Example 2: Cas12L and Trans Cleavage of ssRNA

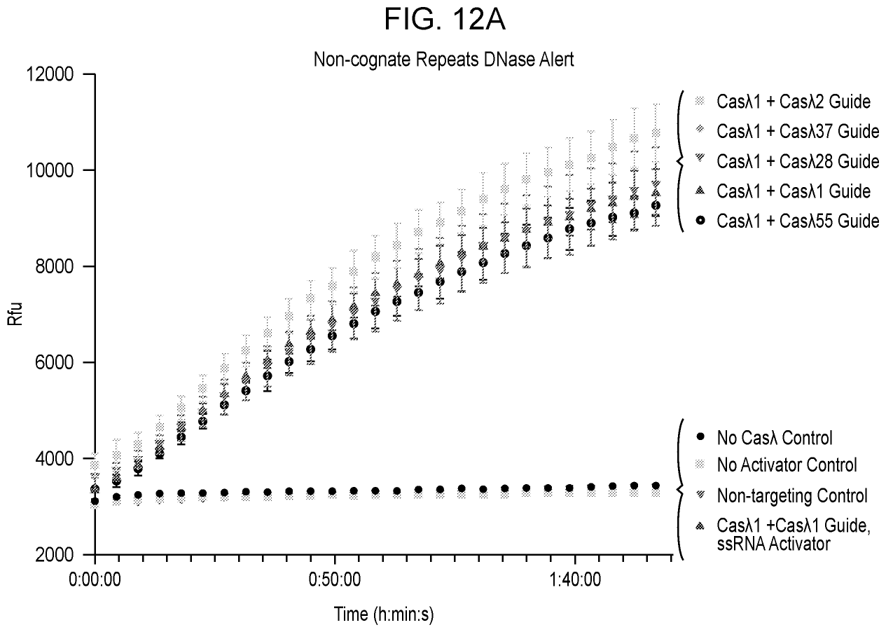

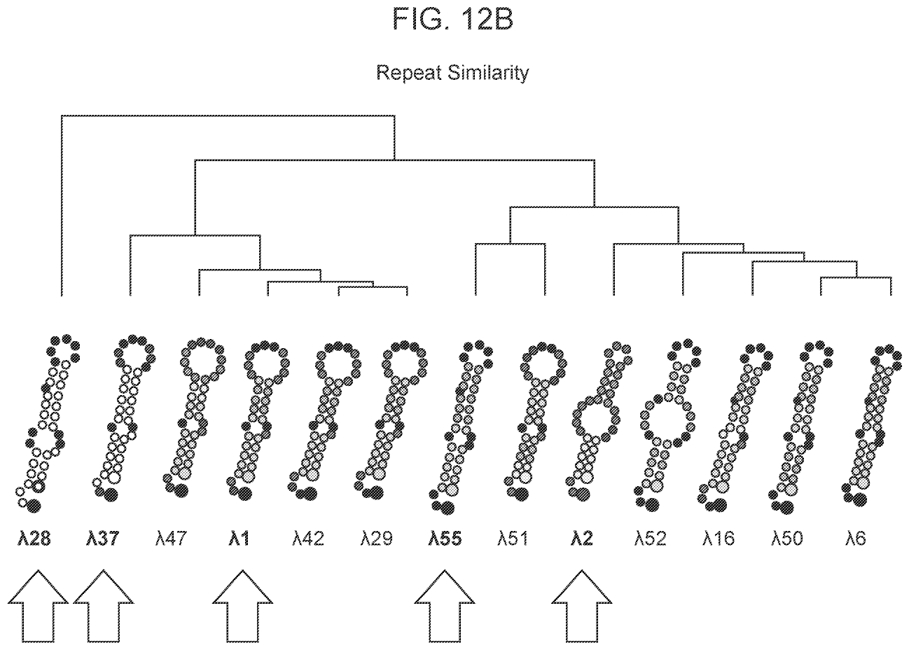

A nucleic acid reporter molecule tagged with a fluorophore and a quencher was added to different concentrations (7 nM and 100 nM) of the Cas12L RNP, consisting of the Cas12L protein and the rBAS14 guide RNA (augcAUUGUUGUAACUCUUAUUUUGUAUGGAGUAAACAACUAGCAUCACCUUCACCCUCU (SEQ ID NO:186)). In the presence of ssDNA activator (dBAS312, AGAGGGTGAAGGTGATGCTA (SEQ ID NO:187)) and at increasing RNP concentrations, higher cleavage of the reporter molecule led to greater Rfu signals, which was not reflected in the controls with no activator and/or no guide RNA, control reactions lacking of Mg2+ in the buffer, or controls with nuclease-inactive Cas12L. Replacing the CRISPR repeat portion of the guide RNA with Repeats from other Cas12L homologs achieved comparable trans-activity (FIG. 12).

Creating artificial mismatches along each position in the ssDNA target revealed the mismatch tolerance of the protein, showing highly tolerated mismatches at the 6^{th} and 14^{th} positions in the protospacer target and beyond the 14^{th} position.

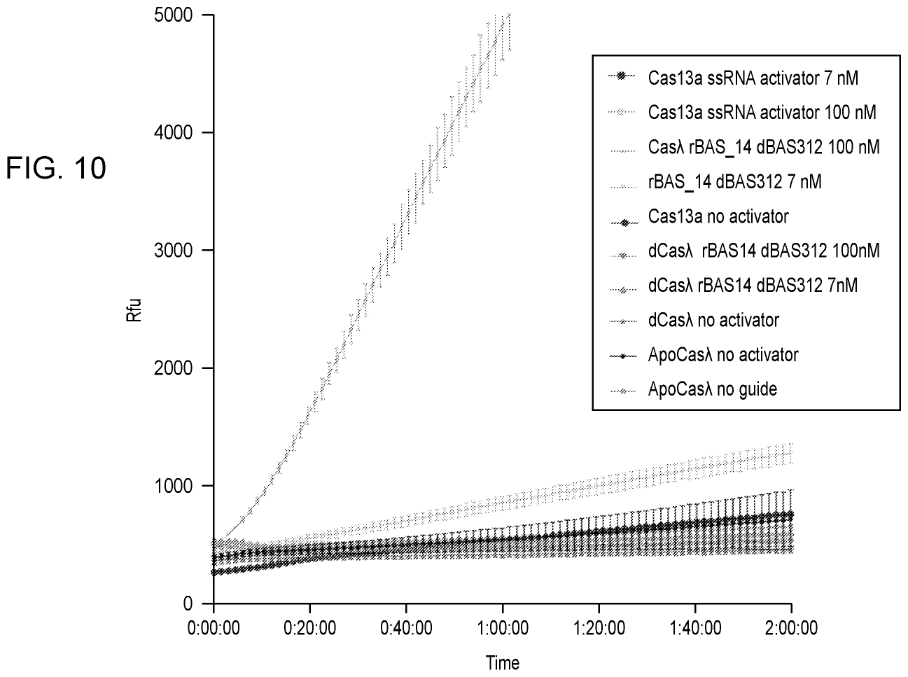

Trans-cleavage activity of Casλ was tested against ssRNA poly-U reporter molecules using ssDNA activators. Different concentrations of protein (100 nM, 7 nM) were tested. The results are depicted in FIG. 10. As shown in FIG. 10. only Casλ (rBAS_14 dBAS312] at 100 nM exhibited substantial trans-cleavage of the ssRNA. poly-uridine reporter molecule.

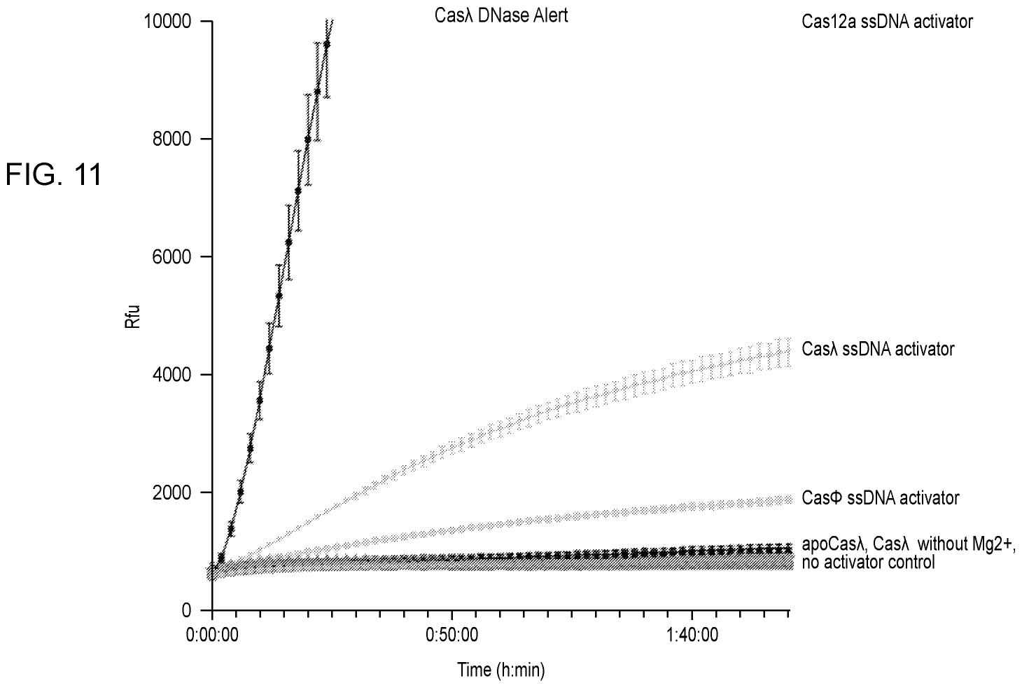

FIG. 11 depicts trans-cleavage activity of Cas) against DNase Alert Fluorophore-Quencher reporters using ssDNA activators.

FIG. 12 depicts trans-cleavage activity of Casλ against DNase Alert Fluorophore-Quencher reporters using ssDNA activators. gRNAs of different Cas. orthologs were used in this assay, showing that the gRNAs of different Casλ proteins can still be used for DNA targeting and are interchangeable and the RNP complex remains functional despite sequence-level dissimilarity.

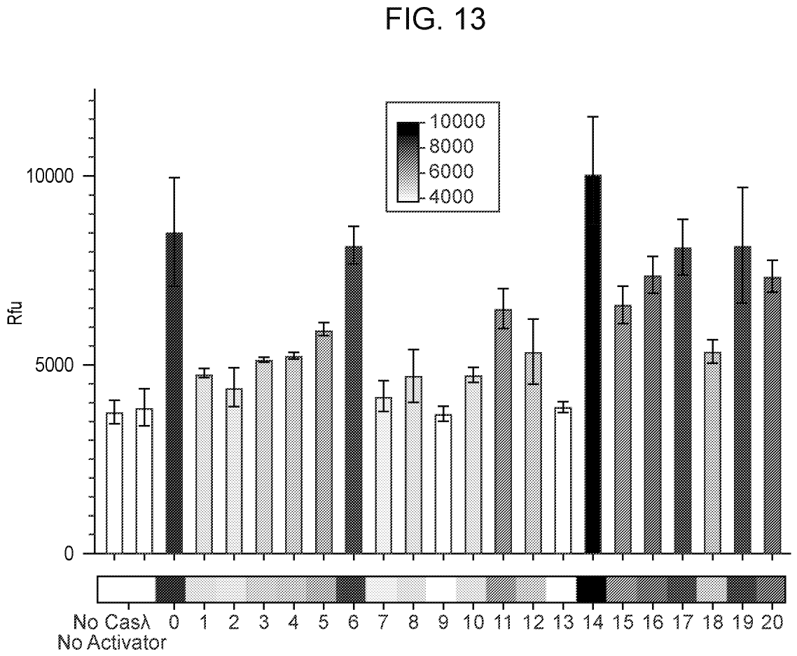

FIG. 13 depicts trans-cleavage activity of Casλ against DNase Alert Fluorophore-Quencher reporters using ssDNA activators. The gRNAs for Cas' contained sequential mismatches at the nucleotide position shown, indicating where mismatches may be tolerated and still result in cutting, and where they are not tolerated and do not lead to DNA cleavage. This mismatch tolerance information lends insight into possible off-target effects with gene editing.

### Example 3: Characterization of Cas12L

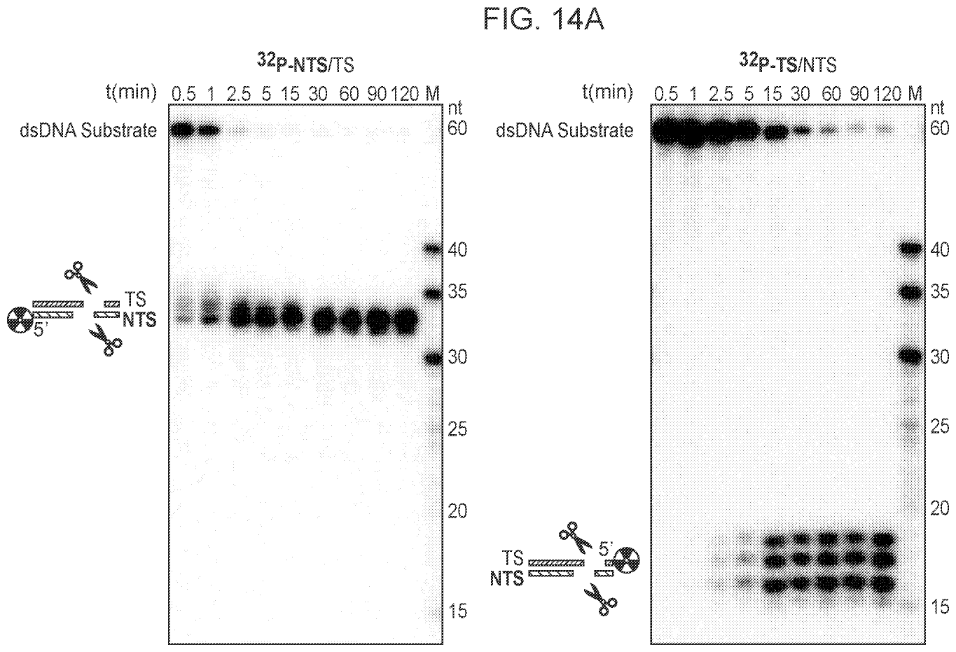

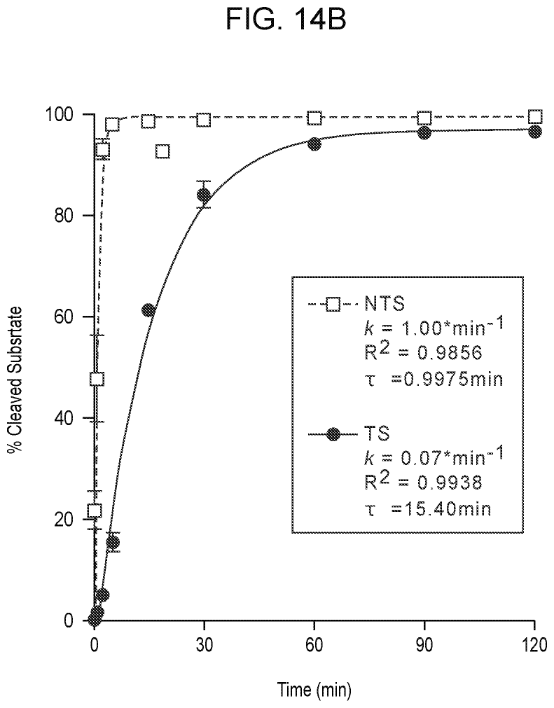

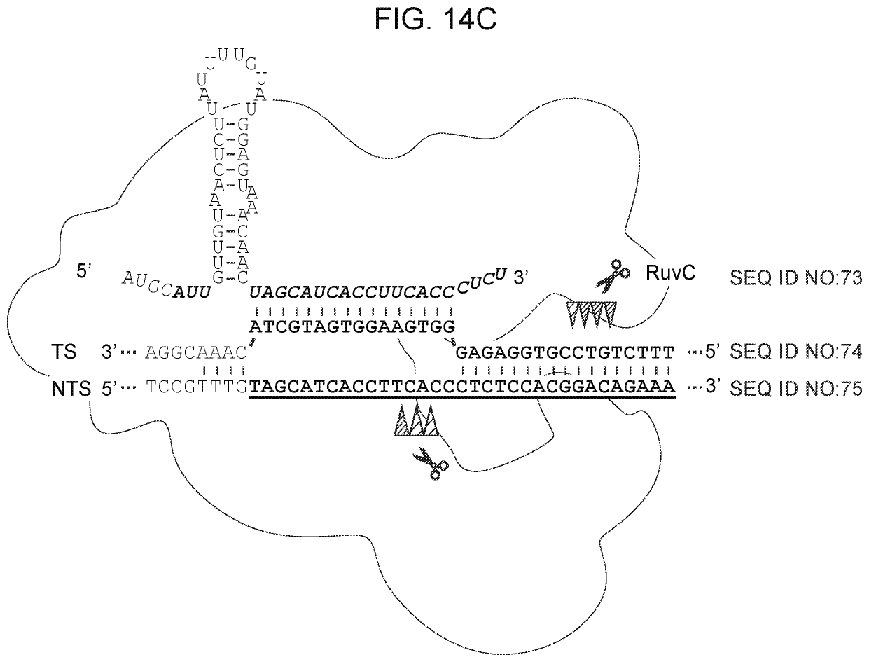

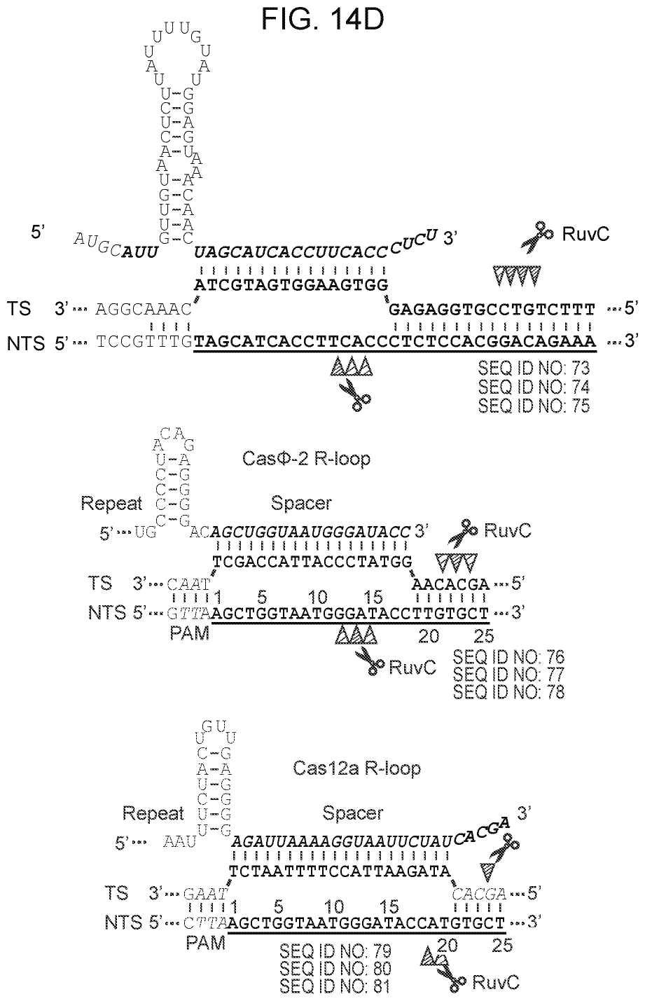

FIG. 14. Cleavage assay assessing the ability of Casλ RNPs to cleave the target strand (TS), and non-target strand (NTS), of a dsDNA oligo duplex. Cleaved fractions are calculated based on the substrate band intensities at t=(0 min) relative to the respective time point and fit to a One Phase Decay model to derive cleavage rate.

Active RNP complexes were assembled in a 1:1.2 molar ratio in RNP assembly buffer and incubated for 30 min at RT. Substrates were 5′-end-labelled using T4-PNK (NEB) in the presence of 32P-y-ATP. Oligoduplex targets were generated by combining 32P-labelled and unlabelled complementary oligonucleotides in a 1:1.5 molar ratio. Oligos were hybridized to a DNA-duplex concentration of 50 nM in hybridization buffer, by heating for 5 min to 95° C. and a slow cool down to RT in a heating block. Cleavage reactions were initiated by combining 200 nM RNP with 2 nM substrate in reaction buffer and subsequently incubated at 37° C. Reactions were stopped by addition of two volumes formamide loading buffer (96% formamide, 100 μg/mL bromophenol blue, 50 μg/mL xylene cyanol, 10 mM EDTA, 50 μg/mL heparin), heated to 95° C. for 5 min, and cooled down on ice before separation on a 12.5% denaturing ureaPAGE. Gels were dried for 4 h at 80° C. before phosphor-imaging visualization using an Amersham Typhoon scanner (GE Healthcare). Technical replicates (n ≥2) and comparable cleavage assays under varied conditions (n ≥3) of biological replicates (n ≥2) showed consistent results.

FIG. 14 depicts 5′ radiolabeling of the Target Strand (TS) and Non-Target Strand (NTS) of DNA complementary to the Casλ gRNA illustrates dsDNA cleavage position and kinetics, and cleavage positions compared to CasΦ polypeptides and Cas12a polypeptides.

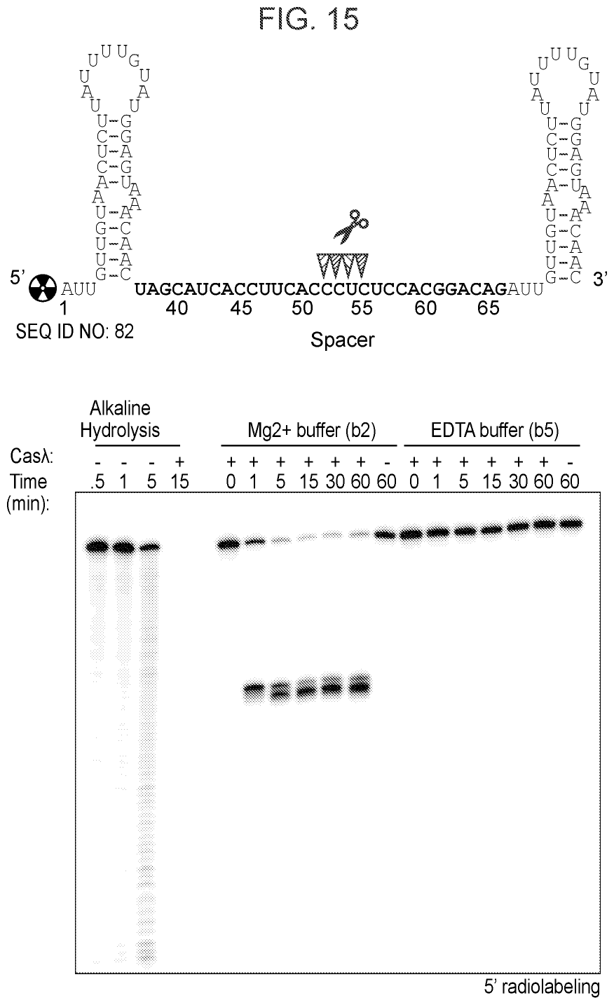

FIG. 15 depicts 5′ radiolabeling of the pre-crRNA and incubation with the Casλ protein illustrates that unlike most Cas12 and Cas9 enzymes, Casλ not only processes its own crRNA to form the mature gRNA, but also uses the same RuvC active site that is used for DNA cleavage to do so, and processes the RNA within the spacer region as opposed to the repeat region. These experiments were performed with Repeat-Spacer--Repeat substrates as well as with Repeat-Spacer substrates with an extra 5′ handle.

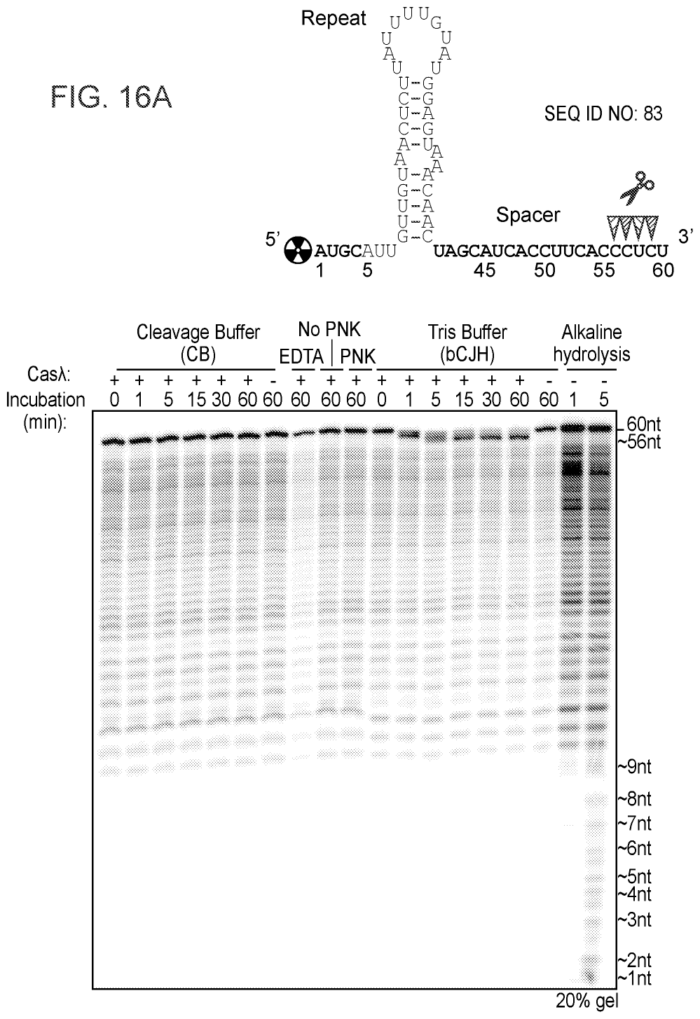

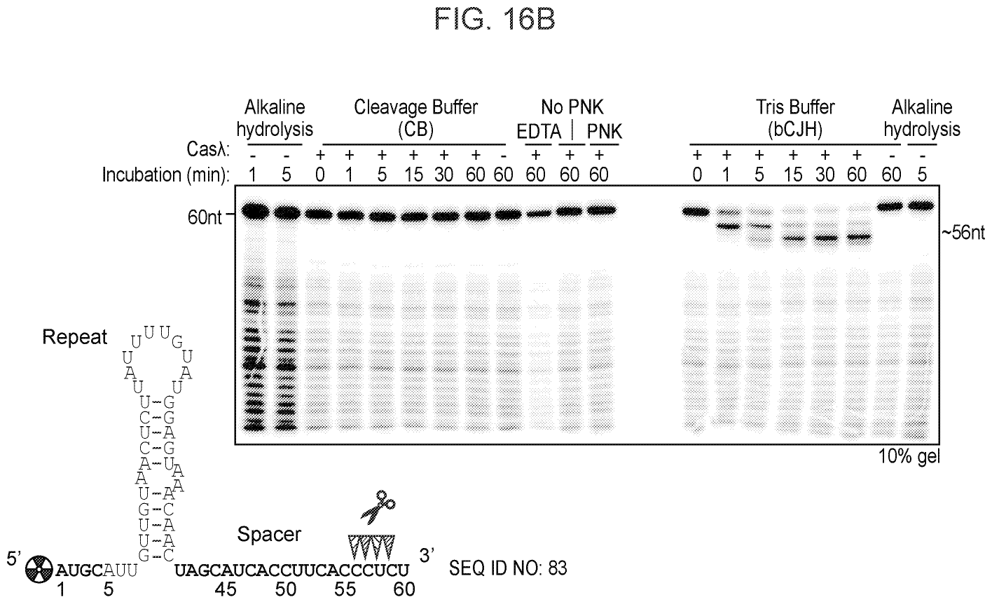

FIG. 16 presents data analogous to that in FIG. 15, but with 10% and 20% polyacrylamide gels, and with modified guides to elongate the repeat region at the 5′ end and to observe whether any nucleotides are cleaved at the 5′ end down to a single nucleotide, or if the size of the mature guide processed from a Repeat-Spacer pair matches what was shown in the Repeat-Spacer-Repeat setup in FIG. 15. The absence of Mg²⁺ ablates the Repeat processing ability, indicating that the RNA processing is likely produced by the RuvC active site also used to cleave DNA targets, and that multiplexing guides requires the presence of Mg²⁺.

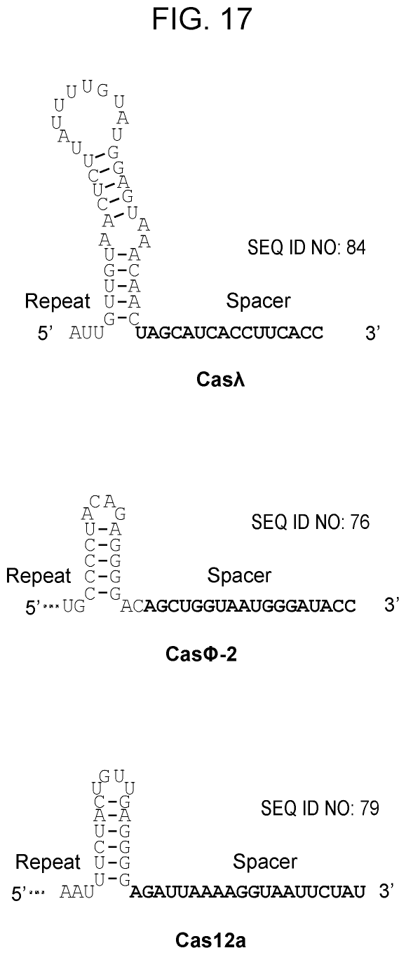

FIG. 17 depicts mature gRNA for Cask, compared to CasΦ gRNA and Cas12a gRNA.

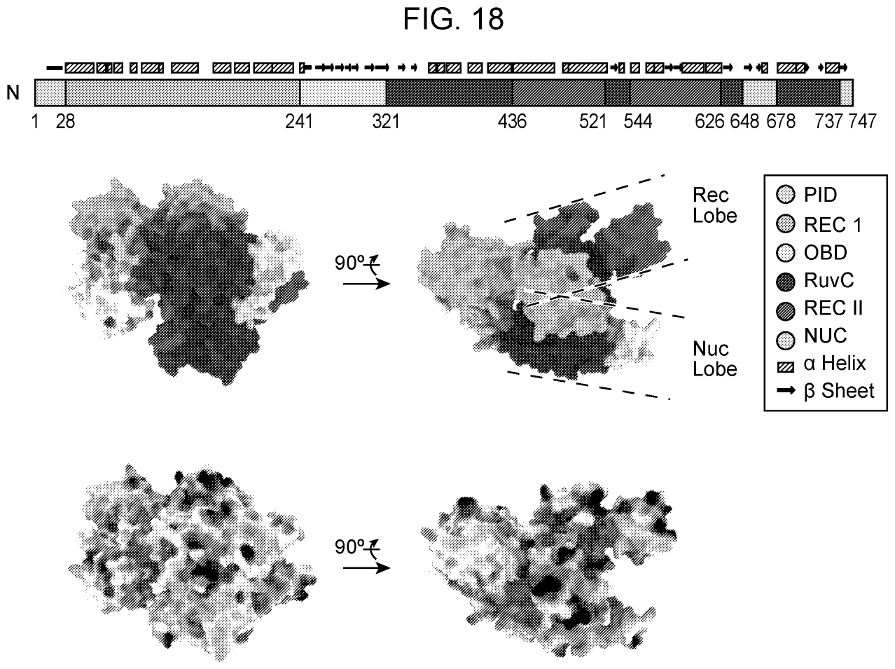

FIG. 18 depicts domain organization for Casλ1 unbound to the gRNA or DNA, and structure of the protein with charged residues that would interact with the gRNA and DNA in blue.

### Example 4: Genome Editing in Human and Plant Cells

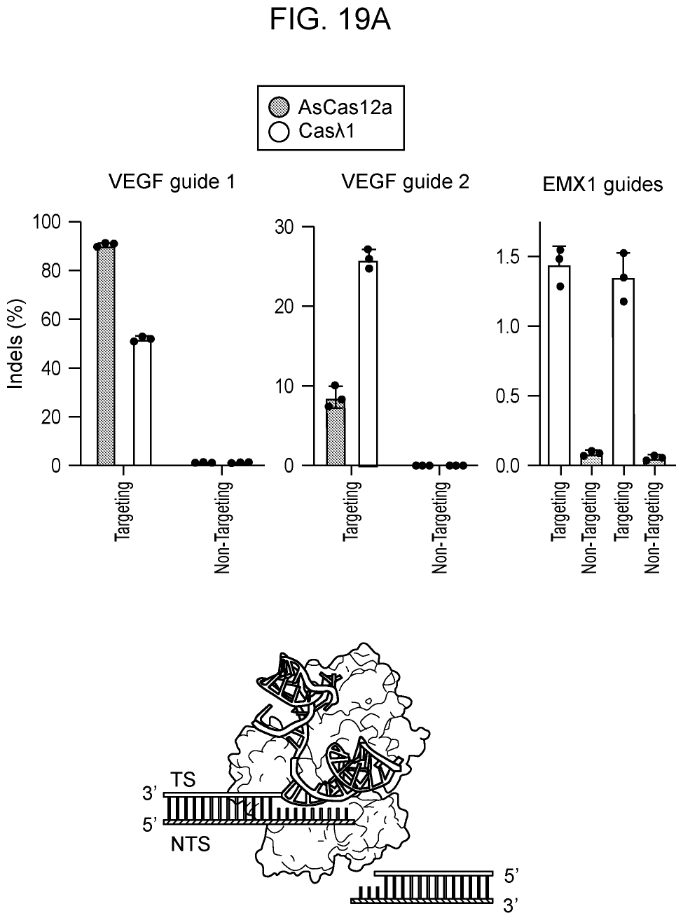

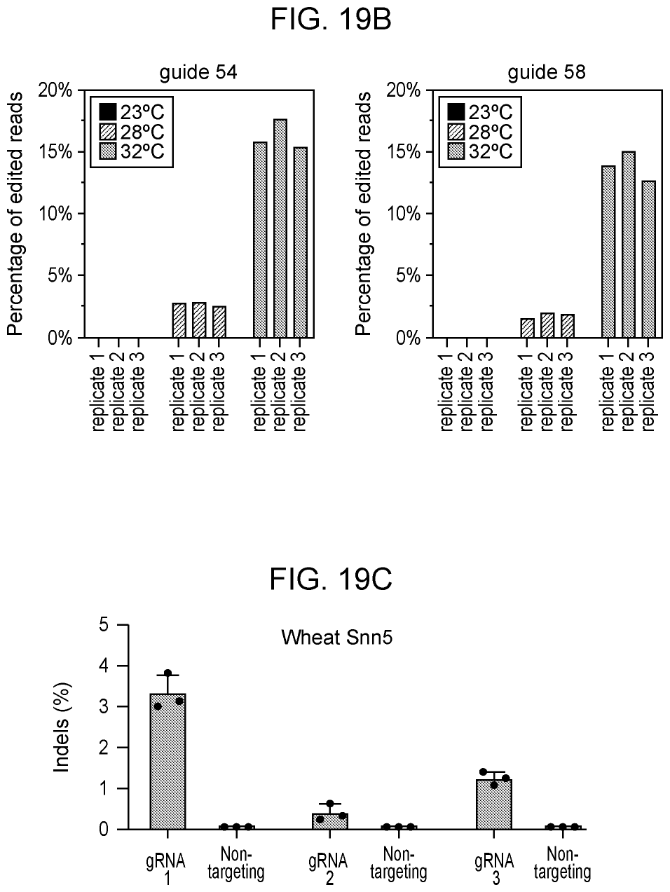

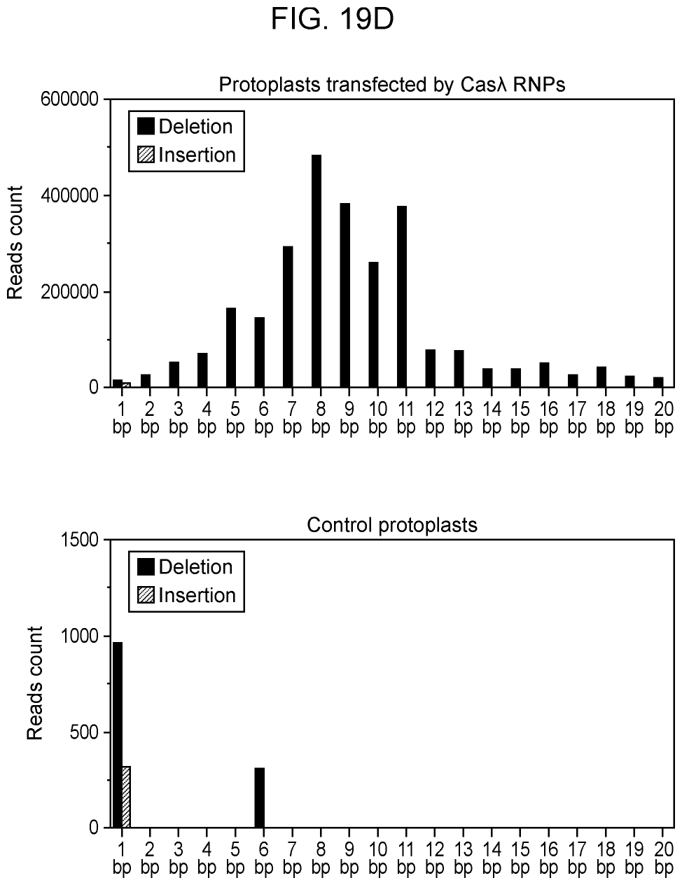

A head-tohead comparison of insertion and deletion efficiencies was conducted, using Casλ and Cas12a ribonucleoproteins (RNPs) with identical guide RNA spacers targeting sequences recognizing Vascular Endothelial Growth Factor A (VEGFA) and Empty Spiracles HomeoboxI (EMX1) genes in HEK293T cells. Despite their miniature size, Casλ RNPs generated promising genome-editing outcomes compared to Cas12a, and in at least one case, exceeded Cas12a indel percentages (FIG. 19A). Extending these experiments to Arabidopsis thaliana, it was confirmed that Casλ exhibited editing efficiencies of up to 18% at the endogenous PDS3 gene (FIG. 19B), notably higher than what was observed previously using CasΦ (Pausch et al. (2020) Science 369:333). The efficiency of editing was highly dependent on temperature, with no editing occurring at 23° C., an intermediate level of editing occurring at 28° C., and the highest level of editing occurring at 32° C. Furthermore, we were able to achieve editing in the endogenous disease resistance gene Snn5 in hexaploid wheat protoplasts, where a genome ˜5× larger than the human genome poses a scanning challenge to achieve successful editing at the target site (FIG. 19C). Next-generation sequencing for both mammalian and plant cells revealed indel profiles with large deletions (FIG. 19D), consistent with the staggered cuts observed in vitro at the PAM distal region.

FIG. 19A-19D. Casλ RNPs are functional for editing endogenous genes in human, Arabidopsis, and wheat cells with large deletion profiles. (A) Indel efficiency using Casλ and Cas12a RNPs with identical spacers targeting VEGF, and Casλ RNPs targeting EMX1 genes in HEK293T cells, and a schematic of the in vitro model of DNA cleavage outcomes following DNA cleavage by Casλ. (B) Indel efficiencies in Arabidopsis thaliana protoplasts show significantly higher levels of editing than previously achieved by CasΦ for the same PDS3 gene, and (C) in wheat protoplasts targeting the disease resistance gene Snn5. (D) Indel profiles generated by Casλ RNP administration show primarily large deletions, and little change without Casλ.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method for modifying a target nucleic acid in a eukaryotic cell, the method comprising contacting the target nucleic acid with:
a) a Cas12L polypeptide; and
b) a Cas12L guide nucleic acid,
whereby said contacting results in modification of the target nucleic acid, and wherein said contacting is carried out at a temperature of from about 28° C. to about 37° C.

2. The method of claim 1, wherein the Cas12L polypeptide comprises an amino acid sequence having at least 80% amino acid identity to the amino acid sequence depicted in any one of FIG. 4A-4Z, FIG. 4AA-4ZZ, and FIG. 4AAA-4FFF.

3. The method of any one of claims 1-2, wherein the Cas12L polypeptide is a fusion Cas12L polypeptide comprising one or more nuclear localization signals (NLS).

4. The method of claim 3, wherein at least one of the one or more nuclear localization signals is an SV40-type NLS.

5. The method of any one of claims 1-4, wherein the cell comprises one or more nucleic acids comprising a nucleotide sequence encoding the Cas12L polypeptide and/or a nucleotide sequence encoding the Cas12L guide nucleic acid.

6. The method of claim 5, wherein the nucleotide sequence encoding the Cas12L polypeptide and/or the nucleotide sequence encoding the Cas12L guide nucleic acid is operably linked to a transcriptional control element that is functional in the eukaryotic cell.

7. The method of claim 6, wherein the transcriptional control element is a promoter.

8. The method of claim 7, wherein the promoter is a UBQ10 promoter.

9. The method of claim 8, wherein the UBQ10 promoter comprises a nucleic acid sequence that is at least 80% identical to SEQ ID NO:85 or SEQ ID NO:72.

10. The method of any one of claims 5-9, wherein the nucleotide sequence encoding the Cas12L guide nucleic acid is operably linked to an RNA Polymerase II promoter.

11. The method of claim 10, wherein the RNA Polymerase II promoter is a CmYLCV promoter or a 2×35S promoter.

12. The method of claim 11, wherein the promoter comprises a nucleic acid sequence that is at least 80% identical to SEQ ID NO:87 or SEQ ID NO:88.

13. The method of claim 1, wherein the eukaryotic cell is maintained at a temperature in the range of about 28° C. to about 32° C.

14. The method of claim 1, further comprising maintaining the eukaryotic cell at a temperature of less than 28° C., optionally maintaining the plant cell at a temperature of from about 17° C. to about 25° C.

15. The method of claim 1, wherein the modification comprises a deletion of one or more nucleotides in the target nucleic acid.

16. The method of claim 15, wherein the deletion comprises deletion of 3-15 nucleotides in the target nucleic acid.

17. The method of claim 16, wherein the deletion comprises deletion of 9 nucleotides in the target nucleic acid.

18. The method of claim 1, wherein the target nucleic acid sequence is located in a region of repressive chromatin.

19. The method of claim 1, wherein the target nucleic acid sequence is located in a region of open chromatin.

20. The method of claim 1, wherein the guide nucleic acid is fused to a ribozyme.

21. The method of claim 1, wherein the eukaryotic cell comprises a genetic background that exhibits reduced susceptibility to transgene silencing.

22. The method of claim 1, comprising:
a) maintaining the eukaryotic cell for a first period of time at a first temperature of from about 17° C. to about 25° C., wherein the target nucleic acid is substantially not modified by the Cas12L polypeptide; and
b) maintaining the eukaryotic cell for a second period of time at a second temperature of from about 25° C. to about 37° C., wherein the target nucleic acid is modified by the Cas12L polypeptide.

23. The method of claim 1, wherein the eukaryotic cell is a plant cell.

24. The method of claim 23, wherein the target nucleic acid comprises a male reproductive pathway gene.

25. The method of claim 23, wherein the target nucleic acid comprises a nucleic acid in a pigment production pathway.

26. The method of claim 23, wherein the target nucleic acid provides for resistance to a disease caused by a fungus or a bacterium.

27. The method of claim 23, wherein the target nucleic acid provides for resistance to a plant pathogen.

28. The method of claim 23, wherein the target nucleic acid is a nucleic acid in the ethylene pathway.

29. The method of claim 1, wherein the eukaryotic cell is an insect cell, an arachnid cell, a mammalian cell, a fish cell, a fungal cell, a yeast cell, an amphibian cell, or an avian cell.


SEQUENCE LISTING (187 sequences)

SEQ ID NO: 1 - protein, 746 aa, synthetic construct
MASHKKTESNQIIKTFSFKIKNANGLSLDVLNDAITEYQNYYNICSDWIKDHLTMKISELYKYIPNEKKNSGYALTLISDEWKDKPMYMMFKKGYPANNRDNAIYETLNTCNTEHYTGNILNFSDTYYRRFGYVASAISNYVTKISKMSTGSRSKNISNDSDVDTIMEQVIYEMEHNGWTSVKDWENQMEYLESKTDSNPNFVYRMTTLYEFYKSHIDEVNSKMETMSIDSLIKFGGCRRKDSKKSMYIMGGSNTPFDITQIGGNSLNIKFSKNLNVDVFGRYDVIKDNTLLVDIINGHGASFVLKIINDEIYIDINVSVPFDKKIATTNKVVGIDVNIKHMLLATNILDDGNVKGYVNIYKEVINDSDFKKVCNSTVMQYFTDFSKFVTFCPLEFDFLFSRVCNQKGIYNDNSAMEKSFSDVLNKLKWNFIETGDNTKRIYIENVMKLRSQMKAYAIVKNAYYKQQSEYDFGKSEEFIQEHPFSNTDKGIEILNKLDNISKKILGCRNNIIQYSYNLFEINGYDMVSLEKLTSSQFKKKPFPTVNSLLKYHKILGCTQEEMEKKDIYSVIKKGYYDIIFDNDVVTDAKLSAKGELSKFKDDFFNLMIKSIHFADIKDYFITLSNNGTAGVSLVPSYFTSQMDSIDHKIYFVQDNKSGKLKLANKHKVRSSQEKHINGLNADYNAARNIAYIMENTDCRNMFMKQSRTDKSLYNKPSYETFIKTQGSAVAKLKKEGFVKILDEASV

SEQ ID NO: 2 - protein, 735 aa, synthetic construct
MAHKKNVGAEIVKTYSFKVKNTNGITMEKLMNAIDEFQSYYNLCSDWICKNLTTMTIGDLDQYIPEKAKGNTYATVLLDEAWKNQPLYKIFGKKYSSNNRNNALYCALSSVIDMTKENVLGFSKTHYIRNDYILNVISNYASKLSKLNTGVKSRAIKETSDEATIIEQVIYEMEHNKWESIEDWKNQIEYLNSKTDYNPTYMERMKTLSAYYSTHKSEVDAKMQEMAVENLVKFGGCRRNNSKKSMFIMGSNTTNYTISYIGGNSFNINFANILNFDVYGRRDVVKNGEVLVDIMANHGDSIVLKIVNGELYADVPCSVTLNKVESNFDKVVGIDVNMKHMLLSTSITDNGSSDFLNIYKEMSNNAEFMALCPEEDRKYYKDISKYVTFAPLELDLLFSRISKQGKVKMEKVYSEILEALKWKFFANGDNKNRIYVESIQKIRQQIKALCVIKNAYYEQQSAYDIDKTQEYIETHPFSLTEKGMSIKSKMDKICQTIIGCRNNIIDYAYSFFERNGYSIIGLEKLTSSQFEKTKSMPTCKSLLNFHKVLGHTLSELETLPINDVVKKGYYTFTTDNEGKITDASLSEKGKVRKMKDDFFNQAIKAIHFADVKDYFATLSNNGQTGIFFVPSQFTSQMDSNTHNLYFENAKNGGLKLAPKYKVRQTQEYHLNGLPADYNAARNIAYIGLDETMRNTFLKKANSNKSLYNQPIYDTGIKKTAGVFSRMKKLKRYEII

SEQ ID NO: 3 - protein, 778 aa, synthetic construct
MRISPHLFYIFFKKIWKCHFFVLSLYQLNQYIMASHEKTESNQIIKTFSFKIKNANGLSLDVLNDAITEYQNYYNICSDWIKDHLTMKIGELYKYIPDEKKNSGYALTLISDEWKDKPMYMMFKKGYPANNRDNAIYETLNTCNTEHYTGNILNFSDTYYRRFGYVASAISNYVTKISKMSTGSRSKNISNDSDVDTIMEQVIYEMEHNGWTSVKDWENQMEYLESKTDSNPNFVYRMTTLYEFYKSHIDEVNSKMETMSIDSLIKFGGCRRKDSKKSMYIMGGSNTPFDITQIGGNSLNIKFSKNLNVDVFGRYDVIKDNTLLVDIINGHGASFVLKIINGEIYIDINVSVPFDKKIATTNKVVGIDVNIKHMLLATNILDDGNVNGYVNIYKEVINDSDFKKVCNSTVMKYFTDFSKFVTFCPLEFDFLFSRVCNQKGIYNDNSAMEKSFSDVLNKLKWNFIETGDNTKRIYIENVMKLRSQMKAYAIVKNAYYKQQSEYDFGKSEEFIQEHPFSNTDKGIEILNKLDNISKKILGCRNNIIQYSYNLFEINGYDMISLEKLTSSQFKKKPFPTVNSLLKYHKILGCTQEEMEKKDIYSVIKKGYYDIIFDNDVVTDAKLSTKGELSKFKDDFFNLMIKSIHFADIKDYFITLSNNGTAGVSLVPSYFTSQMDSIDHKIYFVQDNKSGKLKLANKHKVRSSQEKHINGLNADYNAARNIAYIMENTECRNMFMKQSRTDKSLYNKPSYETFIKTQGSAVAKLKKEGFVKILDEASV

SEQ ID NO: 4 - protein, 778 aa, synthetic construct
MRISPHLFYIFFKKIWKSHFFVLSLYQLNQYIMASHKKTESNQIIKTFSFKIKNANGLSLDVLNDAITEYQNYYNICSDWIKDHLTMKIGELYKYIPDEKKNSGYALTLISDEWKDKPMYMMFKKGYPANNRDNAIYETLNTCNTEHYTGNILNFPDTYYRRFGYVASTISNYVTKISKMSTGSRSKNISNDSDVDTIMEQVIYEMEHNGWTSVKDWENQMEYLESKTDSNPNFVYRMTTLYEFYKSHIDEVNSKMETMSIDSLIKFGGCRRKDSKKSMYIMGGSNTPFDITQIGGNSLNIKFSKNLNVDVFGRYDVIKDNTLLVDIINGHGASFVLKIINGEIYIDINVSVPFDKKIATTNKVVGIDVNIKHMLLATNILDDGNVNGYVNIYKEVINDSDFKKVCNSTVMKYFTDFSKFVTFCPLEFDFLFSRVCNQKGIYNDNSAMEKSFSDVLNKLKWNFIETGDNTKRIYIENVMKLRSQMKAYAIVKNAYYKQQSEYDFGKSEEFIQEHPFSNTDKGIEILNKLDNISKKILGCRNNIIQYSYNLFEINGYDMISLEKLTSSQFKKNPFPTVNSLLKYHKILGCTQEEMEKKDIYSVIKKGYYDIIFDNDVVTDAKLSTKGELSKFKDDFFNLMIKSIHFADIKDYFITLSNNGTAGVSLVPSYFTSQMDSIDHKIYFVQDNKSGKLKLANKHKVRSSQEKHINGLNADYNAARNIAYIMENTECRNMFMKQSRTDKSLYNKPSYETFIKTQGSAVAKLKKEGFVKILDEASV

SEQ ID NO: 5 - protein, 778 aa, synthetic construct
MRISPHLFYIFFKKIWKCHIFVLSLYQLNQYIMASHKKTESNQIIKTFSFKIKNANGLSLDVLNDAITEYQNYYNICSDWIKDHLTMKIGELYKYIPDEKKNSGYALTLISDEWKDKPMYMMFKKGYPANNRDNAIYETLNTCNTEHYTGNILNFSDTYYRRFGYVASTISNYVTKISKMSTGSRSKNISNDSDVDTIMEQVIYEMEHNGWTSVKDWENQMEYLESKTDSNPNFVYRMTTLYEFYKSHIDEVNSKMETMSIDSLIKFGGCRRKDSKKSMYIMGGSNTPFDITQIGGNSLNIKFSKNLNVDVFGRYDVIKDNTLLVDIINGHGASFVLKIINGEIYIDINVSVPFDKKIATTNKVVGVDVNIKHMLLATNILDDGNVNGYVNIYKEVINDSDFKKVCNSTVMKYFTDFSKFVTFCPLEFDFLFSRVCNQKGIYNDNSAMEKSFSDVLNKLKWNFIETGDNTKRIYIENVMKLRSQMKAYAIVKNAYYKQQSEYDFGKSEEFIQEHPFSNTDKGIEILNKLDNISKKILGCRNNIIQYSYNLFEINGYDMISLEKLTSSQFKKKPFPTVNSLLKYHKILGCTQEEMEKKDIYSVIKKGYYDIIFDNDVVTDAKLSTKGELSKFKDDFFNLMIKSIHFADIKDYFITLSNNGTAGVSLVPSYFTSQMDSIDHKIYFVQDNKSGKLKLANKHKVRSSQEKHINGLNADYNAARNIAYIMENTDCRNMFMKQSRTDKSLYNKPSYETFIKTQGSAVAKLKKEGFVKILDEASV

SEQ ID NO: 6 - protein, 351 aa, synthetic construct
QYVTFAPLELDLLFSRISKQGKVKMEKAYSEILEALKWKFFANGDNKNRIYVESIQKIRQQIKALCVIKNAYYEQQSAYDIDKTQEYIETHPFSLTEKGMSIKSKMDKICQTIIGCRNNIIDYAYSFFERNGYTIIGLEKLTSSQFEKTKSMPTCKSLLNFHKVLGHTLSELETLPINDVVKKGYYAFTTDNEGRITDASLSEKGKVRKMKDDFFNQAIKAIHFADVKDYFATLSNNGQTGIFFVPSQFTSQMDSNTHNLYFENAKNGGLKLASKSKVRKSQEYHLNGLPADYNAARNIAYIGLDEIMRNTFLKKANSNKSLYNQPIYDTGIKKTAGVFSRMKKLKKYKVI

SEQ ID NO: 7 - protein, 717 aa, synthetic construct
VLNDAITEYQNYYNICSDWIKDHLTMKIGELYKYIPDEKKNSGYALTLISDEWKDKPMYMMFKKGYPANSRDNAIYEALNTCNTEHYTGNILNFSDTYYRRFGYVASAISNYVTKISKMSTGSRSKNISNDSDVDTIMEQVIYEMEHNGWTSVKDWENQMEYLESKTDSNPNFVYRMTTLYEFYKSHIDEVNSKMETMSIDSLIKFGGCRRKDSKKSMYIMGGSNTPFDITQIGGNSLNIKFSKNLNVDVFGRYDVIKDNTLLVDIINGHGASFVLKIINDEIYIDINVSVPFDKKIATTNKVVGIDVNIKHMLLATNILDDGNVNGYVNIYKEVINDSDFKKVCNSTVMKYFTDFSKFVTFCPLEFDFLFSRVCNQKGIYNDNSAMEKSFSNVLNKLKWNFIETGDNTKRIYIENVMKLRSQMKAYAIVKNAYYKQQSEYDFGKSEEFIQEHPFSNTDKGIEILHKLDNISKKILGCRNNIIQYSYNLFEINGYDMISLEKLTSSQFKKKPFPTVNSLLKYHKILGCTQEEMEKKDIYSVIKKGYYDIIFDNDVVTDAKLSTKGELSKFKDDFFNLMIKSIHFADIKDYFITLSNNGTAGVSLVPSYFTSQMDSIDHKIYFVQDNKSGKLKLANKHKVRSSQEKHINGLNADYNAARNIAYIMENTDCRNMFMKQSRTDKSLYNKPSYETFIKTQGSAVAKLKKEGFMKILDEASV

SEQ ID NO: 8 - protein, 742 aa, synthetic construct
IFFKKIWKCHIFVLSLYQLNQYIMASHKKTESNQIIKTFSFKIKNANGLSLDVLNDAITEYQNYYNICSDWIKDHLTMKIGELYKYIPDEKKNSGYALTLISDEWKDKPMYMMFKKGYPANSRDNAIYEALNTCNTEHYTGNILNFSDTYYRRFGYVASAISNYVTKISKMSTGSRSKNISNDSDVDTIMEQVIYEMEHNGWTSVKDWENQMEYLESKTDSNPNFVYRMTTLYEFYKSHIDEVNSKMETMSIDSLIKFGGCRRKDSKKSMYIMGGSNTPFDITQIDGNSLNIKFSKNLNVDVFGRYDVIKDNTLLVDIINGHGASFVLKIINDEIYIDINVSVPFDKKIATTNKVVGVDVNIKHMLLATNILDDGNVNGYVNIYKEVINDSDFKKVCNSTVMKYFTDFSKFVTFCPLEFDFLFSRVCNQKGIYNDNSAMEKSFSDVLNKLKWNFIETGDNTKRIYIENVMKLRSQMKAYAIVKNAYYKQQSEYDFGKSEEFIQEHPFSNTDKGIEILHKLDNISKKILGCRNNIIQYSYNLFEINGYDMISLEKLTSSQFKKKPFPTVNSLLKYHKILGCTQEEMEKKDIYSVIKKGYYDIIFDNDVVTDAKLSTKGELSKFKDDFFNLMIKSIHFADIKDYFITLSNNGTAGVSLVPSYFTSQMDSIDHKIYFVQDNKSGKLKLANKHKVRSSQEKHINGLNADYNAARNIAYIMENTEQGSAVAKLKKEGFVKILDEASV

SEQ ID NO: 9 - protein, 746 aa, synthetic construct
MASHKKTESNQIIKTFSFKIKNANGLSLDVLNDAITEYQNYYNICSDWIKDHLTMKIGELYKYIPDEKKNSGYALTLISDEWKDKPMYMMFKKGYPANNRDNAIYETLNTCNTEHYTGNILNFSDTYYRRFGYVASAISNYVTKISKMSTGSRSKNISNDSDVDTIMEQVIYEMEHNGWTSVKDWENQMEYLESKTDSNPNFVYRMTTLYEFYKSHIDEVNSKMETMSIDSLIKFGGCRRKDSKKSMYIMGGSNTPFDITQIGGNSLNIKFSKNLNVDVFGRYDVIKDNTLLVDIINGHGASFVLKIINGEIYIDINVSVPFDKKIATTNKVVGVDVNIKHMLLATNILDDGNVNGYVNIYKEVINDSDFKKVCNSTVMKYFTDFSKFVTFCPLEFDFLFSRVCNQKGIYNDNSAMEKSFSDVLNKLKWNFIETGDNTKRIYIENVMKLRSQMKAYAIVKNAYYKQQSEYDFGKSEEFIQEHPFSNTDKGIEILNKLDNISKKILGCRNNIIQYSYNLFEINGYDMISLEKLTSSQFKKKPFPTVNSLLKYHKILGCTQEEMEKKDIYSVIKKGYYDIIFDNDVVTDAKLSAKGELSKFKDDFFNLMIKSIHFADIKDYFITLSNNGTAGVSLVPSYFTSQMDSIDHKIYFVQDNKSGKLKLANKHKVRSSQEKHINGLNADYNAARNIAYIMENTECRNMFMKQSRTDKSLYNKPSYETFIKTQGSAVSKLKKDGFVKILDEASV

SEQ ID NO: 10 - protein, 782 aa, synthetic construct
MMKKMRTNPHLFYICFKKIWKCHFFALSLYQLNQYIMASHKKTESNQIIKTFSFKIKNANGLSLDVLNDAITEYQNYYNICSDWIKDHLTMKIGELYKYIPDEKKNSGYALTLISDEWKDKPMYMMFKKGYPANSRDNAIYEALNTCNTEHYTGNILNFSDTYYRRFGYVASTISNYVTKISKMSTGSRSKNISNDSDVDTIMEQVIYEMEHNGWTSVKDWENQMEYLESKTDSNPNFVYRMTTLYEFYKSHIDEVNSKMETMSIDSLIKFGGCRRKDSKKSMYIMGGSNTPFDITQIGGNSLNIKFSKNLNVDVFGRYDVIKDNTLLVDIINEHGASFVLKIINDEIYIDINVSVPFDKKIATTNKVVGVDVNIKHMLLATNILDDGNVNGYVNIYKEVINDSDFKKVCNSTVMKYFTDFSKFVTFCPLEFDFLFSRVCNQKGIYNDNSAMEKSFSDVLNKLKWNFIETGDNTKRIYIENVMKLRSQMKAYAIVKNAYYKQQSEYDFGKSEEFIQEHPFSNTDKGIEILNKLDNISKKILGCRNNIIQYSYNLFEINGYDMISLEKLTSSQFKKKPFPTVNSLLKYHKILGCTQEEMEKKDIYSVIKKGYYDIIFDNDVVTDAKLSTKGELSKFKDDFFNLMIKSIHFADIKDYFITLSNNGTAGVSLVPSYFTSQMDSIDHKIYFVQDNKSGKLKLANKHKVRSSQEKHINGLNADYNAARNIAYIMENTECRNMFMKQSRTDKSLYNKPSYETFIKTQGSAVAKLKKEGFVKILDEASA

SEQ ID NO: 11 - protein, 92 aa, synthetic construct
NKSGKLKLANKHKVRSSQEKHINGLNADYNAARNIAYIMENTECRNMFMKQSRTDKSLYNKPSYETFIKTQGSAVAKLKKEGFVKILDEASV

SEQ ID NO: 12 - protein, 746 aa, synthetic construct
MASHKKTESNQIIKTFSFKIKNANGLSLDVLNDAITEYQNYYNICSDWIKDHLTMKIGELYKYIPDEKKNSGYALTLISDEWKDKPMYMMFKKGYPANSRDNAIYEALNTCNTEHYTGNILNFSDTYYRRFGYVASTISNYVTKISKMSTGSRSKNISNDSDVDTIMEQVIYEMEHNGWTSVKDWENQMEYLESKTDSNPNFVYRMTTLYEFYKSHIDEVNSKMETMSIDSLIKFGGCRRKDSKKSMYIMGGSNTPFDITQIGGNSLNIKFSKNLNVDVFGRYDVIKDNTLLVDIINGHGASFVLKIINGEIYIDINVSVPFDKKIATTNKVVGVDVNIKHMLLATNILDDGNVNGYVNIYKEVINDSDFKKVCNSTVMKYFTDFSKFVTFCPLEFDFLFSRVCNQKGIYNDNSAMEKSFSDVLNKLKWNFIETGDNTKRIYIENVMKLRSQMKAYAIVKNAYYKQQSEYDFGKSEEFIQEHPFSNTDKGIEILNKLDNISKKILGCRNNIIQYSYNLFEINGYDMISLEKLTSSQFEKKPFPTVNSLLKYHKILGCTQEEMEKKDIYSVIKKGYYDIIFDNDVVTDAKLSTKGELSKFKDDFFNLMIKSIHFADIKDYFITLSNNGTAGVSLVPSYFTSQMDSIDHKIYFVQDNKSGKLKLANKHKVRSSQEKHINGLNADYNAARNIAYIMENTDCRNMFMKQSRTDKSLYNKPSYETFIKTQGSAVAKLKKEGFVKILDEASV

SEQ ID NO: 13 - protein, 427 aa, synthetic construct
VPFDKKIATTNKVVGVDVNIKHMLLATNILDDGNVNGYVNIYKEVINDSDFKKVCNSTVMKYFTDFSKFVTFCPLEFDFLFSRVCNQKGIYNDNSAMEKSFSDVLNKLKWNFIETGDNTKRIYIENVMKLRSQMKAYAIVKNAYYKQQSEYDFGKSEEFIQEHPFSNTDKGIEILNKLDNISKKILGCRNNIIQYSYNLFEINGYDMISLEKLTSSQFKKKSFPTVDSLLKYHKILGCTQEEMEKKDIYSVIKKGYYDIIFDNDVVTDAKLSTKGELSKFKDDFFNLMIKSIHFADIKDYFITLSNNGTAGVSLVPSYFTSQMDSIDHKIYFVQDNKSGKLKLANKHKVRSSQEKHINGLNADYNAARNIAYIMENTDCRNMFMKQSRTDKSLYNKPSYETFIKTQGSAVAKLKKEGFVKILDEASV

SEQ ID NO: 14 - protein, 746 aa, synthetic construct
MASHKKTESNQIIKTFSFKIKNANGLSLDVLNDAITEYQNYYNICSDWIKDHLTMKIGELYKYIPDEKKNSGYALTLISDEWKDKPMYMMFKKGYPANNRDNAIYETLNTCNTEHYTGNILNFSDTYYRRFGYVASAISNYVTKISKMSTGSRSKNISNDSDVDTIMEQVIYEMEHNGWTSVKDWENQMEYLESKTDSNPNFVYRMTTLYEFYKSHIDEVNSKMETMSIDSLIKFGGCRRKDSKKSMYIMGGSNTPFDITQIGGNSLNIKFSKNLNVDVFGRYDVIKDNTLLVDIINGHGASFVLKIINGEIYIDINVSVPFDKKIATTNKVVGVDVNIKHMLLATNILDDGNVNGYVNIYKEVINDSDFKKVCNSTVMKYFTDFSKFVTFCPLEFDFLFSRVCNQKGIYNDNSAMEKSFSDVLNKLKWNFIETGDNTKRIYIENVMKLRSQMKAYAIVKNAYYKQQSEYDFGKSEEFIQEHPFSNTDKGIEILNKLDNISKKILGCRNNIIQYSYNLFEINGYDMISLEKLTSSQFKKKPFPTVNSLLKYHKILGCTQEEMEKKDIYSVIKKGYYDIIFDNDVVTDAKLSTKGELSKFKDDFFNLMIKSIHFADIKDYFITLSNNGTAGVSLVPSYFTSQMDSIDHKIYFVQDNKSGKLKLANKHKVRSSQEKHINGLNADYNAARNIAYIMENTECRNMFMKQSRTDKSLYNKPSYETFIKTQGSAVAKLKKEGFVKILDEASV

SEQ ID NO: 15 - protein, 735 aa, synthetic construct
MAHKKNIGAEIVKTYSFKVKNTNGITMEKLMNAIDEYQSYYNLCSDWICKNLTTMTIGDLDRYIPEKAKDNIYATVLLDEVWKNQPLYKIFGKKYSSNNRINALYCTLSSVIDINKKNILGLSQTYYARNGYILNVISNYASKLSKLNTGVKRHTIKETSDEATIVEQVIYEMEHNKWESIEDWKNQIEYLNSKTDYNPTYMERMKTLSAYYSEHKSEVDAKMQEMAVENLVKFGGCRRNNSKKSMFIMGSSKTTYTISYIGDNCFNINFANILNFDVYGRRDVVKNGEVLVDIMANHGDSIVLKIVNGELYADVPCSTTLNKVESTFDKVAGIDVNMKHMLLSTSVTDNGNSDFVNIYKEMSNNAEFMALCPEEDRKYYKDISQYVTFAPLELDLLFSRISKQGKVKMEKAYSEILEALKWKFFANGDNKNRIYVENIQKIRQQIKALCVIKNAYYEQQSAYDIDKTQEYIEAHPFSLTEKGMSIKSKMDNICRTIIGCRNNIIDYAYSFFERNDYSIIGLEKLTSSQFEKTKSLPTCKSLLNFHKVLGHTLSELETLPINDVVKKGYYTFTTDNEGRITDASLSEKGKVRKMKDDFFNQAIKAIHFADVKDYFATLSNNGQTGIFFVPSQFTSQMDSNTHTLYFENAKNGGLKLASKYKVRKSQEYHLNGLPADYNAARNIAYIGLDEIMRNTFLKKANSNKSLYNQPIYDTGIKKTAGVFSRMKKLKKYKVI

SEQ ID NO: 16 - protein, 746 aa, synthetic construct
MASHKKTESNQIIKTFSFKIKNANGLSLDVLNDAITEYQNYYNICSDWIKDHLTMKIGELYKYIPDEKKNSGYALTLISDEWKDKPMYMMFKKGYPANSRDNAIYEALNTCNTEHYTGNILNFSDTYYRRFGYVASTISNYVTKISKMSTGSRSKNISNDSDVDTIMEQVIYEMEHNGWTSVKDWENQMEYLESKTDSNPNFVYRMTTLYEFYKSHIDEVNSKMETMSIDSLIKFGGCRRKDSKKSMYIMGGSNTPFDITQIGGNSLNIKFSKNLNVDVFGRYDVIKDNTLLVDIINGHGASFVLKIINGEIYIDINVSVPFDKKIATTNKVVGVDVNIKHMLLATNILDDGNVNGYVNIYKEVINDSDFKKVCNSTVMQYFTDFSKFVTFCPLEFDFLFSRVCNQKGIYNDNSAMEKSFSDVLNKLKWNFIETGDNTKRIYIENVMKLRSQMKAYAIVKNAYYKQQSEYDFGKSEEFIQEHPFSNTDKGIEILNKLDNISKKILGCRNNIIQYSYNLFEINGYDMISLEKLTSSQFKKKPFPTVNSLLKYHKILGCTQEEMEKKDIYSVIKKGYYDIIFDNDVVTDAKLSTKGELSKFKDDFFNLMIKSIHFADIKDYFITLSNNGTAGVSLVPSYFTSQMDSIDHKIYFVQDNKSGKLKLANKHKVRSSQEKHINGLNADYNAARNIAYIMENTECRNMFMKQSRTDKSLYNKPSYETFIKTQGSAVAKLKKEGFVKIIDEASV

SEQ ID NO: 17 - protein, 746 aa, synthetic construct
MASHKKTESNQIIKTFSFKIKNANGLSLDVLNDAITEYQNYYNICSDWIKDHLTMKIGELYKYIPDEKKNSGYALTLISDEWKDKPMYMMFKKGYPANNRDNAIYEALNTCNTEHYTGNILNFSDTYYRRFGYVASTISNYVTKISKMSTGSRSKNISNDSDVDTIMEQVIYEMEHNGWTSVKDWENQMEYLESKTDSNPNFVYRMTTLYEFYKSHIDEVNSKMETMSIDSLIKFGGCRRKDSKKSMYIMGGSNTPFDITQIGGNSLNIKFSKNLNVDVFGRYDVIKDNTLLVDIINGHGASFVLKIINGEIYIDINVSVPFDKKIATTNKVVGVDVNIKHMLLATNILDDGNVNGYVNIYKEVINDSDFKKVCNSTVMKYFTDFSKFVTFCPLEFDFLFSRVCNQKGIYNDNSAMEKSFSDVLNKLKWNFIETGDNTKRIYIENVMKLRSQMKAYAIVKNAYYKQQSEYDFGKSEEFIQEHPFSNTDKGIEILNKLDNISKKILGCRNNIIQYSYNLFEINGYDMISLEKLTSSQFKKKPFPTVNSLLKYHKILGCTQEEMEKKDIYSVIKKGYYDIIFDNGVVTDAKLSTKGELSKFKDDFFNLMIKSIHFADIKDYFITLSNNGTAGVSLVPSYFTSQMDSIDHKIYFVQDNKSGKLKLANKHKVRSSQEKHINGLNADYNAARNIAYIMENTECRNMFMKQSRTDKSLYNKPSYETFIKTQGSAVAKLKKEGFVKILDEASV

SEQ ID NO: 18 - protein, 680 aa, synthetic construct
EKKNSGYALTLISDEWKDKPMYMMFKKGYPANNRDNAIYETLNTCNTEHYTGNILNFSDTYYRRFGYVASAISNYVTKISKMSTGSRSKNISNDSDVDTIMEQVIYEMEHNGWTSVKDWENQMEYLESKTDSNPNFVYRMTTLYEFYKSHIDEVNSKMETMSIDSLIKFGGCRRKDSKKSMYIMGGSNTPFDITQIGGNSLNIKFSKNLNVDVFGRYDVIKDNTLLVDIINEHGASFVLKIINDEIYIDINVSVPFDKKIATTNKVVGVDVNIKHMLLATNILDDGNVNGYVNIYKEVINDSDFKKVCNSTVMKYFTDFSKFVTFCPLEFDFLFSRVCNQKGIYNDNSAMEKSFSDVLNKLKWNFIETGDNTKRIYIENVMKLRSQMKAYAIVKNAYYKQQSEYDFGKSEEFIQEHPFSNTDKGIEILNKLDNISKKILGCRNNIIQYSYNLFEINGYDMISLEKLTSSQFKKKPFPTVNSLLKYHKILGCTQEEMEKKDIYSVIKKGYYDIIFDNDVVTDAKLSTKGELSKFKDDFFNLMIKSIHFADIKDYFITLSNNGTVGVSLVPSYFTSQMDSIDHKIYFVQDNKSGKLKLANKHKVRSSQEKHINGLNADYNAARNIAYIMENNECRNMFMKQSRTDKSLYNKPSYETFIKTQGSAVAKLKKEGFVKILDEASV

SEQ ID NO: 19 - protein, 516 aa, synthetic construct
DAKMQEMAVENLVKFGGCRRNNSKKSMFIMGSSKTTYTISYIGDNCFNINFANILNFDVYGRRDVVKNGEVLVDIMANHGDSIVLKIVNGELYADVPCSTTLNKVESTFDKVAGIDVNMKHMLLSTSVTDNGNSDFVNIYKEISNNAEFMALCPEEDRKYYKDISQYVTFAPLELDLLFSRISKQGKVKMEKAYSEILETLKWKFFANGDNKNRIYVESIQKIRQQIKALCVIKNAYYEQQSAYDIDKTQEYIEAHPFSLTEKGMSIKSKMDNICRTIIGCRNNIIDYAYSFFERNDYSIIGLEKLTSSQFEKTKSLPTCKSLLNFHKVLGHTLSELETLPINDVVKKGYYTFTTDNEGRITDASLSEKGKVRKMKDDFFNQAIKAIHFADVKDYFATLSNNGQTGIFFVPSQFTSQMDSNTHTLYFENAKNGGLKLASKYQVRQTQEYHLNGLPADYNAARNIAYIGLDETMRNTFLKKANSNKSLYNQPIYDTGIKKTAGVFSRMKKLKRYEII

SEQ ID NO: 20 - protein, 585 aa, synthetic construct
DVDTIMEQVIYEMEHNGWTSVKDWENQMEYLESKTDSNPNFVYRMTTLYEFYKSHIDEVNSKMETMSIDSLIKFGGCRRKDSKKSMYIMGGSNTPFDITQIGGNSLNIKFSKNLNVDVFGRYDVIKDNTLLVDIINGHGASFVLKIINGEIYIDINVSVPFDKKIATTNKVVGVDVNIKHMLLATNILDDGNVNGYVNIYKEVINDSDFKKVCNSTVMKYFTDFSKFVTFCPLEFDFLFSRVCNQKGIYNDNSAMEKSFSDVLNKLKWNFIETGDNTKRIYIENVMKLRSQMKAYAIVKNAYYKQQSEYDFGKSEEFIQEHPFSNTDKGIEILNKLDNISKKILGCRNNIIQYSYNLFEINGYDMISLEKLTSSQFKKKPFPTVNSLLKYHKILGCTQEEMEKKDIYSVIKKGYYDIIFDNDVVTDAKLSTKGELSKFKDDFFNLMIKSIHFADIKDYFITLSNNGTAGVSLVPSYFTSQMDSIDHKIYFVQDNKSGKLKLANKHKVRSSQEKHINGLNADYNAARNIAYIMENTECRNMFMKQSRTDKSLYNKPSYETFIKTQGSAVAKLKKEGFVKIIDEASV

SEQ ID NO: 21 - protein, 746 aa, synthetic construct
MASHKKTESNQIIKTFSFKIKNANGLSLDVLNDAITEYQNYYNICSDWIKDHLTMKIGELYKYIPDEKKNSGYALTLISDEWKDKPMYMMFKKGYPANSRDNAIYEALNTCNTEHYTGNILNFSDTYYRRFGYVASAISNYVTKISKMSTGSRSKNISNDSDVDTIMEQVIYEMEHNGWTSVKDWENQMEYLESKTDSNPNFVYRMTTLYEFYKSHIDEVNSKMETMSIDSLIKFGGCRRKDSKKSMYIMGGSNTPFDITQIGGNSLNIKFSKNLNVDVFGRYDVIKDNTLLVDIINEHGASFVLKIINDEIYIDINVSVPFDKKIATTNKVVGVDVNIKHMLLATNILDDGNVNGYVNIYKEVINDSDFKKVCNSTVMKYFTDFSKFVTFCPLEFDFLFSRVCNQKGIYNDNSAMEKSFSDVLNKLKWNFIETGDNTKRIYIENVMKLRSQMKAYAIVKNAYYKQQSEYDFGKSEEFIQEHPFSNTDKGIEILNKLDNISKKILGCRNNIIQYSYNLFEINGYDMISLEKLTSSQFEKKPFPTVNSLLKYHKILGCTQEEMEKKDIYSVIKKGYYDIIFDNDVVTDAKLSTKGELSKFKDDFFNLMIKSIHFADIKDYFITLSNNGTAGVSLVPSYFTSQMDSIDHKIYFVQDNKSGKLKLANKHKVRSSQEKHINGLNADYNAARNIAYIMENTECRNMFMKQSRTDKSLYNKPSYETFIKTQGSAVAKLKKEGFVKILDEASV

SEQ ID NO: 22 - protein, 585 aa, synthetic construct
DVDTIMEQVIYEMEHNGWTSVKDWENQMEYLESKTDSNPNFVYRMTTLYEFYKSHIDEVNSKMETMSIDSLIKFGGCRRKDSKKSMYIMGGSNTPFDITQIGGNSLNIKFSKNLNVDVFGRYDVIKDNTLLVDIINEHGASFVLKIINDEIYIDINVSVPFDKKIATTNKVVGVDVNIKHMLLATNILDDGNVNGYVNIYKEVINDSDFKKVCNSTVMKYFTDFSKFVTFCPLEFDFLFSRVCNQKGIYNDNSAMEKSFSDVLNKLKWNFIETGDNTKRIYIENVMKLRSQMKAYAIVKNAYYKQQSEYDFGKSEEFIQEHPFSNTDKGIEILNKLDNISKKILGCRNNIIQYSYNLFEINGYDMISLEKLTSSQFKKKPFPTVDSLLKYHKILGCTQEEMEKKDIYSVIKKGYYDIIFDNDVVTDAKLSTKGELSKFKDDFFNLMIKSIHFADIKDYFITLSNNGTAGVSLVPSYFTSQMDSIDHKIYFVQDNKSGKLKLANKHKVRSSQEKHINGLNADYNAARNIAYIMENTDCRNMFMKQSRTDKSLYNKPSYETFIKTQGSAVAKLKKEGFVKILDEASV

SEQ ID NO: 23 - protein, 758 aa, synthetic construct
FVLSLYQLNQYIMASHKKTESNQIIKTFSFKIKNANGLSLDVLNDAITEYQNYYNICSDWIKDHLTMKIGELYKYIPDEKKNSGYALTLISDEWKDKPMYMMFKKGYPANNRDNAIYETLNTCNTEHYTGNILNFSDTYYRRFGYVASTISNYVTKISKMSTGSRSKNISNDSDVDTIMEQVIYEMEHNGWTSVKDWENQMEYLESKTDSNPNFVYRMTTLYEFYKSHIDEVNSKMETMSIDSLIKFGGCRRKDSKKSMYIMGGSNTPFDITQIGGNSLNIKFSKNLNVDVFGRYDVIKDNTLLVDIINGHGASFVLKIINGEIYIDINVSVPFDKKIATTNKVVGVDVNIKHMLLATNILDDGNVNGYVNIYKEVINDSDFKKVCNSTVMKYFTDFSKFVTFCPLEFDFLFSRVCNQKGIYNDNSAMEKSFSDVLNKLKWNFIETGDNTKRIYIENVMKLRSQMKAYAIVKNAYYKQQSEYDFGKSEEFIQEHPFSNTDKGIEILNKLDNISKKILGCRNNIIQYSYNLFEINGYDMISLEKLTSSQFKKKSFPTVDSLLKYHKILGCTQEEMEKKDIYSVIKKGYYDIIFDNDVVTDAKLSTKGELSKFKDDFFNLMIKSIHFADIKDYFITLSNNGTAGVSLVPSYFTSQMDSIDHKIYFVQDNKSGKLKLANKHKVRSSQEKHINGLNADYNAARNIAYIMENNECRNMFMKQSRTDKSLYNKPSYETFIKTQGSAVAKLKKEGFVKILDEASV

SEQ ID NO: 24 - protein, 596 aa, synthetic construct
GSRSKNISNDSDVDTIMEQVIYEMEHNGWTSVKDWENQMEYLESKTDSNPNFVYRMTTLYEFYKSHIDEVNSKMETMSIDSLIKFGGCRRKDSKKSMYIMGGSNTPFDITQIGGNSLNIKFSKNLNVDVFGRYDVIKDNTLLVDIINGHGASFVLKIINGEIYIDINVSVPFDKKIATTNKVVGVDVNIKHMLLATNILDDGNVNGYVNIYKEVINDSDFKKVCNSTVMKYFTDFSKFVTFCPLEFDFLFSRVCNQKGIYNDNSAMEKSFSDVLNKLKWNFIETGDNTKRIYIENVMKLRSQMKAYAIVKNAYYKQQSEYDFGKSEEFIQEHPFSNTDKGIEILNKLDNISKKILGCRNNIIQYSYNLFEINGYDMISLEKLTSSQFKKKSFPTVDSLLKYHKILGCTQEEMEKKDIYSVIKKGYYDIIFDNDVVTDAKLSTKGELSKFKDDFFNLMIKSIHFADIKDYFITLSNNGTAGVSLVPSYFTSQMDSIDHKIYFVQDNKSGKLKLANKHKVRSSQEKHINGLNADYNAARNIAYIMENNECRNMFMKQSRTDKSLYNKPSYETFIKTQGSAVAKLKKEGFVKILDEASV

SEQ ID NO: 25 - protein, 640 aa, synthetic construct
MASHKKTESNQIIKTFSFKIKNANGLSLDVLNDAITEYQNYYNICSDWIKDHLTMKIGELYKYIPDEKKNSGYALTLISDEWKDKPMYMMFKKGYPANNRDNAIYETLNTCNTEHYTGNILNFSDTYYRRFGYVASTISNYVTKISKMSTGSRSKNISNDSDVDTIMEQVIYEMEHNGWTSVKDWENQMEYLESKTDSNPNFVYRMTTLYEFYKSHIDEVNSKMETMSIDSLIKFGGCRRKDSKKSMYIMGGSNTPFDITQIDGNSLNIKFSKNLNVDVFGRYDVIKDNTLLVDIINGHGASFVLKIINGEIYIDINVSVPFDKKIATTNKVVGVDVNIKHMLLATNILDDGNVNGYVNIYKEVINDSDFKKVCNSTVMKYFTDFSKFVTFCPLEFDFLFSRVCNQKGIYNDNSAMEKSFSDVLNKLKWNFIETGDNTKRIYIENVMKLRSQMKAYAIVKNAYYKQQSEYDFGKSEEFIQEHPFSNTDKGIEILNKLDNISKKILGCRNNIIQYSYNLFEINGYDMISLEKLTSSQFKKKPFPTVNSLLKYHKILGCTQEEMEKKDIYSVIKKGYYDIIFDNGVVIDAKLSAKGELSKFKDDFFNLMIKSIHFADIKDYFITLSNNGTAGVSLVDLFCPR

SEQ ID NO: 26 - protein, 759 aa, synthetic construct
MAHKKNLEGENLQVKTICLKANLSKEEVKEKWLPVINEYNVYYNRMSDYICSLLGTNITIGEFAEQLSIEKRKNGYFTICQDDKFKNESLYKIFHKSFPINHGTNIINNIISEKNIDQYDGNTLGFRPTMYRLRGYVDSVIGNYRTTIRTIKPSVKRHKISVDSSFDEKMEQCIYEIQKGNLKTVSEWNNKIDYLLSKSDMNPLTIDRFNLLRDFYVDNETEVNEKSNNSSIEQLVKFGGCHRKGDNMTLSLTEANFSIEEIDDSYGYLLTLHTDSGDYKIPLMGSKMLKKGDKCLIDFVNCKKGKSLTAKIDNDYNLYFHFVVYSNFEKIEDDNINNVVGVDVNSKHMLLMTNVIDDNIDGYVNIYKALVNDDEFKSLVTKSEYDDYVTMSKYVTFCPIELKYLYARYCVQKDYPISNKDVAIEQCISRVIDKLCKETLDSRANNYLCMVRRIRHYYKSYYVLKMTYYDKMSEYDTNMEYNDISTTSKETMDQRRFENSFRETDCAKEILSKLDKIGNDILGCRNNILTYAYKLFEELGYDTIALENLESSQFDKMKSLPSCQSMLKYHKLEGKTMEEVMSNTSVKSLIENEYYDFSLNDNKTVENITYTKNGLMKKGFDEFFNLFMKIIHFADIKDKFLQLYNNGSVKVILVPSYFTSQMDSSNHSIYMEKSKNDKLVFASKHKVRKTQETHLNGLNADYNAACNIAYIAKDIKWREKFCKKTSNNGYSTPFYDCATKNQIEMVKRIKQLNAIKMLA

SEQ ID NO: 27 - protein, 746 aa, synthetic construct
MASHKKTESNQIIKTFSFKIKNANGLSLDVLNDAITEYQNYYNICSDWIKDHLTMKIGELYKYIPDEKKNSGYALTLISDEWKDKPMYMMFKKGYPANSRDNAIYEALNTCNTEHYTGNILNFSDTYYRRFGYVASTISNYVTKISKMSTGSRSKNISNDSDVDTIMEQVIYEMEHNGWTSVKDWENQMEYLESKTDSNPNFVYRMTTLYEFYKSHIDEVNSKMETMSIDSLIKFGGCRRKDSKKSMYIMGGSNTPFDITQIGGNSLNIKFSKNLNVDVFGRYDVIKDNTLLVDIINGHGASFVLKIINDEIYIDINVSVPFDKKIATTNKVVGVDVNIKHMLLATNILDDGNVNGYVNIYKEVINDSDFKKVCNSTVMKYFTDFSKFVTFCPLEFDFLFSRVCNQKGIYNDNSAMEKSFSDVLNKLKWNFIETGDNTKRIYIENVMKLRSQMKAYAIVKNAYYKQQSEYDFGKSEEFIQEHPFSNTDKGIEILNKLDNISKKILGCRNNIIQYSYNLFEINGYDMISLEKLTSSQFKKKSFPTVDSLLKYHKILGCTQEEMEKKDIYSVIKKGYYDIIFDNDVVTDAKLSTKGELSKFKDDFFNLMIKSIHFADIKDYFITLSNNGTAGVSLVPSYFTSQMDSIDHKIYFVQDNKSGKLKLANKHKVRSSQEKHINGLNADYNAARNIAYIMENTECRNMFMKQSRTDKSLYNKPSYETFIKTQGSAVAKLKKEGFVKILDEASV

SEQ ID NO: 28 - protein, 746 aa, synthetic construct
MASHKKTESNQIIKTFSFKIKNANGLSLDVLNDAITEYQNYYNICSDWIKDHLTMKIGELYKYIPDEKKNSGYALTLISDEWKDKPMYMMFKKGYPANSRDNAIYEALNTCNTEHYTGNILNFSDTYYRRFGYVASAISNYVTKISKMSTGSRSKNISNDSDVDTIMEQVIYEMEHNGWTSVKDWENQMEYLESKTDSNPNFVYRMTTLYEFYKSHIDEVNSKMETMSIDSLIKFGGCRRKDSKKSMYIMGGSNTPFDITQIDGNSLNIKFSKNLNVDVFGRYDVIKDNTLLVDIINGHGASFVLKIINDEIYIDINVSVPFDKKIATTNKVVGIDVNIKHMLLATNILDDGNVNGYVNIYKEVINDSDFKKVCNSTVMKYFTDFSKFVTFCPLEFDFLFSRVCNQKGIYNDNSAMEKSFSNVLNKLKWNFIETGDNTKRIYIENVMKLRSQMKAYAIVKNAYYKQQSEYDFGKSEEFIQEHPFSNTDKGIEILNKLDNISKKILGCRNNIIQYSYNLFEINGYDMISLEKLTSSQFKKKPFPTVNSLLKYHKILGCTQEEMEKKDIYSVIKKGYYDIIFDNDVVTDAKLSAKGELSKFKDDFFNLMIKSIHFADIKDYFITLSNNGTAGVSLVPSYFTSQMDSIDHKIYFVQDNKSGKLKLANKHKVRSSQEKHINGLNADYNAARNIAYIMENTDCRNMFMKQSRTDKSLYNKPSYETFIKTQGSAVAKLKKEGFVKILDEASV

SEQ ID NO: 29 - protein, 746 aa, synthetic construct
MASHKKTESNQIIKTFSFKIKNANGLSLDVLNDAITEYQNYYNICSDWIKDHLTMKIGELYKYIPDEKKNSGYALTLISDEWKDKPMYMMFKKGYPANNRDNAIYETLNTCNTEHYTGNILNFSDTYYRRFGYVASTISNYVTKISKMSTRSRSKNISNDSDVDTIMEQVIYEMEHNGWTSVKDWENQMEYLESKTDSNPNFVYRMTTLYEFYKSHIDEVNSKMETMSIDSLIKFGGCRRKDSKKSMYIMGGSNTPFDITQIGGNSLNIKFSKNLNVDVFGRYDVIKDNTLLVDIINGHGASFVLKIINGEIYIDINVSVPFDKKIATTNKVVGIDVNIKHMLLATNILDDGNVNGYVNIYKEVINDSDFKKVCNSTVMKYFTDFSKFVTFCPLEFDFLFSRVCNQKGIYNDNSAMEKSFSDVLNKLKWNFIETGDNTKRIYIENVMKLRSQMKAYAIVKNAYYKQQSEYDFGKSEEFIQEHPFSNTDKGIEILNKLDNISKKILGCRNNIIQYSYNLFEINGYDMISLEKLTSSQFKKKPFPTVNSLLKYHKILGCTQEEMEKKDIYSVIKKGYYDIIFDNDVVTDAKLSTKGELSKFKDDFFNLMIKSIHFADIKDYFITLSNNGTAGVSLVPSYFTSQMDSIDHKIYFVQDNKSGKLKLANKHKVRSSQEKHINGLNADYNAARNIAYIMENTDCRNMFMKQSRTDKSLYNKPSYETFIKTQGSAVAKLKKEGFVKILDEASV

SEQ ID NO: 30 - protein, 47 aa, synthetic construct
NMFMKQSRTDKSLYNKPSYETFIKTQGSAVAKLKKEGFVKILDEASV

SEQ ID NO: 31 - protein, 746 aa, synthetic construct
MASHKKTESNQIIKTFSFKIKNANGLSLDVLNDAITEYQNYYNICSDWIKDHLTMKIGELYKYIPDEKKNSGYALTLISDEWKDKPMYMMFKKGYPANNRDNAIYETLNTCNTEHYTGNILNFSDTYYRRFGYVASTISNYVTKISKMSTGSRSKNISNDSDVDTIMEQVIYEMEHNGWTSVKDWENQMEYLESKTDSNPNFVYRMTTLYEFYKSHIDEVNSKMETMSIDSLIKFGGCRRKDSKKSMYIMGGSNTPFDITQIGGNFLNIKFSKNLNVDVFGRYDVIKDNTLLVDIINEHGASFVLKIINDEIYIDINVSVPFDKKIATTNKVVGVDVNIKHMLLATNILDDGNVNGYVNIYKEVINDSDFKKVCNSTVMKYFTDFSKFVTFCPLEFDFLFSRVCNQKGIYNDNSAMEKSFSDVLNKLKWNFIETGDNTKRIYIENVMKLRSQMKAYAIVKNAYYKQQSEYDFGKSEEFIQEHPFSNTDKGIEILHKLDNISKKILGCRNNIIQYSYNLFEINGYDMISLEKLTSSQFKKKPFPTVNSLLKYHKILGCTQEEMEKKDIYSVIKKGYYDIIFDNDVVTDAKLSTKGELSKFKDDFFNLMIKSIHFADIKDYFITLSNNGTAGVSLVPSYFTSQMDSIDHKIYFVQDNKSGKLKLANKHKVRSSQEKHINGLNADYNAARNIAYIMENTECRNMFMKQSRTDKSLYNKPSYETFIKTQGSAVAKLKKEGFVKILDEASV

SEQ ID NO: 32 - protein, 245 aa, synthetic construct
KKILGCRNNIIQYSYNLFEINGYDMISLEKLTSSQFKKKPFPTVNSLLKYHKILGCTQEEMEKKDIYSVIKKGYYDIIFDNDVVTDAKLSTKGELSKFKDDFFNLMIKSIHFADIKDYFITLSNNGTAGVSLVPSYFTSQMDSIDHKIYFVQDNKSGKLKLANKHKVRSSQEKHINGLNADYNAARNIAYIMENTECRNMFMKQSRTDKSLYNKPSYETFIKTQGSAVAKLKKEGFVKILDEASV

SEQ ID NO: 33 - protein, 178 aa, synthetic construct
NAYYKQQSEYDFGKSEEFIQEHPFSNTDKGIEILHKLDNISKKILGCRNNIIQYSYNLFEINGYDMISLEKLTSSQFEKKPFPTVNSLLKYHKILGCTQEEMEKKDIYSVIKKGYYDIIFDNDVVTDAKLSTKGELSKFKDDFFNLMIKSIHFADIKDYFITLSNNGTVGVSLVPSTV

SEQ ID NO: 34 - protein, 85 aa, synthetic construct
MANKHKVRSSQEKHINGLNADYNAARNIAYIIENTECRNMFMKQSRTDKSLYNKPSYETFIKTQGSTVAKLKKEGFVKILDEASV

SEQ ID NO: 35 - protein, 767 aa, synthetic construct
MAHKKQKEENEIIKTISLKVKDYAGYPIVEAMREYTKYYNKISQWINSNLLTIKIGELSAFMPDECKTHNYYTYMMSPDWVNEPLYKMFMKGFHAQHCDNILFNVVKTLNIDEYAGNSLGLSASCFRRSGYFQNVVSNYKSKFANPHISIRRKNLSDLPTEDELVEQCIYEIQNGLSSKTKWEEQIEYLKERDDSKQIYLTRLNTLFMYYKANKDFVDEQIQIKSVESLANFGGCVRKDDKLSMNLVFSSNSPYKVVLNEKRNGYILSYSNNFSIELYGNRMGLLNGVEVFNVGDKHSNNITFKMDNDELFVNIPVSVNFVKKANETNKVVGVDVNLKHSIFATNIIDDGKLDGFVNIYRELLNDVDFVKSCPNELLNFILDVEKYAFFMPLELGLLSSRVMNQCGYSTIGKYEKLFTTEEHFFRVLRQLEKRFQESGENQKRIYIENVIKMRAQVKAYFTLKYAYNKANKDYDLKMGFVDESTANKETMDQRRFENQFVNTYTAKEILGKMRRIANVITSCRNNIICYMYKIFENNGYGVVALEKLQSSQMKKEKRIPSLLSLLKKQKVEGYTINELKDKSVFKFIERGYYTFDFDDDNKITGVQFSDAGEVVNMETELHNLALKTIHFADAKDYFVTLSNNGSVSVALVPSQFTSQMDSTKHVLYAKKNNKGKLGIVSEHEVRPKQECHINGLNGDYNAACNIAYIFENDEWRNAFMKMNPNEYGKALFETNMESTSTIINTLKKINPDNIISFDEYEKTKKVAA

SEQ ID NO: 36 - protein, 652 aa, synthetic construct
YPANSRDNAIYEALNTCNTEHYTGNILNFSDTYYRRFGYVASAISNYVTKISKMSTGSRSKNISNDSDVDTIMEQVIYEMEHNGWTSVKDWENQMEYLESKTDSNPNFVYRMTTLYEFYKSHIDEVNSKMETMSIDSLIKFGGCRRKDSKKSMYIMGGSNTPFDITQIGGNSLNIKFSKNLNVDVFGRYDVIKDNTLLVDIINGHGASFVLKIINGEIYIDINVSVPFDKKIATTNKVVGVDVNIKHMLLATNILDDGNVKGYVNIYKEVINDSDFKKVCNSTVMKYFTDFSKFVTFCPLEFDFLFSRVCNQKGIYNDNSAMEKSFSDVLNKLKWNFIETGDNTKRIYIENVMKLRSQMKAYAIVKNAYYKQQSEYDFGKSEEFIQEHPFSNTDKGIEILNKLDNISKKILGCRNNIIQYSYNLFEINGYDMISLEKLTSSQFKKKSFPTVNSLLKYHKILGCTQEEMEKKDIYSVIKKGYYDIIFDNDVVTDAKLSTKGELSKFKDDFFNLMIKSIHFADIKDYFITLSNNGTAGVSLVPSYFTSQMDSIDHKIYFVQDNKSGKLKLANKHKVRSSQEKHINGLNADYNAARNIAYIMENTECRNMFMKQSRTDKSLYNKPSYETFIKTQGSAVAKLKKEGFVKILDEASV

SEQ ID NO: 37 - protein, 782 aa, synthetic construct
MMKKMRISPHLFYIFFKKIWKCHFFVLSLYQLNQYIMASHKKTESNQIIKTFSFKIKNANGLSLDVLNDAITEYQNYYNICSDWIKDHLTMKIGELYKYIPDEKKNSGYALTLISDEWKDKPMYMMFKKGYPANNRDNAIYETLNTCNTEHYTGNILNFSDTYYRRFGYVASAISNYVTKISKMSTGSRSKNISNDSDVDTIMEQVIYEMEHNGWTSVKDWENQMEYLESKTDSNPNFVYRMTTLYEFYKSHIDEVNSKMETMSIDSLIKFGGCRRKDSKKSMYIMGGSNTPFDITQIGGNSLNIKFSKNLNVDVFGRYDVIKDNTLLVDIINEHGASFVLKIINDEIYIDINVSVPFDKKIATTNKVVGVDVNIKHMLLATNILDDGNVNGYVNIYKEVINDSDFKKVCNSTVMKYFTDFSKFVTFCPLEFDFLFSRVCNQKGIYNDNSAMEKSFSNVLNKLKWNFIETGDNTKRIYIENVMKLRSQMKAYAIVKNAYYKQQSEYDFGKSEEFIQEHPFSNTDKGIEILNKLDNISKKILGCRNNIIQYSYNLFEINGYDMISLEKLTSSQFKKKPFPTVNSLLKYHKILGCTQEEMEKKDIYSVIKKGYYDIIFDNDVVTDAKLSTKGELSKFKDDFFNLMIKSIHFADIKDYFITLSNNGTAGVSLVPSYFTSQMDSIDHKIYFVQDNKSGKLKLANKHKVRSSQEKHINGLNADYNAARNIAYIMENTDCRNMFMKQSRTDKSLYNKPSYETFIKTQGSAVAKLKKEGFVKILDEASV

SEQ ID NO: 38 - protein, 889 aa, synthetic construct
WTDEDYCKFFAKYGMSEECQKWMCRDVYDYRIKDFVDYEKFDDSKIEEEQEEYSEIPVESEDSSPSTSETLPKSIYDLDLRRLDPNPEIPEDEKYNEEDLKNAYPDKYERFEKDGEDYSYIFFKKIWKCHFFVLSLYQLNQYIMASHKKTESNQIIKTFSFKIKNANGLSLDVLNDAITEYQNYYNICSDWIKDHLTMKIGELYKYIPDEKKNSGYALTLISDEWKDKPMYMMFKKGYPANNRDNAIYEALNTCNTEHYTGNILNFSDTYYRRFGYVASTISNYVTKISKMSTGSRSKNISNDSDVDTIMEQVIYEMEHNGWTSVKDWENQMEYLESKTDSNPNFVYRMTTLYEFYKSHIDEVNSKMETMSIDSLIKFGGCRRKDSKKSMYIMGGSNTPFDITQIDGNSLNIKFSKNLNVDVFGRYDVIKDNTLLVDIINGHGASFVLKIINDEIYIDINVSVPFDKKIATTNKVVGVDVNIKHMLLATNILDDGNVNGYVNIYKEVINDSDFKKVCNSTVMKYFTDFSKFVTFCPLEFDFLFSRVCNQKGIYNDNSAMEKSFSDVLNKLKWNFIETGDNTKRIYIENVMKLRSQMKAYAIVKNAYYKQQSEYDFGKSEEFIQEHPFSNTDKGIEILNKLDNISKKILGCRNNIIQYSYNLFEINGYDMISLEKLTSSQFEKKPFPTVNSLLKYHKILGCTQEEMEKKDIYSVIKKGYYDIIFDNGVVTDAKLSTKGELSKFKDDFFNLMIKSIHFADIKDYFITLSNNGTAGVSLVPSYFTSQMDSIDHKIYFVQDNKSGKLKLANKHKVRSSQEKHINGLNADYNAARNIAYIMENTECRNMFMKQSRTDKSLYNKPSYETFIKTQGSAVAKLKKEGFVKILDEASV

SEQ ID NO: 39 - protein, 223 aa, synthetic construct
YDMISLEKLTSSQFKKKPFPTVNSLLKYHKILGCTEEEMEKKDIYSVIKKGYYDIIFDNDVVTDAKLSTKGELSKFKDDFFNLMIKSIHFADIKDYFITLSNNGTAGVSLVPSYFTSQMDSIDHKIYFVQDNKSGKLKLANKHKVRSSQEKHINGLNADYNAARNIAYIMENTECRNMFMKQSRTDKSLYNKPSYETFIKTQGSAVAKLKKEGFVKILDEASA

SEQ ID NO: 40 - protein, 439 aa, synthetic construct
MKIGELYKYIPDEKKNSGYALTLISDEWKDKPMYMMFKKGYPANNRDNAIYETLNTCNTEHYTGNILNFSDTYYRRFGYVASAISNYVTKISKMSTGSRSKNISNDSDVDTIMEQVIYEMEHNGWTSVKDWENQMEYLESKTDSNPNFVYRMTTLYEFYKSHIDEVNSKMETMSIDSLIKFGGCRRKDSKKSMYIMGGSNTPFDITQIDGNSLNIKFSKNLNVDVFGRYDVIKDNTLLVDIINGHGASFVLKIINGEIYIDINVSVPFDKKIATTNKVVGVDVNIKHMLLATNILDDGNVNGYVNIYKEVINDSDFKKVCNSTVMKYFTDFSKFVTFCPLEFDFLFSRVCNQKGIYNDNSAMEKSFSDVLNKLKWNFIETGDNTKRIYIENVMKLRSQMKAYAIVKNAYYKQQSEYDFGKSEEFIQEYDFFGETYQFSI

SEQ ID NO: 41 - protein, 196 aa, synthetic construct
MISLEKLTSSQFKKKPFPTVNSLLKYHKILGCTQEEMEKKKGYYDIIFDNDVVTDAKLSTKGELSKFKDDFFNLMIKSIHFADIKDYFITLSNNGTAGVSLVPSYFTSQMDRANKHKVRSSQEKHINGLNADYNAARNIAYIMENTECRNMFMKQSRTDKSLYNKPSYETFIKTQGSAVAKLKKEGFVKILDEASV

SEQ ID NO: 42 - protein, 748 aa, synthetic construct
YIMASHKKTESNQIIKTFSFKIKNANGLSLDVLNDAITEYQNYYNICSDWIKDHLTMKIGELYKYIPDEKKNSGYALTLISDEWKDKPMYMMFKKGYPANSRDNAIYETLNTCNTEHYTGNILNFSDTYYRRFGYVASTISNYVTKISKMSTGSRSKNISNDSDVDTIMEQVIYEMEHNGWTSVKDWENQMEYLESKTDSNPNFVYRMTTLYEFYKSHIDEVNSKMETMSIDSLIKFGGCRRKDSKKSMYIMGGSNTPFDITQIGGNSLNIKFSKNLNVDVFGRYDVIKDNTLLVDIINGHGASFVLKIINGEIYIDINVSVPFDKKIATTNKVVGVDVNIKHMLLATNILDDGNVNGYVNIYKEVINDSDFKKVCNSTVMKYFTDFSKFVTFCPLEFDFLFSRVCNQKGIYNDNSAMEKSFSNVLNKLKWNFIETGDNTKRIYIENVMKLRSQMKAYAIVKNAYYKQQSEYDFGKSEEFIQEHPFSNTDKGIEILNKLDNISKKILGCRNNIIQYSYNLFEINGYDMISLEKLTSSQFEKKPFPTVNSLLKYHKILGCTQEEMEKKDIYSVIKKGYYDIIFDNGVVTDAKLSTKGELSKFKDDFFNLMIKSIHFADIKDYFITLSNNGTAGVSLVPSYFTSQMDSIDHKIYFVQDNKSGKLKLANKHKVRSSQEKHINGLNADYNAARNIAYIMENTECRNMFMKQSRTDKSLYNKPSYETFIKTQGSAVAKLKKEGFVKILDEASV

SEQ ID NO: 43 - protein, 481 aa, synthetic construct
SLNIKFSKNLNVDVFGRYDVIKDNTLLVDIINGHGASFVLKIINGEIYIDINVSVPFDKKIATTNKVVGVDVNIKHMLLATNILDDGNVNGYVNIYKEVINDSDFKKVCNSTVMKYFTDFSKFVTFCPLEFDFLFSRVCNQKGIYNDNSAMEKSFSDVLNKLKWNFIETGDNTKRIYIENVMKLRSQMKAYAIVKNAYYKQQSEYDFGKSEEFIQEHPFSNTDKGIEILNKLDNISKKILGCRNNIIQYSYNLFEINGYDMISLEKLTSSQFKKKPFPTVNSLLKYHKILGCTQEEMEKKDIYSVIKKGYYDIIFDNGVVIDAKLSAKGELSKFKDDFFNLMIKSIHFADIKDYFITLSNNGTAGVSLVPSYFTSQMDSIDHKIYFVQDNKSGKLKLANKHKVRSSQEKHINGLNADYNAARNIAYIMENTECRNMFMKQSRTDKSLYNKPSYETFIKTQGSAVAKLKKEGFVKILDEASV

SEQ ID NO: 44 - protein, 746 aa, synthetic construct
MASHKKTESNQIIKTFSFKIKNANGLSLDVLNDAITEYQNYYNICSDWIKDHLTMKIGELYKYIPDEKKNSGYALTLISDEWKDKPMYMMFKKGYPANNRDNAIYETLNTCNTEHYTGNILNFSDTYYRRFGYVASAISNYVTKISKMSTGSRSKNISNDSDVDTIMEQVIYEMEHNGWTSVKDWENQMEYLESKTDSNPNFVYRMTTLYEFYKSHIDEVNSKMETMSIDSLIKFGGCRRKDSKKSMYIMGGSNTPFDITQIGGNSLNIKFSKNLNVDVFGRYDVIKDNTLLVDIINEHGASFVLKIINDEIYIDINVSVPFDKKIATTNKVVGIDVNIKHMLLATNILDDGNVNGYVNIYKEVINDSDFKKVCNSTVMKYFTDFSKFVTFCPLEFDFLFSRVCNQKGIYNDNSAMEKSFSDVLNKLKWNFIETGDNTKRIYIENVMKLRSQMKAYAIVKNAYYKQQSEYDFGKSEEFIQEHPFSNTDKGIEILNKLDNISKKILGCRNNIIQYSYNLFEINGYDMISLEKLTSSQFKKKSFPTVDSLLKYHKILGCTQEEMEKKDIYSVIKKGYYDIIFDNDVVTDAKLSTKGELSKFKDDFFNLMIKSIHFADIKDYFITLSNNGTAGVSLVPSYFTSQMDSIDHKIYFVQDNKSGKLKLANKHKVRSSQEKHINGLNADYNAARNIAYIMENTECRNMFMKQSRTDKSLYNKPSYETFIKTQGSAVAKLKKEGFVKILDEASV

SEQ ID NO: 45 - protein, 746 aa, synthetic construct
MASHKKTESNQIIKTFSFKIKNANGLSLDVLNDAITEYQNYYNICSDWIKDHLTMKIGELYKYIPDEKKNSGYALTLISDEWKDKPMYMMFKKGYPANNRDNAIYETLNTCNTEHYTGNILNFSDTYYRRFGYVASAISNYVTKISKMSTGSRSKNISNDSDVDTIMEQVIYEMEHNGWTSVKDWENQMEYLESKTDSNPNFVYRMTTLYEFYKSHIDEVNSKMETMSIDSLIKFGGCRRKDSKKSMYIMGGSNTPFDITQIDGNSLNIKFSKNLNVDVFGRYDVIKDNTLLVDIINGHGASFVLKIINDEIYIDINVSVPFDKKIATTNKVVGVDVNIKHMLLATNILDDGNVNGYVNIYKEVINDSDFKKVCNSTVMKYFTDFSKFVTFCPLEFDFLFSRVCNQKGIYNDNSAMEKSFSDVLNKLKWNFIETGDNTKRIYIENVMKLRSQMKAYAIVKNAYYKQQSEYDFGKSEEFIQEHPFSNTDKGIEILNKLDNISKKILGCRNNIIQYSYNLFEINGYDMISLEKLTSSQFKKKPFPTVNSLLKYHKILGCTQEEMEKKDIYSVIKKGYYDIIFDNDVVTDAKLSTKGELSKFKDDFFNLMIKSIHFADIKDYFITLSNNGTAGVSLVPSYFTSQMDSIDHKIYFVQDNKSGKLKLANKHKVRSSQEKHINGLNADYNAARNIAYIMENTECRNMFMKQSRTDKSLYNKPSYETFIKTQGSAVAKLKKEGFMKILDEASV

SEQ ID NO: 46 - protein, 746 aa, synthetic construct
MASHKKTESNQIIKTFSFKLKNANGLSLDVLNDAITEYQNYYNICSDWIKDHLTMKIGELYKYIPDEKKNSGYALTLISDEWKDKPMYMMFKKGYPANNRDNAIYEALNTCNTEHYTGNILNFSDTYYRRFGYVASTISNYVTKISKMSTGSRSKNISNDSDVDTIMEQVIYEMEHNGWTSVKDWENQMEYLESKTDSNPNFVYRMTTLYEFYKSHIDEVNSKMETMSIDSLIKFGGCRRKDSKKSMYIMGGSNTPFDITQIGSNSLNIKFSKNLNVDVFGRYDVIKDNTLLVDIIKGHGASFALKIINDEIYIDINVSVPFDKKIATTNKVVGIDVNIKHMLLATNILDDGNVNGYVNIYKEVINDSDFKKVCNSTVMKYFTDFSKFVTFCPLEFDFLFSRVCNQKGIYNDNSAMEKSFSDVLNKLKWNFIETGDNTKRIYIENVMKLRSQMKAYAIVKNAYYKQQSEYDFGKSEEFIQEHPFSNTDKGIEILHKLDNISKKILGCRNNIIQYSYNLFEINGYDMISLEKLTSSQFKKKPFPTVNSLLKYHKILGCTQEEMEKKDIYSVIKKGYYDIIFDNDVVTDAKLSTKGELSKFKDDFFNLMIKSIHFADIKDYFITLSNNGTAGVSLVPSFFTSQMDSIDHKIYFVQDNKSGKLKLANKHKVRSSQEKHINGLNADYNAARNIAYIMENTECRNMFMKQSRTDKSLYNKPSYETFIKTQGSAVAKLKKEGFVKILDEASV

SEQ ID NO: 47 - protein, 735 aa, synthetic construct
MAHKKNIGAEIVKTYSFKVKNTNGITMEKLMNAIDEYQSYYNLCSDWICKNLTTMTIGDLDQYIPEKAKDNTYATVLLDEAWKNQPLYKIFGKKYSSNNRDAVLYHALSSIVNASEKNILGISKTYYARKGYVLNVASNYASKLSKLNTGVKSRAIKETSDEETIIEQVIYEMEHNKWESIEDWKNQIEYLNSKTDYNPTYMERMKTLSAYYSEHKSEVDAKMQEMAVENLVKFGGCRRNNSKKSMFIMGTTNTNYTISYIGGNSFNINFANILNFDVYGRRDVVKNGEVLVDIMANHGDSIVLKIVNGELYADVPCSVTLNKVESNFDKVVGIDVNMKHMLLSTSVTDNGSLDFLNIYKEMSNNAEFMALCSEDDRKYYKDISQYVTFAPLELDLLFSRISKQGKVKMEKAYSEILEALKWKFFANGDNKNRIYVESIQKIRQQIKALCIIKNAYYEQQSAYDIDKTQEYIEAHPFSLTEKGMSIKSKMDNICRTIIGCRNNIIDYAYSFFERNDYSIIGLEKLTSAQFEKTKSLPTCKSLLNFHKVLGHTLSELGTLPINDVVKKGYYTFTTDNEGRITDASLSEKGKVRKMKDDFFNQTIKAIHFADVKDYFATLSNNGQTGIFFVPSQFTSQMDSSTHNLYFENAKNGGLKLAPKYKVRQMQEYHLNGLPADYNAARNIAYIGLDETMRNTFLKKANSNKSLYNQPIYDTGIKKTAGVFYRMKKLKRYEII

SEQ ID NO: 48 - protein, 779 aa, synthetic construct
MKRNQKHIKNIEESETIKTISFKVKDYAGIPIVEAMHEYRNYYNRLSRFINSKLLTMTIGELASLLPERCKSKGYYLYMTSDEWVNEYVYKMFMESFNSQSCDNIWFNYIKMNNPEEYNGNILGISDSYYRRNGYFINVISNYKTKFKSPQINVKSKKLSESPTEEELKEQCVYEYVKHNLHSKKDWEEQIKYLDERGESKINILERIRTLYQYYKENIPTIKEYIELKSIESIEKFGGCVRKEDKLSMSLQYVSTHNYEIKLNDTRNGYIISGISKDLSFEVYGNRMGLLGGEEILNIPEKHSTSITFVMRNNSLYVDIPVAVPFSKVINDCDGKTVGIDVNLKHALFATSEVDNGQFYDYVNVYAELLKDENFVKVCHKELLDYIKDVSKYVFFAPIELNLLLSRVMKQKGYENIDNYKKLYKVEEAYLCVLDKLQKRFIDEGNNTKRIYIENLKKMRAQMKAYYILKDTYSKYQKDYDIEMGFVDESTESKETMDARRSENPFRSTDIAQDILKKMNNVGKTVEACRNNIIAYIYKVFENSDFATIVLEKLQSSQMKKHKRIPTVNSLLKYHHVEGHTIEETKEMKIYDVVEKGYYNFIVNEKNEIIDATLTDKGKVIMIEAEFYNFALKSIHFADAKDYFITLSNNGSVNIALVPSQFTSQMDSIRHAIFVTKGKKGKKVIVDKKYVRPKQEKHINGLNGDYNASRNIAYIFENEELREELLKKEEEYNKYGKVLYDTLIKFPSGVINKLKKFGDKYMTTIENLDEIQVEDVAYV

SEQ ID NO: 49 - protein, 592 aa, synthetic construct
MSKLNTGVKRHTIKETSDEATIVEQVIYEMEHNKWESIEDWKNQIEYLNSKTDYNPTYMERMKTLSAYYSEHKSEVDAKMQEMAVENLVKFGGCRRNNSKKSMFIMGSSKTTYTISYIGDNCFNINFANILNFDVYGRRDVVKNGEVLVDIMANHGDSIVLKIVNGELYADVPCSTTLNKVESTFDKVAGIDVNMKHMLLSTSVTDNGNSDFVNIYKEMSNNAEFMALCPEEDRKYYKDISQYVTFAPLELDLLFSRISKQGKVKMEKAYSEILETLKWKFFANGDNKNRIYVESIQKIRQQIKALCVIKNAYYEQQSAYDIDKTQEYIEAHPFSLTEKGMSIKSKMDNICRTIIGCRNNIIDYAYSFFERNDYSIIGLEKLTSSQFEKTKSLPTCKSLLNFHKVLGHTLSELETLPINDVVKKGYYTFTTDNEGRITDASLSEKGKVRKMKDDFFNQAIKAIHFADVKDYFATLSNNGQTGIFFVPSQFTSQMDSNTHTLYFENAKNGGLKLASKYKVRQTQEYHLNGLPADYNAARNIAYIGLDETMRNTFLKKANSNKSLYNQPIYDTGIKKTAGVFSRMKKLKRYEII

SEQ ID NO: 50 - protein, 746 aa, synthetic construct
MASHKKTESNQIIKTFSFKIKNANGLSLDVLNDAITEYQNYYNICSDWIKDHLTMKIGELYKYIPDEKKNSGYALTLISDEWKDKPMYMMFKKGYPANNRDNAIYEALNTCNTEHYTGNILNFSDTYYRRFGYVASAISNYVTKISKMSTGSRSKNISNDSDVDTIMEQVIYEMEHNGWTSVKDWENQMEYLESKTDSNPNFVYRMTTLYEFYKSHIDEVNSKMETMSIDSLIKFGGCRRKDSKKSMYIMGGSNTPFDITQIGGNSLNIKFSKNLNVDVFGRYDVIKDNTLLVDIINGHGASFVLKIINDEIYIDINVSVPFDKKIATTNKVVGIDVNIKHMLLATNILDDGNVNGYVNIYKEVINDSDFKKVCNSTVMKYFTDFSKFVTFCPLEFDFLFSRVCNQKGIYNDNSAMEKSFSDVLNKLKWNFIETGDNTKRIYIENVMKLRSQMKAYAIVKNAYYKQQSEYDFGKSEEFIQEHPFSNTDKGIEILNKLDNISKKILGCRNNIIQYSYNLFEINGYDMISLEKLTSSQFKKKPFPTVNSLLKYHKILGCTQEEMEKKDIYSVIKKGYYDIIFDNDVVTDAKLSTKGELSKFKDDFFNLMIKSIHFADIKDYFITLSNNGTAGVSLVPSYFTSQMDSIDHKIYFVQDNKSGKLKLANKHKVRSSQEKHINGLNADYNAARNIAYIMENTDCRNMFMKQSRTDKSLYNKPSYETFIKTQGSAVAKLKKEGFVKILDEASV

SEQ ID NO: 51 - protein, 29 aa, synthetic construct
YETFIKTQGSAVAKLKKEGFVKILDEASV

SEQ ID NO: 52 - protein, 738 aa, synthetic construct
MASHSSLSNNQIFKTFSFKVKSSNLSKDFFDVIKEYQEYYNKCSDVILENLTCIKIGDIFDMIPEKSKKSDYAQYAISDEWKNVPLYNIFSKAFAPMHRDNLLYIWLTKLVPYTGNLLKISDTFYKRNGFIKSVISNYTTSFTNIKPKVKFQKLTGDDSHEMLLTQTICDMVKFNLYDIKSWEEMVSYFEMKSETSEDTLNRIHTLFDFYKNNTPEVEDKYNELVTESLSKFGGCRRDMSKLTMSIQLSKKVIKVTHGYNTLNYKYGKLIDLELWGRKDIINNDELLINLENVCEMIVFKIKNGEIYVDIPFKVDFIKKDQTIDKIAGVDANIKHMLLSTSVKDENLIGYTNIYKEVINDSDFQKVCDNKTMKILQEISNYVTFAPIEFDMLFSRISKQREMKDKYINMEIAFTNVLNKLKQKFIVNSDNKNRIYIESILKIRSQLKSYAILEEVKYKKASEYDSAIVEEFGVEYLETHPFKDTETYKEINKKILNISENIIGCRNNIIQYAYKIFESNGFDMISLENLTNSNFKKEKNMPTIKSLLSYHHVLGKTNEEIEKLDVYSVIKKGYYTFEYKDGKVVNAKLSEIGEMIKIKTTMFNMMIKSIHFAEIKDYFITLANNGEVGVSLVPSYYTSQMDSTDHKVFGLLSKKGKWTLVDKRKVRKNQETHINGLNADYNAAKNIAFILSDEVWRNKFTKKTKTPKYNTPSYYTSINSQGKMLRALKSLKAFKEFKI

SEQ ID NO: 53 - protein, 692 aa, synthetic construct
MKIGELYKYIPDEKKNSGYALTLISDEWKDKPMYMMFKKGYPANNRDNAIYEALNTCNTEHYTGNILNFSDTYYRRFGYVASAISNYVTKISKMSTGSRSKNISNDSDVDTIMEQVIYEMEHNGWTSVKDWENQMEYLESKTDSNPNFVYRMTTLYEFYKSHIDEVNSKMETMSIDSLIKFGGCRRKDSKKSMYIMGGSNTPFDITQIGGNSLNIKFSKNLNVDVFGRYDVIKDNTLLVDIINEHGASFVLKIINDEIYIDINVSVPFDKKIATTNKVVGIDVNIKHMLLATNILDDGNVNGYVNIYKEVINDSDFKKVCNSTVMQYFTDFSKFVTFCPLEFDFLFSRVCNQKGIYNDNSAMEKSFSDVLNKLKWNFIETGDNTKRIYIENVMKLRSQMKAYAIVKNAYYKQQSEYDFGKSEEFIQEHPFSNTDKGIEILNKLDNISKKILGCRNNIIQYSYNLFEINGYDMISLEKLTSSQFKKKPFPTVNSLLKYHKILGCTQEEMEKKDIYSVIKKGYYDIIFDNDVVTDAKLSTKGELSKFKDDFFNLMIKSIHFADIKDYFITLSNNGTAGVSLVPSYFTSQMDSIDHKIYFVQDNKSGKLKLANKHKVRSSQEKHINGLNADYNAARNIAYIMENTECRNMFMKQSRTDKSLYNKPSYETFIKTQGSAVAKLKKEGFVKILDEASV

SEQ ID NO: 54 - protein, 441 aa, synthetic construct
KIINDEIYIDINVSVPFDKKIATTNKVVGIDVNIKHMLLATNILDDGNVNGYVNIYKEVINDSDFKKVCNSTVMKYFTDFSKFVTFCPLEFDFLFSRVCNQKGIYNDNSAMEKSFSDVLNRLKWNFIETGDNTKRIYIENVMKLRSQMKAYAIVKNAYYKQQSEYDFGKSEEFIQEHPFSNTDKGIEILNKLDNISKKILGCRNNIIQYSYNLFEINGYDMISLEKLTSSQFKKKPFPTVNSLLKYHKIRGGTQEEMEKKDIYSVIKKGYYDIIFDNDVVTDAKLSTKGELSKFKDDFFNLMIKSIHFADIKDYFITLSNNGTAGVSLVPSYFTSQMDSIDHKIYFVQDNKSGKLKLANKHKVRSSQEKHINGLNADYNAARNIAYIMENNECRNMFMKQSRTDKSLYNKPSYETFIKTQGSAVAKLKKEGFVKILDEASV

SEQ ID NO: 55 - protein, 397 aa, synthetic construct
DDGNVNGYVNIYKEVINDSDFKKVCNSTVMKYFTDFSKFVTFCPLEFDFLFSRVCNQKGIYNDNSAMEKSFSDVLNKLKWNFIETGDNTKRIYIENVMKLRSQMKAYAIVKNAYYKQQSEYDFGKSEEFIQEHPFSNTDKGIEILNKLDNISKKILGCRNNIIQYSYNLFEINGYDMISLEKLTSSQFKKKPFPTVNSLLKYHKILGCTQEEMEKKDIYSVIKKGYYDIIFDNDVVTDAKLSTKGELSKFKDDFFNLMIKSIHFADIKDYFITLSNNGTAGVSLVPSYFTSQMDSIDHKIYFVQDNKSGKLKLANKHKVRSSQEKHINGLNADYNAARNIAYIMENTECRNMFMKQSRTDKSLYNKPSYETFIKTQGSAVAKLKKDGFVKILDEASA

SEQ ID NO: 56 - protein, 749 aa, synthetic construct
MASHKKTESNQIIKTFSFKIKNANGLSLDVLNDAITEYQNYYNICSDWIKDHLTMKIGELYKYIPDEKKNSGYALTLISDEWKDKPMYMMFKKGYPANSRDNAIYEALNTCNTEHYTGNILNFSDTYYRRFGYVASAISNYVTKISKMSTGSRSKNISNDSDVDTIMEQVIYEMEHNGWTSVKDWENQMEYLESKTDSNPNFVYRMTTLYEFYKSHIDEVNSKMETMSIDSLIKFGGCRRKDSKKSMYIMGGSNTPFDITQIDGNSLNIKFSKNLNVDVFGRYDVIKDNTLLVDIINGHGASFVLKIINGEIYIDINVSVPFDKKIATTNKVVGIDVNIKHMLLATNILDDGNVNGYVNIYKEVINDSDFKKVCNSTVMKYFTDFSKFVTFCPLEFDFLFSRVCNQKGIYNDNSAMEKSFSDVLNKLKWNFIETGDNTKRIYIENVMKLRSQMKAYAIVKNAYYKQQSEYDFGKSEEFIQEHPFSNTDKGIEILNKLDNISKKILGCRNNIIQYSYNLFEINGYDMISLEKLTSSQFKKKPFPTVNSLLKYHKILGCTQEEMEKKDIYSVIKKGYYDIIFDNDVVTDAKLSTKGELSKFKDDFFNLMIKSIHFADIKDYFITLSNNGTAGVSLVPSYFTSQMDSIDHKIYFVQDNKSGKLKLANKHKVRSSQEKHINGLNADYNAARNIAYIMENTECRNMFMKQSRTDKSLYNKPSYETFIKTQGSAVAKLKKEGFVKILDEASVREF

SEQ ID NO: 57 - DNA, 36 bp, synthetic construct
  source 1..36: mol_type = other DNA
attgttgtaactcttattttgtatggagtaaacaac

SEQ ID NO: 58 - DNA, 36 bp, synthetic construct
  source 1..36: mol_type = other DNA
attgttgtagacctctttttataaggattgaacaac

SEQ ID NO: 59 - DNA, 36 bp, synthetic construct
  source 1..36: mol_type = other DNA
attgttgtagatacctttttgtaaggattgaacaac

SEQ ID NO: 60 - DNA, 37 bp, synthetic construct
  source 1..37: mol_type = other DNA
tattgttgtagatacctttttgtaaggattaaacaac

SEQ ID NO: 61 - DNA, 39 bp, synthetic construct
  source 1..39: mol_type = other DNA
aatgttgtagatgcctttttataaggattaaacaacttg

SEQ ID NO: 62 - DNA, 36 bp, synthetic construct
  source 1..36: mol_type = other DNA
attgttgaaatagtacttttatagtctatatacaac

SEQ ID NO: 63 - DNA, 37 bp, synthetic construct
  source 1..37: mol_type = other DNA
tattgttgtaactcttattttgtatggagtaaacaac

SEQ ID NO: 64 - DNA, 36 bp, synthetic construct
  source 1..36: mol_type = other DNA
attgttgtaacatctattttgtaaggtgtaaacaac

SEQ ID NO: 65 - DNA, 37 bp, synthetic construct
  source 1..37: mol_type = other DNA
aattgttgtaactcttattttgtatggagtaaacaac

SEQ ID NO: 66 - DNA, 36 bp, synthetic construct
  source 1..36: mol_type = other DNA
attgttgtaacttttattttgtatggagtaaacaac

SEQ ID NO: 67 - DNA, 36 bp, synthetic construct
  source 1..36: mol_type = other DNA
aatgttgtagatacctttttgtaaggattgaacaac

SEQ ID NO: 68 - DNA, 36 bp, synthetic construct
  source 1..36: mol_type = other DNA
attgttgtaatactatttttgtaaagtataaacaac

SEQ ID NO: 69 - DNA, 36 bp, synthetic construct
  source 1..36: mol_type = other DNA
aatgttgtagatgcctttttataaggattaaacaac

SEQ ID NO: 70 - DNA, 36 bp, synthetic construct
  source 1..36: mol_type = other DNA
attgttgtaatacactttttataaggtatgaacaac

SEQ ID NO: 71 - DNA, 37 bp, synthetic construct
  misc_feature 14: note = n is a, c, g, or t
  source 1..37: mol_type = other DNA
wattgttgtarmwnyywttttrtawggwktraacaac

SEQ ID NO: 72 - DNA, 1986 bp, Arabidopsis thaliana
  source 1..1986: mol_type = other DNA
agtctagctcaacagagcttttaacccaaattggtacaatagaatacaactttagatcataattctcaaaagaaagagattccttagctattctatctgccactccatttccttctcggcttgtatgcacaagcataaaatcctcaaacttgctaagtagatactttatgtcttggataattggattgagacttgacaagcataactttcatgtaaccaaagacacaagttgctgagaatccacctcaaaaatgatcttcctataattgaatcgggataatgacagcacagcccatctaagagcctccacttctacttccagcacgcttcttacttttaccacagctcttgcacctaaccataacaccttccctgtatgatcgcgaagcacccaccctaagccacattttaatccttctgttggccatgccccatcaaagttgcacttaacccaagattgtggtggagcttcccatgtttctcgtctgtcccgacggtgttgtggttggtgctttccttacattctgagcctctttccttctaatccactcatctgcatcttcttgtgtccttactaatacctcattggttccaaattccctccctttaagcaccagctcgtttctgttcttccacagcctcccaagtatccaagggactaaagcctccacattcttcagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacag

SEQ ID NO: 73 - RNA, 38 nt, synthetic construct
  source 1..38: mol_type = other RNA
agcaggaaccagaggagaaacaacagcacacccacccc

SEQ ID NO: 74 - DNA, 41 bp, synthetic construct
  source 1..41: mol_type = other DNA
aggcaaacatcgtagtggaagtgggagaggtgcctgtcttt

SEQ ID NO: 75 - DNA, 41 bp, synthetic construct
  source 1..41: mol_type = other DNA
tccgtttgtagcatcaccttcaccctctccacggacagaaa

SEQ ID NO: 76 - RNA, 30 nt, synthetic construct
  source 1..30: mol_type = other RNA
gccccacagaggggacagcggaagggaacc

SEQ ID NO: 77 - DNA, 29 bp, synthetic construct
  source 1..29: mol_type = other DNA
caattcgaccattaccctatggaacacga

SEQ ID NO: 78 - DNA, 29 bp, synthetic construct
  source 1..29: mol_type = other DNA
gttaagctggtaatgggataccttgtgct

SEQ ID NO: 79 - RNA, 30 nt, synthetic construct
  source 1..30: mol_type = other RNA
aacacggaggggagaaaaaggaacacacga

SEQ ID NO: 80 - DNA, 29 bp, synthetic construct
  source 1..29: mol_type = other DNA
gaattctaattttccattaagatacacga

SEQ ID NO: 81 - DNA, 29 bp, synthetic construct
  source 1..29: mol_type = other DNA
cttaagctggtaatgggataccatgtgct

SEQ ID NO: 82 - RNA, 67 nt, synthetic construct
  source 1..67: mol_type = other RNA
aggaaccagaggagaaacaacagcacacccaccccccacggacagaaggaaccagaggagaaacaac

SEQ ID NO: 83 - RNA, 35 nt, synthetic construct
  source 1..35: mol_type = other RNA
aggaaccagaggagaaacaacagcacacccacccc

SEQ ID NO: 84 - RNA, 33 nt, synthetic construct
  source 1..33: mol_type = other RNA
aggaaccagaggagaaacaacagcacacccacc

SEQ ID NO: 85 - DNA, 633 bp, Arabidopsis thaliana
  source 1..633: mol_type = other DNA
cgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtagattaatctgagtttttctgattaaca

SEQ ID NO: 86 - skipped

SEQ ID NO: 87 - DNA, 465 bp, Cestrum yellow leaf curling virus
  source 1..465: mol_type = other DNA
tggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagctt

SEQ ID NO: 88 - DNA, 769 bp, Cauliflower mosaic virus
  source 1..769: mol_type = other DNA
ggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgactctagaggatccc

SEQ ID NO: 89 - DNA, 257 bp, Homo sapiens
  source 1..257: mol_type = other DNA
aaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacg

SEQ ID NO: 90 - skipped

SEQ ID NO: 91 - DNA, 448 bp, Arabidopsis thaliana
  source 1..448: mol_type = other DNA
aagcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgatt

SEQ ID NO: 92 - RNA, 21 nt, synthetic construct
  source 1..21: mol_type = other RNA
aggaaaagacaagcaaacaac

SEQ ID NO: 93 - RNA, 22 nt, synthetic construct
  source 1..22: mol_type = other RNA
aggaacacagaagggaaacaac

SEQ ID NO: 94 - RNA, 21 nt, synthetic construct
  source 1..21: mol_type = other RNA
aggaaccagaggagaaacaac

SEQ ID NO: 95 - RNA, 22 nt, synthetic construct
  source 1..22: mol_type = other RNA
aaggaaccagaggagaaacaac

SEQ ID NO: 96 - RNA, 21 nt, synthetic construct
  source 1..21: mol_type = other RNA
aggaaccagaggagaaacaac

SEQ ID NO: 97 - RNA, 20 nt, synthetic construct
  source 1..20: mol_type = other RNA
aggaacagaggagaaacaac

SEQ ID NO: 98 - RNA, 22 nt, synthetic construct
  source 1..22: mol_type = other RNA
aggagacccaaaggagaacaac

SEQ ID NO: 99 - RNA, 23 nt, synthetic construct
  source 1..23: mol_type = other RNA
aaggagagccaaaggaaaacaac

SEQ ID NO: 100 - RNA, 23 nt, synthetic construct
  source 1..23: mol_type = other RNA
aaggagaaccgaaggagaacaac

SEQ ID NO: 101 - RNA, 22 nt, synthetic construct
  source 1..22: mol_type = other RNA
aggagaaccgaaggaaaacaac

SEQ ID NO: 102 - RNA, 22 nt, synthetic construct
  source 1..22: mol_type = other RNA
aggagaaccgaaggagaacaac

SEQ ID NO: 103 - RNA, 21 nt, synthetic construct
  source 1..21: mol_type = other RNA
aggaaacagaaagaaaacaac

SEQ ID NO: 104 - RNA, 22 nt, synthetic construct
  source 1..22: mol_type = other RNA
aggaaacacaaaggagaacaac

SEQ ID NO: 105 - RNA, 51 nt, synthetic construct
  misc_feature 1..30: note = n is a, c, g, or u
  source 1..51: mol_type = other RNA
  variation 1..30: note = Where up to 10 copies of N can be deleted
nnnnnnnnnnnnnnnnnnnnnnnnnnnnnnaggaaccagaggagaaacaac

SEQ ID NO: 106 - RNA, 52 nt, synthetic construct
  misc_feature 1..30: note = n is a, c, g, or u
  source 1..52: mol_type = other RNA
  variation 1..30: note = Where up to 10 copies of N can be deleted
nnnnnnnnnnnnnnnnnnnnnnnnnnnnnnaggagacccaaaggagaacaac

SEQ ID NO: 107 - RNA, 52 nt, synthetic construct
  misc_feature 1..30: note = n is a, c, g, or u
  source 1..52: mol_type = other RNA
  variation 1..30: note = Where up to 10 copies of N can be deleted
nnnnnnnnnnnnnnnnnnnnnnnnnnnnnnaggagaaccgaaggagaacaac

SEQ ID NO: 108 - RNA, 52 nt, synthetic construct
  misc_feature 1..30: note = n is a, c, g, or u
  source 1..52: mol_type = other RNA
  variation 1..30: note = Where up to 10 copies of N can be deleted
nnnnnnnnnnnnnnnnnnnnnnnnnnnnnnaggagaaccgaaggaaaacaac

SEQ ID NO: 109 - RNA, 54 nt, synthetic construct
  misc_feature 1..30: note = n is a, c, g, or u
  source 1..54: mol_type = other RNA
  variation 1..30: note = Where up to 10 copies of N can be deleted
nnnnnnnnnnnnnnnnnnnnnnnnnnnnnnaaggagagccaaaggaaaacaacg

SEQ ID NO: 110 - RNA, 51 nt, synthetic construct
  misc_feature 1..30: note = n is a, c, g, or u
  source 1..51: mol_type = other RNA
  variation 1..30: note = Where up to 10 copies of N can be deleted
nnnnnnnnnnnnnnnnnnnnnnnnnnnnnnaggaaaagacaagcaaacaac

SEQ ID NO: 111 - RNA, 51 nt, synthetic construct; identical to SEQ ID NO: 105
  misc_feature 1..30: note = n is a, c, g, or u
  source 1..51: mol_type = other RNA
  variation 1..30: note = Where up to 10 copies of N can be deleted

SEQ ID NO: 112 - RNA, 52 nt, synthetic construct
  misc_feature 1..30: note = n is a, c, g, or u
  source 1..52: mol_type = other RNA
  variation 1..30: note = Where up to 10 copies of N can be deleted
nnnnnnnnnnnnnnnnnnnnnnnnnnnnnnaggaacacagaagggaaacaac

SEQ ID NO: 113 - RNA, 52 nt, synthetic construct
  misc_feature 1..30: note = n is a, c, g, or u
  source 1..52: mol_type = other RNA
  variation 1..30: note = Where up to 10 copies of N can be deleted
nnnnnnnnnnnnnnnnnnnnnnnnnnnnnnaaggaaccagaggagaaacaac

SEQ ID NO: 114 - RNA, 50 nt, synthetic construct
  misc_feature 1..30: note = n is a, c, g, or u
  source 1..50: mol_type = other RNA
  variation 1..30: note = Where up to 10 copies of N can be deleted
nnnnnnnnnnnnnnnnnnnnnnnnnnnnnnaggaacagaggagaaacaac

SEQ ID NO: 115 - RNA, 53 nt, synthetic construct
  misc_feature 1..30: note = n is a, c, g, or u
  source 1..53: mol_type = other RNA
  variation 1..30: note = Where up to 10 copies of N can be deleted
nnnnnnnnnnnnnnnnnnnnnnnnnnnnnnaaggagaaccgaaggagaacaac

SEQ ID NO: 116 - RNA, 51 nt, synthetic construct
  misc_feature 1..30: note = n is a, c, g, or u
  source 1..51: mol_type = other RNA
  variation 1..30: note = Where up to 10 copies of N can be deleted
nnnnnnnnnnnnnnnnnnnnnnnnnnnnnnaggaaacagaaagaaaacaac

SEQ ID NO: 117 - RNA, 53 nt, synthetic construct
  misc_feature 1..30: note = n is a, c, g, or u
  source 1..53: mol_type = other RNA
  variation 1..30: note = Where up to 10 copies of N can be deleted
nnnnnnnnnnnnnnnnnnnnnnnnnnnnnnaaggagagccaaaggaaaacaac

SEQ ID NO: 118 - RNA, 52 nt, synthetic construct
  misc_feature 1..30: note = n is a, c, g, or u
  source 1..52: mol_type = other RNA
  variation 1..30: note = Where up to 10 copies of N can be deleted
nnnnnnnnnnnnnnnnnnnnnnnnnnnnnnaggaaacacaaaggagaacaac

SEQ ID NO: 119 - protein, 84 aa, unidentified
MASMISSSAVTTVSRASRGQSAAMAPFGGLKSMTGFPVRKVNTDITSITSNGGRVKCMQVWPPIGKKKFETLSYLPPLTRDSRA

SEQ ID NO: 120 - protein, 57 aa, unidentified
MASMISSSAVTTVSRASRGQSAAMAPFGGLKSMTGFPVRKVNTDITSITSNGGRVKS

SEQ ID NO: 121 - protein, 85 aa, unidentified
MASSMLSSATMVASPAQATMVAPFNGLKSSAAFPATRKANNDITSITSNGGRVNCMQVWPPIEKKKFETLSYLPDLTDSGGRVNC

SEQ ID NO: 122 - protein, 76 aa, unidentified
MAQVSRICNGVQNPSLISNLSKSSQRKSPLSVSLKTQQHPRAYPISSSWGLKKSGMTLIGSELRPLKVMSSVSTAC

SEQ ID NO: 123 - protein, 76 aa, unidentified
MAQVSRICNGVWNPSLISNLSKSSQRKSPLSVSLKTQQHPRAYPISSSWGLKKSGMTLIGSELRPLKVMSSVSTAC

SEQ ID NO: 124 - protein, 72 aa, unidentified
MAQINNMAQGIQTLNPNSNFHKPQVPKSSSFLVFGSKKLKNSANSMLVLKKDSIFMQLFCSFRISASVATAC

SEQ ID NO: 125 - protein, 69 aa, unidentified
MAALVTSQLATSGTVLSVTDRFRRPGFQGLRPRNPADAALGMRTVGASAAPKQSRKPHRFDRRCLSMVV

SEQ ID NO: 126 - protein, 77 aa, unidentified
MAALTTSQLATSATGFGIADRSAPSSLLRHGFQGLKPRSPAGGDATSLSVTTSARATPKQQRSVQRGSRRFPSVVVC

SEQ ID NO: 127 - protein, 57 aa, unidentified
MASSVLSSAAVATRSNVAQANMVAPFTGLKSAASFPVSRKQNLDITSIASNGGRVQC

SEQ ID NO: 128 - protein, 65 aa, unidentified
MESLAATSVFAPSRVAVPAARALVRAGTVVPTRRTSSTSGTSGVKCSAAVTPQASPVISRSAAAA

SEQ ID NO: 129 - protein, 72 aa, unidentified
MGAAATSMQSLKFSNRLVPPSRRLSPVPNNVTCNNLPKSAAPVRTVKCCASSWNSTINGAAATTNGASAASS

SEQ ID NO: 130 - protein, 20 aa, synthetic construct
  SITE 4: note = Where X can be K, H, or R
  SITE 8: note = Where X can be K, H, or R
  SITE 11: note = Where X can be K, H, or R
  SITE 15: note = Where X can be K, H, or R
  SITE 19: note = Where X can be K, H, or R
GLFXALLXLLXSLWXLLLXA

SEQ ID NO: 131 - protein, 20 aa, synthetic construct
GLFHALLHLLHSLWHLLLHA

SEQ ID NO: 132 - protein, 167 aa, synthetic construct
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 133 - protein, 178 aa, synthetic construct
MRRAFITGVFFLSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 134 - protein, 160 aa, Staphylococcus aureus
MGSHMTNDIYFMTLAIEEAKKAAQLGEVPIGAIITKDDEVIARAHNLRETLQQPTAHAEHIAIERAAKVLGSWRLEGCTLYVTLEPCVMCAGTIVMSRIPRVVYGADDPKGGCSGSLMNLLQQSNFNHRAIVDKGVLKEACSTLLTTFFKNLRANKKSTN

SEQ ID NO: 135 - protein, 161 aa, Bacillus subtilis
MTQDELYMKEAIKEAKKAEEKGEVPIGAVLVINGEIIARAHNLRETEQRSIAHAEMLVIDEACKALGTWRLEGATLYVTLEPCPMCAGAVVLSRVEKVVFGAFDPKGGCSGTLMNLLQEERFNHQAEVVSGVLEEECGGMLSAFFRELRKKKKAARKNLSE

SEQ ID NO: 136 - protein, 183 aa, Salmonella typhimurium
MPPAFITGVTSLSDVELDHEYWMRHALTLAKRAWDEREVPVGAVLVHNHRVIGEGWNRPIGRHDPTAHAEIMALRQGGLVLQNYRLLDTTLYVTLEPCVMCAGAMVHSRIGRVVFGARDAKTGAAGSLIDVLHHPGMNHRVEIIEGVLRDECATLLSDFFRMRRQEIKALKKADRAEGAGPAV

SEQ ID NO: 137 - protein, 164 aa, Shewanella putrefaciens
MDEYWMQVAMQMAEKAEAAGEVPVGAVLVKDGQQIATGYNLSISQHDPTAHAEILCLRSAGKKLENYRLLDATLYITLEPCAMCAGAMVHSRIARVVYGARDEKTGAAGTVVNLLQHPAFNHQVEVTSGVLAEACSAQLSRFFKRRRDEKKALKLAQRAQQGIE

SEQ ID NO: 138 - protein, 173 aa, Haemophilus influenzae
MDAAKVRSEFDEKMMRYALELADKAEALGEIPVGAVLVDDARNIIGEGWNLSIVQSDPTAHAEIIALRNGAKNIQNYRLLNSTLYVTLEPCTMCAGAILHSRIKRLVFGASDYKTGAIGSRFHFFDDYKMNHTLEITSGVLAEECSQKLSTFFQKRREEKKIEKALLKSLSDK

SEQ ID NO: 139 - protein, 161 aa, Caulobacter crescentus
MRTDESEDQDHRMMRLALDAARAAAEAGETPVGAVILDPSTGEVIATAGNGPIAAHDPTAHAEIAAMRAAAAKLGNYRLTDLTLVVTLEPCAMCAGAISHARIGRVVFGADDPKGGAVVHGPKFFAQPTCHWRPEVTGGVLADESADLLRGFFRARRKAKI

SEQ ID NO: 140 - protein, 179 aa, Geobacter sulfurreducens
MSSLKKTPIRDDAYWMGKAIREAAKAAARDEVPIGAVIVRDGAVIGRGHNLREGSNDPSAHAEMIAIRQAARRSANWRLTGATLYVTLEPCLMCMGAIILARLERVVFGCYDPKGGAAGSLYDLSADPRLNHQVRLSPGVCQEECGTMLSDFFRDLRRRKKAKATPALFIDERKVPPEP

SEQ ID NO: 141 - protein, 198 aa, synthetic construct [sequence omitted]

SEQ ID NO: 142 - protein, 188 aa, synthetic construct
MDSLLMNRRKFLYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKENHERTFKAWEGLHENSVRLSRQLRRILLPLYEVDDLRDAFRTLGL

SEQ ID NO: 143 - protein, 7 aa, SV40 virus
PKKKRKV

SEQ ID NO: 144 - protein, 16 aa, Xenopus laevis
KRPAATKKAGQAKKKK

SEQ ID NO: 145 - protein, 9 aa, unidentified
PAAKRVKLD

SEQ ID NO: 146 - protein, 11 aa, unidentified
RQRRNELKRSP

SEQ ID NO: 147 - protein, 38 aa, unidentified
NQSSNFGPMKGGNFGGRSSGPYGGGGQYFAKPRNQGGY

SEQ ID NO: 148 - protein, 42 aa, unidentified
RMRIZFKNKGKDTAELRRRRVEVSVELRKAKKDEQILKRRNV

SEQ ID NO: 149 - protein, 8 aa, unidentified
VSRKRPRP

SEQ ID NO: 150 - protein, 8 aa, unidentified
  REGION 1..8: note = synthetic sequence
PPKKARED

SEQ ID NO: 151 - protein, 8 aa, Homo sapiens
PQPKKKPL

SEQ ID NO: 152 - protein, 12 aa, Mus musculus
SALIKKKKKMAP

SEQ ID NO: 153 - protein, 5 aa, influenza virus
DRLRR

SEQ ID NO: 154 - protein, 7 aa, influenza virus
PKQKKRK

SEQ ID NO: 155 - protein, 10 aa, Hepatitis virus
RKLKKKIKKL

SEQ ID NO: 156 - protein, 10 aa, Mus musculus
REKKKFLKRR

SEQ ID NO: 157 - protein, 20 aa, Homo sapiens
KRKGDEVDGVDEVAKKKSKK

SEQ ID NO: 158 - protein, 17 aa, Homo sapiens
RKCLQAGMNLEARKTKK

SEQ ID NO: 159 - protein, 11 aa, Human immunodeficiency virus 1
YGRKKRRQRRR

SEQ ID NO: 160 - protein, 12 aa, synthetic construct
RRQRRTSKLMKR

SEQ ID NO: 161 - protein, 27 aa, synthetic construct
GWTLNSAGYLLGKINLKALAALAKKIL

SEQ ID NO: 162 - protein, 33 aa, synthetic construct
KALAWEAKLAKALAKALAKHLAKALAKALKCEA

SEQ ID NO: 163 - protein, 16 aa, synthetic construct
RQIKIWFQNRRMKWKK

SEQ ID NO: 164 - protein, 9 aa, synthetic construct
RKKRRQRRR

SEQ ID NO: 165 - protein, 8 aa, synthetic construct
RKKRRQRR

SEQ ID NO: 166 - protein, 11 aa, synthetic construct
YARAAARQARA

SEQ ID NO: 167 - protein, 11 aa, synthetic construct
THRLPRRRRRR

SEQ ID NO: 168 - protein, 11 aa, synthetic construct
GGRRARRRRRR

SEQ ID NO: 169 - protein, 50 aa, synthetic construct
  VARIANT 1..50: note = Where up to 9 copies of GGGGS can be deleted
GGGGSGGGGSGGGGSGGGGSGGGGSGGGGSGGGGSGGGGSGGGGSGGGGS

SEQ ID NO: 170 - protein, 4 aa, synthetic construct
GGSG

SEQ ID NO: 171 - protein, 5 aa, synthetic construct
GGSGG

SEQ ID NO: 172 - protein, 5 aa, synthetic construct
GSGSG

SEQ ID NO: 173 - protein, 5 aa, synthetic construct
GSGGG

SEQ ID NO: 174 - protein, 5 aa, synthetic construct
GGGSG

SEQ ID NO: 175 - protein, 5 aa, synthetic construct
GSSSG

SEQ ID NO: 176 - protein, 9 aa, synthetic construct
SSGPPPGTG

SEQ ID NO: 177 - protein, 12 aa, synthetic construct
AEAAAKEAAAKA

SEQ ID NO: 178 - protein, 16 aa, synthetic construct
SGSETPGTSESATPES

SEQ ID NO: 179 - RNA, 56 nt, synthetic construct
  misc_feature 1..35: note = n is a, c, g, or u
  source 1..56: mol_type = other RNA
nnnnnnnnnnnnnnnnnnnnnnnnnnnnnnnnnnnaggaaccagaggagaaacaac

SEQ ID NO: 180 - RNA, 24 nt, synthetic construct
  source 1..24: mol_type = other RNA
aaggagagccaaaggaaaacaacg

SEQ ID NO: 181 - DNA, 20 bp, Arabidopsis thaliana
  source 1..20: mol_type = other DNA
gttttcagaccaagctcttt

SEQ ID NO: 182 - DNA, 20 bp, Arabidopsis thaliana
  source 1..20: mol_type = other DNA
tatctttaacacataattga

SEQ ID NO: 183 - DNA, 20 bp, Arabidopsis thaliana
  source 1..20: mol_type = other DNA
tgagactacaaggtataact

SEQ ID NO: 184 - DNA, 20 bp, Arabidopsis thaliana
  source 1..20: mol_type = other DNA
catattttctgtaagtccaa

SEQ ID NO: 185 - DNA, 20 bp, Arabidopsis thaliana
  source 1..20: mol_type = other DNA
attacccatcctaaagtatg

SEQ ID NO: 186 - RNA, 60 nt, synthetic construct
  source 1..60: mol_type = other RNA
atgcattgttgtaactcttattttgtatggagtaaacaactagcatcaccttcaccctct

SEQ ID NO: 187 - RNA, 16 nt, synthetic construct
  source 1..16: mol_type = other RNA
agaggggaagggagca